(19) United States
(12) Patent Application Publication
(10) Pub. No.: US 2024/0091762 A1
(43) Pub. Date: Mar. 21, 2024

(54) HYBRIDIZATION CHAIN REACTION LATERAL FLOW ASSAYS FOR AMPLIFIED DETECTION OF A TARGET IN A SAMPLE

(71) Applicant: California Institute of Technology, Pasadena, CA (US)

(72) Inventors: Samuel J. Schulte, Pasadena, CA (US); Jining Huang, Pasadena, CA (US); Nikhil T. Linaval, Pasadena, CA (US); Niles A. Pierce, South Pasadena, CA (US)

(21) Appl. No.: 18/466,726

(22) Filed: Sep. 13, 2023

Related U.S. Application Data
(60) Provisional application No. 63/407,086, filed on Sep. 15, 2022.

(51) Int. Cl. B01L 3/00

(52) U.S. Cl. CPC B01L 3/5023; B01L 2300/069; B01L 2300/0825

(57) ABSTRACT

The present disclosure relates to an HCR lateral flow device for amplified detection of a target in a sample and methods of using the same. In some embodiments, the device and methods of use combine the sensitivity of HCR signal amplification with the simplicity of the lateral flow assay format to facilitate user-friendly amplified detection of a target in a sample.

### REFERENCE TO RELATED APPLICATIONS

This application claims priority to U.S. Provisional Patent Application No. 63/407,086, filed Sep. 15, 2022, the disclosure of which is hereby incorporated by reference in its entirety herein.

### STATEMENT REGARDING FEDERALLY SPONSORED R&D

This invention was made with government support under Grant No. EB006192 awarded by the National Institutes of Health and under Grant No. NNX16AO69A awarded by NASA Johnson Space Center. The government has certain rights in the invention.

### BACKGROUND

### Field

The sensitivity of hybridization chain reaction (HCR) signal amplification is combined with the simplicity of the lateral flow assay format to facilitate user-friendly amplified detection of a target in a sample.

### SUMMARY

In accordance with some implementations, HCR lateral flow devices for amplified detection of a target in a sample are provided, wherein the devices comprise one or more membranes, a test region, one or more probe sets each comprising one or more probes, and an HCR amplifier comprising two or more HCR hairpins wherein one or more of the HCR hairpins is labeled with one or more reporters, wherein the sample and one or more reagents flow through the one or more membranes across the test region, and wherein the one or more probe sets and the HCR amplifier are configured to produce tethered reporter-decorated HCR amplification polymers to directly or indirectly generate an amplified signal in the test region if the target is present in the sample. In accordance with some implementations, the one or more probe sets comprise a capture probe set comprising one or more capture probes, wherein a capture probe comprises a target-binding domain configured to bind directly or indirectly to the target and an immobilization domain configured to bind directly or indirectly to the test region. In accordance with some implementations, the one or more probe sets comprise a signal probe set comprising one or more signal probes, wherein a signal probe comprises a target-binding domain configured to bind directly or indirectly to the target and an amplification domain that is configured to directly or indirectly trigger a chain reaction in which HCR hairpins self-assemble to form a tethered HCR amplification polymer decorated with reporters. In accordance with some implementations, the signal probe set comprises: a) one or more initiator-labeled probes wherein an initiator-labeled probe comprises a target-binding domain and an amplification domain comprising one or more HCR initiators, or b) one or more probe units, each comprising two or more fractional-initiator probes, wherein a fractional-initiator probe comprises a target-binding domain and an amplification domain comprising a fractional initiator.

In accordance with some implementations, if the target is present in the sample, the target binds in a complex between the capture probe set and the signal probe set, wherein the capture probe set immobilizes the probe/target sandwich in the test region, and wherein the amplification domains on the signal probes trigger the self-assembly of reporter-labeled HCR hairpins into tethered HCR amplification polymers decorated with reporters to directly or indirectly generate an amplified signal in the test region. In accordance with some implementations, the one or more probe sets comprise a readout probe set comprising one or more readout probes, wherein a readout probe comprises a reporter-binding domain configured to bind directly or indirectly to a reporter on an HCR amplification polymer, and further comprising one or more auxiliary reporters wherein the auxiliary reporters contribute directly or indirectly to the generation of an amplified signal in the test region. In accordance with some implementations, the auxiliary-reporter-labeled readout probes comprising one or more auxiliary reporters bind the reporters decorating the tethered HCR amplification polymers that are immobilized in the test region, directly or indirectly generating an amplified signal in the test region. In accordance with some implementations, the reporters and/or auxiliary reporters decorating an HCR amplification polymer directly or indirectly mediate catalytic reporter deposition (CARD) to generate an amplified signal in the vicinity of the HCR amplification polymer within the test region. In accordance with some implementations, the amplified signal in the test region is visible to the human eye.

In accordance with some implementations, HCR lateral flow devices for amplified detection of a target in a sample are provided, wherein the devices comprise one or more membranes, a test region, one or more probe sets each comprising one or more probes, and an HCR amplifier comprising two or more HCR hairpins wherein one or more of the HCR hairpins is labeled with one or more reporters, wherein the sample and one or more reagents flow through the one or more membranes across the test region, and wherein the one or more probe sets and the HCR amplifier are configured to produce tethered reporter-decorated HCR amplification polymers to directly or indirectly generate an amplified signal in the test region if the target is not present in the sample. In accordance with some implementations, the device comprises a target-mimic probe set comprising one or more target-mimic probes each comprising a competitive domain configured to compete with the target for binding partners, and an immobilization domain configured to bind directly or indirectly to the test region. In accordance with some implementations, the one or more probe sets comprise a signal probe set comprising one or more signal probes, wherein a signal probe comprises a target-binding domain configured to bind directly or indirectly to the target or to the competitive domain of a target-mimic probe, and further comprises an amplification domain that is configured to directly or indirectly trigger a chain reaction in which HCR hairpins self-assemble to form a tethered HCR amplification polymer decorated with reporters. In accordance with some implementations, the signal probe set comprises: a) one or more initiator-labeled probes wherein an initiator-labeled probe comprises a target-binding domain and an amplification domain comprising one or more HCR initiators, or b) one or more probe units, each comprising two or more fractional-initiator probes, wherein a fractional-initiator probe comprises a target-binding domain and an amplification domain comprising a fractional initiator. In accordance with some implementations, if the target is not present in the sample, the target-mimic probe set binds the signal probe set to form a competitive complex, wherein the target-mimic probe set immobilizes the competitive complex in the test region, and wherein the amplification domains on the signal probes trigger the self-assembly of reporter-labeled HCR hairpins into tethered HCR amplification polymers decorated with reporters to directly or indirectly generate an amplified signal in the test region. In accordance with some implementations, the one or more probe sets comprise a readout probe set comprising one or more readout probes, wherein a readout probe comprises a reporter-binding domain configured to bind directly or indirectly to a reporter on an HCR amplification polymer, and further comprising one or more auxiliary reporters wherein the auxiliary reporters contribute directly or indirectly to the generation of an amplified signal in the test region. In accordance with some implementations, the auxiliary-reporter-labeled readout probes comprising one or more auxiliary reporters bind the reporters decorating the tethered HCR amplification polymers that are immobilized in the test region, directly or indirectly generating an amplified signal in the test region. In accordance with some implementations, the reporters and/or auxiliary reporters decorating an HCR amplification polymer directly or indirectly mediate catalytic reporter deposition (CARD) to generate an amplified signal in the vicinity of the HCR amplification polymer within the test region. In accordance with some implementations, the amplified signal in the test region is visible to the human eye.

In accordance with some implementations of any of the foregoing, the devices comprise a control region downstream of the test region so that the sample and reagents flow through the test region before reaching the control region. In accordance with some implementations, the device comprises a control capture probe set, a control signal probe set, a control HCR amplifier comprising one or more control reporters, and optionally a control readout probe set comprising one or more control auxiliary reporters. In accordance with some implementations, if the sample and reagents successfully flow through the control region, the control capture probe set binds to and immobilizes the control signal probe set in the test region, the control signal probe set triggers the self-assembly of tethered control HCR amplification polymers decorated with control reporters to directly or indirectly generate an amplified control signal in the control region, and optionally the control-auxiliary-reporter-labeled control readout probe set binds the control reporters to directly or indirectly generate an amplified control signal in the control region. In accordance with some implementations, the control readout probe set is the same as the readout probe set. In accordance with some implementations, the control HCR amplifier is the same as the HCR amplifier. In accordance with some implementations, the control signal probe set is the same as the signal probe set. In accordance with some implementations, the absence of signal in the control region is an indication that the test did not function properly.

In accordance with some implementations of any of the foregoing, a probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above. In accordance with some implementations of any of the foregoing, the one or more reporters, primary reporters, auxiliary reporters, control reporters, and/or control auxiliary reporters comprise a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, digoxigenin (DIG), fluorescein isothiocyanate (FITC), biotin, dinitrophenol, aniline, an enzyme, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, any combination thereof, or any other molecule that directly or indirectly facilitates measurement of a signal. In accordance with some implementations of any of the foregoing, the target comprises a protein, a peptide, an amino acid, a nucleic acid, a DNA, an RNA, a chemically modified nucleic acid, a synthetic nucleic acid analog, a material capable of base-pairing, a chemical linker, a molecule, a biomolecule, a chemical, a toxin, a pathogen, a disease marker, a pollutant, a complex of any of the above, a diseased cell, a cancer cell, a human cell, a eukaryotic cell, a prokaryote, a bacterium, a virus, or any combination of the above. In accordance with some implementations of any of the foregoing, the target is viral or bacterial in origin. In accordance with some implementations of any of the foregoing, the sample comprises a bodily fluid, milk, water, urine, blood, serum, saliva, cells, tissue, tissue homogenate, a medical sample, an environmental sample, wash water from an agricultural crop, gray water, brown water, sea water, pollutants, polluted water, a waste sample, sewage, plants, bacteria, sludge, homogenized plants, organic matter, animal feed, an industrial sample, liquid fuel, solid fuel, propellants, a chemical solution, and/or chemicals. In accordance with some implementations of any of the foregoing, the sample is obtained by swabbing, nasal swabbing, nasopharyngeal swabbing, anterior nasal swabbing, throat swabbing, or swabbing of a material, mixture, solution, or substance to be analyzed. In accordance with some implementations of any of the foregoing, the device comprises one or more sample pads, one or more conjugate pads, and/or one or more wicking pads. In accordance with some implementations, the one or more membranes, and one or more sample pads, one or more conjugate pads, and/or one or more wicking pads comprise nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.

In accordance with some implementations of any of the foregoing, reagents automatically arrive in the test region and the optional control region in two or more successive stages. In accordance with some implementations, reagents automatically arrive in the test region and the optional control region in two or more successive stages using a membrane comprising two or more channels. In accordance with some implementations, the two or more channels have different lengths that lead into a unified channel before reaching the test region. In accordance with some implementations, the reagents for each channel are dried down on a separate conjugate pad for each channel. In accordance with some implementations of any of the foregoing, the sample is mixed with prep buffer before or after adding the sample to the device. In accordance with some implementations, the reagents dried down in each conjugate pad are resuspended when the user adds the sample, prep buffer, or mixture of sample and prep buffer to the device. In accordance with some implementations of any of the foregoing, the device takes the form of a folding card comprising a sample pad, one or more conjugate pads, a membrane comprising two or more channels, and a wicking pad, wherein closing the folding card starts the test. In accordance with some implementations of any of the foregoing, each conjugate pad is fluidly connected to a single channel.

In accordance with some implementations, HCR lateral flow devices for amplified detection of a target in a sample are provided, wherein the devices comprise one or more membranes, a test region, a signal probe set comprising one or more signal probes, and an HCR amplifier comprising two or more HCR hairpins wherein one or more of the HCR hairpins is labeled with one or more reporters, wherein the sample and one or more reagents flow through the one or more membranes across the test region, and wherein the signal probe set and the HCR amplifier are configured to produce a tethered reporter-decorated HCR amplification polymer to directly or indirectly generate an amplified signal in the test region if the signal probe set is immobilized in the test region.

In accordance with some implementations, HCR lateral flow devices for amplified detection of N targets in a sample (where N is an integer greater than or equal to two) are provided, wherein the devices comprise one or more membranes, N test regions (one per target), N capture probe sets (one per target), N signal probe sets (one per target), N HCR amplifiers (one per target) each labeled with one or more reporters, and optionally N readout probe sets (one per target) each labeled with one or more auxiliary reporters, wherein the sample and one or more reagents flow through the one or more membranes across the N test regions, wherein the capture probe sets, signal probe sets, and HCR amplifiers are configured to produce a tethered reporter-decorated HCR amplification polymer to directly or indirectly generate an amplified signal in a given test region if the corresponding target is present in the sample, and wherein optionally the auxiliary-reporter-labeled readout probe sets are configured to bind the reporters to directly or indirectly generate an amplified signal in a given test region if the corresponding target is present in the sample. In accordance with some implementations, the N readout probe sets are the same for all targets. In accordance with some implementations, the N HCR amplifiers are the same for all targets.

In accordance with some implementations, HCR lateral flow devices for amplified detection of N targets in a sample are provided (where N is an integer greater than or equal to two), wherein the device comprises one or more membranes, N test regions (one per target), N target-mimic probe sets (one per target), N signal probe sets (one per target), N HCR amplifiers (one per target) each labeled with one or more reporters, and optionally N readout probe sets (one per target) each labeled with one or more auxiliary reporters, wherein the sample and one or more reagents flow through the one or more membranes across the N test regions, wherein the target-mimic probe sets, signal probe sets, and HCR amplifiers are configured to produce a tethered reporter-decorated HCR amplification polymer to directly or indirectly generate an amplified signal in a given test region if the corresponding target is not present in the sample, and wherein optionally the auxiliary-reporter-labeled readout probe sets are configured to bind the reporters to directly or indirectly generate an amplified signal in a given test region if the corresponding target is not present in the sample. In accordance with some implementations, the N readout probe sets are the same for all targets. In accordance with some implementations, the N HCR amplifiers are the same for all targets.

In accordance with some implementations, methods for amplified detection of one or more targets in a sample are provided, wherein the user: 1) optionally preps the sample by mixing the sample with a prep buffer, 2) adds the sample to an HCR lateral flow device as described herein, 3) waits for a prescribed amount of time, for example for a prescribed number of minutes, while the sample and reagents flow through the one or more membranes across the test region to produce a tethered reporter-decorated HCR amplification polymer that directly or indirectly generates an amplified signal in the test region if the target is present in the sample, as described herein, 4) and reads out the rest result by examining the test region for a signal visible to the naked eye. In some embodiments the HCR lateral flow device comprises one or more membranes, a test region, one or more probe sets each comprising one or more probes, an HCR amplifier comprising two or more HCR hairpins wherein one or more of the HCR hairpins is labeled with one or more reporters.

In accordance with some implementations, methods for amplified detection of one or more targets in a sample are provided, wherein the user: 1) optionally preps the sample by mixing the sample with a prep buffer, 2) adds the sample to an HCR lateral flow device comprising one or more membranes, a test region, one or more probe sets each comprising one or more probes, an HCR amplifier comprising two or more HCR hairpins wherein one or more of the HCR hairpins is labeled with one or more reporters, 3) waits for a prescribed amount of time, such as a prescribed number of minutes, while the sample and reagents flow through the one or more membranes across the test region to produce a tethered reporter-decorated HCR amplification polymer that directly or indirectly generates an amplified signal in the test region if the target is not present in the sample, as described herein, 4) and reads out the rest result by examining the test region for a signal visible to the naked eye.

### BRIEF DESCRIPTION OF THE DRAWINGS

FIGS. 1A-1C depict binding interactions at the test line and corresponding signal outcomes for conventional unamplified lateral flow assays.

FIGS. 2A-2B depict the function of conventional unamplified lateral flow assays.

FIGS. 3A-3C depict some embodiments of HCR RNA-FISH using initiator-labeled probes.

FIGS. 4A-4B depict some embodiments of HCR RNA-FISH using fractional-initiator probes.

FIG. 5 depicts a folding card HCR lateral flow device for amplified detection of a target in a sample.

FIG. 6 depicts a half-strip HCR lateral flow device for amplified detection of a target in a sample.

FIGS. 7A-7J depict some embodiments of binding interactions in the test region of an HCR lateral flow device for amplified detection of a target in a sample using a sandwich format.

FIGS. 8A-8D depict some embodiments of binding events in the test region of an HCR lateral flow device for amplified detection of a target in a sample using a sandwich format.

FIGS. 9A-9E depict some embodiments of binding interactions in the test region of an HCR lateral flow device for amplified detection of a target protein in a sample using a sandwich format.

FIGS. 10A-10D depict some embodiments of binding events in the test region of an HCR lateral flow device for amplified detection of a target protein in a sample using a sandwich format.

FIGS. 11A-11E depict some embodiments of binding interactions in the test region of an HCR lateral flow device for amplified detection of a target nucleic acid in a sample using a sandwich format.

FIGS. 12A-12D depict some embodiments of binding events in the test region of an HCR lateral flow device for amplified detection of a target nucleic acid in a sample using a sandwich format.

FIG. 13 depicts some embodiments of test feature shapes in the test region of an HCR lateral flow device.

FIG. 14 depicts some embodiments of HCR amplification using two HCR hairpins. 1050 in FIG. 14 depicts full HCR initiator i1 formed by two fractional-initiator probes colocalized by a target. Only a part of the fractional initiator probes is depicted.

FIG. 15 depicts some embodiments of HCR amplification using two HCR hairpins.

FIGS. 16A-16B depicts some embodiments of HCR amplification using four HCR hairpins.

FIGS. 17A-17F depict some embodiments of HCR amplifiers.

FIGS. 18A-18N depict some embodiments of initiator-labeled probes.

FIGS. 19A-19F depict some embodiments of initiator-labeled probes.

FIGS. 20A-20E depict some embodiments of fractional-initiator probes colocalized by a target RNA.

FIGS. 21A-21D depict some embodiments of fractional-initiator probes colocalized by a target.

FIGS. 22A-22E depict some embodiments of fractional-initiator probes colocalized by a target complex.

FIG. 23 depicts some embodiments of fractional-initiator probes colocalized by a target RNA.

FIGS. 24A-24R depict some embodiments of fractional-initiator probes colocalized by a target either direction or indirectly.

FIGS. 25A-25E depict some embodiments for using HCR amplification to mediate CARD signal amplification for different targets and signal probes.

FIGS. 26A-26C depict some embodiments for using HCR amplification to mediate CARD signal amplification for generic targets and signal probes.

FIGS. 27A-27B depict some embodiments for using HCR amplification with reporter-labeled or fractional-reporter-labeled HCR hairpins to mediate CARD signal amplification.

FIG. 28 depicts some embodiments of binding interactions in the test region of an HCR lateral flow device for amplified detection of a target complex in a sample using a sandwich format.

FIG. 29 depicts some embodiments of binding interactions in the test region of an HCR lateral flow device for amplified detection of a target whole virus in a sample using a sandwich format.

FIGS. 30A-30B depict some embodiments of multi-channel membranes for automated delivery of reagents in successive stages to the test region of an HCR lateral flow device for amplified detection of a target in a sample using a sandwich format.

FIGS. 31A-31B depict some embodiments of multi-channel membranes for automated delivery of reagents in successive stages to the test region of an HCR lateral flow device for amplified detection of a target protein in a sample using a sandwich format.

FIGS. 32A-32B depict some embodiments of multi-channel membranes for automated delivery of reagents in successive stages to the test region of an HCR lateral flow device for amplified detection of a target nucleic acid in a sample using a sandwich format.

FIGS. 33A-33F depict some embodiments of devices for delivery of reagents to the test region of an HCR lateral flow device for amplified detection of a target in a sample using a sandwich format.

FIGS. 34A-34C depict some embodiments of binding interactions in the test region and control region of an HCR lateral flow device for amplified detection of a target in a sample using a sandwich format.

FIGS. 35A-35C depict some embodiments of binding events in the test region and control region of an HCR lateral flow device for amplified detection of a target in a sample using a sandwich format.

FIGS. 36A-36C depict some embodiments of multi-channel membranes for delivery of reagents to the test region and control region of an HCR lateral flow device for amplified detection of a target in a sample using a sandwich format.

FIGS. 37A-37B depict some embodiments of test feature shapes and control feature shapes in the test region and control region of an HCR lateral flow device.

FIGS. 38A-38F depict some embodiments of obtaining, prepping, and/or adding samples to an HCR lateral flow device.

FIG. 39 depicts some embodiments of binding interactions in the test region of an HCR lateral flow device for amplified detection of a target nucleic acid in a sample using a sandwich format.

FIGS. 40A-40B depict some embodiments of binding events in the test region of an HCR lateral flow device for detecting a target nucleic acid in a sample using a partner probe set using a sandwich format.

FIGS. 41A-41D depict some embodiments of binding interactions in two test regions of an HCR lateral flow device for amplified multiplexed detection of two targets in a sample using a sandwich format.

FIGS. 42A-42D depict some embodiments of multi-channel membranes for delivery of reagents to two test regions of an HCR lateral flow device for amplified multiplexed detection of two targets in a sample using a sandwich format.

FIG. 43 depicts some embodiments of binding interactions in two test regions and one control region of an HCR lateral flow device for amplified multiplexed detection of two targets in a sample using a sandwich format.

FIG. 44 depicts some embodiments of multi-channel membranes for delivery of reagents to two test regions and one control region of an HCR lateral flow device for amplified multiplexed detection of two targets in a sample using a sandwich format.

FIGS. 45A-45D depict some embodiments of membranes for delivery of reagents to the test region of an HCR lateral flow device for amplified multiplexed detection of a target in a sample using pre-formed HCR amplification polymers using a sandwich format.

FIGS. 46A-46J depict some embodiments of binding interactions in the test region of an HCR lateral flow device for amplified detection of a target in a sample using a competitive format.

FIGS. 47A-47D depict some embodiments of binding events in the test region of an HCR lateral flow device for amplified detection of a target in a sample using a competitive format.

FIGS. 48A-48B depict some embodiments of multi-channel membranes for automated delivery of reagents in successive stages to the test region of an HCR lateral flow device for amplified detection of a target in a sample using a competitive format.

FIGS. 49A-49C depict some embodiments of binding interactions in the test region and control region of an HCR lateral flow device for amplified detection of a target in a sample using a competitive format.

FIGS. 50A-50D depict some embodiments of probe sets comprising one or more probe units and optionally comprising one or more helper probes.

FIGS. 51A-51C depict some embodiments of probe units comprising fractional initiator probes.

FIG. 52 depicts binding events in the test region of an HCR lateral flow device for amplified detection of SARS-CoV-2 nucleocapsid protein N in a sample.

FIG. 53 depicts a three-channel membrane for automated delivery of reagents in three successive stages to the test region of an HCR lateral flow device for amplified detection of SARS-CoV-2 nucleocapsid protein N in a sample.

FIG. 54 demonstrates detection of SARS-CoV-2 nucleocapsid protein N in a sample using an HCR lateral flow assay.

FIGS. 55A-55C demonstrate performance of an HCR lateral flow device for amplified detection of SARS-CoV-2 nucleocapsid protein N in a sample.

FIGS. 56A-56C demonstrate performance across replicate tests of an HCR lateral flow device for amplified detection of SARS-CoV-2 nucleocapsid protein N in a sample.

FIGS. 57A-57F depict dimensions for a prototype folding card HCR lateral flow device.

FIGS. 58A-58C depict assembly of a prototype folding card HCR lateral flow device.

FIGS. 59A-59E demonstrate the limits of detection of five commercial lateral flow devices for unamplified detection of SARS-CoV-2 nucleocapsid protein N in a sample.

FIG. 60 demonstrates the improved limit of detection of an amplified HCR lateral flow test compared to five commercial unamplified lateral flow tests.

FIG. 61 depicts binding events in the test region of an HCR lateral flow device for amplified detection of the SARS-CoV-2 RNA genome in a sample.

FIG. 62 depicts a three-channel membrane for automated delivery of reagents in three successive stages to the test region of an HCR lateral flow device for amplified detection of the SARS-CoV-2 RNA genome in a sample.

FIG. 63 demonstrates detection of the SARS-CoV-2 RNA genome in a sample using an HCR lateral flow assay.

FIGS. 64A-64C demonstrate performance of an HCR lateral flow device for amplified detection of the SARS-CoV-2 RNA genome in a sample.

FIGS. 65A-65C demonstrate performance across replicate tests of an HCR lateral flow device for amplified detection of the SARS-CoV-2 RNA genome in a sample.

FIG. 66 depicts dimensions for a prototype half-strip HCR lateral flow device.

FIG. 67 demonstrates growth of long HCR amplification polymers in ten minutes.

FIG. 68 demonstrates growth of HCR amplification polymers in the context of HCR lateral flow assays for detection of SARS-CoV-2 nucleocapsid protein N in a sample.

FIG. 69 demonstrates amplification gain in the context of HCR lateral flow assays for detection of SARS-CoV-2 nucleocapsid protein (N) in a sample.

FIG. 70 demonstrates amplification gain in the context of HCR lateral flow assays for detection of the SARS-CoV-2 RNA genome in a sample.

FIGS. 71A-71C depict some embodiments of binding events in the test region and control region of an HCR lateral flow device for amplified detection of a target in a sample using a competitive format.

FIGS. 72A-72C depict some embodiments of multi-channel membranes for delivery of reagents to the test region and control region of an HCR lateral flow device for amplified detection of a target in a sample using a competitive format.

FIGS. 73A-73F depict some embodiments of devices for delivery of reagents to the test region of an HCR lateral flow device for amplified detection of a target in a sample using a competitive format.

FIG. 74 demonstrates detection of the Zika RNA genome in a sample using an HCR lateral flow assay.

FIG. 75 demonstrates detection of the MERS RNA genome in a sample using an HCR lateral flow assay.

FIG. 76 demonstrates detection of H3N2 (Influenza A) nucleocapsid protein (NP) in a sample using an HCR lateral flow assay.

FIG. 77A-77B demonstrate detection of H5N1 (bird flu) hemagglutinin protein in a sample using either of two HCR lateral flow assays.

FIG. 78 demonstrates multiplex detection of SARS-CoV-2 nucleocapsid protein N and H3N2 (Influenza A) nucleocapsid protein (NP) in a sample using an HCR lateral flow assay.

### DETAILED DESCRIPTION

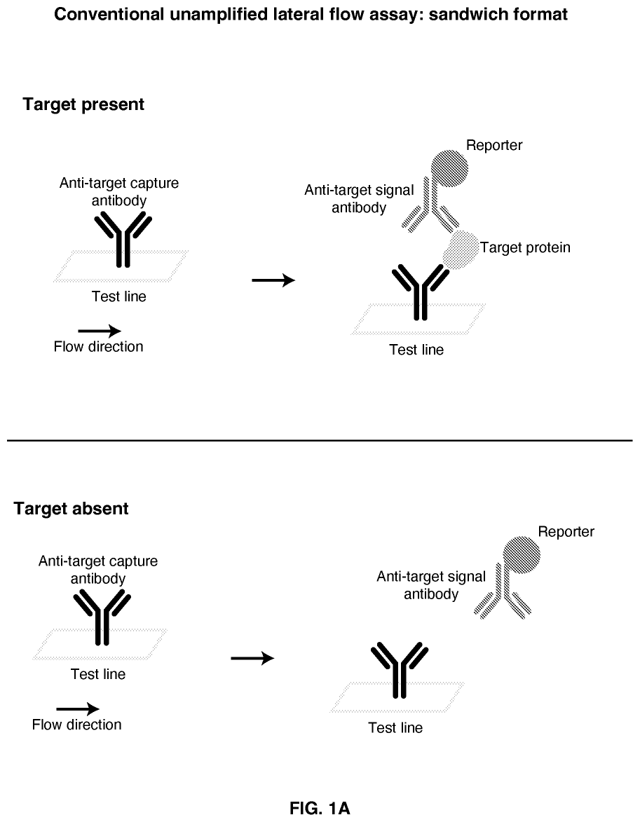

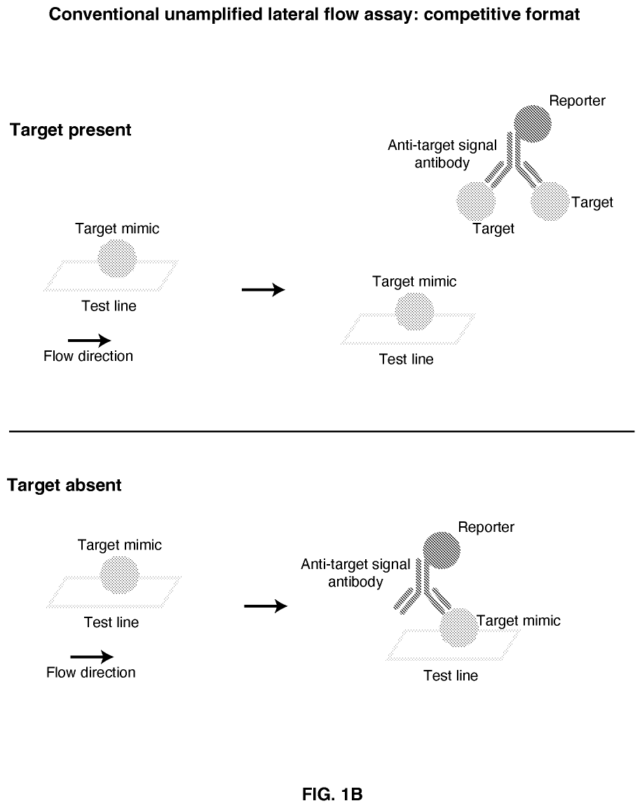

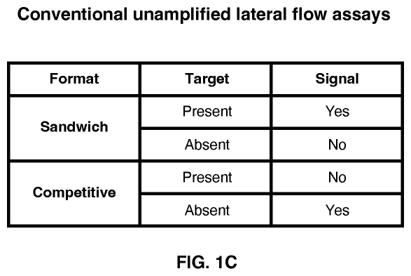

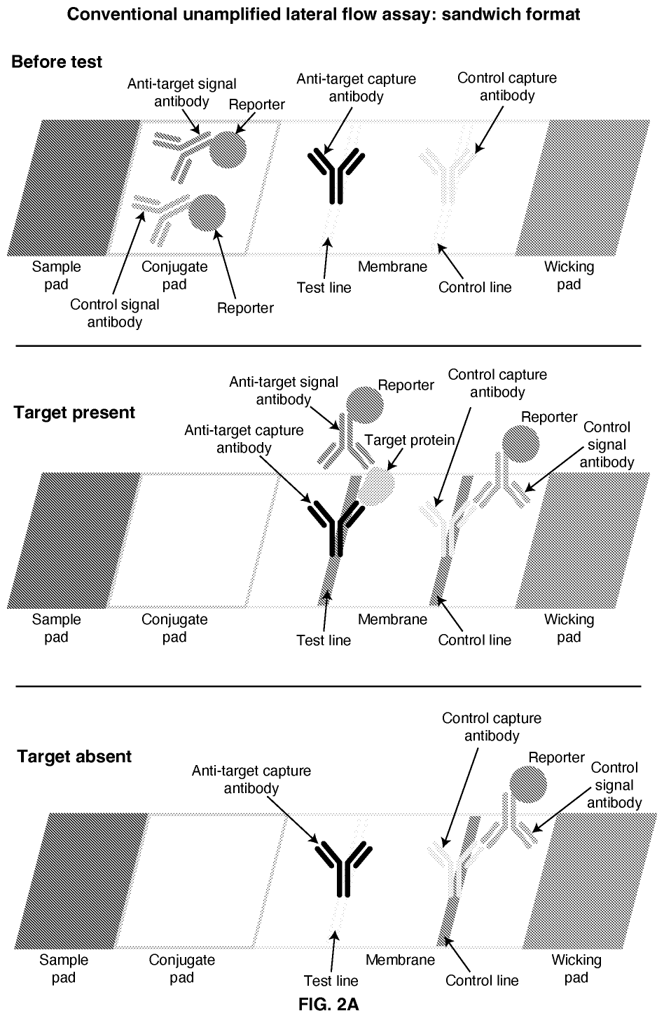

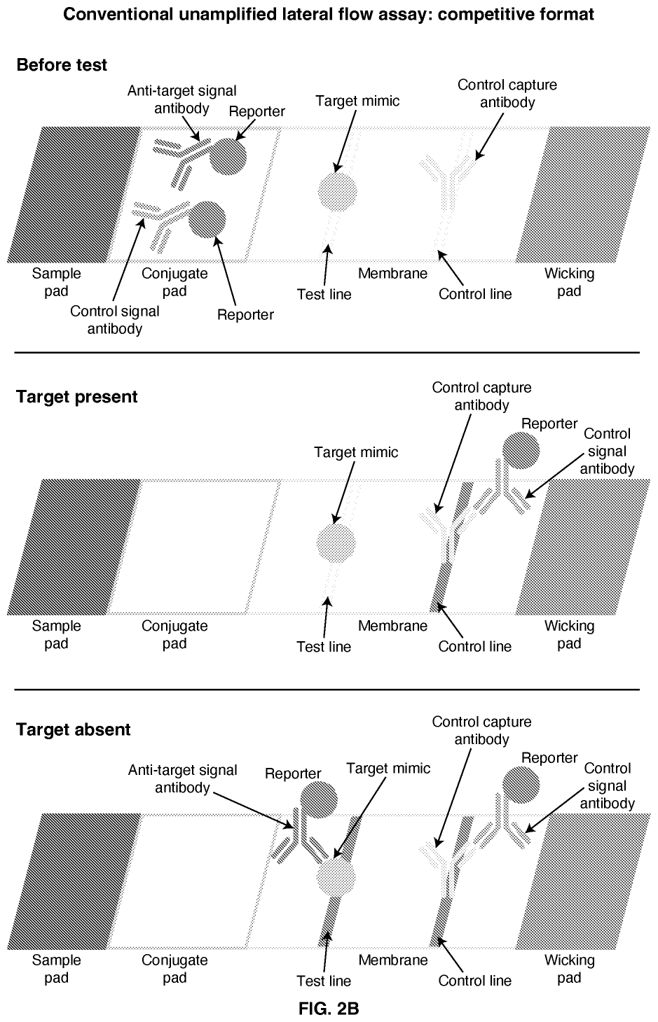

Lateral flow assays can have many advantages over other diagnostic methods and devices. For example, they may allow user-friendly at-home testing for a target in a sample. In a typical lateral flow assay, a target protein moves via capillary forces through a porous substrate, binding in a complex, or ‘sandwich’, between an anti-target signal agent, such as an antibody, labeled with a colored reporter, and an anti-target capture agent, such as an antibody, immobilized at a test line, concentrating the reporter at the test line to generate a signal that may be visible to the naked eye (for example, see FIGS. 1A and 2A). For example, at-home pregnancy tests employ a lateral flow assay format to detect a target protein abundant in urine during early pregnancy.¹ As another example, at-home SARS-CoV-2 rapid antigen tests detect the SARS-CoV-2 nucleocapsid protein (N) to diagnose COVID-19.² With conventional lateral flow assays, the resulting signal is unamplified (i.e., one labeled antibody generates signal for one detected target protein), placing limits on sensitivity. However, the striking simplicity of lateral flow assays makes them attractive for home use. To take a test, the user simply adds the sample to the disposable device and then checks by eye for a colored signal at the test line after a prescribed number of minutes.

Due to the absence of signal amplification, the simplicity of conventional lateral flow assays comes at a cost in sensitivity. If the target is not sufficiently abundant, then the signal may not be detectable. For example, a signal may not be visible to the human eye at the test line even when the target is present in the sample. In this case, the shortfall in sensitivity leads to a false-negative test result. For example, if the lateral flow assay is a SARS-CoV-2 test, a false-negative result gives the user the mistaken impression that they do not have COVID-19, potentially allowing the user to unknowingly transmit the SARS-CoV-2 virus to other people.

The sensitivity limitations for typical lateral flow assays can pose challenges. For example, the sensitivity challenge for SARS-CoV-2 stems from the relative scarcity of SARS-CoV-2 virions in readily sampled biological fluids. The protein that serves as a pregnancy marker in urine rises to ˜10¹⁰ copies/μL during the first month of pregnancy,^{3,4} with unamplified commercial lateral flow assays typically providing limits of detection of ˜10⁷ copies/μL.^{4,5} By comparison, SARS-CoV-2 studies have reported median viral loads of 158 and 3300 virions/μL in saliva.^{6,7} Lab-based tests using reverse transcription quantitative PCR (PCR tests) have been established with limits of detection of 0.1-0.6 copies/μL^{8,9} and represent the gold standard for detecting SARS-CoV-2 infections. By contrast, unamplified lateral flow assays for at-home SARS-CoV-2 testing are easy to use and highly reliable when they return a positive result (e.g., 96-100%),^{10,11} but sensitivity limitations lead to high false-negative rates (e.g., 25-50%).¹⁰⁻¹² With the looming threat of a bird flu pandemic (H5N1; 56% human mortality rate)¹³ or a pandemic due to another emergent pathogen, there is an urgent need for amplified instrument-free at-home tests for pathogen detection.

### Conventional Unamplified Lateral Flow Assays

A typical lateral flow assay comprises a sample pad, a conjugate pad onto which a reporter-labeled anti-target signal antibody and reporter-labeled control signal antibody have been dried down, a membrane, a test line on which an anti-target capture antibody has been pre-immobilized, a control line on which a control capture antibody has been pre-immobilized, and a wicking pad (for example, see FIG. 2A).¹ Upon introducing the sample to the sample pad, the sample flows via capillary forces along a single channel from the sample pad, through the conjugate pad, through the membrane, across the test line, across the control line, and to the wicking pad. The wicking pad absorbs liquid to induce continued capillary flow after the sample reaches the wicking pad. As the flow crosses the conjugate pad, the dried down reporter-labeled anti-target signal antibody and the dried down reporter-labeled control signal antibody are resuspended. If the target is present in the sample, it binds the reporter-labeled anti-target signal antibody and subsequently binds the anti-target capture antibody that is pre-immobilized at the test line, immobilizing a reporter-labeled antibody/target sandwich to generate a signal at the test line (for example, see FIGS. 1A and 1C). If the target is not present in the sample, the antibody/target sandwich does not form, the reporter-labeled anti-target signal antibody is not immobilized at the test line, and a signal is not generated at the test line (for example, see FIGS. 1A and 1C). If the sample and reagents successfully flow across the control line, the reporter-labeled control signal antibody binds the immobilized control capture antibody, immobilizing reporters at the control line to generate a signal at the control line (for example, see FIG. 2A). The test produces a positive result if both the test line and the control line produce visible signals. The test produces a negative result if signal is observed at the control line but not the test line. Absence of signal at the control line is an indication that the test did not function properly. Using a conventional sandwich lateral flow assay, the signal at the test line is unamplified (i.e., one reporter-labeled anti-target signal antibody generates signal for one target protein), placing limits on sensitivity, but the striking simplicity of lateral flow assays makes them attractive for home use. Within a lateral flow assay, reagents can flow to the test line through one or more membrane channels.^{14,15} Efforts to enhance sensitivity by introducing amplification into SARS-CoV-2 lateral flow assays, including use of loop-mediated isothermal amplification (LAMP)^{16,17} and CRISPR/Cas,^{18,19} have led to compromises on simplicity, cost, and/or robustness, requiring multiple user steps, dedicated instrumentation, and/or enzymes with strict storage requirements.

The lateral flow assay of FIG. 2A uses a sandwich format in which a probe/target sandwich forms to generate a signal at the test line if the target is present in the sample (for example, see FIGS. 1A and 1C). With a sandwich lateral flow assay, if the target is not present in the sample, the probe/target sandwich does not form and a signal is not generated at the test line (for example, see FIGS. 1A and 1C).

By contrast, the lateral flow assay of FIG. 2B uses a competitive format in which a signal is generated at the test line if the target is not present in the sample (for example, see FIGS. 1B and 1C). With a competitive lateral flow assay, if the target is present in the sample, a signal is not generated at the test line (for example, see FIGS. 1B and 1C). A typical competitive lateral flow assay comprises a sample pad, a conjugate pad onto which a reporter-labeled anti-target signal antibody has been dried down, a membrane, a test line on which a target mimic has been pre-immobilized, a control line on which a control capture antibody has been pre-immobilized, and a wicking pad (for example, see FIG. 2B).¹ Upon introducing the sample to the sample pad, the sample flows via capillary forces along a single channel from the sample pad, through the conjugate pad, through the membrane, across the test line, across the control line, and to the wicking pad. The wicking pad absorbs liquid to induce continued capillary flow after the sample reaches the wicking pad. As the flow crosses the conjugate pad, the dried down reporter-labeled anti-target signal antibody is resuspended. If the target is present in the sample, it binds the reporter-labeled anti-target signal antibody to inhibit binding of the reporter-labeled anti-target signal antibody to the target mimic that is pre-immobilized at the test line, preventing generation of a signal at the test line (for example, see FIGS. 1B and 1C). If the target is not present in the sample, the reporter-labeled anti-target signal antibody binds the target mimic that is pre-immobilized at the test line to generate a signal at the test line (for example, see FIGS. 1B and 1C). If the sample and reagents successfully flow across the control line, the reporter-labeled control signal antibody binds the immobilized control capture antibody, immobilizing reporters at the control line to generate a signal at the control line (for example, see FIG. 2B). The test produces a positive result if signal is observed at the control line but not the test line. The test produces a negative result if both the test line and the control line produce visible signals. Absence of signal at the control line is an indication that the test did not function properly. Using a conventional competitive lateral flow assay, the signal at the test line is unamplified (i.e., one reporter-labeled anti-target signal antibody generates signal for one target mimic), again placing limits on sensitivity.

### HCR Signal Amplification for In Situ Hybridization and Immunofluorescence

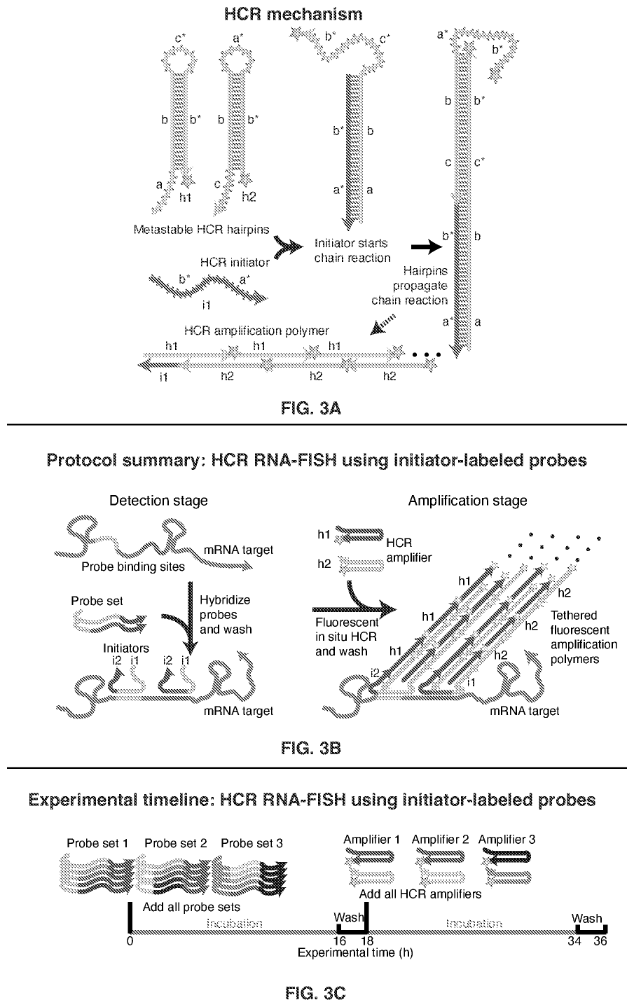

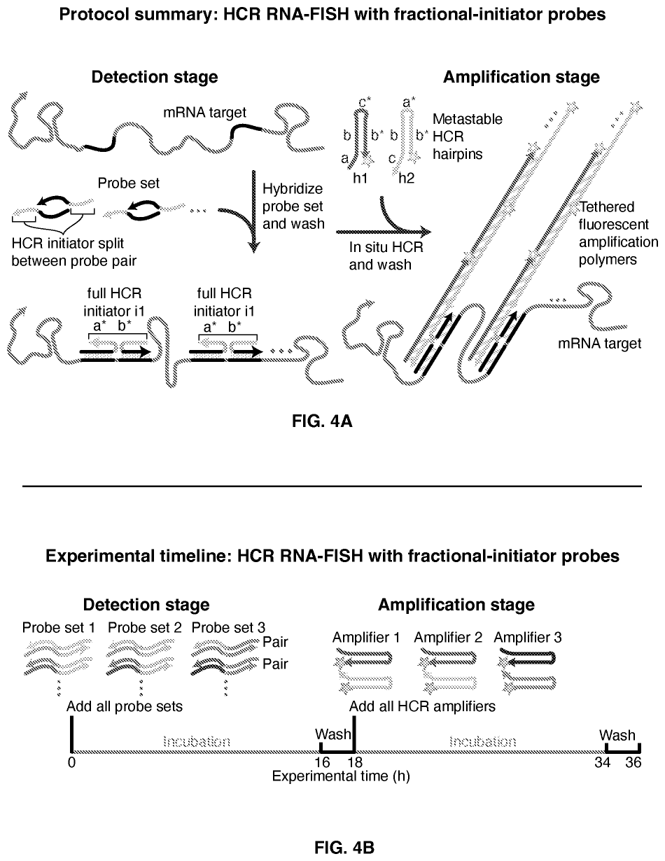

For bioimaging applications, signal amplification based on the mechanism of hybridization chain reaction (HCR)²⁰ has previously been used to provide in situ signal amplification for imaging RNA and protein targets within fixed biological specimens.²¹⁻²⁴ For HCR imaging, target molecules are detected within the fixed specimen using probes that trigger chain reactions in which fluorophore-labeled HCR hairpins self-assemble into tethered fluorescent HCR amplification polymers, generating amplified signals at the locations of target molecules in situ within a cell, tissue, embryo, or organism (for example, see FIGS. 3A-3B and 4A).²¹⁻²⁵ The specimen is then imaged with a fluorescence microscope to map the expression pattern of the target molecule in an anatomical context. HCR imaging protocols typically involve multiple stages in which different reagents are washed into or out of the sample (for example, see FIGS. 3C and 4B).^{15,19} For example, in a detection stage, probes can be added to the fixed sample, incubated to allow probes to bind targets, and then a wash can be used to remove unused probes from the sample to reduce background resulting from non-specific probe binding; in a subsequent amplification stage, HCR amplifiers comprising fluorophore-labeled HCR hairpins can be added to the sample, incubated to allow HCR signal amplification to occur, and then a wash can be used to remove unused HCR hairpins from the sample to reduce background resulting from non-specific hairpin binding (for example, see FIGS. 3C and 4B). HCR signal amplification has some properties that are favorable for use in an at-home test platform, including: HCR polymerization is isothermal and operates efficiently at room temperature, the resulting amplification polymers are tethered so as to concentrate the amplified signal at the target location, and HCR reagents are robust and do not require cold-storage. On the other hand, some aspects of HCR imaging protocols are unfavorable when contemplating development of an at-home testing platform, including the reliance on multiple hands-on steps (probe addition, incubation, and removal, followed by amplifier addition, incubation, and removal; for example, see FIGS. 3B and 4A), the overall duration of typical protocols (involving overnight probe incubation followed by overnight amplifier incubation; for example, see FIGS. 3C and 4B), and the need for a fluorescence microscope to image the results.²¹⁻²⁵ Additional information about HCR and uses thereof can be found in Dirks, R. and Pierce, N. Proc. Natl. Acad. Sci. USA 101(43): 15275-15278 (2004), incorporated herein by reference in its entirety. Further information about HCR and uses thereof can be found in U.S. Pat. No. 7,632,641B2, filed on Mar. 22, 2005 with the title “Hybridization Chain Reaction”, and WO2021221789A2 filed on Mar. 4, 2021 with the title “Analysis of Target Molecules within a Sample via Hybridization Chain Reaction”, hereby expressly incorporated by reference in its entirety. Additional information about fractional initiators and uses thereof can be found in U.S. Ser. No. 10/450,599B2, filed on Jun. 30, 2017, with the title “Fractional Initiator Hybridization Chain Reaction”, hereby expressly incorporated by reference in its entirety.

### HCR Lateral Flow Assays for Amplified Detection of a Target in a Sample

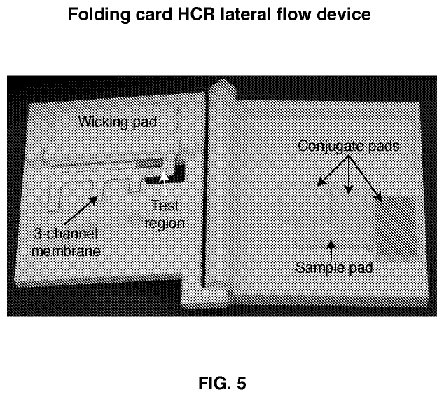

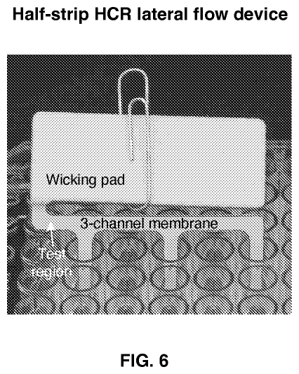

In some embodiments amplified HCR lateral flow devices are provided and may be used to detect a target in a sample. In some embodiments, an HCR lateral flow device comprises one or more membranes, one or more conjugate pads, one or more sample pads, one or more wicking pads, one or more test regions, and/or one or more control regions (for example, see FIGS. 5 and 6). In some embodiments, the one or more membranes, conjugate pads, sample pads, and/or wicking pads, independently comprise nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate. In some embodiments, a lateral flow device comprises an optional sample pad, an optional conjugate pad, a membrane, a test region, an optional control region, and a wicking pad. In some embodiments, each conjugate pad is fluidly connected to a single channel.

In some embodiments, an amplified HCR lateral flow device comprises one or more probe sets, wherein each probe set comprises one or more probes. In some embodiments, a probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, or any combination thereof.

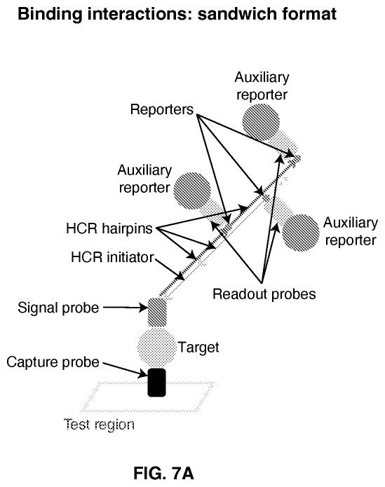

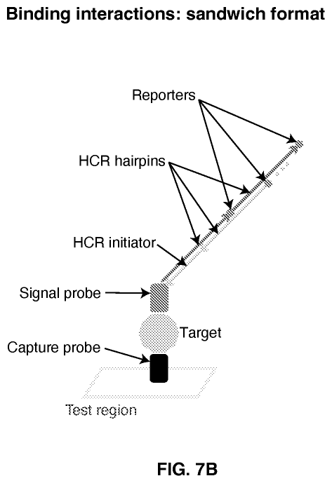

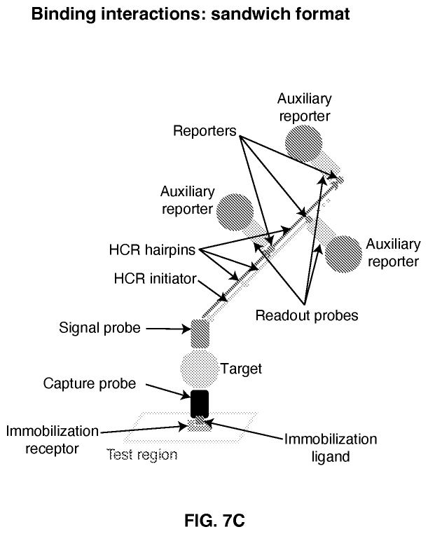

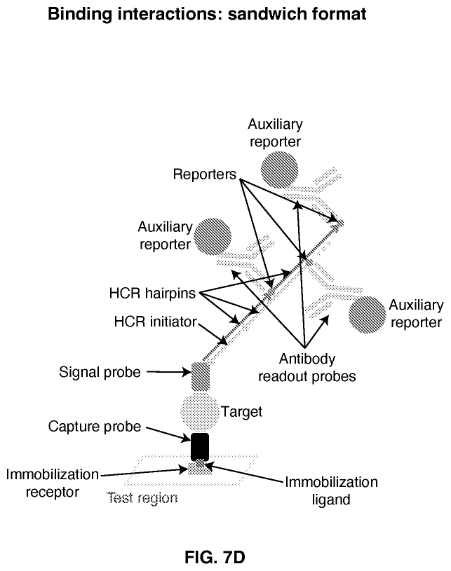

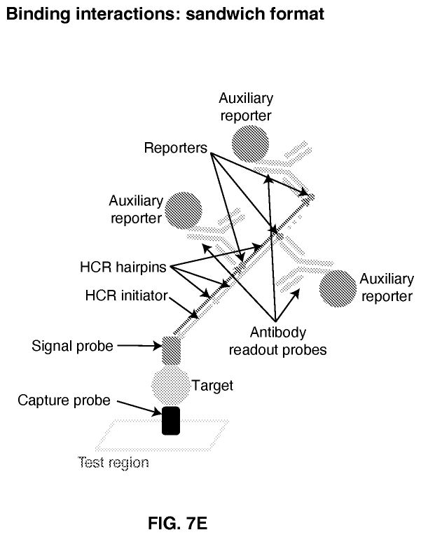

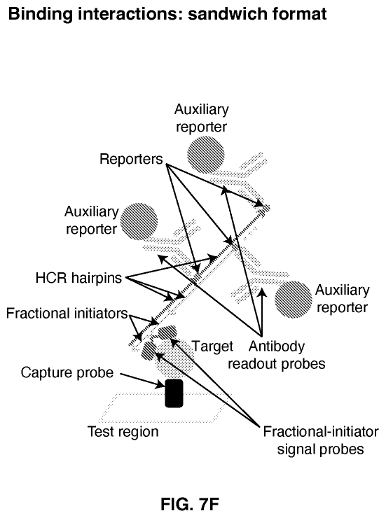

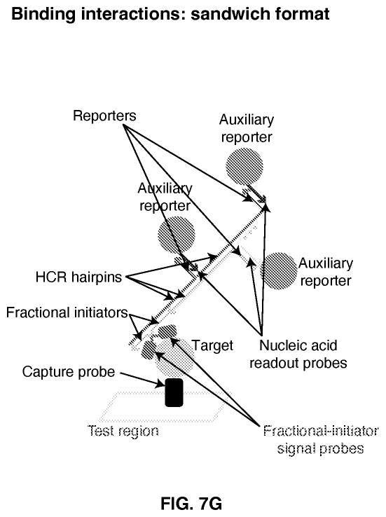

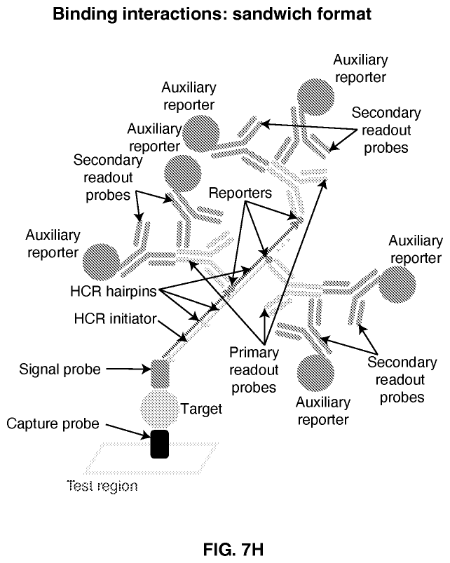

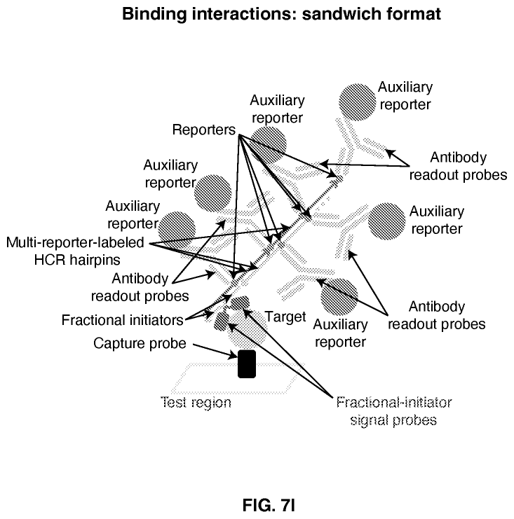

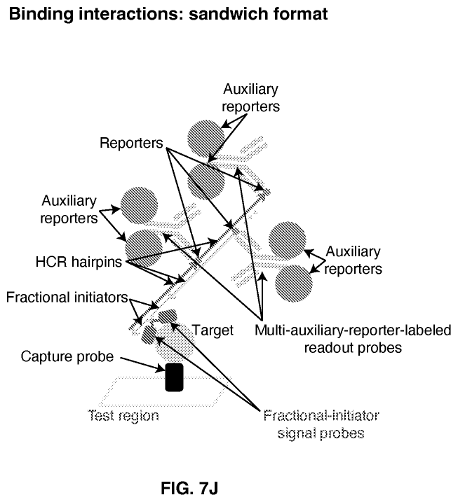

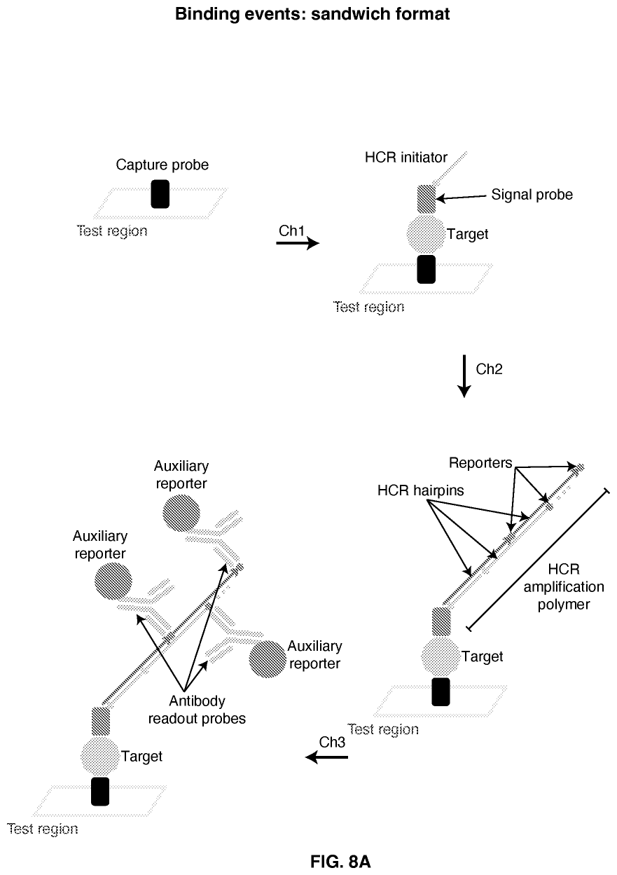

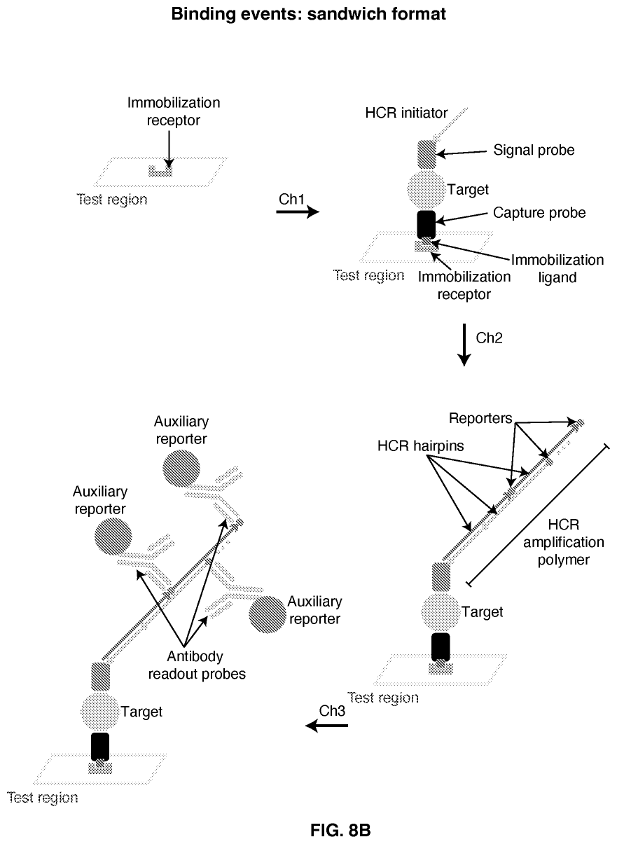

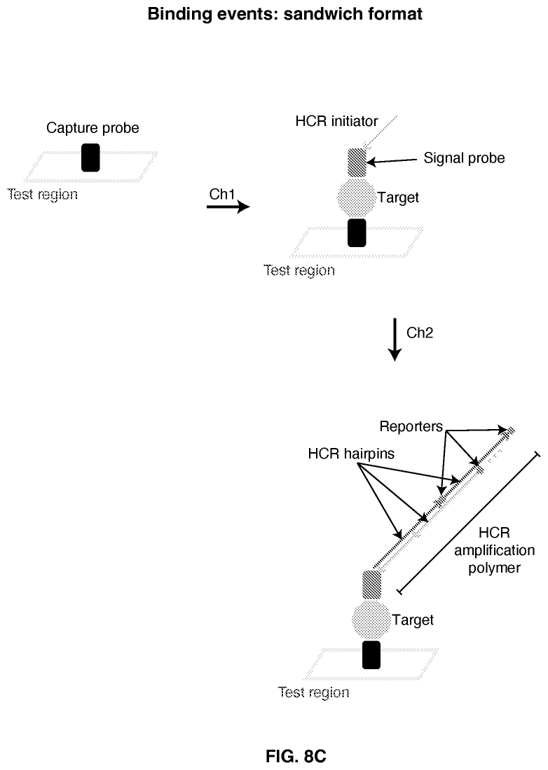

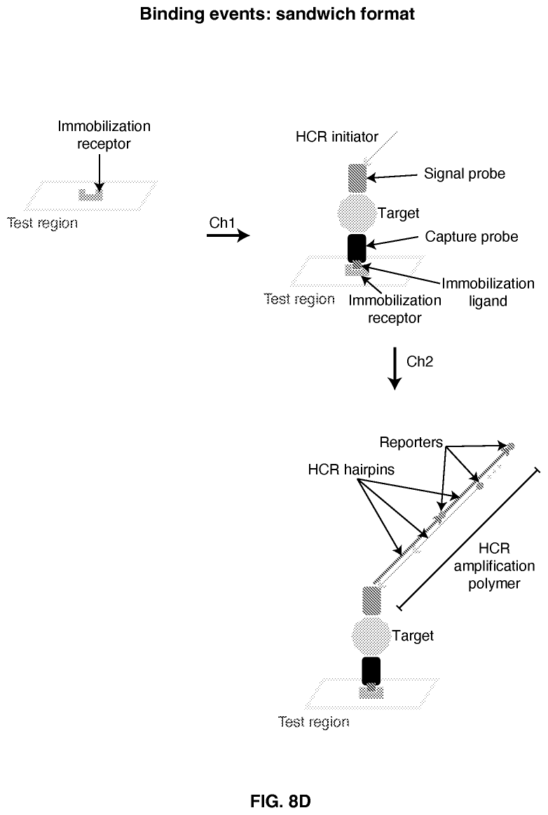

In some embodiments, a device comprises a test region (for example, see FIGS. 7A-7J, 9A-9E, and 11A-11E). In some embodiments, the one or more probe sets comprise a capture probe set comprising one or more capture probes, wherein a capture probe comprises a target-binding domain configured to bind directly or indirectly to the target and an immobilization domain configured to bind directly or indirectly to the test region (for example, see FIGS. 7A-7J, 9A-9E, and 11A-11E). In some embodiments, the one or more capture probes are pre-immobilized in the test region (for example, see FIGS. 8A, 8C, 10A, 10C, 12A, and 12C). In some embodiments, the one or more capture probes are labeled with an immobilization ligand that binds an immobilization receptor that is pre-immobilized in the test region (for example, see FIGS. 8B, 8D, 10B, 10D, 12B, and 12D). In some embodiments, the immobilization ligand is biotin and the immobilization receptor is a biotin-binding molecule (for example, polystreptavidin R (PR), streptavidin, avidin, NeutrAvidin, CaptAvidin, or another biotin-binding molecule), or the immobilization ligand is a hapten and the immobilization receptor is an anti-hapten antibody, or the immobilization ligand is an anti-hapten antibody and the immobilization receptor is a hapten, or the immobilization ligand is a nucleic acid and the immobilization receptor is a nucleic acid, or the immobilization ligand is an antigen and the immobilization receptor is an anti-antigen antibody, or the immobilization ligand is a molecule that is recognized by an immobilization receptor that is a zinc finger, or the immobilization ligand is another molecule or complex that can be recognized by an immobilization receptor. In some embodiments, a capture probe comprises a primary capture probe comprising a target-binding domain and a secondary capture probe comprising an immobilization domain configured to bind directly or indirectly to the test region wherein the secondary capture probe is configured to bind the primary capture probe directly or indirectly (for example, a primary capture probe that is a primary antibody and a secondary capture probe that is a secondary antibody configured to bind the primary antibody).

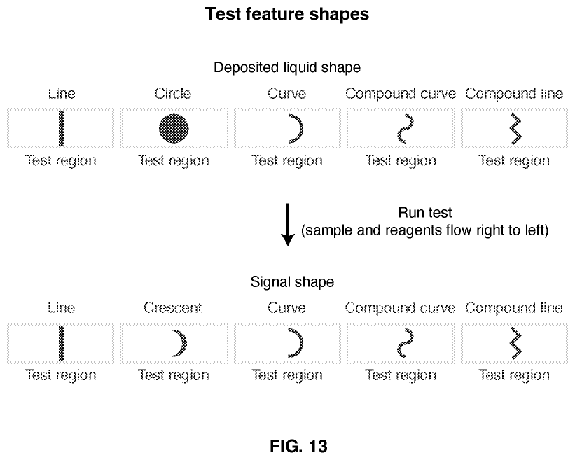

In some embodiments, the capture probe set is pre-immobilized in the test region in a test feature shape that is a straight line, a curve, a zigzag, a compound line, a compound curve, a cross, a filled circle, an open circle, a filled shape, an open shape, or another feature shape (for example, see FIG. 13). In some embodiments, the capture probe set is immobilized in the test region by binding an immobilization receptor that is pre-immobilized in the test region in a test feature shape that is a straight line, a curve, a zigzag, a compound line, a compound curve, a cross, a filled circle, an open circle, a filled shape, an open shape, or another feature shape. In some embodiments, if the target is present in the sample, the amplified signal generated in the test region takes the form of a straight line, a curve, or another shape depending on the test feature shape. In some embodiments, one test feature shape can be easier for the human eye to detect than another test feature shape; for example, if the target is present in the sample at very low abundance and the amplified signal is faint. In some embodiments, the amplified signal can be detected with a reader device.

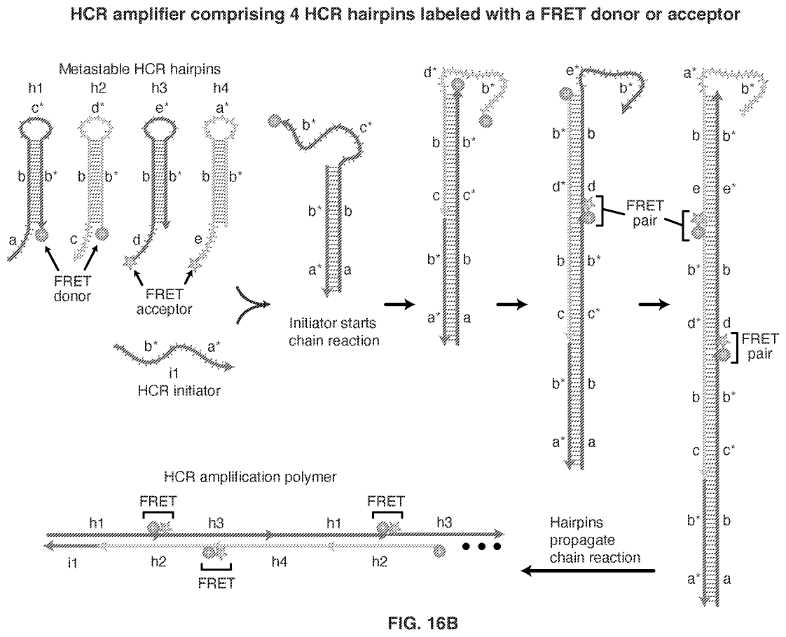

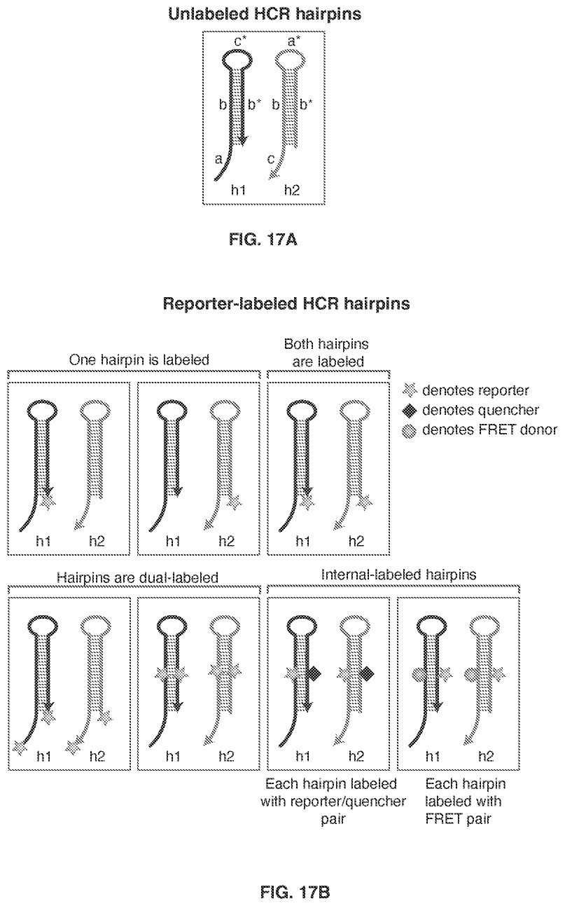

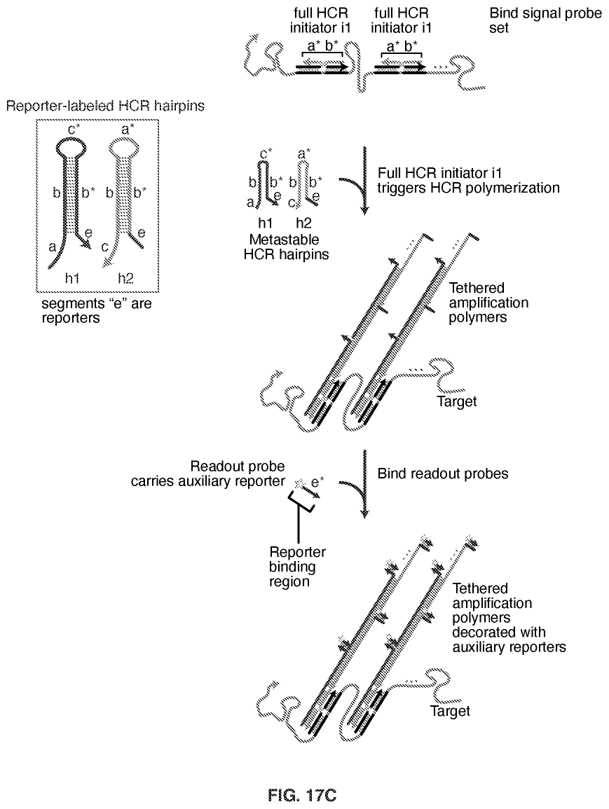

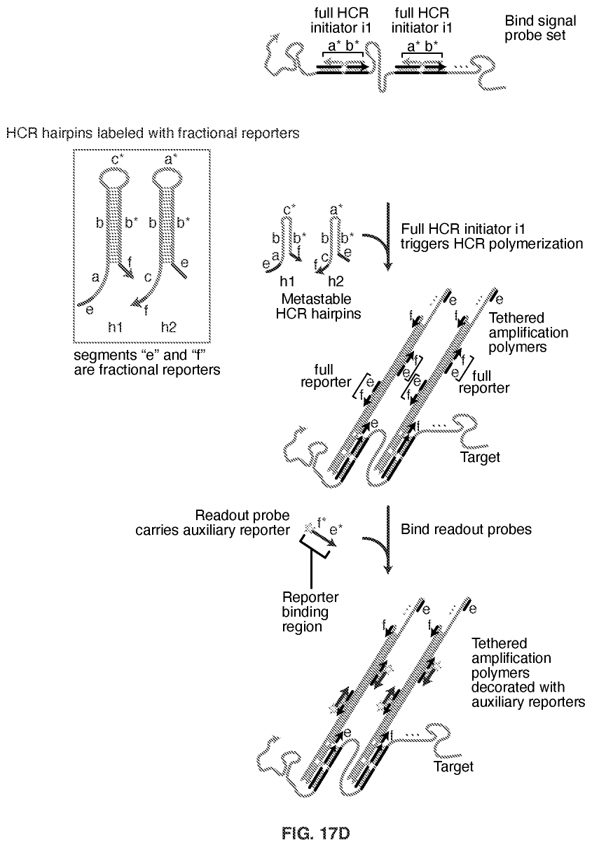

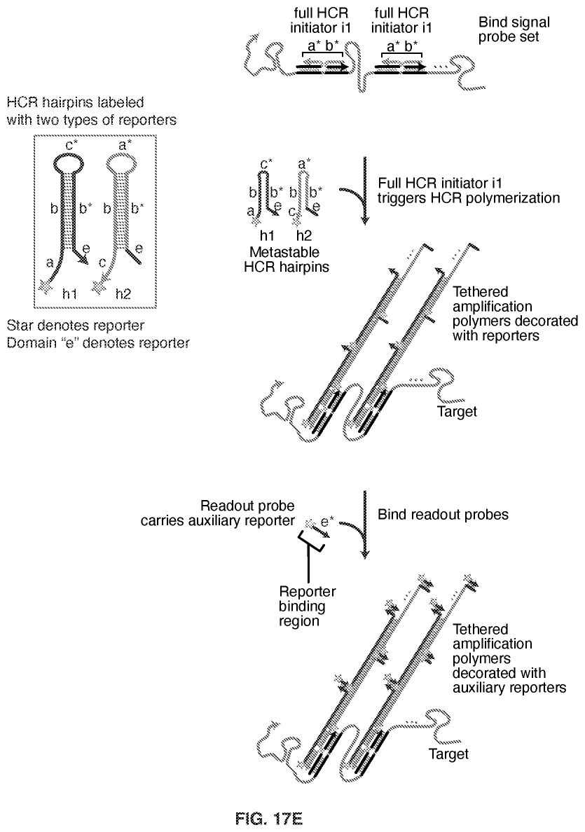

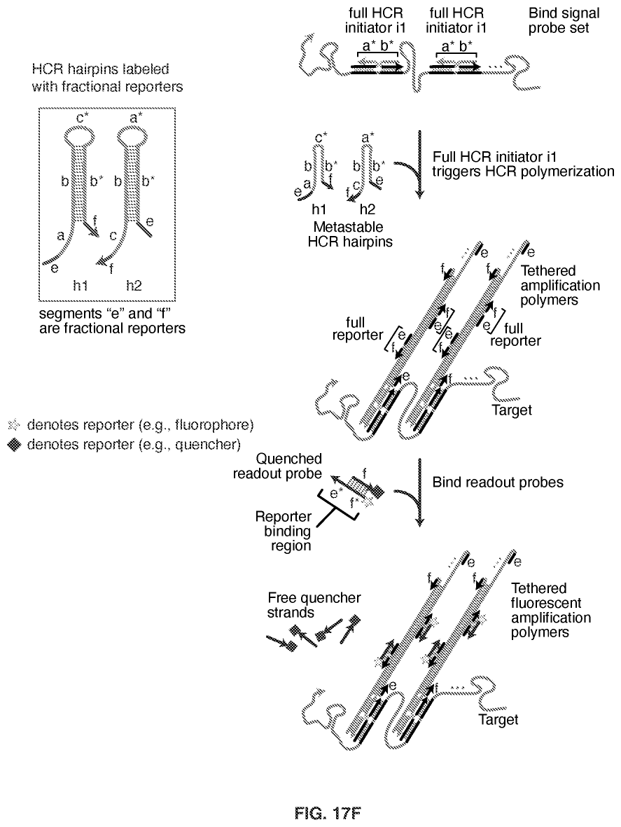

In some embodiments, the device comprises an HCR amplifier comprising two or more HCR hairpins (for example, see FIGS. 3A, 14, 15, and 16A-16B). In some embodiments, one or more of the HCR hairpins are labeled with one or more reporters (for example, see FIG. 17A-17F). In some embodiments, the HCR hairpins of the HCR amplifier are configured not to polymerize in the absence of an HCR initiator. In some embodiments, the HCR hairpins are configured such that presence of an HCR initiator triggers the HCR hairpins to polymerize to form a tethered HCR amplification polymer decorated with reporters (for example, see FIGS. 3A, 14, 15, and 16A-16B). In some embodiments, the HCR amplification polymer can be tethered to the HCR initiator. In some embodiments, the HCR amplification polymer can further comprise reporters. In some embodiments, the HCR hairpins of the HCR amplifier are configured such that polymerization to form a tethered HCR amplification polymer occurs in the presence of a signal probe.

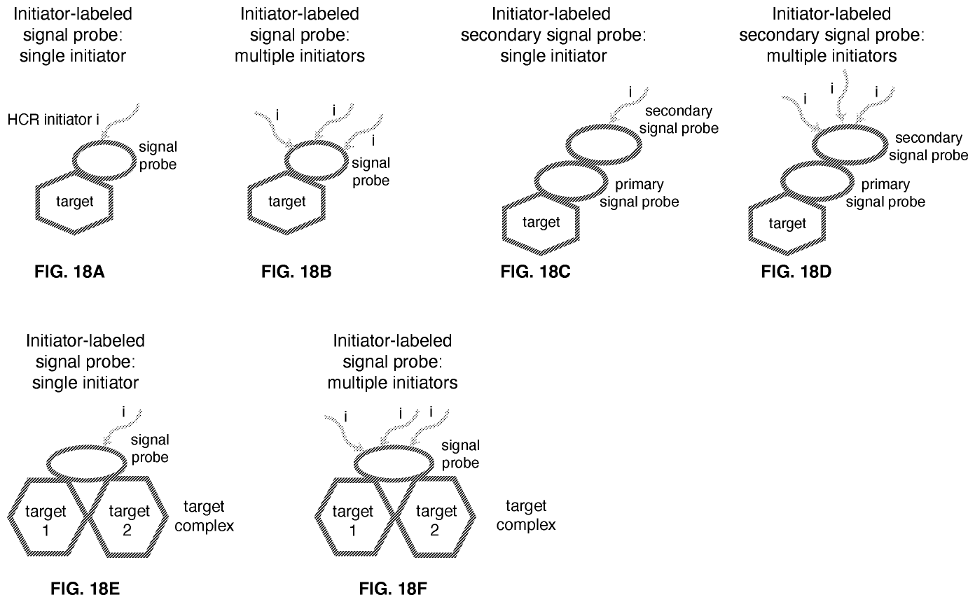

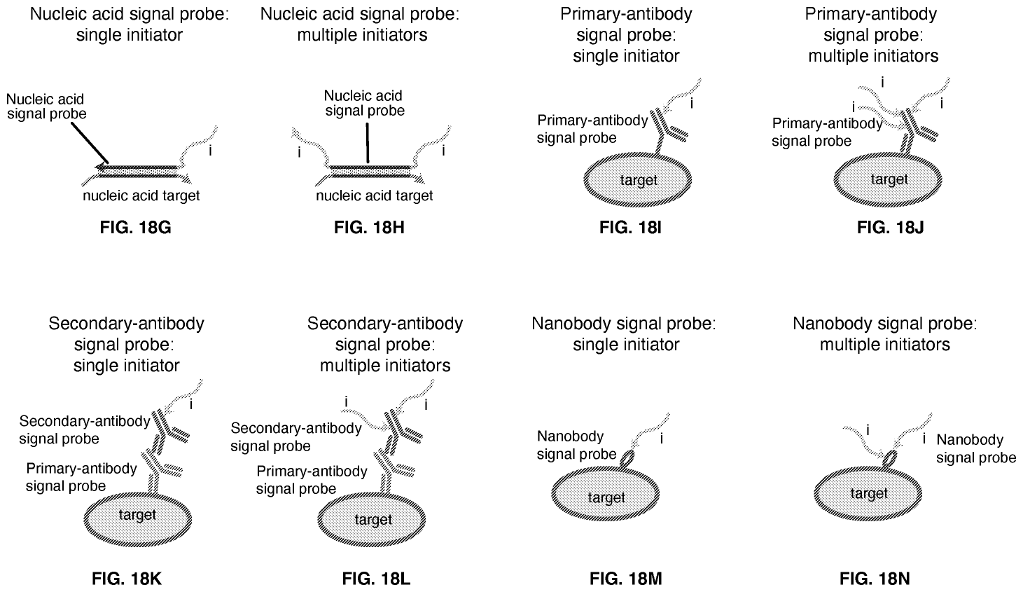

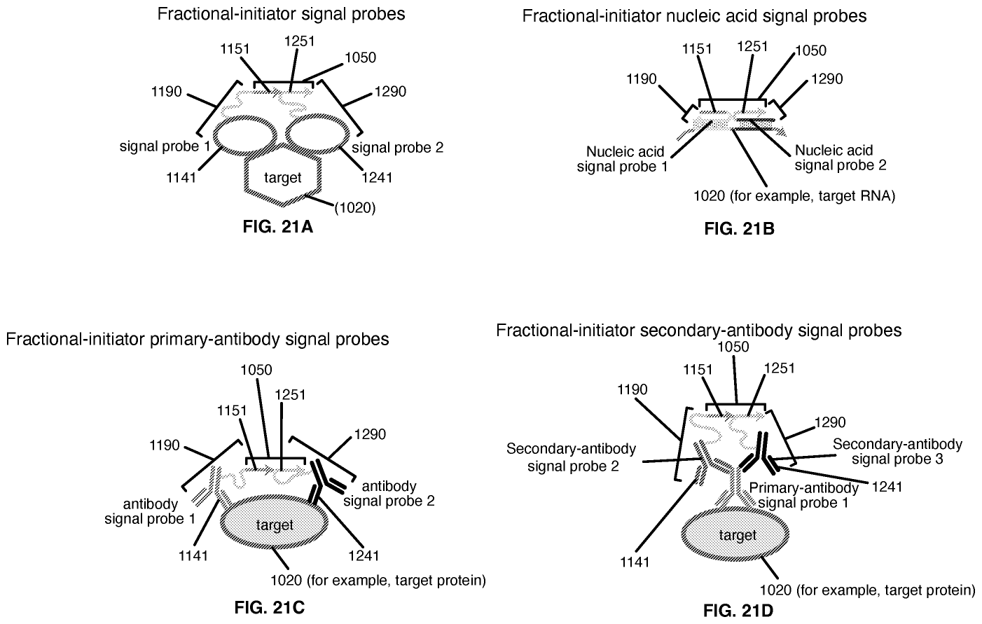

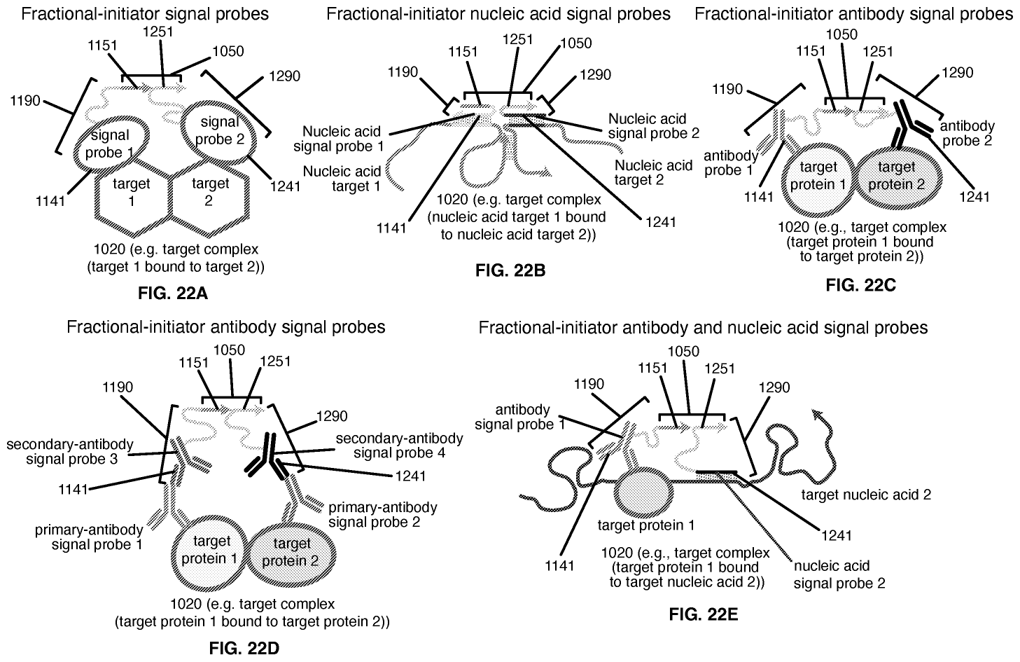

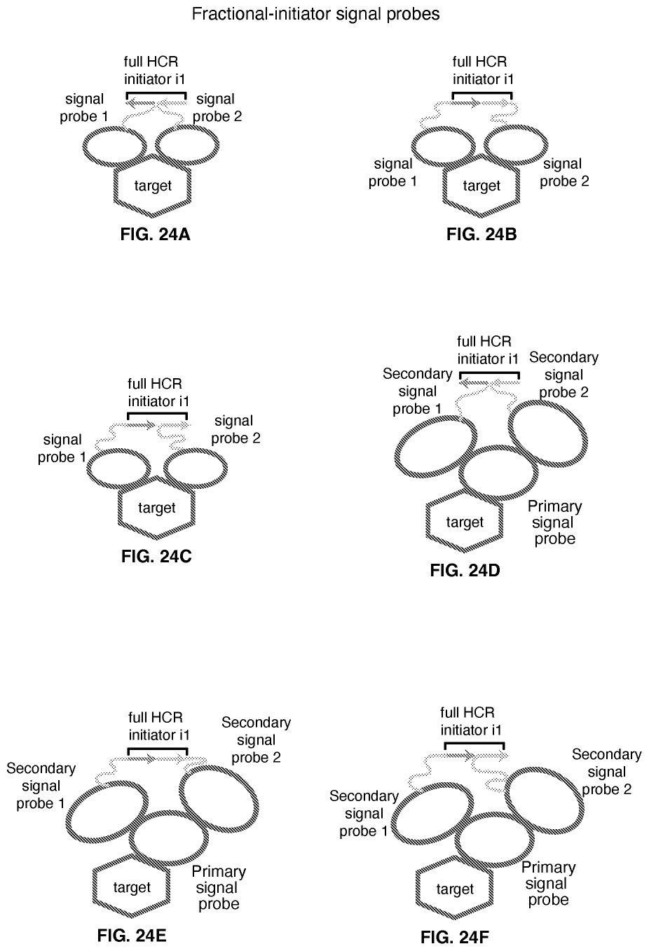

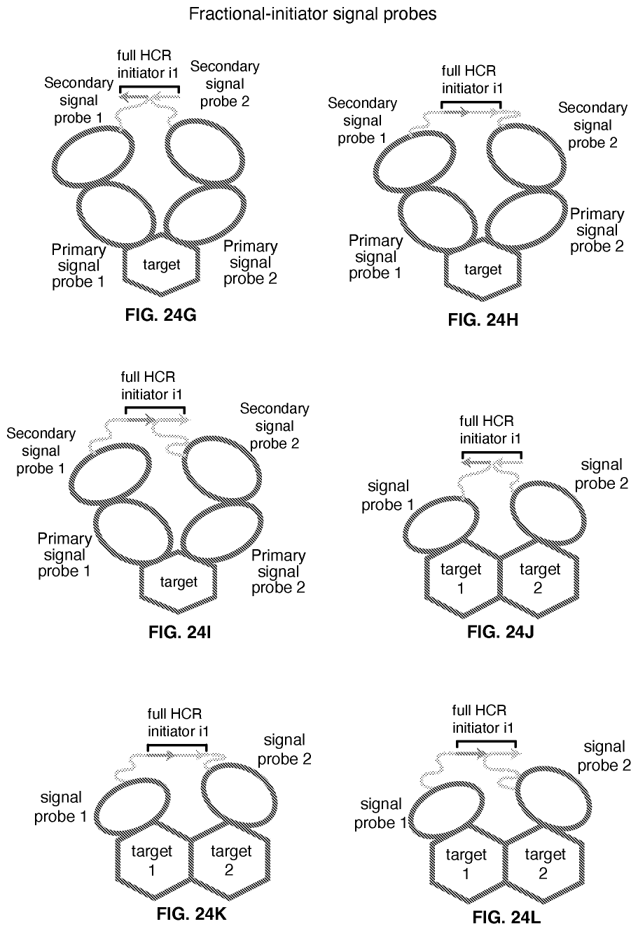

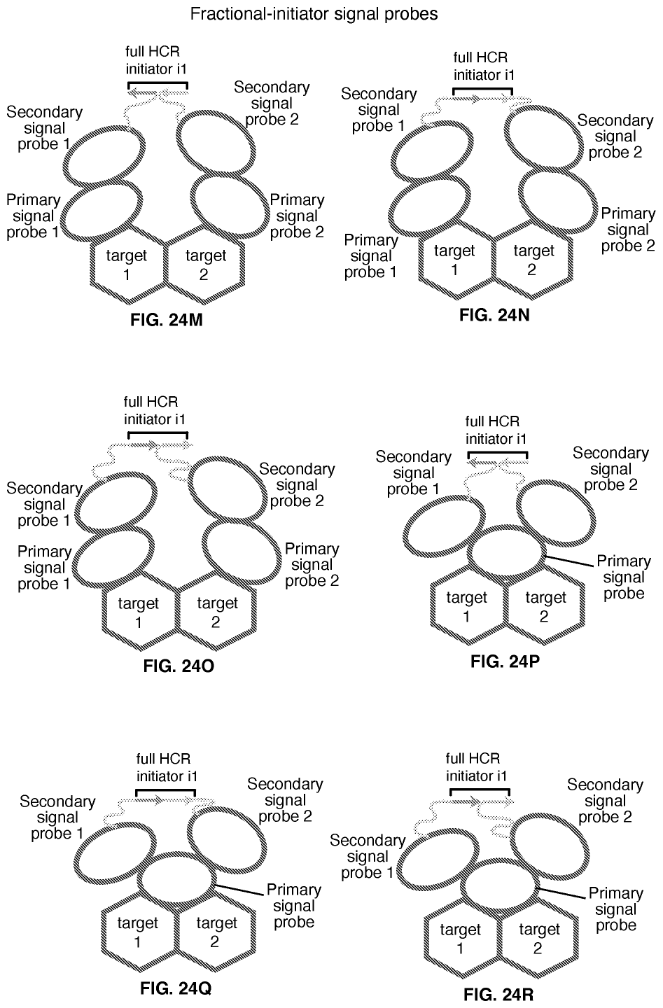

In some embodiments, the one or more probe sets comprise a signal probe set comprising one or more signal probes, wherein a signal probe comprises a target-binding domain configured to bind directly or indirectly to the target and an amplification domain that is configured to directly or indirectly trigger HCR signal amplification by initiating a chain reaction in which HCR hairpins self-assemble to form a tethered HCR amplification polymer decorated with reporters (for example, see FIGS. 7A-7J, 9A-9E, and 11A-11E). In some embodiments, the signal probe set comprises: a) one or more initiator-labeled probes wherein an initiator-labeled probe comprises a target-binding domain and an amplification domain comprising one or more HCR initiators (for example, see FIGS. 3B, 7A-7E, 7H, 9A-9E, 11B, 18A-18N, and 19A-19F), or b) one or more probe units, each comprising two or more fractional-initiator probes, wherein a fractional-initiator probe comprises a target-binding domain and an amplification domain comprising a fractional initiator. In some embodiments, an individual fractional-initiator probe that binds non-specifically will not trigger HCR, but specific binding of the probes within a probe unit to adjacent cognate binding sites on the target colocalizes a full HCR initiator capable of triggering HCR (for example, see FIGS. 4A, 7F-7G, 7I-7J, 11A, 11C-11E, 20A-20E, 21A-21D, 22A-22E, 24A-24R). In some embodiments, a signal probe comprises a primary signal probe comprising a target-binding domain and a secondary signal probe comprising an amplification domain (for example, see FIGS. 18C-18D, 18K-18L, 21D, 22D, 24D-24I, 24M-24R) wherein the secondary signal probe is configured to bind the primary signal probe directly or indirectly (for example, a primary signal probe that is a primary antibody and a secondary signal probe that is a secondary antibody configured to bind the primary antibody). In some embodiments, the primary signal probe comprises a target-binding domain without an amplification domain. In some embodiments, the secondary signal probe comprises an amplification domain without a target-binding domain. In some embodiments, the secondary signal probe comprises an amplification domain that is configured to directly or indirectly trigger HCR signal amplification by initiating a chain reaction in which HCR hairpins self-assemble to form a tethered HCR amplification polymer decorated with reporters.

In some embodiments, the one or more probe sets comprise a readout probe set comprising one or more readout probes, wherein a readout probe comprises a reporter-binding domain configured to bind directly or indirectly to a reporter on an HCR amplification polymer (for example, see FIGS. 7A, 7C-7J, 9A-9E, and 11A-11E). In some embodiments, readout probes comprise one or more auxiliary reporters wherein the auxiliary reporters contribute directly or indirectly to the generation of an amplified signal in the test region (for example, see FIGS. 7A, 7C-7J, 9A-9E, and 11A-11E). In some embodiments, the amplified signal generated by the auxiliary reporters on the readout probes is visible to the human eye. In some embodiments, a readout probe comprises a primary readout probe comprising a reporter-binding domain and a secondary readout probe comprising an auxiliary reporter (for example, see FIG. 7H) wherein the secondary readout probe is configured to bind the primary readout probe directly or indirectly (for example, a primary readout probe that is a primary antibody and a secondary readout probe that is a secondary antibody configured to bind the primary antibody).

In some embodiments, if the target is present in the sample the target binds in a sandwich between a capture probe set and a signal probe set (for example, see FIGS. 7A-7J, 9A-9E, and 11A-11E). In some embodiments, the capture probe set immobilizes the probe/target sandwich in the test region (for example, see FIGS. 8A-8D, 10A-10D, and 12A-12D). In some embodiments, if the target is not present in the sample, the probe/target sandwich does not form, the signal probes are not immobilized in the test region, the growth of a tethered HCR amplification polymer is not triggered in the test region, and an amplified signal is not generated in the test region.

In some embodiments, after the probe/target sandwich is immobilized in the test region, the amplification domains on the signal probes trigger the self-assembly of reporter-labeled HCR hairpins into tethered HCR amplification polymers decorated with reporters (for example, see FIGS. 8A-8D, 10A-10D, and 12A-12D). In some embodiments, the reporters directly or indirectly lead to generation of an amplified signal in the test region.

In some embodiments, the readout probe set comprising one or more readout probes comprises primary-reporter-labeled readout probes that comprise one or more primary reporters.

In some embodiments, auxiliary-reporter-labeled readout probes that comprise one or more auxiliary reporters bind the reporters decorating the tethered HCR amplification polymers that are immobilized in the test region, directly or indirectly generating an amplified signal in the test region (for example, see FIGS. 8A-8B, 10A-10B, and 12A-12B).

In some embodiments, the amplified signal is visible to the human eye. In some embodiments, the signal is read using a fluorescence microscope, a fluorescence scanner, a camera, a mobile phone camera, a mass spectrometer, a mass spectrometry microscope, a radioactive scanner, or another instrument suitable for detecting reporters and/or auxiliary reporters.

In some embodiments, a reporter and/or an auxiliary reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a reporter and/or auxiliary reporter is a hapten, a ligand, an oligonucleotide, digoxigenin (DIG), fluorescein isothiocyanate (FITC), a fluorophore, biotin, dinitrophenol, aniline, an enzyme, or another molecule or complex that can be recognized by a binding partner to facilitate generation of a signal.

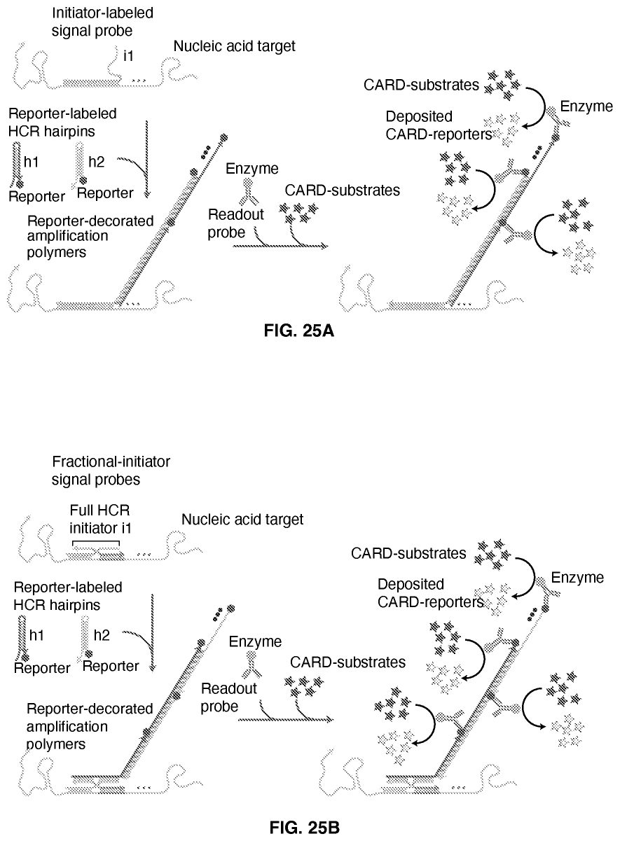

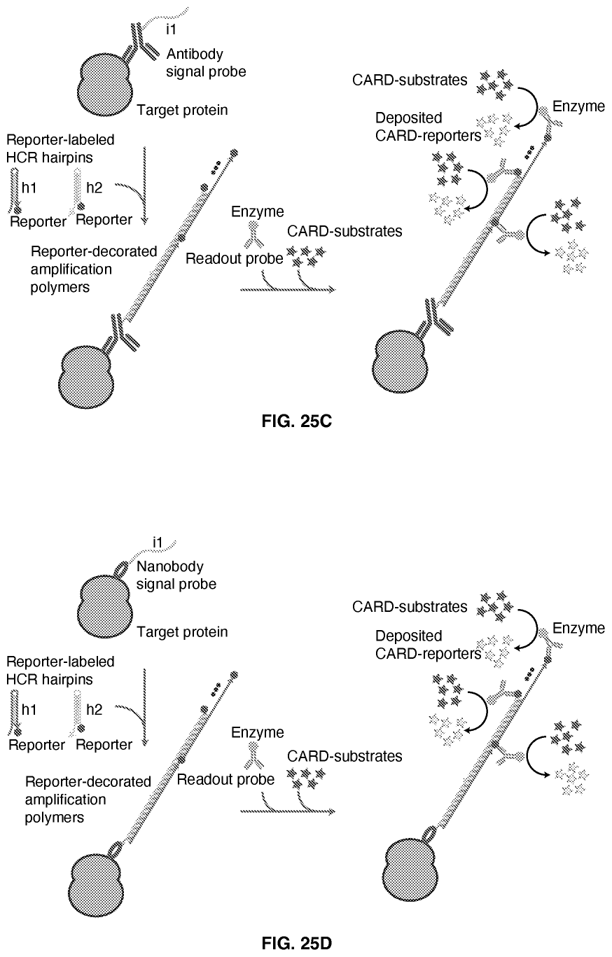

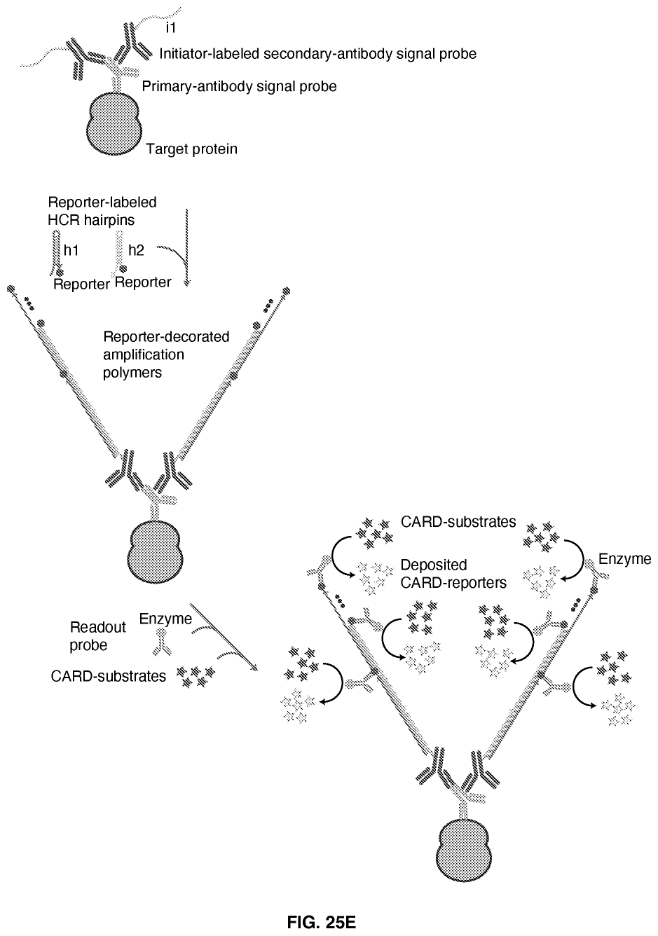

In some embodiments, the reporters and/or auxiliary reporters decorating an HCR amplification polymer directly or indirectly mediate catalytic reporter deposition (CARD) to generate an amplified signal in the vicinity of the HCR amplification polymer within the test region. In some embodiments, CARD is mediated by an enzyme (for example, see FIGS. 25A-25E, 26A-26C, 27A-27B).

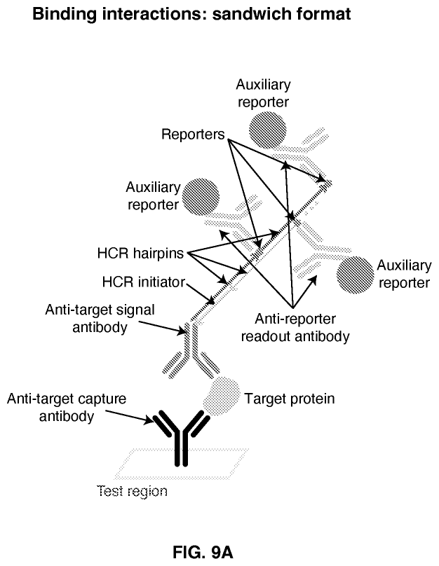

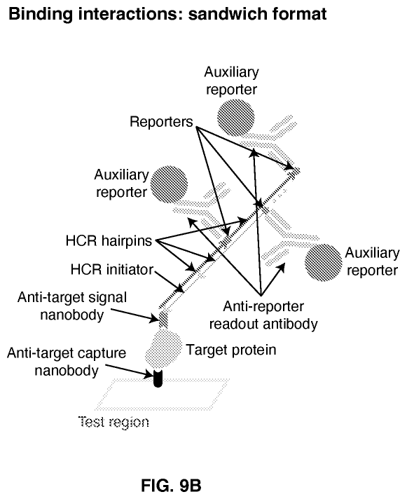

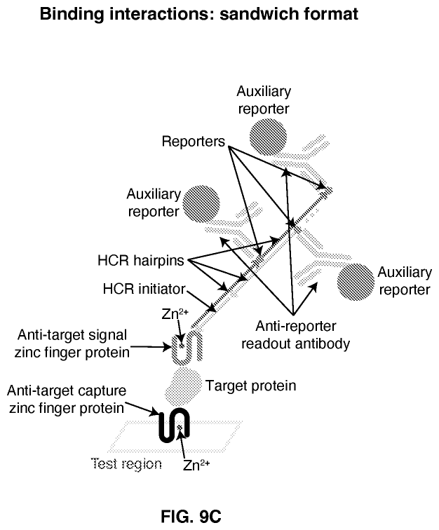

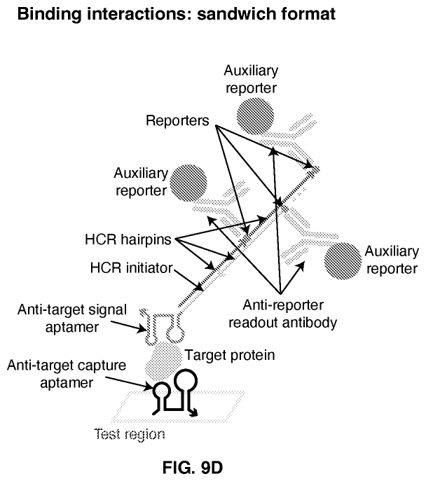

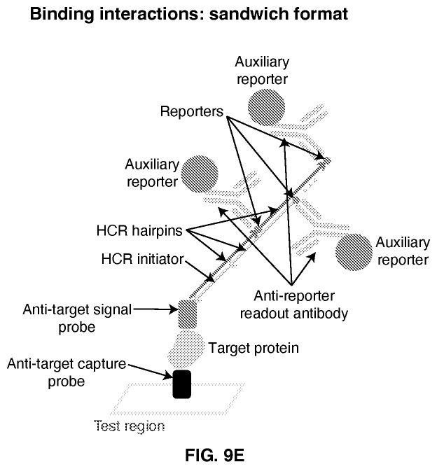

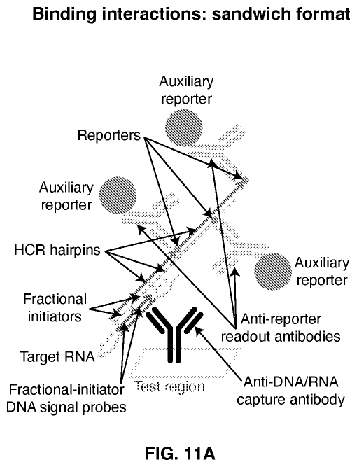

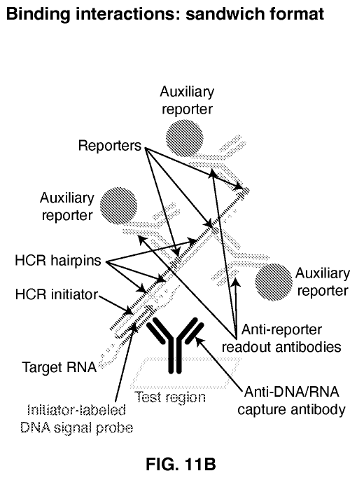

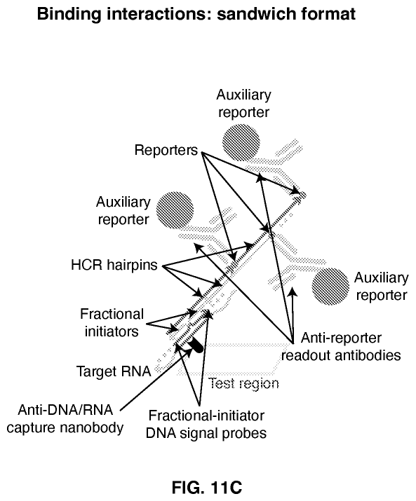

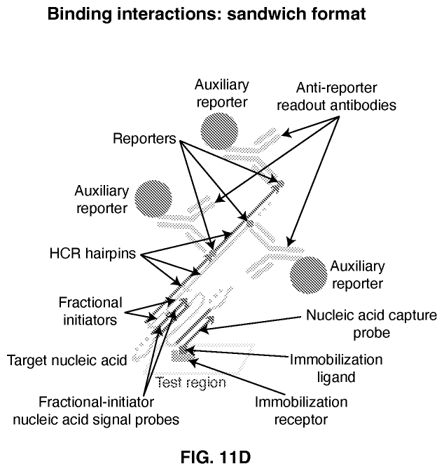

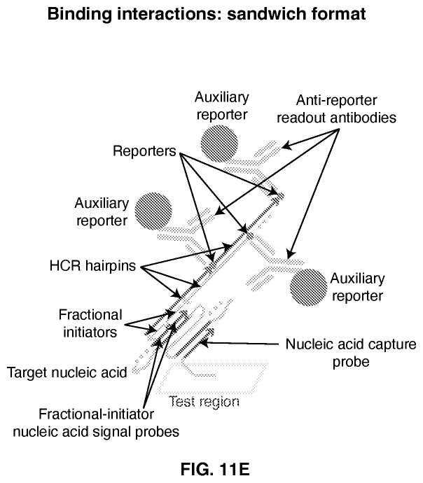

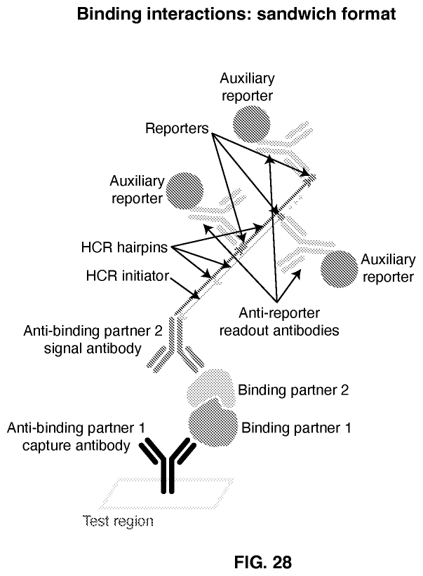

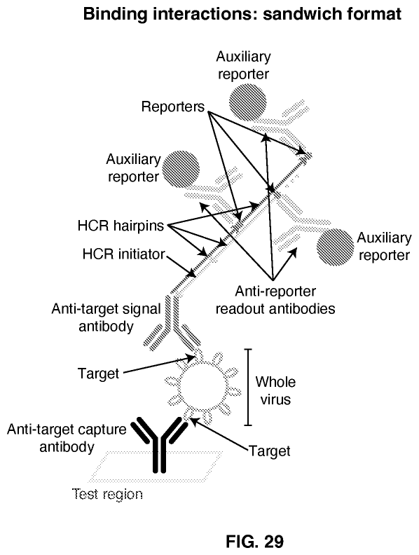

In some embodiments, the target comprises (for example, see FIGS. 7A-7J) a protein (for example, see FIGS. 9A-9E), a peptide, an amino acid, a nucleic acid (for example, see FIGS. 11A-11E), a DNA, an RNA, a chemically modified nucleic acid, a synthetic nucleic acid analog, a material capable of base-pairing, a chemical linker, a molecule, a biomolecule, a chemical, a toxin, a pathogen, a disease marker, a pollutant, a complex of any of the above (for example, see FIG. 28), a diseased cell, a cancer cell, a human cell, a eukaryotic cell, a prokaryote, a virus (for example, see FIG. 29), or any combination of the above. In some embodiments, the target is bacterial or viral in origin. Additional targets are discussed below.

In some embodiments, the sample comprises a bodily fluid, milk, mucus, water, urine, blood, serum, saliva, cells, tissue, tissue homogenate, a medical sample, an environmental sample, washwater from an agricultural crop, gray water, brown water, sea water, pollutants, polluted water, a waste sample, sewage, plants, bacteria, sludge, homogenized plants, organic matter, animal feed, an industrial sample, liquid fuel, solid fuel, propellants, a chemical solution, and/or chemicals. In some embodiments, the sample is obtained by swabbing, nasal swabbing, nasopharyngeal swabbing, anterior nasal swabbing, throat swabbing, or swabbing of a material, mixture, solution, or substance to be analyzed.

In some embodiments an HCR lateral flow device comprises a test region, a capture probe set comprising one or more capture probes wherein a capture probe comprises a target-binding domain and an immobilization domain, a signal probe set comprising one or more signal probes wherein a signal probe comprises a target-binding domain and an amplification domain, an HCR amplifier comprising two or more HCR hairpins wherein one or more HCR hairpins comprise one or more reporters, and optionally a readout probe set comprising one or more readout probes wherein a readout probe comprises a reporter-binding domain and one or more auxiliary reporters (for example, see FIGS. 7A-7J, 9A-9E, and 11A-11E).

In some embodiments, the device is configured so that the reagents automatically arrive in the test region in one or more successive stages without user interaction, generating an amplified signal in the test region if the target is present in the sample (for example, see FIGS. 8A-8D, 10A-10D, 12A-12D, 30A-30B, 31A-31B, 32A-32B). In some embodiments, reagents are automatically delivered using a multi-channel membrane.

In some embodiments, the automated delivery of reagents to the test and/or control regions in successive stages is achieved by using channels with different lengths, such that reagents traveling via longer channels arrive to the test and/or control region after reagents traveling via shorter channels (for example, see FIGS. 5, 6, 30A-30B, 31A-31B, 32A-32B, 33C, 33F, 73C, and 73F). In some embodiments, the automated delivery of reagents to the test and/or control regions in successive stages occurs by altering the speed that reagents flow through one or more channels. In some embodiments, the speed of reagent flow is altered using one or more speed additives that alter the rate of flow through the membrane, for example sucrose, fructose, trehalose, a sugar, dextran sulfate, water-insoluble ink, agarose, or another molecule. In some embodiments, the speed of reagent flow is altered using one or more speed modifications to the channel, for example a dissolvable barrier, a wax barrier, sponge, valves, pressurized paper, or gravitational orientation. In some embodiments, the speed of reagent flow is altered using membranes with one or more different material properties, including different porosities, pressures, densities, hydrophobicity, chemical composition, chemical reactivities, or other properties. In some embodiments, the speed of reagent flow is altered by using different geometries for different channels, including width, thickness, curvature, and length. In some embodiments, the automated delivery of reagents to the test and/or control region in successive stages is achieved by leveraging the properties of the reagents themselves, including size, charge, hydrophobicity, chemical reactivity, or other properties. In some embodiments, larger reagents flow through a membrane channel more slowly than smaller reagents, allowing for arrival of the smaller reagents at the test region and/or control region before the larger reagents. In some embodiments, any subset of the above approaches can be used in combination to speed up and/or slow down flow speed for one or more reagents so as to achieve automated delivery of one or more reagents to the test and/or control regions in a prescribed order. In some embodiments, an HCR lateral flow device comprises a wash channel that is used to deliver a wash solution to the test region to wash one or more reagents from the test region (for example, to remove a chemical denaturant, a probe, a reporter, and/or a molecule from the test region). In some embodiments, an HCR lateral flow device comprises a wash channel that enables an automated wash step at the test region. In some embodiments, a multi-channel HCR lateral flow devices enables staged automated delivery of one or more reagents to the test region and one or more staged automated wash steps of the test region.

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 7A-7J, 9A-9E, and 11A-11E). In some embodiments, the device is configured so that the reagents automatically arrive in the test region in one or more successive stages, generating an amplified signal in the test region if the target is present in the sample (for example, see FIGS. 8A-8D, 10A-10D, 12A-12D, 30A-30B, 31A-31B, 32A-32B).

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 7A-7J, 9A-9E, and 11A-11E). In some embodiments, the device is configured so that the reagents automatically arrive in the test region in a prescribed order, generating an amplified signal in the test region if the target is present in the sample (for example, see FIGS. 8A-8D, 10A-10D, 12A-12D, 30A-30B, 31A-31B, 32A-32B).

In some embodiments, reagents are automatically delivered to the test region in multiple successive stages using a multi-channel membrane (for example, see FIGS. 5, 6, 30A-30B, 31A-31B, 32A-32B, and 33A-33C, and 33F). In some embodiments, reagents are automatically delivered to the test region in successive stages using a multi-channel membrane in which channels of different lengths lead into a unified channel before reaching the test region (for example, see FIGS. 5, 6, 30A-30B, 31A-31B, 32A-32B, and 33C). In some embodiments, channels of differing lengths deliver reagents to the test region in order according to their lengths, with the reagents from the shortest channel arriving in the test region first, the reagents from the longest channel arriving in the test region last, and channels of intermediate lengths arriving in the test region at intermediate times (for example, see FIGS. 5, 6, 30A-30B, 31A-31B, 32A-32B, and 33C).

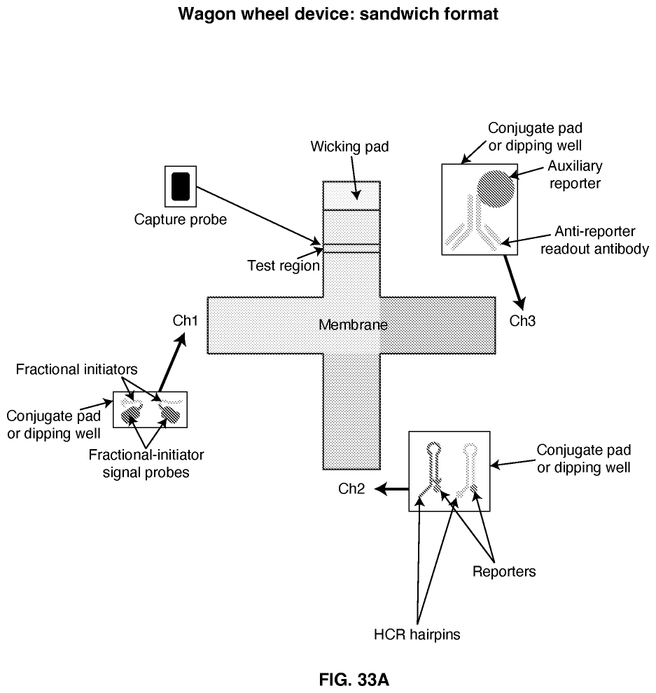

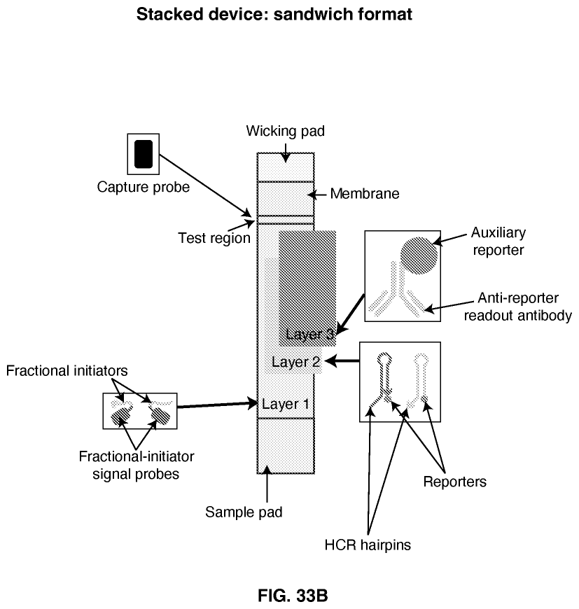

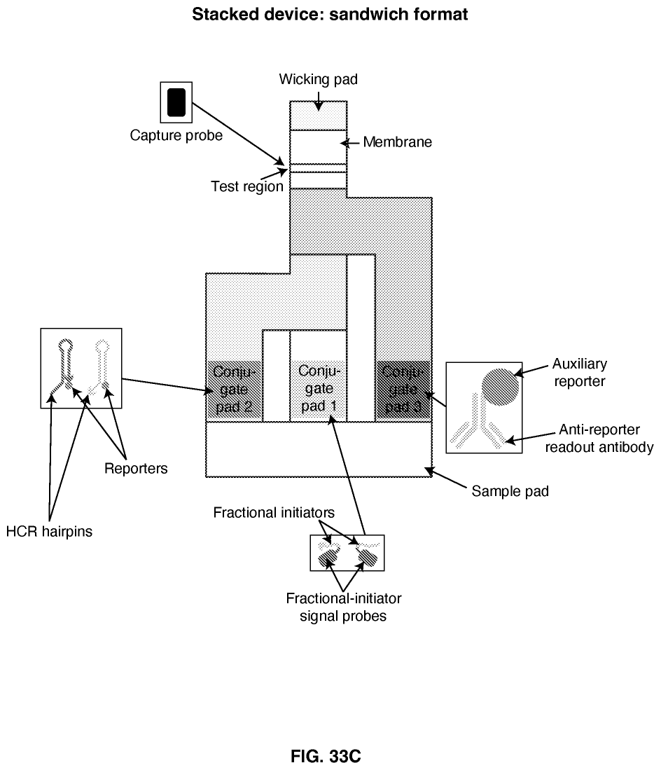

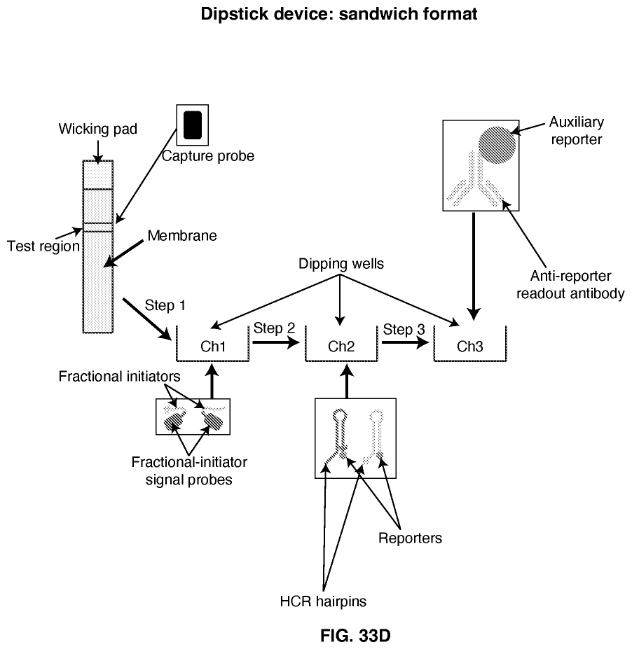

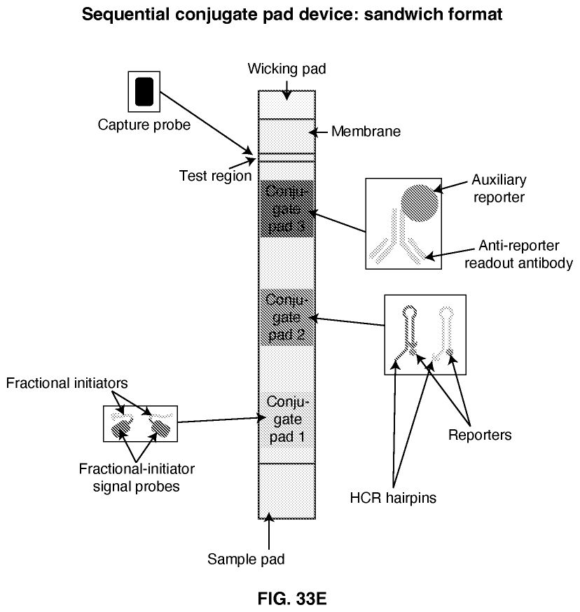

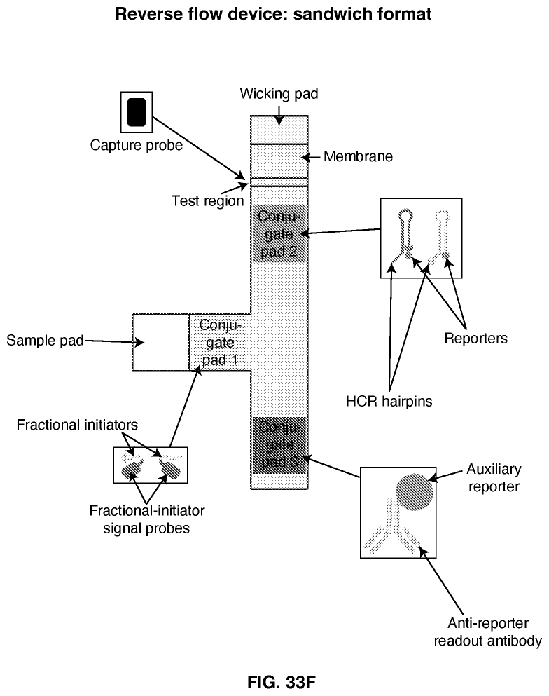

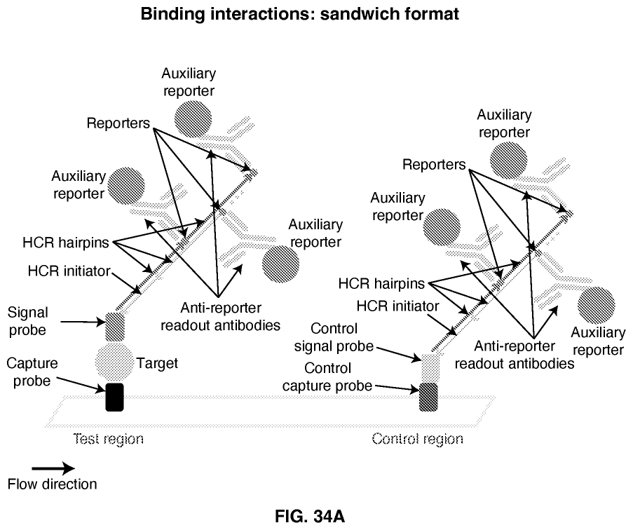

In some embodiments, channels are pre-treated with speed additives that create different flow speeds in different channels, allowing for delivery of reagents to the test region in successive stages. In some embodiments, the speed additives comprise sucrose, fructose, trehalose, a sugar, dextran sulfate, a dissolvable barrier, a wax barrier, water-insoluble ink, sponge, valves, pressurized paper, agarose, gravitational orientation, or another agent. In some embodiments, multiple channels are stacked on top of each other to form a multi-layer membrane (for example, see FIGS. 33B-33C).

In some embodiments, the sample arrives in different channels at different times, leading to successive delivery of reagents to the test region. In some embodiments, dissolvable barriers dissolve at different rates to introduce the sample into different channels at different times, leading to successive delivery of reagents to the test region. In some embodiments, different reagents migrate through the same or different channels at different rates leading to successive staged arrival times in the test region. For example, auxiliary-reporter-labeled readout probes labeled with bulky auxiliary reporters migrate through the membrane more slowly than reporter-labeled HCR hairpins labeled with small reporters, allowing for the readout probes to arrive in the test region after the HCR hairpins. In some embodiments, the size of different reagents affects their migration speeds through the same channel, allowing for successive delivery of reagents to the test region.

In some embodiments, a multi-channel membrane is configured so that reagents in each channel arrive in the test region (or another location) in a prescribed order (for example, see FIGS. 5, 6, 30A-30B, 31A-31B, 32A-32B, and 33A-33C, and 33F).

In some embodiments, reagents for each channel are dried onto separate conjugate pads (for example, see FIGS. 5, 30A-30B, 31A-31B, 32A-32B, 33A-33C, and 33E-33F). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user interaction. In some embodiments, draining of a first conjugate pad allows draining of a second conjugate pad, which in turn allows draining of a third conjugate pad, and so on (for example, see FIGS. 5, 30A-30B, 31A-31B, and 32A-32B). In some embodiments, draining of the first conjugate pad frees a unified channel to allow draining of a second conjugate pad, which in turn frees a unified channel to allow draining of a third conjugate pad, and so on (for example, see FIGS. 5, 30A-30B, 31A-31B, and 32A-32B).

In some embodiments, a membrane, a sample pad, a conjugate pad, and/or a wicking pad comprises nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.

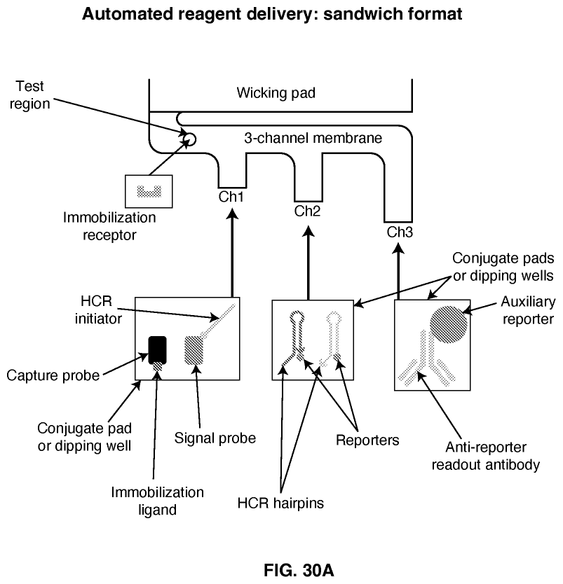

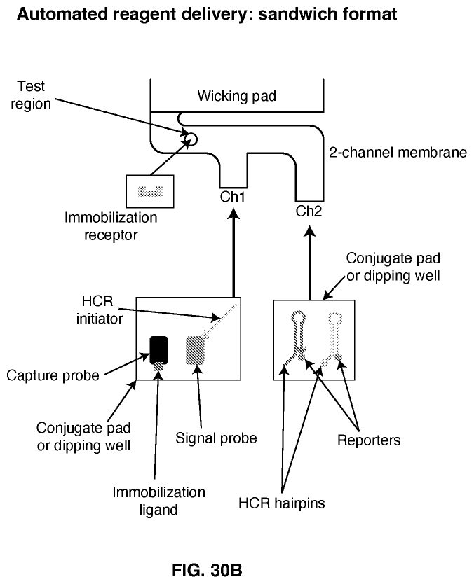

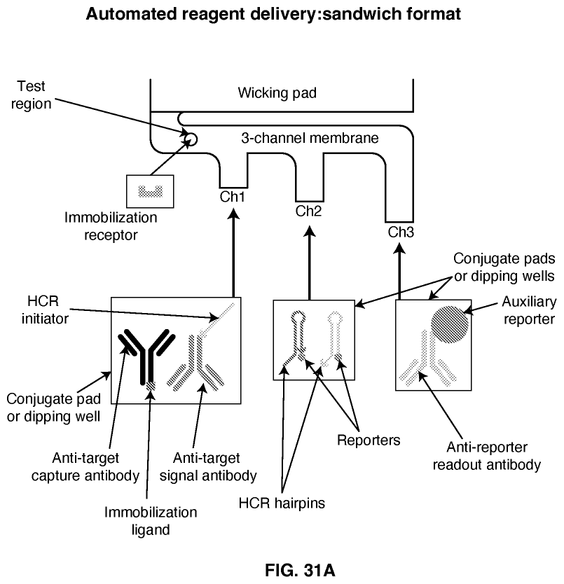

In some embodiments, reagents are automatically delivered to the test region in two successive stages using a two-channel membrane in which channels of different lengths lead into a unified channel before reaching the test region (for example, see FIGS. 30B, 31B, and 32B). In some embodiments, the signal probes bind the target and travel via the shorter channel (Channel 1) to reach the test region first where the target additionally binds capture probes that are pre-immobilized in the test region to form an immobilized probe/target sandwich (for example, see FIGS. 8C, 10C, and 12C). In some embodiments, the signal probes and capture probes bind the target and travel via the shorter channel (Channel 1) to reach the test region first, where the probe/target sandwich is immobilized via binding of immobilization-ligand-labeled capture probes to immobilization receptors pre-immobilized in the test region (for example, see FIGS. 8D, 10D, 12D, 30B, 31B, and 32B). In some embodiments, reporter-labeled HCR hairpins travel via a longer channel (Channel 2) and reach the test region next, where amplification domains on the signal probes trigger growth of tethered HCR amplification polymers decorated with reporters to generate an amplified signal in the test region (for example, see FIGS. 8C-8D, 10C-10D, 12C-12D, 30B, 31B, and 32B). In some embodiments, reagents for each channel are dried onto two separate conjugate pads (for example, see FIGS. 30B, 31B, and 32B). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user interaction. In some embodiments, draining of the first conjugate pad allows draining of the second conjugate pad. In some embodiments, draining of the first conjugate pad frees a unified channel to allow draining of the second conjugate pad. In some embodiments, reagents in the two channels arrive in the test region (or another location) in successive stages in a prescribed order due to channel length, channel geometry, speed additives, dissolvable barriers, or other conditions (for example, see FIGS. 30B, 31B, 32B).

In some embodiments, reagents are automatically delivered to the test region in three successive stages using a three-channel membrane in which channels of different lengths lead into a unified channel before reaching the test region (for example, see FIGS. 30A, 31A, and 32A). In some embodiments, the signal probes bind the target and travel via the shortest channel (Channel 1) to reach the test region first where the target additionally binds capture probes that are pre-immobilized in the test region to form an immobilized probe/target sandwich (for example, see FIGS. 8A, 10A, and 12A). In some embodiments, the signal probes and capture probes bind the target and travel via the shortest channel (Channel 1) to reach the test region first, where the probe/target sandwich is immobilized via binding of immobilization-ligand-labeled capture probes to immobilization receptors pre-immobilized in the test region (for example, see FIGS. 8B, 10B, 12B, 30A, 31A, and 32A). In some embodiments, reporter-labeled HCR hairpins travel via a channel of intermediate length (Channel 2) and reach the test region next, where amplification domains on the signal probes trigger growth of tethered HCR amplification polymers decorated with reporters (for example, see FIGS. 8A-8B, 10A-10B, 12A-12B, 30A, 31A, and 32A). In some embodiments, auxiliary-reporter-labeled readout probes travel via the longest channel (Channel 3) and arrive in the test region last, where they bind the reporters decorating the HCR amplification polymers to generate an amplified signal in the test region (for example, see FIGS. 8A-8B, 10A-10B, 12A-12B, 30A, 31A, and 32A). In some embodiments, reagents for each channel are dried onto separate conjugate pads (for example, see FIGS. 30A, 31A, and 32A). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of the first conjugate pad allows draining of the second conjugate pad, which in turn allows draining of the third conjugate pad, and so on. In some embodiments, draining of the first conjugate pad frees the unified channel to allow draining of the second conjugate pad, which in turn frees the unified channel to allow draining of the third conjugate pad. In some embodiments, reagents in the three channels arrive in the test region (or another location) in successive stages in a prescribed order due to channel length, channel geometry, speed additives, dissolvable barriers, or other conditions (for example, see FIGS. 30A, 31A, and 32A).

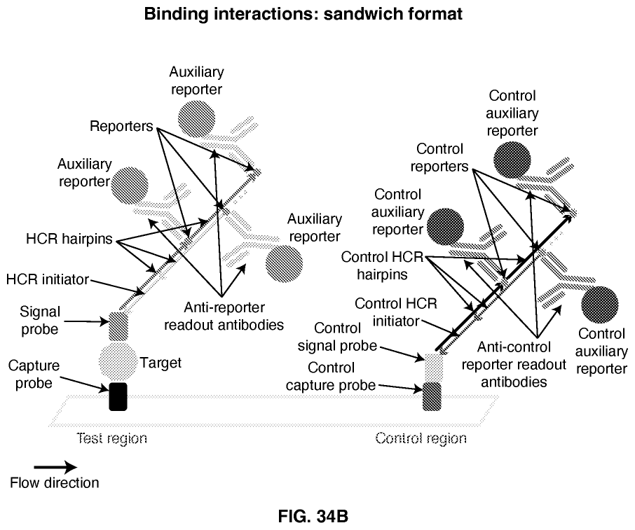

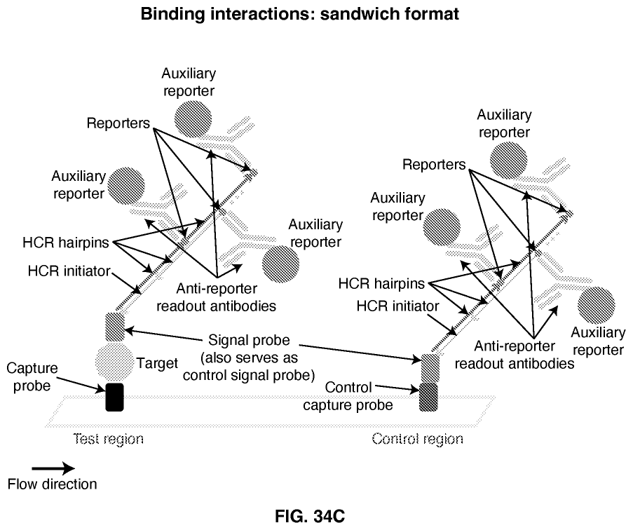

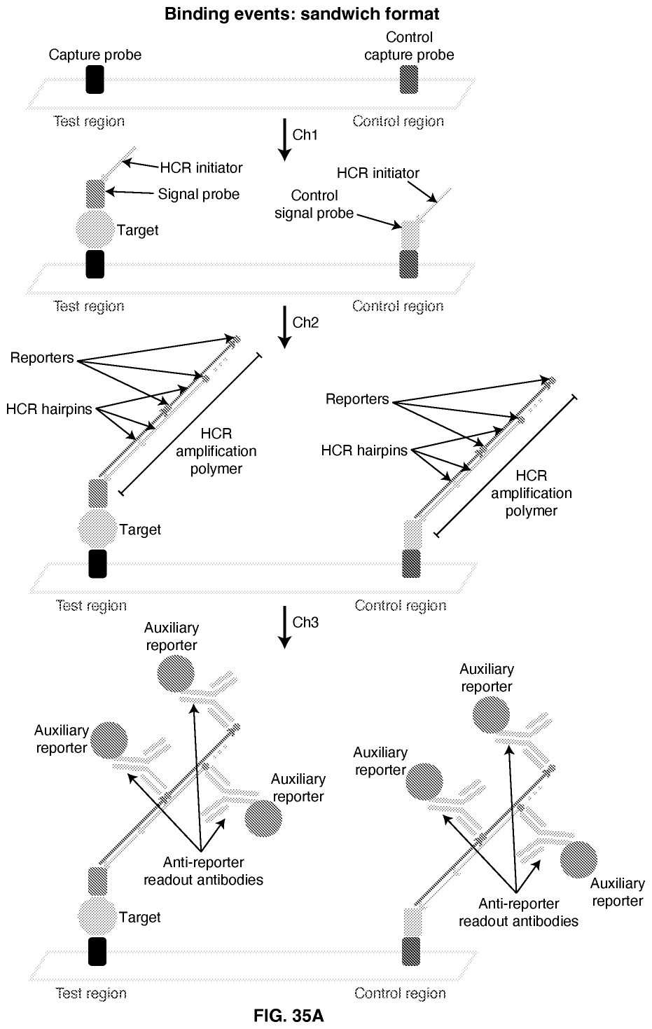

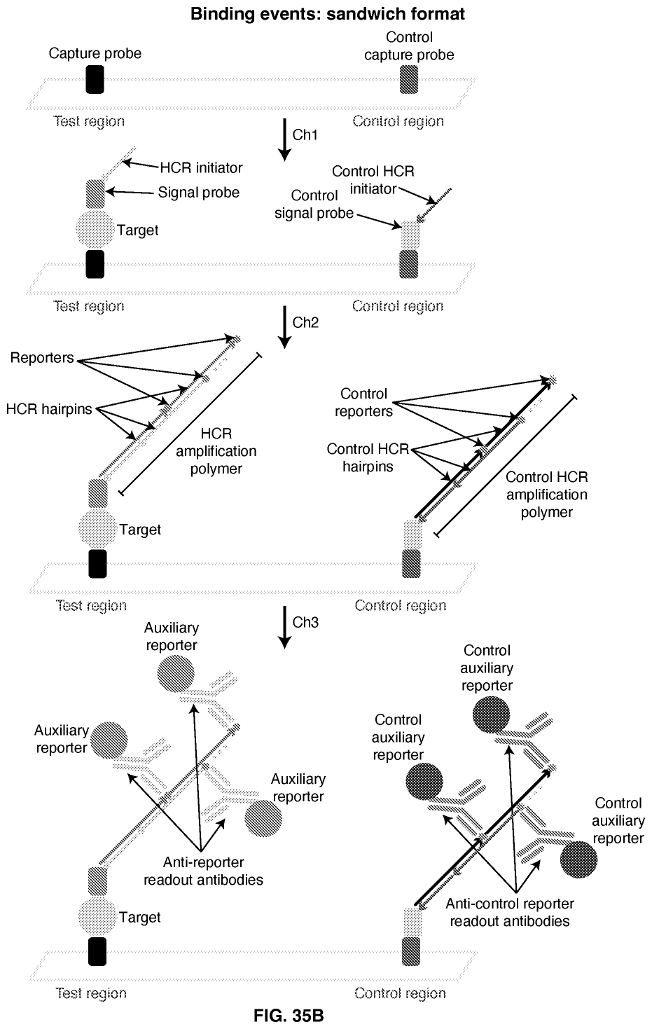

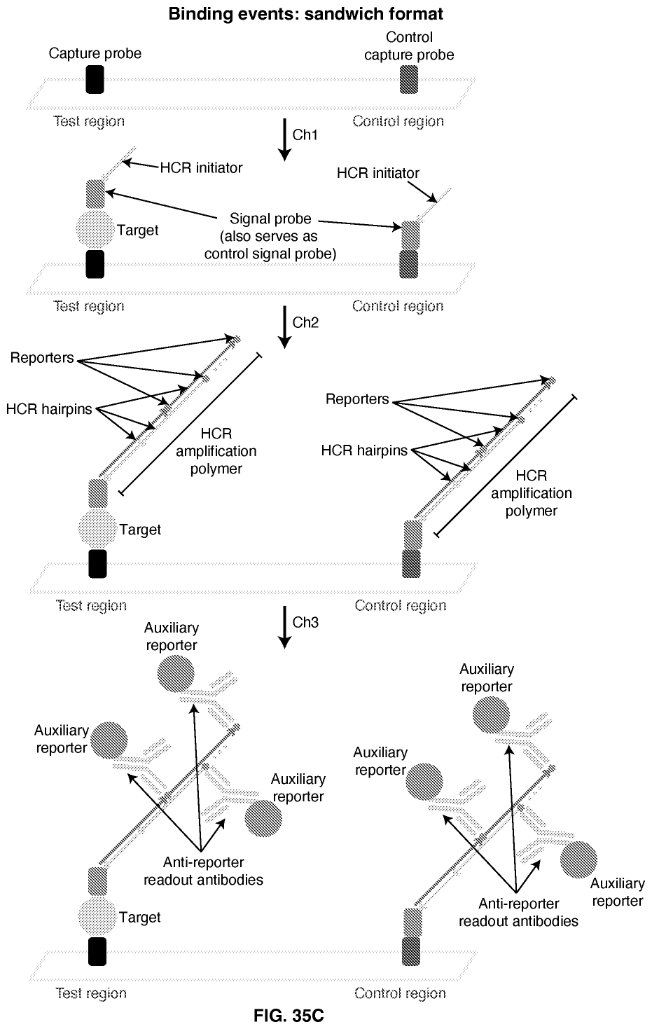

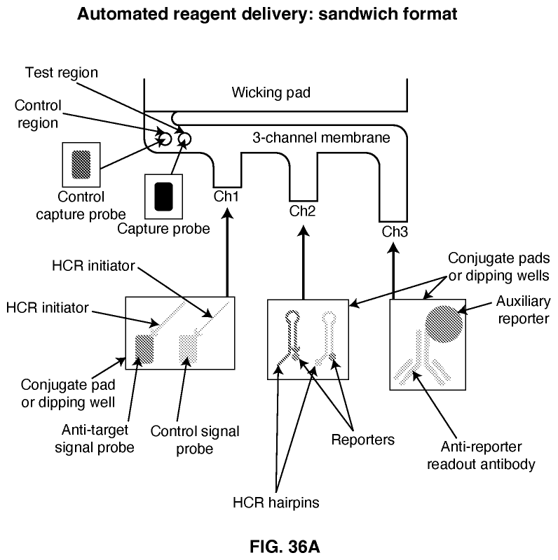

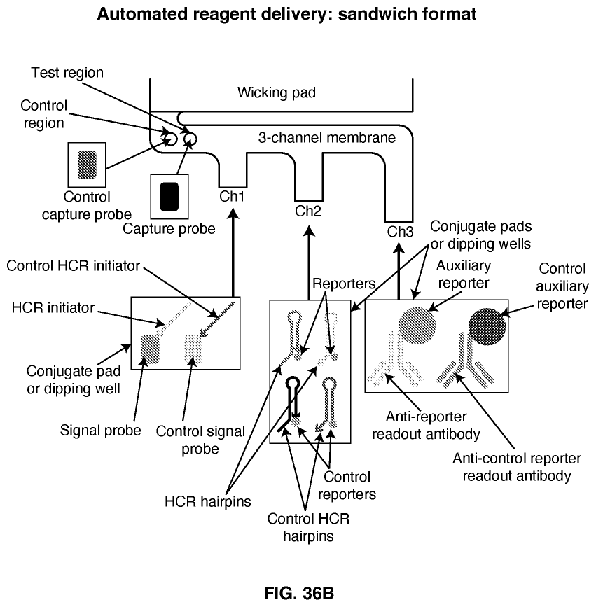

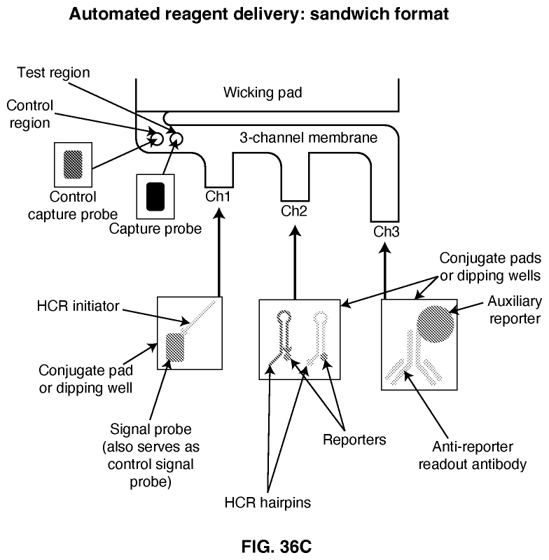

In some embodiments, the device comprises a control region (for example, see FIGS. 34A-34C, 35A-35C, and 36A-36C). In some embodiments, the device comprises a control capture probe set comprising one or more control capture probes, wherein a control capture probe comprises a control immobilization domain configured to directly or indirectly bind the control region and a control substrate configured to bind directly or indirectly to a control signal probe (for example, see FIGS. 34A-34C, 35A-35C, and 36A-36C). In some embodiments, the one or more control capture probes are pre-immobilized in the control region (for example, see FIGS. 35A-35C and 36A-36C). In some embodiments, the control capture domain of the one or more control capture probes comprises a control immobilization ligand that binds a control immobilization receptor that is pre-immobilized in the control region. In some embodiments, the control immobilization ligand is biotin and the control immobilization receptor is a biotin-binding molecule (for example, polystreptavidin R (PR), streptavidin, avidin, NeutrAvidin, CaptAvidin, or another biotin-binding molecule), or the control immobilization ligand is a hapten and the control immobilization receptor is an anti-hapten antibody, or the control immobilization ligand is an anti-hapten antibody and the control immobilization receptor is a hapten, or the control immobilization ligand is a nucleic acid and the control immobilization receptor is a nucleic acid, or the control immobilization ligand is an antigen and the control immobilization receptor is an anti-antigen antibody, or the control immobilization ligand is a molecule that is recognized by a control immobilization receptor that is a zinc finger, or the control immobilization ligand is another molecule or complex that can be recognized by a control immobilization receptor. In some embodiments, a control capture probe comprises a primary control capture probe comprising a target-binding domain and a secondary control capture probe comprising a control immobilization domain configured to bind directly or indirectly to the control region wherein the secondary control capture probe is configured to bind the primary control capture probe directly or indirectly (for example, a primary control capture probe that is a primary antibody and a secondary control capture probe that is a secondary antibody configured to bind the primary antibody). In some embodiments, the primary control capture probe comprises a target-binding domain without an immobilization domain. In some embodiments, the secondary control capture probe comprises an immobilization domain without a target-binding domain.

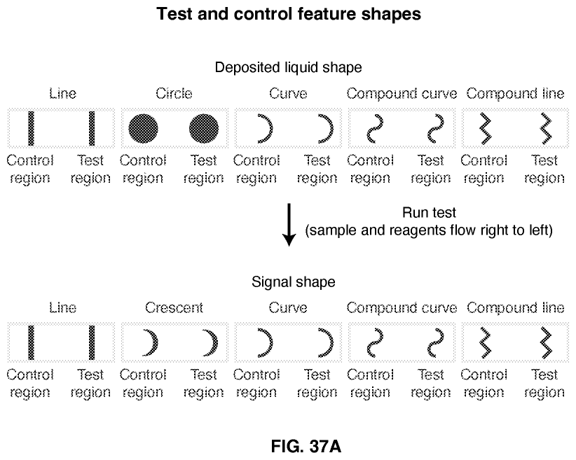

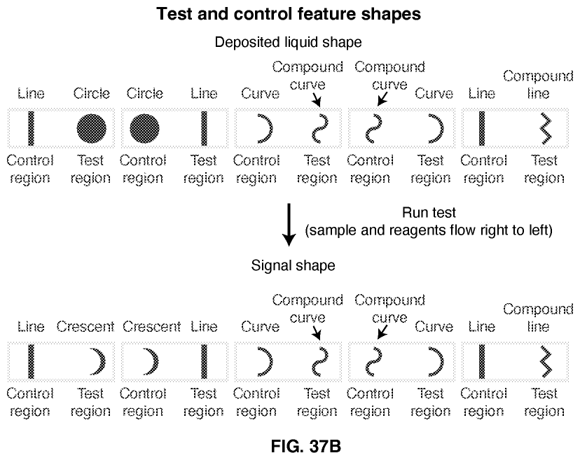

In some embodiments, the control capture probe set is pre-immobilized in the control region in a control feature shape that is a straight line, a curve, a zigzag, a compound line, a compound curve, a cross, a filled circle, an open circle, a filled shape, an open shape, or another feature shape (for example, see FIGS. 37A-37B). In some embodiments, the control capture probe set is immobilized in the control region by binding a control immobilization receptor that is pre-immobilized in the control region in a control feature shape that is a straight line, a curve, a zigzag, a compound line, a compound curve, a cross, a filled circle, an open circle, a filled shape, an open shape, or another feature shape. In some embodiments, the amplified control signal generated in the control region takes the form of a straight line, a curve, or another feature shape (for example, see FIG. 37A). In some embodiments, the control feature shape is the same as the test feature shape. In some embodiments, the control feature shape is different from the test feature shape (for example, see FIG. 37B).

In some embodiments, the device comprises a control HCR amplifier comprising two or more control HCR hairpins (for example, see FIGS. 34B, 35B, 36B). In some embodiments, one or more of the control HCR hairpins are labeled with one or more control reporters. In some embodiments, the control HCR hairpins are configured not to polymerize in the absence of a control HCR initiator. In some embodiments, control HCR hairpins are configured such that detection of a control HCR initiator triggers the control HCR hairpins to polymerize to form a tethered control HCR amplification polymer decorated with control reporters (for example, see FIGS. 34B and 35B). In some embodiments, the control HCR amplifier is the same as the HCR amplifier (for example, see FIGS. 34A, 34C, 35A, 35C, 36A, 36C).

In some embodiments, the device comprises a control signal probe set comprising one or more control signal probes, wherein a control signal probe comprises a control-substrate-binding domain configured to bind directly or indirectly to the substrate on a control capture probe, and an amplification domain that is configured to directly or indirectly trigger HCR signal amplification by initiating a chain reaction in which the control HCR hairpins self-assemble to form a tethered control HCR amplification polymer decorated with control reporters (for example, see FIGS. 34A-34B, 35A-35B, and 36A-36B). In some embodiments, the control signal probe set comprises: a) one or more control-initiator-labeled probes wherein a control-initiator-labeled probe comprises a control-substrate-binding domain and a control amplification domain comprising one or more control HCR initiators, or b) one or more control probe units, each comprising two or more control-fractional-initiator probes, wherein a control-fractional-initiator probe comprises a control-substrate-binding domain and a control amplification domain comprising a control fractional initiator. In some embodiments, an individual control-fractional-initiator probe that binds non-specifically will not trigger HCR, but specific binding of the probes within a control probe unit to adjacent cognate binding sites on the control substrate colocalizes a full control HCR initiator capable of triggering HCR. In some embodiments, a control signal probe comprises a primary control signal probe comprising a control-substrate-binding domain and a secondary control signal probe comprising an amplification domain wherein the secondary control signal probe is configured to bind the primary control signal probe directly or indirectly (for example, a primary control signal probe that is a primary antibody and a secondary control signal probe that is a secondary antibody configured to bind the primary antibody). In some embodiments, the amplification domain on the control signal probe set is that same as the amplification domain on the signal probe set. In some embodiments, the signal probe set and the control signal probe set are the same (for example, see FIGS. 34C, 35C, and 36C). For example, in some embodiments, the signal probe set comprises a primary anti-target antibody and the control capture probe set comprises a secondary antibody configured to bind the primary anti-target antibody. In some embodiments, the primary control signal probe comprises a control-substrate-binding domain without an amplification domain. In some embodiments, the secondary control signal probe comprises an amplification domain without a control-substrate-binding domain.

In some embodiments, the device comprises a control readout probe set comprising one or more control readout probes, wherein a control readout probe comprises a control-reporter-binding domain configured to bind directly or indirectly to a control reporter on a control HCR amplification polymer (for example, see FIGS. 34B, 35B, and 36B). In some embodiments, control readout probes comprise a control auxiliary reporter wherein the control auxiliary reporter contributes directly or indirectly to the generation of an amplified control signal in the control region. In some embodiments, the amplified control signal generated by the control auxiliary reporters on the control readout probes is visible to the human eye. In some embodiments, a control readout probe comprises a primary control readout probe comprising a control-reporter-binding domain and a secondary control readout probe comprising a control auxiliary reporter wherein the secondary control readout probe is configured to bind the primary control readout probe directly or indirectly (for example, a primary control readout probe that is a primary antibody and a secondary control readout probe that is a secondary antibody configured to bind the primary antibody). In some embodiments, the control readout probe set is the same as the readout probe set (for example, see FIGS. 34A, 34C, 35A, 35C, 36A, 36C). In some embodiments, the primary control readout probe comprises a control-reporter-binding domain without a control auxiliary reporter. In some embodiments, the secondary control readout probe comprises a control auxiliary reporter without a control-reporter-binding domain.

In some embodiments, the control capture probe set binds the control signal probe set to form a control complex (for example, see FIGS. 34A-34C, 35A-35C, and 36A-36C). In some embodiments, the control complex is immobilized through the control capture probe set in the control region (for example, see FIGS. 34A-34C and 35A-35C). In some embodiments, after the control complex is immobilized in the control region, the control amplification domains on the control signal probes trigger the self-assembly of control-reporter-labeled control HCR hairpins into tethered control HCR amplification polymers decorated with control reporters (for example, see FIGS. 34A-34C and 35A-35C). In some embodiments, the control reporters directly or indirectly lead to generation of an amplified control signal in the control region. In some embodiments, control-auxiliary-reporter-labeled control readout probes that comprise one or more control auxiliary reporters bind the control reporters decorating the control HCR amplification polymers immobilized in the control region to directly or indirectly lead to generation of an amplified control signal in the control region (for example, see FIGS. 34B and 35B). In some embodiments, the control readout probe set is the same as the readout probe set (for example, see FIGS. 34A, 34C, 35A, and 35C).

In some embodiments, the amplified control signal is visible to the human eye. In some embodiments, the control signal is read using a fluorescence microscope, a fluorescence scanner, a camera, a mobile phone camera, a mass spectrometer, a mass spectrometry microscope, a radioactive scanner, or another instrument suitable for detecting control reporters.

In some embodiments, a control reporter and/or a control auxiliary reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a control reporter and/or control auxiliary reporter is a hapten, a ligand, digoxigenin (DIG), fluorescein isothiocyanate (FITC), a fluorophore, biotin, dinitrophenol, aniline, or another molecule or complex that can be recognized by a binding partner to facilitate generation of a signal. In some embodiments, the control reporters and/or control auxiliary reporters decorating the control HCR amplification polymer directly or indirectly mediate catalytic reporter deposition (CARD) to generate an amplified control signal in the vicinity of the control HCR amplification polymer within the control region. In some embodiments, CARD is mediated by an enzyme.

In some embodiments, the control region is downstream of the test region so that the sample and reagents flow through the test region before reaching the control region (for example, see FIGS. 34A-34C and 36A-36C). In some embodiments, if the sample and reagents successfully flow through the control region, the control capture probe set binds to and immobilizes the control signal probe set in the test region, the control amplification domains on the control signal probe set trigger the self-assembly of tethered control HCR amplification polymers decorated with control reporters to directly or indirectly generate an amplified control signal in the control region, and optionally the control-auxiliary-reporter-labeled control readout probe set binds the control reporters to directly or indirectly generate an amplified control signal in the control region (for example, see FIG. 35B). In some embodiments, the test produces a positive result if both the test region and the control region produce detectable signals, for example visible signals. In some embodiments, the test produces a negative result if signal is observed in the control region but not in the test region. In some embodiments, absence of signal in the control region is an indication that the test did not function properly. In some embodiments, the control region is upstream of the test region.

In some embodiments, the capture probe set, control capture probe set, signal probe set, and control signal probe set are all dried down in the same conjugate pad and/or flow to the test region and control region through the same membrane channel. In some embodiments, the capture probe set is pre-immobilized in the test region and the control capture probe set is pre-immobilized in the control region (for example, see FIGS. 36A-36B). In some embodiments, the signal probe set and control signal probe set are dried down in the same conjugate pad and/or flow to the test region and the control region through the same membrane channel (for example, see FIGS. 36A-36B).

In some embodiments, the signal probe set and the control signal probe set are the same (for example, see FIG. 36C). In some embodiments, the HCR amplifier and control HCR amplifier are dried down in the same conjugate pad and/or flow to the test region and the flow region through the same membrane channel (for example, see FIG. 36B). In some embodiments, the HCR amplifier and the control amplifier are the same (for example, see FIGS. 36A and 36C). In some embodiments, the readout probe set and control readout probe set are dried down in the same conjugate pad and/or flow to the test region and the control region through the same membrane channel (for example, see FIG. 36B). In some embodiments, the readout probe set and the control readout probe set are the same (for example, see FIGS. 36A and 36C).

In some embodiments, the control HCR amplifiers and control readout probe set used to directly or indirectly generate an amplified control signal in the control region are the same as those used to generate an amplified signal in the test region (for example, see FIGS. 34A, 34C, 35A, 35C, 36A, and 36C). For example, the same HCR amplifier comprising the same HCR hairpins labeled with the same reporters, and optionally the same readout probe set comprising the same auxiliary reporters can be used to directly or indirectly generate an amplified signal in the test region and an amplified control signal in the control region (for example, see FIGS. 34A, 34C, 35A, 35C, 36A, and 36C).

In some embodiments, the control signal can be unamplified instead of amplified. For example, a control signal probe set can comprise one or more control signal probes that omit the amplification domain and instead are directly labeled with one or more control reporters. In some embodiments, immobilization of control-reporter-labeled control signal probes in the control region directly or indirectly generates an unamplified control signal in the control region.

In some embodiments an HCR lateral flow device comprises a test region, a capture probe set comprising one or more capture probes wherein a capture probe comprises a target-binding domain and an immobilization domain, a signal probe set comprising one or more signal probes wherein a signal probe comprises a target-binding domain and an amplification domain, an HCR amplifier comprising two or more HCR hairpins wherein one or more HCR hairpins comprise one or more reporters, and optionally a readout probe set comprising one or more readout probes wherein a readout probe comprises a reporter-binding domain and one or more auxiliary reporters (for example, see FIGS. 34A-34C, 35A-35C, and 36A-36C).

In some embodiments the device further comprises a control region, a control capture probe set comprising one or more control capture probes wherein a control capture probe comprises a control substrate domain and a control immobilization domain, a control signal probe set comprising one or more control signal probes wherein a control signal probe comprises a control-substrate-binding domain and a control amplification domain, a control HCR amplifier comprising two or more control HCR hairpins wherein one or more control HCR hairpins comprise one or more control reporters, and optionally a control readout probe set comprising one or more control readout probes wherein a control readout probe comprises a control-reporter-binding domain and one or more control auxiliary reporters (for example, see FIGS. 34B, 35B, and 36B).

In some embodiments, the device is configured so that the reagents automatically arrive in the test region and the control region in one or more successive stages, generating an amplified signal in the test region if the target is present in the sample, and generating an amplified control signal in the control region if the sample and reagents flow through the device properly (for example, see FIGS. 36A-36C).

In some embodiments, the amplification domains on the signal probe set and the control signal probe set are the same (for example, see FIGS. 34A, 34C, 35A, 35C, 36A, and 36C). In some embodiments, the HCR amplifier and the control HCR amplifier are the same (for example, see FIGS. 34A, 34C, 35A, 35C, 36A, and 36C). In some embodiments, the optional readout probe set and optional control readout probe set are the same (for example, see FIGS. 34A, 34C, 35A, 35C, 36A, and 36C). In some embodiments, the signal probe set and the control signal probe set are the same (for example, see FIGS. 34C, 35C, 36C.)

In some embodiments the device comprises a test region, a control region, a capture probe set comprising one or more capture probes wherein a capture probe comprises a target-binding domain and an immobilization domain, a signal probe set comprising one or more signal probes wherein a signal probe comprises a target-binding domain and an amplification domain, a control capture probe set comprising one or more control capture probes wherein a control capture probe comprises a control substrate domain and a control immobilization domain, a control signal probe set comprising one or more control signal probes wherein a control signal probe comprises a control-substrate-binding domain and a control amplification domain that is the same as the amplification domain for the signal probes, an HCR amplifier comprising two or more HCR hairpins wherein one or more HCR hairpins comprise one or more reporters, and optionally a readout probe set comprising one or more readout probes wherein a readout probe comprises a reporter-binding domain and one or more auxiliary reporters (for example, see FIGS. 34A, 35A, 36A). In some embodiments, the control signal probe set is the same as the signal probe set (for example, see FIGS. 34C, 35C, 36C). In some embodiments, the device is configured so that the reagents automatically arrive in the test region and the control region in one or more successive stages (for example, see FIG. 36A, 36C), generating an amplified signal in the test region if the target is present in the sample, and generating an amplified control signal in the control region if the sample and reagents flow through the device properly.

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 34A-34C, 35A-35C, and 36A-36C). In some embodiments the device further comprises a control region, a control capture probe set, a control signal probe set, a control HCR amplifier comprising one or more control reporters, and optionally a control readout probe set comprising one or more control auxiliary reporters (for example, see FIGS. 34B, 35B, and 36B). In some embodiments, the device is configured so that the reagents automatically arrive in the test region and the control region in one or more successive stages (for example, see FIG. 36A-36C), generating an amplified signal in the test region if the target is present in the sample, and generating an amplified control signal in the control region if the sample and reagents flow through the device properly. In some embodiments, the amplification domains on the signal probe set and the control signal probe set are the same (for example, see FIGS. 34A, 34C, 35A, 35C, 36A, and 36C). In some embodiments, the HCR amplifier and the control HCR amplifier are the same (for example, see FIGS. 34A, 34C, 35A, 35C, 36A, and 36C). In some embodiments, the optional readout probe set and the optional control readout probe set are the same (for example, see FIGS. 34A, 34C, 35A, 35C, 36A, and 36C). In some embodiments, the signal probe set and the control signal probe set are the same (for example, see FIGS. 34C, 35C, and 36C).

In some embodiments an HCR lateral flow device comprises a test region, a control region, a capture probe set, a signal probe set, a control capture probe set, a control signal probe set, an HCR amplifier comprising one or more reporters, and optionally a readout probe set comprising one or more auxiliary reporters (for example, see FIGS. 34A, 35A, 36A). In some embodiments, the device is configured so that the reagents automatically arrive in the test region and the control region in one or more successive stages (for example, see FIGS. 36A and 36C), generating an amplified signal in the test region if the target is present in the sample, and generating an amplified control signal in the control region if the sample and reagents flow through the device properly. In some embodiments, the control signal probe set is the signal probe set (for example, see FIGS. 34C, 35C, 36C).

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 34A-34C, 35A-35C, and 36A-36C). In some embodiments the device further comprises a control region, a control capture probe set, a control signal probe set, a control HCR amplifier comprising one or more control reporters, and optionally a control readout probe set comprising one or more control auxiliary reporters (for example, see FIGS. 34B, 35B, and 36B). In some embodiments, the device is configured so that the reagents automatically arrive in the test region and the control region in a prescribed order (for example, see FIGS. 36A-36C), generating an amplified signal in the test region if the target is present in the sample, and generating an amplified control signal in the control region if the sample and reagents flow through the device properly. In some embodiments, the signal probe set and the control signal probe set are the same (for example, see FIGS. 34C, 35C, and 36C). In some embodiments, the amplification domains on the signal probe set and the control signal probe set are the same (for example, see FIGS. 34A, 34C, 35A, 35C, 36A, and 36C). In some embodiments, the HCR amplifier and the control HCR amplifier are the same (for example, see FIGS. 34A, 34C, 35A, 35C, 36A, and 36C). In some embodiments, the optional readout probe set and the optional control readout probe set are the same (for example, see FIGS. 34A, 36C, 35A, 35C, 36A, and 36C).

In some embodiments an HCR lateral flow device comprises a test region, a control region, a capture probe set, a signal probe set, a control capture probe set, a control signal probe set, an HCR amplifier comprising one or more reporters, and optionally a readout probe set comprising one or more auxiliary reporters (for example, see FIGS. 34A, 35A, 36A). In some embodiments, the control signal probe set is the signal probe set (for example, see FIGS. 34C, 35C, 36C). In some embodiments, the device is configured so that the reagents automatically arrive in the test region and the control region in a prescribed order (for example, see FIGS. 36A and 36C), generating an amplified signal in the test region if the target is present in the sample, and generating an amplified control signal in the control region if the sample and reagents flow through the device properly.

In some embodiments an HCR lateral flow device comprises a test region, a control region, a capture probe set, a signal probe set, a control capture probe set, an HCR amplifier comprising one or more reporters, and optionally a readout probe set comprising one or more auxiliary reporters (for example, see FIGS. 34C, 35C, and 36C). In some embodiments, the device is configured so that the reagents automatically arrive in the test region and the control region in a prescribed order (for example, see FIGS. 36A and 36C), generating an amplified signal in the test region if the target is present in the sample, and generating an amplified control signal in the control region if the sample and reagents flow through the device properly.

In some embodiments the device comprises a sample pad, one or more conjugate pads, a membrane, a test region, an optional control region, and a wicking pad (for example, see FIGS. 5, 33B-33C, 33E-33F, 73B-73C, and 73E-73F). In some embodiments the device comprises one or more sample pads, one or more conjugate pads, one or more membranes, one or more test regions, optionally one or more control regions, and/or one or more wicking pads. In some embodiments, the device has a half-strip assay format and comprises a membrane, a test region, an optional control region, and a wicking pad (for example, see FIGS. 6, 33D, and 73D).

In some embodiments, the device comprises a folding card geometry (for example, see FIG. 5). In some embodiments, one page of the card contains a sample pad and one or more conjugate pads. In some embodiments, the other page of the card contains a membrane comprising one or more channels, a test region, an optional control region, and a wicking pad. In some embodiments, the wicking pad absorbs liquid to induce continued capillary flow through the channels after the sample reaches the wicking pad. In some embodiments, one page is functionalized with prongs that disconnect the sample pad from the one or more conjugate pads upon folding the card. In some embodiments, disconnecting the sample pad from the one or more conjugate pads limits the volume that flows from the one or more conjugate pads and prevents flow between conjugate pads.

In some embodiments, to perform a test, the user adds a sample to a tube containing prep buffer, adds the prepped sample to the sample pad, and closes the card to create contact between the membrane and the conjugate pads. After a prescribed amount of time, the user reads the result.

In some embodiments, a device comprises a test region, an optional control region, and a multi-channel membrane configured in a comb shape (for example, see FIGS. 5, 6, 30A-30B, 31A-31B, 32A-32B), wherein the membrane comprises multiple reagent channels that successively enter a unified channel at different locations after which the unified channel leads in succession to the test region, the optional control region, and an optional wicking pad. In some embodiments, one or more of the reagent channels contacts one or more conjugate pads (for example, see FIGS. 5, 30A-30B, 31A-31B, 32A-32B), dipping wells (for example, see FIG. 6), or other entities containing reagents. In some embodiments, reagents automatically arrive at the test region and the optional control region in a prescribed order without user interaction. In some embodiments, reagents that flow through the reagent channel that offers the shortest path to the test region and optional control region arrive first, reagents that flow through the reagent channel that offers the longest path to the test region and optional control region arrive last, and reagents that flow through reagent channels that offer paths of intermediate length to the test region and optional control region arrive at intermediate times according to the path length. In some embodiments, a reagent is introduced to a reagent channel by wetting a conjugate pad in contact with the channel to resuspend reagents that have been dried down in the conjugate pad. In some embodiments, a reagent is introduced to a reagent channel by dipping into a well containing reagents in solution (for example, see FIG. 6). In some embodiments, some reagent channels have conjugate pads and other reagent channels do not have conjugate pads. In some embodiments, some conjugate pads do not contain dried down reagents. In some embodiments, the reagents will flow through the test and optional control regions based on the order that the reagent channels enter the unified channel and/or based on the length, width, or thickness of each reagent channel before it enters the unified channel and/or based on the speed additives, membrane modifications, and/or membrane material properties in each channel.

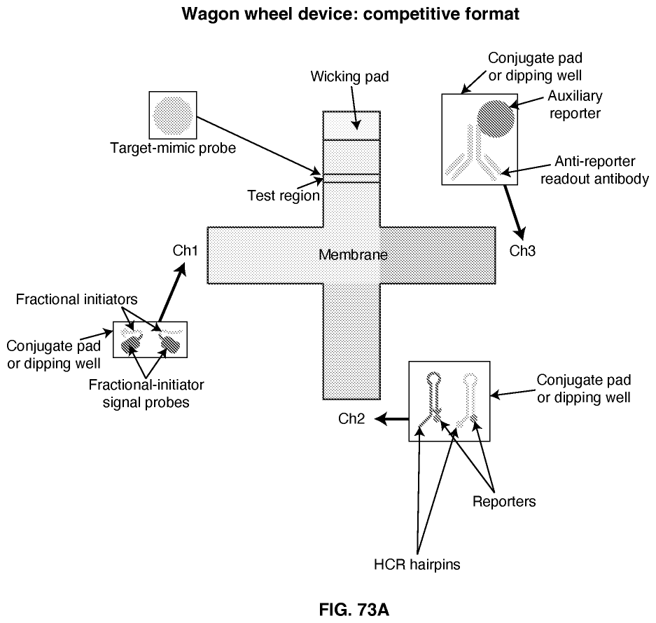

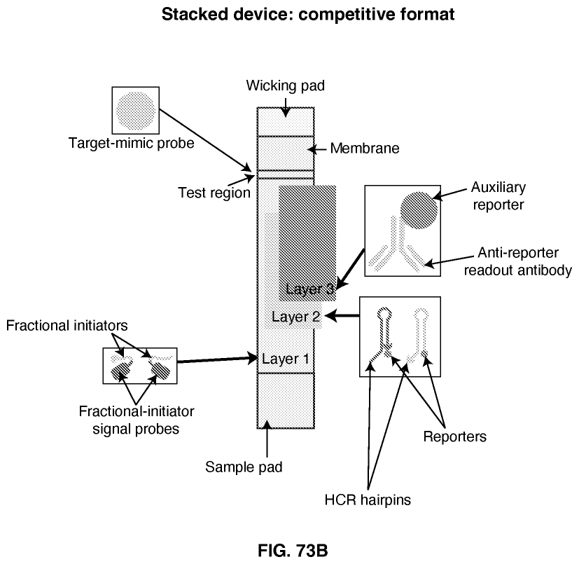

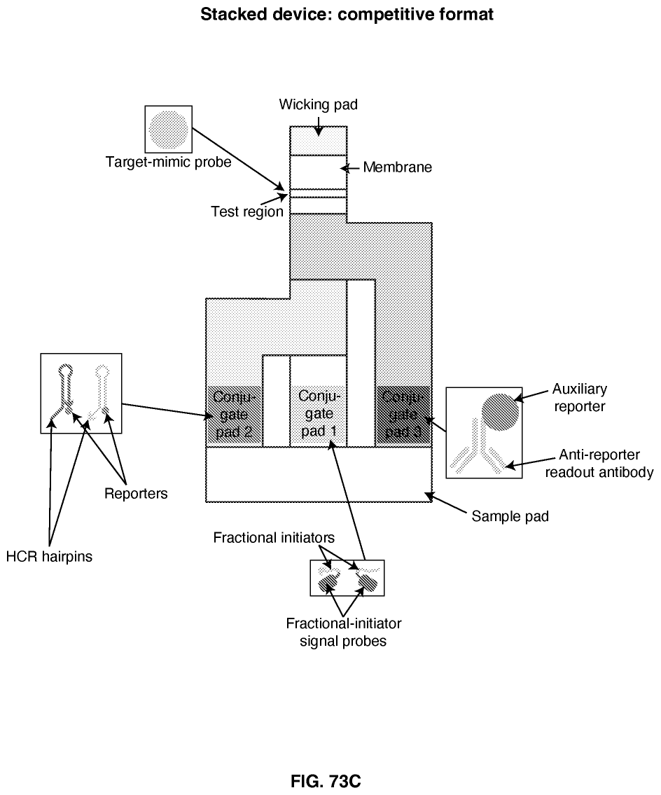

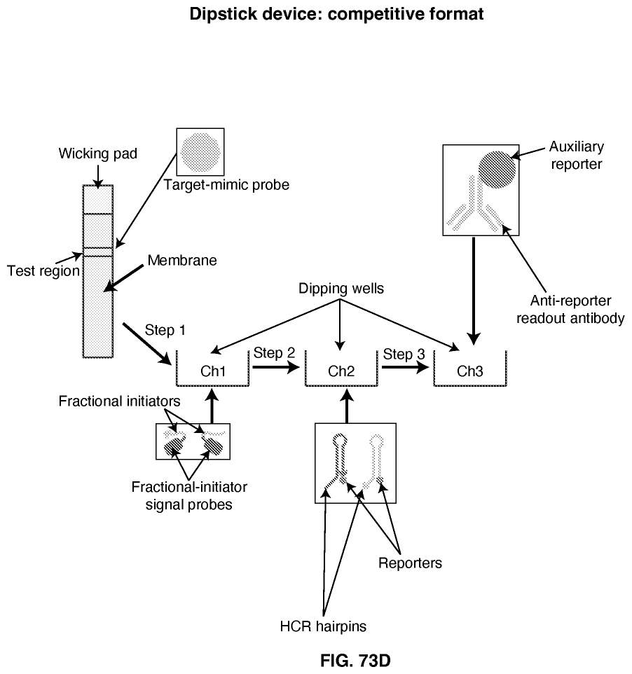

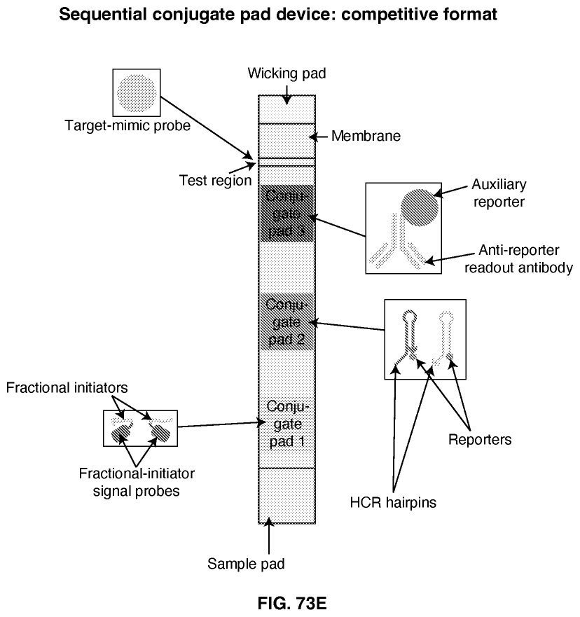

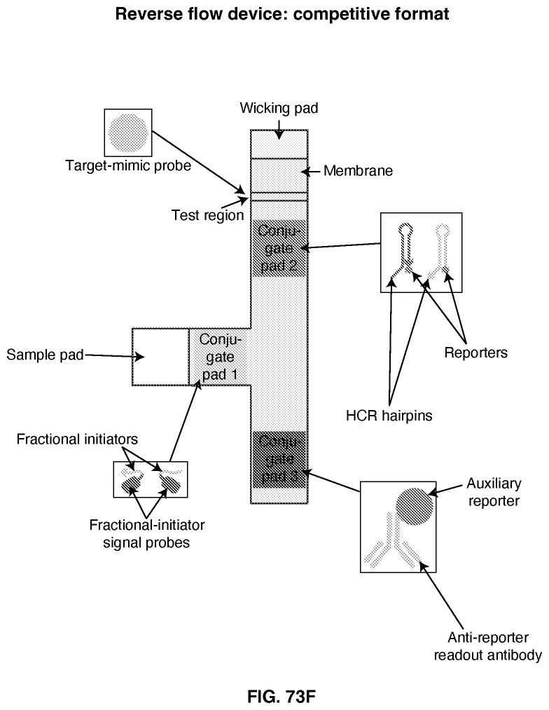

In some embodiments, a device comprises a test region, an optional control region, and a multi-channel membrane configured in a wagon wheel shape (for example, see FIGS. 33A and 73A), wherein the membrane comprises multiple reagent channels that enter a unified channel at one location after which the unified channel leads in succession to the test region, the optional control region, and an optional wicking pad. In some embodiments, one or more of the reagent channels contacts one or more conjugate pads, dipping wells, or other entities containing reagents. In some embodiments, reagents automatically arrive at the test region and the optional control region in a prescribed order without user interaction. In some embodiments, reagents that flow through the reagent channel that offers the shortest path to the test region and optional control region arrive first, reagents that flow through the reagent channel that offers the longest path to the test region and optional control region arrive last, and reagents that flow through reagent channels that offer paths of intermediate length to the test region and optional control region arrive at intermediate times according to the path length. In some embodiments, a reagent is introduced to a reagent channel by wetting a conjugate pad in contact with the channel to resuspend reagents that have been dried down in the conjugate pad. In some embodiments, a reagent is introduced to a reagent channel by dipping into a well containing reagents in solution. In some embodiments, some reagent channels have conjugate pads and other reagent channels do not have conjugate pads. In some embodiments, some conjugate pads do not contain dried down reagents. In some embodiments, the reagents will flow through the test and optional control regions based on the order that the reagent channels enter the unified channel and/or based on the length, width, or thickness of each reagent channel before it enters the unified channel and/or based on the speed additives, membrane modifications, and/or membrane material properties in each channel.

In some embodiments, the device comprises a test region, an optional control region, one or more optional conjugate pads, and two or more membranes stacked on top of each other (for example, see FIGS. 33B-33C and 73B-73C) wherein optionally some membranes are separated by impermeable barriers. In some embodiments, the stacked device comprises multiple reagent channels, one or more of which contacts one or more conjugate pads, dipping wells, or other entities containing reagents. In some embodiments, one or more of the reagent channels is overlaid on top of a unified channel with an impermeable barrier between each pair of channels, except optionally at certain locations where flow is permitted between channels by contact between the membranes. In some embodiments, the test region, the optional control region, and an optional wicking pad follow in succession after the entry point of one or more reagent channels into the unified channel. In some embodiments, one or more reagent channels originate in different locations and make contact with the unified channel to flow reagents into the unified channel. In some embodiments, two or more reagent channels join the unified channel at different locations. In some embodiments, two or more reagent channels join the unified channel at one location. In some embodiments, reagents automatically arrive at the test region and the optional control region in a prescribed order without user interaction. In some embodiments, reagents that flow through the reagent channel that offers the shortest path to the test region and optional control region arrive first, reagents that flow through the reagent channel that offers the longest path to the test region and optional control region arrive last, and reagents that flow through reagent channels that offer paths of intermediate length to the test region and optional control region arrive at intermediate times according to the path length. In some embodiments, a reagent is introduced to a reagent channel by wetting a conjugate pad in contact with the channel to resuspend reagents that have been dried down in the conjugate pad. In some embodiments, a reagent is introduced to a reagent channel by dipping into a well containing reagents in solution. In some embodiments, some reagent channels have conjugate pads and other reagent channels do not have conjugate pads. In some embodiments, some conjugate pads do not contain dried down reagents. In some embodiments, the reagents will flow through the test and optional control regions based on the order that the reagent channels enter the unified channel and/or based on the length, width, or thickness of each reagent channel before it enters the unified channel and/or based on the speed additives, membrane modifications, and/or membrane material properties in each channel.

In some embodiments, the device comprises a half-strip assay format (for example, see FIG. 6) comprising a test region, an optional control region, and a membrane comprising one or more reagent channels and a unified channel. In some embodiments, the membrane is laid out in a linear geometry, a comb shape, a wagon wheel shape, a stacked configuration, or another configuration. In some embodiments, the test region, the optional control region, and an optional wicking pad follow in succession after the entry point of one or more reagent channels into the unified channel. In some embodiments, there is only one reagent channel and it is the same channel as the unified channel. In some embodiments, reagents are introduced to one or more reagent channels by dipping the end of the channel into a well containing reagents in solution. In some embodiments, the end of a reagent channel is dipped one or more times into one or more wells containing reagents in solution. In some embodiments, reagents automatically arrive at the test region and the optional control region in a prescribed order without user interaction. In some embodiments, reagents that flow through the reagent channel that offers the shortest path to the test region and optional control region arrive first, reagents that flow through the reagent channel that offers the longest path to the test region and optional control region arrive last, and reagents that flow through reagent channels that offer paths of intermediate length to the test region and optional control region arrive at intermediate times according to the path length. In some embodiments, the reagents will flow through the test and optional control regions based on the order that the reagent channels enter the unified channel and/or based on the length, width, or thickness of each reagent channel before it enters the unified channel and/or based on the speed additives, membrane modifications, and/or membrane material properties in each channel. In some embodiments, reagents automatically arrive at the test region and the optional control region in a prescribed order with user interaction. In some embodiments, reagents will flow through the test and optional control regions based on the relative timing with which the end of one or more reagent channels is dipped in one or more wells containing reagents in solution.

In some embodiments, the device comprises a dipstick assay (for example, see FIGS. 33D and 73D) comprising along one channel, in succession, a membrane, a test region, an optional control region, and an optional wicking pad. In some embodiments, reagents are introduced into the channel by dipping the end of the membrane into one or more wells containing reagents in solution. In some embodiments, the end of the membrane is dipped into multiple wells in succession for a prescribed amount of time in each well. In some embodiments, the end of the membrane is dipped into multiple wells in succession, moving on to the next well after exhausting the reagents in the previous well. In some embodiments, the reagents flow through the test and optional control regions in the order that the end of the membrane is dipped in different reagent dipping wells.

In some embodiments, the device comprises a membrane, two or more conjugate pads, a test region, an optional control region, and an optional wicking pad. In some embodiments, reagents automatically arrive at the test region and the optional control region by placing two or more conjugate pads at different locations along the membrane (for example, see FIGS. 33E and 73E), such that liquid flowing through the membrane sequentially encounters conjugate pads loaded with different reagents, carrying those reagents to the test region and optional control region in a prescribed order. In some embodiments, the automated delivery of reagents to the test region and optional control region occurs by stacking two or more conjugate pads such that liquid flowing in the membrane sequentially rehydrates the reagents in the stacked conjugate pads, carrying those reagents to the test region and optional control region in a prescribed order. In some embodiments, the automated delivery of reagents to the test region and optional control region occurs by utilizing stacked dissolvable and/or sequentially accessible layers loaded with reagents, such that liquid flowing in the membrane sequentially dissolves the stacked layers to rehydrate their loaded reagents and deliver those reagents to the test region and optional control region in a prescribed order.

In some embodiments, the device comprises a membrane, two or more sets of conjugate pads each comprising one or more conjugate pads, a test region, an optional control region, and a wicking pad. In some embodiments, the first set of one or more conjugate pads is positioned so that the initial flow toward them is in the direction of the test region and optional control region, wherein the flow encounters the first set of conjugate pads loaded with reagents and carries those reagents to the test region and optional control region in a prescribed order before then reaching the wicking pad. In some embodiments, the second set of one or more of the conjugate pads loaded with reagents is positioned so that the initial flow through the membrane toward them is directed away from the test region and optional control region (for example, see FIGS. 33F and 73F), wherein once the flow through the first set of conjugate pads reaches the wicking pad, the direction of flow through the second set of conjugate pads is reversed so that the flow from the second set of conjugate pads travels across the test region and optional control region to the wicking pad, allowing automated delivery of the reagents from the second set of conjugate pads to the test region and optional control region after delivery of the reagents from the first set of conjugate pads.

In some embodiments, a conjugate pad is pre-loaded with reagents (for example, see FIGS. 30A-30B, 31A-31B, 36A-36C, and 72A-72C). In some embodiments, reagents have been dried down into a conjugate pad (for example, see FIGS. 30A-30B, 31A-31B, 36A-36C, and 72A-72C). In some embodiments, when flow through a channel encounters a conjugate pad into which reagents have been pre-loaded or dried down, the reagents are resuspended in solution and travel with the flow through the membrane.

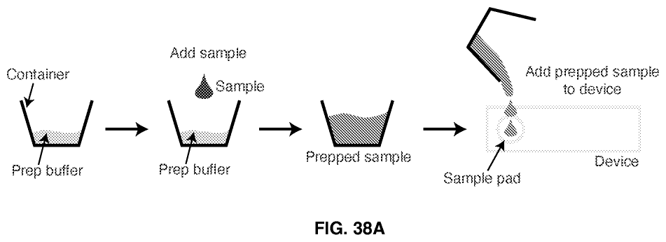

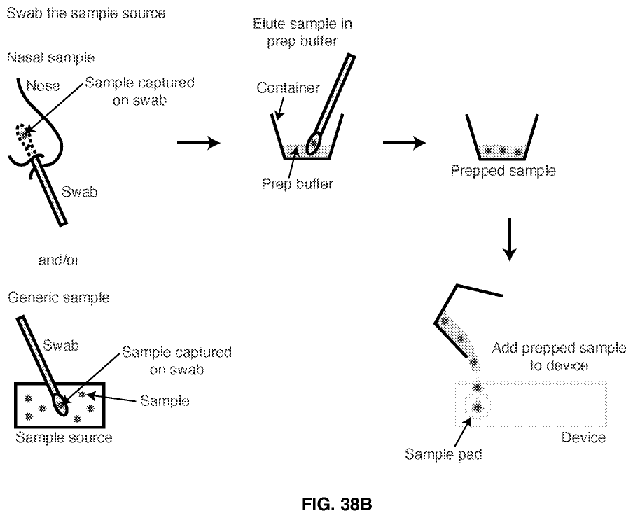

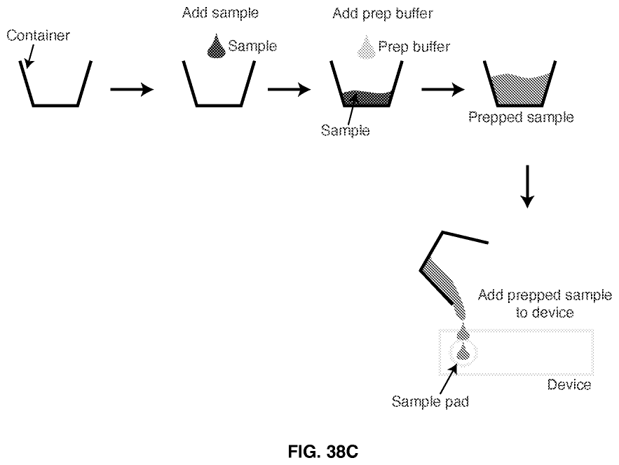

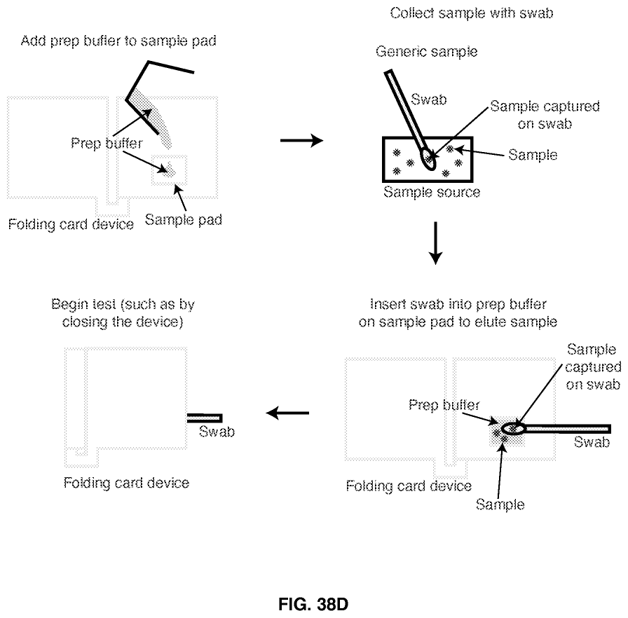

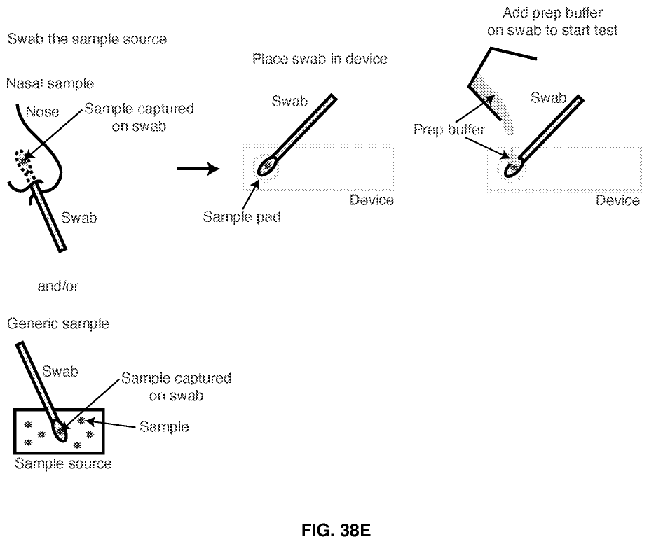

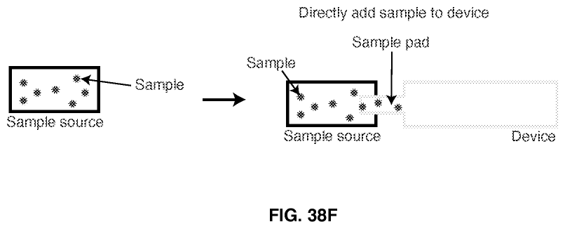

In some embodiments, the user preps the sample by adding the sample to a prep buffer (for example, see FIG. 38A-38B). In some embodiments, the user preps the sample by adding prep buffer to the sample (for example, see FIG. 38C). In some embodiments, the user preps the sample by heating the sample in solution. In some embodiments, the prep buffer is an extraction buffer that exposes the target (for example, by disrupting a viral envelope or cell membrane). In some embodiments, the prep buffer comprises a denaturant.

In some embodiments, the user adds the prepped sample to a sample pad to start a test. In some embodiments, the user adds the prep buffer to the sample pad and adds the sample to the prepped sample pad (for example, see FIG. 38D). In some embodiments, the user adds sample directly to the sample pad (for example, by putting a swab in contact with the sample pad) and then adds the prep buffer to the sample and sample pad (for example, see FIG. 38E). In some embodiments, the user adds the sample to the device (for example, see FIG. 38F).

In some embodiments, the user closes a folding card device to start the test (for example, see FIG. 38D). In some embodiments, closing the card creates contacts between the membrane and one or more conjugate pads, initiating the flow of liquid in one or more channels. In some embodiments, the reagents are delivered to the test region in successive stages via one or more channels without user interaction.

In some embodiments, the user reads either a positive result (visible signal in the test region) or a negative result (no visible signal in the test region) with the naked eye. In some embodiments, the user or a health care professional reads a result using an instrument to scan the test region (for example, a fluorescence scanner, camera, radiation scanner, mass spectrometer, or other device that allows acquisition of a signal from the test region).

In some embodiments, a folding card device is disposable. In some embodiments, a folding card device can be reused. In some embodiments, the folding card device is printed with a 3D printer (for example, see FIG. 5). In some embodiments, the disposable folding card device comprises cardboard. In some embodiments, the folding card device is injection molded. In some embodiments, the folding card device is machined. In some embodiments, the folding card is made of metal, paper, cardboard, plastic, glass, or any other material.

In some embodiments, the sample is introduced to the device using a swab (for example, see FIGS. 38B, 38D, and 38E). The swabbed sample can be obtained, for example, by nasopharyngeal swabbing, anterior nasal swabbing, swabbing of a cat, a dog, a livestock animal, tree sap, and environmental sample (such as swabbing a toilet, a sewer, or an aquifer) or swabbing of any other human and/or non-human body and/or non-body part or specimen. In some embodiments, the swab is mixed with a prep buffer by dipping, swirling, and/or physical mixing to create a prepped sample solution. In some embodiments, the prepped sample solution is added to the device using a dropper, pipette, or any other liquid handling mechanism. In some embodiments, the device receives the prepped sample solution in a sample pad or a reagent well. In some embodiments, the device takes the form of a comb, wagon wheel, stacked device, half-strip, dipstick, reverse-flow device, any combination thereof, or another geometry.

In some embodiments, the sample is saliva, sputum, nasal discharge, urine, blood, plasma, tears, synovial fluid, bioaerosol, for example collected from breath, derived from humans, cats, dogs, or any other species, lake water, sewage, an aquifer or any other direct and/or processed human and/or non-human bodily or non-bodily fluids.

In some embodiments, the sample is a liquid or solid that is collected and added to a container containing prep buffer before being loaded onto the device (for example, see FIG. 38A-38B). In some embodiments, the liquid or solid sample is added to and mixed with prep buffer via dropper, pipette, swirling, or any other liquid handling mechanism. In some embodiments, the sample is collected using a swab or other material and eluted into prep buffer to create a prepped sample solution before loading the prepped sample solution onto the device (for example, see FIG. 38B). In some embodiments, the device receives the prepped sample solution in a sample pad (for example, see FIG. 38A-38F) or a reagent well. In some embodiments, the device takes the form of a comb, wagon wheel, stacked device, half-strip, dipstick, reverse-flow device, any combination thereof, or another geometry (for example, see FIGS. 5, 6, 30A-30B, 31A-31B, 32A-32B, 33A-33F, 36A-36C, 48A-48B, 72A-72C, and 73A-73F).

### HCR Lateral Flow Assays for Amplified Detection of a Target Protein in a Sample

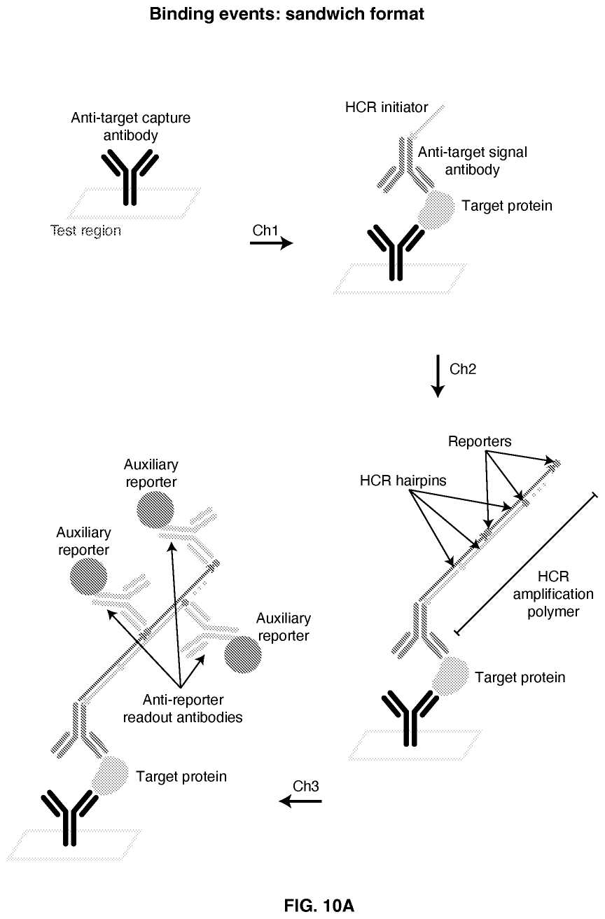

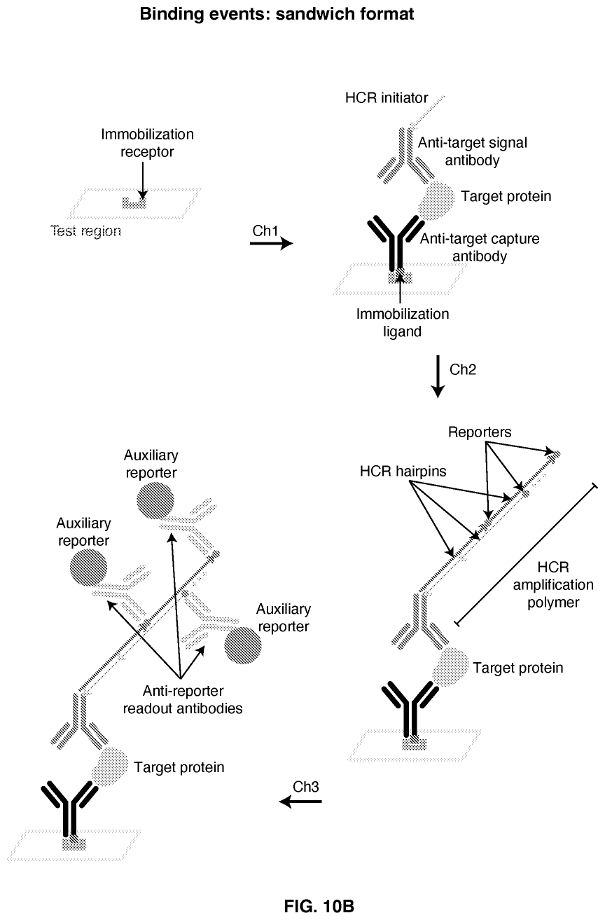

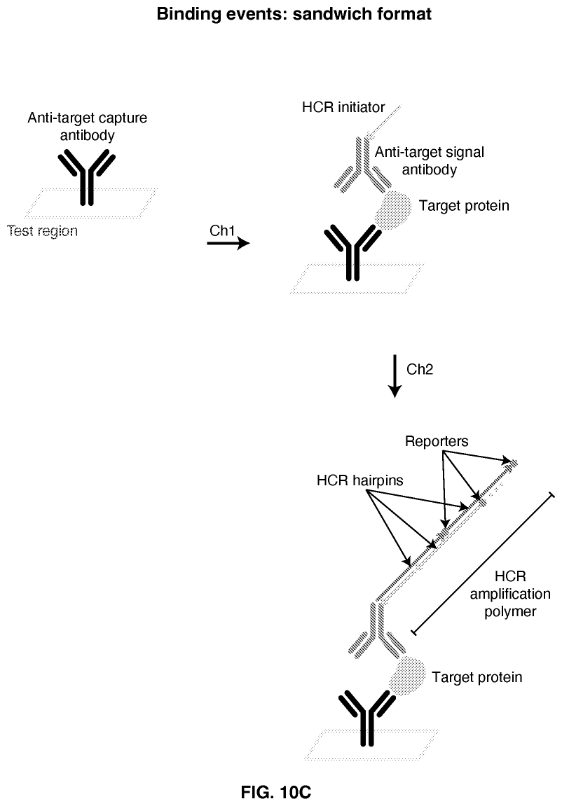

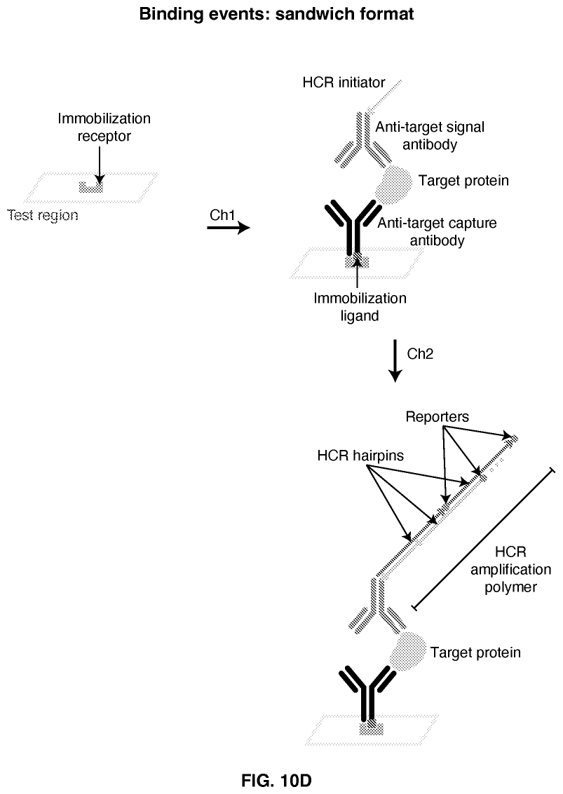

In some embodiments an amplified HCR lateral flow device is used to detect a target protein in a sample. In some embodiments, the device comprises a test region (for example, see FIGS. 7A-7J and 9A-9E). In some embodiments, the device comprises a capture probe set comprising an anti-target capture antibody comprising a target-binding domain configured to bind directly or indirectly to the target and an immobilization domain configured to bind directly or indirectly to the test region (for example, see FIGS. 7A-7J and 9A-9E). In some embodiments, the anti-target capture antibody is pre-immobilized in the test region (for example, see FIGS. 8A, 8C, 10A, and 10C). In some embodiments, the anti-target capture antibody is labeled with an immobilization ligand that binds an immobilization receptor that is pre-immobilized in the test region (for example, see FIGS. 8B, 8D, 10B, and 10D). In some embodiments, the immobilization ligand is biotin and the immobilization receptor is a biotin-binding molecule (for example, polystreptavidin R (PR), streptavidin, avidin, NeutrAvidin, CaptAvidin, or another biotin-binding molecule), or the immobilization ligand is a hapten and the immobilization receptor is an anti-hapten antibody, or the immobilization ligand is an anti-hapten antibody and the immobilization receptor is a hapten, or the immobilization ligand is a nucleic acid and the immobilization receptor is a nucleic acid, or the immobilization ligand is an antigen and the immobilization receptor is an anti-antigen antibody, or the immobilization ligand is a molecule that is recognized by an immobilization receptor that is a zinc finger, or the immobilization ligand is another molecule or complex that can be recognized by an immobilization receptor to facilitate generation of a signal. In some embodiments, the anti-target capture antibody comprises a primary capture antibody comprising a target-binding domain and a secondary capture antibody comprising an immobilization domain configured to bind directly or indirectly to the test region, wherein the secondary capture antibody is configured bind the primary capture antibody directly or indirectly. In some embodiments, the primary capture antibody comprises a target-binding domain without an immobilization domain. In some embodiments, the secondary capture antibody comprises an immobilization domain without a target-binding domain.

In some embodiments, the device comprises an HCR amplifier comprising two or more HCR hairpins (for example, see FIGS. 3A, 14, 15, and 16A-16B). In some embodiments, one or more of the HCR hairpins are labeled with one or more reporters (for example, see FIG. 17A-17F). In some embodiments, the HCR hairpins comprising an HCR amplifier are configured not to polymerize in the absence of an HCR initiator. In some embodiments, HCR hairpins are configured such that detection of an HCR initiator triggers the HCR hairpins to polymerize to form a tethered HCR amplification polymer decorated with reporters (for example, see FIGS. 3A, 14, 15, and 16A-16B).

In some embodiments, the device comprises a signal probe set comprising an anti-target signal antibody comprising a target-binding domain configured to bind directly or indirectly to the target and an amplification domain that is configured to directly or indirectly trigger HCR signal amplification by initiating a chain reaction in which HCR hairpins self-assemble to form a tethered HCR amplification polymer decorated with reporters (for example, see FIGS. 7A-7J and 9A-9E). In some embodiments, the signal probe set comprises: a) one or more initiator-labeled probes wherein an initiator-labeled probe comprises a target-binding domain and an amplification domain comprising one or more HCR initiators (for example, see FIGS. 3B, 7A-7E, 7H, 9A-9E, 18A-18N, and 19A-19F), or b) one or more probe units, each comprising two or more fractional-initiator probes, wherein a fractional-initiator probe comprises a target-binding domain and an amplification domain comprising a fractional initiator. In some embodiments, an individual fractional-initiator probe that binds non-specifically will not trigger HCR, but specific binding of the probes within a probe unit to adjacent cognate binding sites on the target colocalizes a full HCR initiator that will trigger HCR when colocalized and in the presence of the other components (for example, see FIGS. 4A, 7F-7G, 7I-7J, 20A-20E, 21A, 21C-21D, 22A, 22C-22E, 24A-24R). In some embodiments, the anti-target signal antibody comprises a primary signal antibody comprising a target-binding domain and a secondary signal antibody comprising an amplification domain (for example, see FIGS. 18C-18D, 18K-18L, 21D, 22D, 24D-24I, 24M-24R) wherein the secondary signal antibody is configured to bind the primary signal antibody directly or indirectly.

In some embodiments, the device comprises a readout probe set comprising an anti-reporter readout antibody comprising a reporter-binding domain configured to bind directly or indirectly to a reporter on an HCR amplification polymer (for example, see FIGS. 7A, 7C-7J, and 9A-9E). In some embodiments, the anti-reporter readout antibody comprises one or more auxiliary reporters wherein the auxiliary reporters contribute directly or indirectly to the generation of an amplified signal in the test region (for example, see FIGS. 7A, 7C-7J, and 9A-9E). In some embodiments, the amplified signal generated by the auxiliary reporters on the anti-reporter readout antibody is visible to the human eye. In some embodiments, the anti-reporter readout antibody comprises a primary readout antibody comprising a reporter-binding domain and a secondary readout antibody comprising an auxiliary reporter (for example, see FIG. 7H) wherein the secondary readout antibody is configured to bind the primary readout antibody directly or indirectly.

In some embodiments, if the target protein is present in the sample, the target protein binds in a sandwich between the capture probe set comprising an anti-target capture antibody and the signal probe set comprising an anti-target signal antibody (for example, see FIGS. 7A-7J and 9A-9E). In some embodiments, the anti-target capture antibody immobilizes the antibody/target sandwich in the test region (for example, see FIGS. 8A-8D and 10A-10D). In some embodiments, if the target is not present in the sample, the antibody/target sandwich does not form, the anti-target signal antibody is not immobilized in the test region, the growth of tethered HCR amplification polymers is not triggered in the test region, and an amplified signal is not generated in the test region. In some embodiments, after the antibody/target sandwich is immobilized in the test region, the amplification domains on the anti-target signal antibody trigger the self-assembly of reporter-labeled HCR hairpins into tethered HCR amplification polymers decorated with reporters (for example, see FIGS. 8A-8D and 10A-10D).

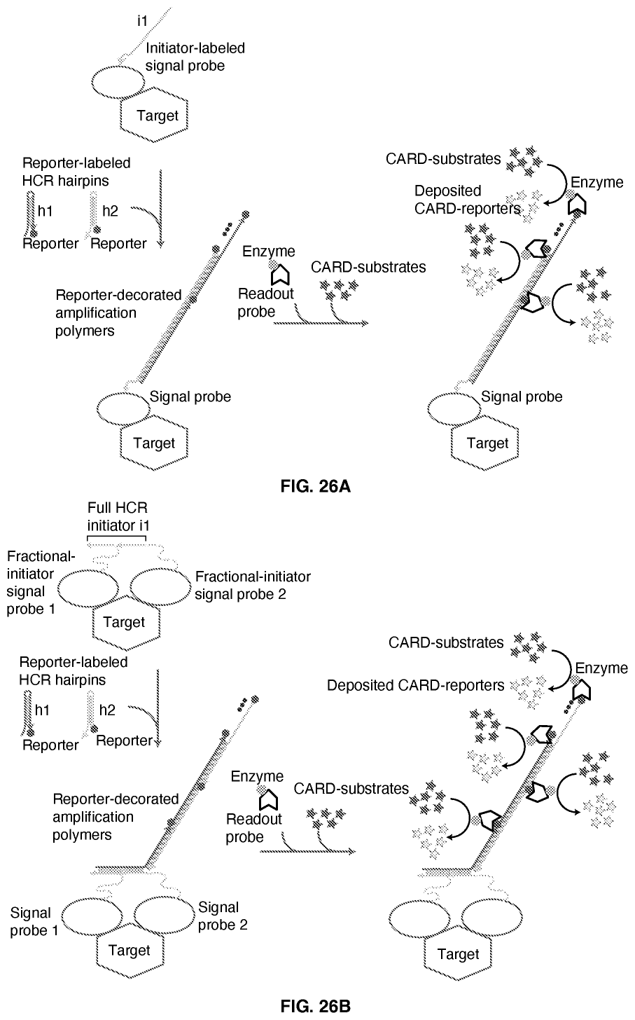

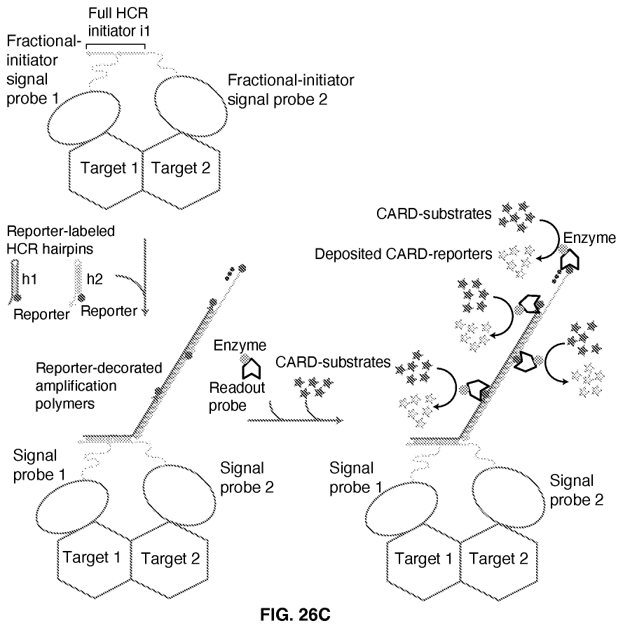

In some embodiments, the reporters directly or indirectly lead to generation of an amplified signal in the test region. In some embodiments, auxiliary-reporter-labeled anti-reporter readout antibodies that comprise one or more auxiliary reporters bind the reporters decorating the tethered HCR amplification polymers that are immobilized in the test region, directly or indirectly generating an amplified signal in the test region (for example, see FIGS. 8A-8B and 10A-10B). In some embodiments, the amplified signal is visible to the human eye. In some embodiments, the signal is read using a fluorescence microscope, a fluorescence scanner, a camera, a mobile phone camera, a mass spectrometer, a mass spectrometry microscope, a radioactive scanner, or another instrument suitable for detecting reporters and/or auxiliary reporters. In some embodiments, a reporter and/or an auxiliary reporter comprises a fluorophore, a chromophore, a luminophore, a phosphor, a FRET pair, a member of a FRET pair, a quencher, a fluorophore/quencher pair, a rare earth element or compound, a radioactive molecule, an enzyme, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a reporter and/or auxiliary reporter is a hapten, a ligand, an oligonucleotide, digoxigenin (DIG), fluorescein isothiocyanate (FITC), a fluorophore, biotin, dinitrophenol, aniline, or another molecule or complex that can be recognized by a binding partner to facilitate generation of a signal. In some embodiments, the reporters and/or auxiliary reporters decorating an HCR amplification polymer directly or indirectly mediate catalytic reporter deposition (CARD) to generate an amplified signal in the vicinity of the HCR amplification polymer within the test region. In some embodiments, CARD is mediated by an enzyme (for example, see FIGS. 25C-25E, 26A-26C).

In some embodiments an HCR lateral flow device comprises a test region, a capture probe set comprising an anti-target capture antibody comprising a target-binding domain and an immobilization domain, a signal probe set comprising an anti-target signal antibody comprising a target-binding domain and an amplification domain, an HCR amplifier comprising two or more HCR hairpins wherein one or more HCR hairpins comprise one or more reporters, and optionally a readout probe set comprising an anti-reporter readout antibody comprising a reporter-binding domain and one or more auxiliary reporters (for example, see FIGS. 7A-7J and 9A-9E). In some embodiments, the device is configured so that the reagents automatically arrive in the test region in one or more successive stages, generating an amplified signal in the test region if the target is present in the sample (for example, see FIGS. 8A-8D, 10A-10D, 30A-30B, and 31A-31B).

In some embodiments an HCR lateral flow device comprises a test region, a capture probe set comprising a capture antibody, a signal probe set comprising a signal antibody, an HCR amplifier comprising one or more reporters, and optionally a readout probe set comprising a readout antibody comprising one or more auxiliary reporters (for example, see FIGS. 7A-7J and 9A-9E). In some embodiments, the device is configured so that the reagents automatically arrive in the test region in one or more successive stages, generating an amplified signal in the test region if the target is present in the sample (for example, see FIGS. 8A-8D, 10A-10D, 30A-30B, and 31A-31B).

In some embodiments an HCR lateral flow device comprises a test region, a capture probe set comprising a capture antibody, a signal probe set comprising a signal antibody, an HCR amplifier comprising one or more reporters, and optionally a readout probe set comprising a readout antibody comprising one or more auxiliary reporters (for example, see FIGS. 7A-7J and 9A-9E). In some embodiments, the device is configured so that the reagents automatically arrive in the test region in a prescribed order, generating an amplified signal in the test region if the target is present in the sample (for example, see FIGS. 8A-8D, 10A-10D, 30A-30B, and 31A-31B).

In some embodiments, reagents are automatically delivered to the test region in multiple successive stages using a multi-channel membrane (for example, see FIGS. 5, 30A-30B, and 31A-31B, and 33A-33C, and 33F). In some embodiments, reagents are automatically delivered to the test region in successive stages using a multi-channel membrane in which channels of different lengths lead into a unified channel before reaching the test region (for example, see FIGS. 5, 30A-30B, 31A-31B, and 33C). In some embodiments, channels of differing lengths deliver reagents to the test region in order according to their lengths, with the reagents from the shortest channel arriving in the test region first, the reagents from the longest channel arriving in the test region last, and channels of intermediate lengths arriving in the test region at intermediate times (for example, see FIGS. 5, 30A-30B, 31A-31B, and 33C). In some embodiments, channels are pre-treated with speed additives that create different flow speeds in different channels, allowing for delivery of reagents to the test region in successive stages. In some embodiments, the speed additives comprise sucrose, fructose, trehalose, a sugar, dextran sulfate, a dissolvable barrier, a wax barrier, water-insoluble ink, sponge, valves, pressurized paper, agarose, gravitational orientation, or another agent. In some embodiments, multiple channels are stacked on top of each other to form a multi-layer membrane (for example, see FIGS. 33B-33C). In some embodiments, the sample arrives in different channels at different times, leading to successive delivery of reagents to the test region. In some embodiments, dissolvable barriers dissolve at different rates to introduce the sample into different channels at different times, leading to successive delivery of reagents to the test region. In some embodiments, different reagents migrate through the same or different channels at different rates leading to successive staged arrival times in the test region. For example, auxiliary-reporter-labeled readout antibodies labeled with bulky auxiliary reporters migrate through the membrane more slowly than reporter-labeled HCR hairpins labeled with small reporters, allowing for the readout probes to arrive in the test region after the HCR hairpins. In some embodiments, the size of different reagents affects their migration speeds through the same channel, allowing for successive delivery of reagents to the test region. In some embodiments, a multi-channel membrane is configured so that reagents in each channel arrive in the test region (or another location) in a prescribed order (for example, see FIGS. 5, 30A-30B, 31A-31B, and 33A-33F).

In some embodiments, reagents for each channel are dried onto separate conjugate pads (for example, see FIGS. 5, 30A-30B, 31A-31B, 33A-33C, and 33E-33F). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of a first conjugate pad allows draining of a second conjugate pad, which in turn allows draining of a third conjugate pad, and so on (for example, see FIGS. 5, 30A-30B, and 31A-31B). In some embodiments, draining of the first conjugate pad frees a unified channel to allow draining of a second conjugate pad, which in turn frees a unified channel to allow draining of a third conjugate pad, and so on (for example, see FIGS. 5, 30A-30B, and 31A-31B).

In some embodiments, a membrane, a sample pad, a conjugate pad, and/or a wicking pad comprise nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.

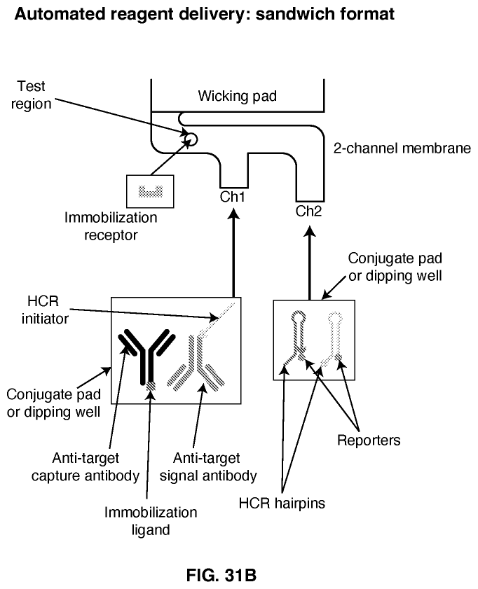

In some embodiments, reagents are automatically delivered to the test region in two successive stages using a two-channel membrane in which channels of different lengths lead into a unified channel before reaching the test region (for example, see FIGS. 30B, 31B). In some embodiments, the anti-target signal antibody binds the protein target and travels via the shorter channel (Channel 1) to reach the test region first where the target additionally binds the anti-target capture antibody pre-immobilized in the test region to form an immobilized antibody/target sandwich (for example, see FIGS. 8C and 10C). In some embodiments, the anti-target signal antibody and anti-target capture antibody bind the target protein and travel via the shorter channel (Channel 1) to reach the test region first, where the antibody/target sandwich is immobilized via binding of immobilization-ligand-labeled anti-target capture antibody to immobilization receptors pre-immobilized in the test region (for example, see FIGS. 8D, 10D, 30B, and 31B). In some embodiments, reporter-labeled HCR hairpins travel via a longer channel (Channel 2) and reach the test region next, where amplification domains on the anti-target signal antibody trigger growth of tethered HCR amplification polymers decorated with reporters to generate an amplified signal in the test region (for example, see FIGS. 8C-8D, 10C-10D, 30B, and 31B). In some embodiments, reagents for each channel are dried onto two separate conjugate pads. In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user interaction. In some embodiments, draining of the first conjugate pad allows draining of the second conjugate pad. In some embodiments, draining of the first conjugate pad frees a unified channel to allow draining of the second conjugate pad. In some embodiments, reagents in the two channels arrive in the test region (or another location) in successive stages in a prescribed order due to channel length, channel geometry, speed additives, dissolvable barriers, or other conditions (for example, see FIGS. 30B and 31B).

In some embodiments, reagents are automatically delivered to the test region in three successive stages using a three-channel membrane in which channels of different lengths lead into a unified channel before reaching the test region (for example, see FIGS. 30A and 31A). In some embodiments, the anti-target signal antibody binds the target and travels via the shortest channel (Channel 1) to reach the test region first where the target additionally binds the anti-target capture antibody that is pre-immobilized in the test region to form an immobilized antibody/target sandwich (for example, see FIGS. 8A and 10A). In some embodiments, the anti-target signal antibody and the anti-target capture antibody bind the target and travel via the shortest channel (Channel 1) to reach the test region first, where the antibody/target sandwich is immobilized via binding of the immobilization-ligand-labeled anti-target capture antibody to immobilization receptors pre-immobilized in the test region (for example, see FIGS. 8B, 10B, 30A, and 31A). In some embodiments, reporter-labeled HCR hairpins travel via a channel of intermediate length (Channel 2) and reach the test region next, where amplification domains on the anti-target signal antibody triggers growth of tethered HCR amplification polymers decorated with reporters (for example, see FIGS. 8A-8B, 10A-10B, 30A, and 31A). In some embodiments, an auxiliary-reporter-labeled anti-reporter readout antibody travels via the longest channel (Channel 3) and arrives in the test region last, where it binds the reporters decorating the HCR amplification polymers to generate an amplified signal in the test region (for example, see FIGS. 8A-8B, 10A-10B, 30A, and 31A). In some embodiments, reagents for each channel are dried onto separate conjugate pads (for example, see FIGS. 30A and 31A). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of the first conjugate pad allows draining of the second conjugate pad, which in turn allows draining of the third conjugate pad, and so on. In some embodiments, draining of the first conjugate pad frees the unified channel to allow draining of the second conjugate pad, which in turn frees the unified channel to allow draining of the third conjugate pad. In some embodiments, reagents in the three channels arrive in the test region (or another location) in successive stages in a prescribed order due to channel length, channel geometry, speed additives, dissolvable barriers, or other conditions (for example, see FIGS. 30A and 31A).

In some embodiments, the device comprises a control region (for example, see FIGS. 34A-34B, 35A-35B, and 36A-36B). In some embodiments, the control region is downstream of the test region so that the sample and reagents flow through the test region before reaching the control region. In some embodiments an HCR lateral flow device comprises a test region, a control region, a capture probe set comprising an anti-target antibody, a signal probe set comprising an anti-target antibody, a control capture probe set comprising a control capture antibody, a control signal probe set comprising a control signal antibody, an HCR amplifier comprising one or more reporters, and optionally a readout probe set comprising a readout antibody comprising one or more auxiliary reporters (for example, see FIGS. 34A-34B, 35A-35B, and 36A-36B). In some embodiments, the device is configured so that the reagents automatically arrive in the test region and the control region in one or more successive stages (for example, see FIGS. 36A-36B), generating an amplified signal in the test region if the target is present in the sample, and generating an amplified control signal in the control region if the sample and reagents flow through the device properly. In some embodiments, if the sample and reagents successfully flow through the control region, the control capture antibody binds to and immobilizes the control signal antibody in the control region, the control amplification domains on the control signal antibody trigger the self-assembly of tethered HCR amplification polymers decorated with reporters to directly or indirectly generate an amplified control signal in the control region, and optionally the auxiliary-reporter-labeled readout antibody binds the reporters to directly or indirectly generate an amplified control signal in the control region. In some embodiments, the test produces a positive result if both the test region and the control region produce visible signals. In some embodiments, the test produces a negative result if signal is observed in the control region but not in the test region. In some embodiments, absence of signal in the control region is an indication that the test did not function properly.

In some embodiments, the device comprises a folding card geometry (for example, see FIG. 5). In some embodiments, one page of the card contains a sample pad and one or more conjugate pads. In some embodiments, the other page of the card contains a membrane comprising one or more channels, a test region, an optional control region, and a wicking pad. In some embodiments, the wicking pad absorbs liquid to induce continued capillary flow through the channels after the sample reaches the wicking pad. In some embodiments, one page is functionalized with prongs that disconnect the sample pad from the one or more conjugate pads upon folding the card. In some embodiments, disconnecting the sample pad from the one or more conjugate pads limits the volume that flows from the one or more conjugate pads and prevents flow between conjugate pads. In some embodiments, the device takes the form of a comb, wagon wheel, stacked device, half-strip, dipstick, reverse-flow device, any combination thereof, or another geometry.

In some embodiments, the user preps the sample by adding the sample to a prep buffer (for example, see FIG. 38A-38B). In some embodiments, the user preps the sample by adding prep buffer to the sample (for example, see FIG. 38C). In some embodiments, the user preps the sample by heating the sample in solution. In some embodiments, the prep buffer is an extraction buffer that exposes the target (for example, by disrupting a viral envelope or cell membrane). In some embodiments, the prep buffer is a denaturant. In some embodiments, the user adds the prepped sample to a sample pad to start a test. In some embodiments, the user adds the prep buffer to the sample pad and adds the sample to the prepped sample pad (for example, see FIG. 38D). In some embodiments, the user adds sample to the sample pad (for example, by putting a swab in contact with the sample pad) and then adds the prep buffer to the sample and sample pad (for example, see FIG. 38E). In some embodiments, the user adds the sample to the device (for example, see FIG. 38F). In some embodiments, the user closes a folding card device to start the test (for example, see FIG. 38D). In some embodiments, closing the card creates contacts between the membrane and one or more conjugate pads, initiating the flow of liquid in one or more channels. In some embodiments, the reagents are delivered to the test region in successive stages via one or more channels without user interaction. In some embodiments, the user reads either a positive result (visible signal in the test region) or a negative result (no visible signal in the test region) with the naked eye. In some embodiments, the user or a health care professional reads a result using an instrument to scan the test region (for example, a fluorescence scanner, camera, radiation scanner, mass spectrometer, or other device that allows acquisition of a signal from the test region).

In some embodiments, the target protein binds a capture probe set comprising one or more capture probes, wherein a capture probe comprises a target-binding domain configured to bind directly or indirectly to the target protein and an immobilization domain configured to directly or indirectly immobilize the target in a test region (for example, see FIGS. 7A-7J and 9A-9E). In some embodiments, the capture probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above. In some embodiments, the immobilization domain binds directly to the test region. In some embodiments, the capture probe set is pre-immobilized in the test region (for example, see FIGS. 8A, 8C, 10A, and 10C). In some embodiments, the immobilization domain comprises an immobilization ligand. In some embodiments, the immobilization ligand binds an immobilization receptor that is pre-immobilized in the test region (for example, see FIGS. 8B, 8D, 10B, and 10D).

In some embodiments, the target protein binds a signal probe set comprising one or more signal probes, wherein a signal probe comprises a target-binding domain configured to bind directly or indirectly to the target protein and an amplification domain that is configured to directly or indirectly initiate a chain reaction in which the HCR hairpins self-assemble to form an HCR amplification polymer (for example, see FIGS. 7A-7J and 9A-9E). In some embodiments, the signal probe set comprises: a) one or more initiator-labeled probes wherein an initiator-labeled probe comprises a target-binding domain and an amplification domain comprising one or more HCR initiators (for example, see FIGS. 3B, 7A-7E, 7H, 9A-9E, 18A-18N, and 19A-19F), or b) one or more probe units, each comprising two or more fractional-initiator probes, wherein a fractional-initiator probe comprises a target-binding domain and an amplification domain comprising a fractional initiator (for example, see FIGS. 4A, 7F-7G, 7I-7J, 20A-20E, 21A, 21C-21D, 22A, 22C-22E, 24A-24R). In some embodiments, a capture probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above.

### HCR Lateral Flow Assays for Amplified Detection of a Target Nucleic Acid in a Sample

In some embodiments, an amplified HCR lateral flow device is used to detect a target nucleic acid in a sample. In some embodiments, the device comprises a test region (for example, see FIGS. 7A-7J and 11A-11E).

In some embodiments, the device comprises an HCR amplifier comprising two or more HCR hairpins (for example, see FIGS. 3A, 14, 15, and 16A-16B). In some embodiments, one or more of the HCR hairpins are labeled with one or more reporters (for example, see FIG. 17A-17F). In some embodiments, the HCR hairpins comprising an HCR amplifier are configured not to polymerize in the absence of an HCR initiator. In some embodiments, HCR hairpins are configured such that detection of an HCR initiator triggers the HCR hairpins to polymerize to form a tethered HCR amplification polymer decorated with reporters (for example, see FIGS. 3A, 14, 15, and 16A-16B).

In some embodiments, the device comprises a signal probe set comprising one or more nucleic acid signal probes configured to bind to different subsequences along the target (for example, see FIGS. 7A-7J and 11A-11E). In some embodiments, the signal probe set comprises: a) one or more initiator-labeled probes wherein an initiator-labeled probe comprises a target-binding domain and an amplification domain comprising one or more HCR initiators (for example, see FIGS. 3B, 7A-7E, 7H, 11B, 18A-18N, and 19A-19F), or b) one or more probe units, each comprising two or more fractional-initiator probes, wherein a fractional-initiator probe comprises a target-binding domain and an amplification domain comprising a fractional initiator. In some embodiments, any individual fractional-initiator probe that binds non-specifically will not trigger HCR, but specific hybridization of the probes comprising a probe unit to adjacent cognate binding sites along the target nucleic acid will colocalize a full HCR initiator capable of triggering HCR (for example, see FIGS. 4A, 7F-7G, 7I-7J, 11A, 11C-11E, 20A-20E, 21B, 22B, 22E, 24A-24R).

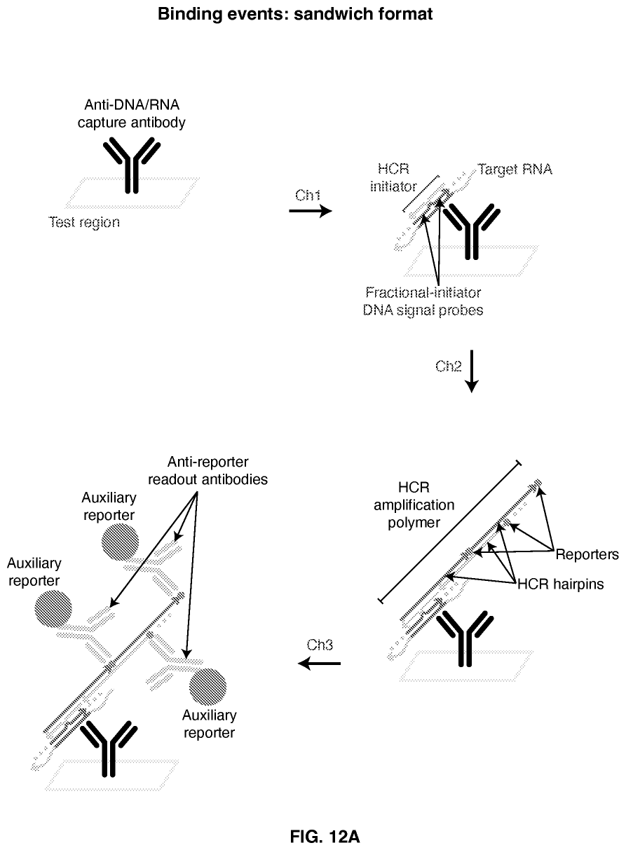

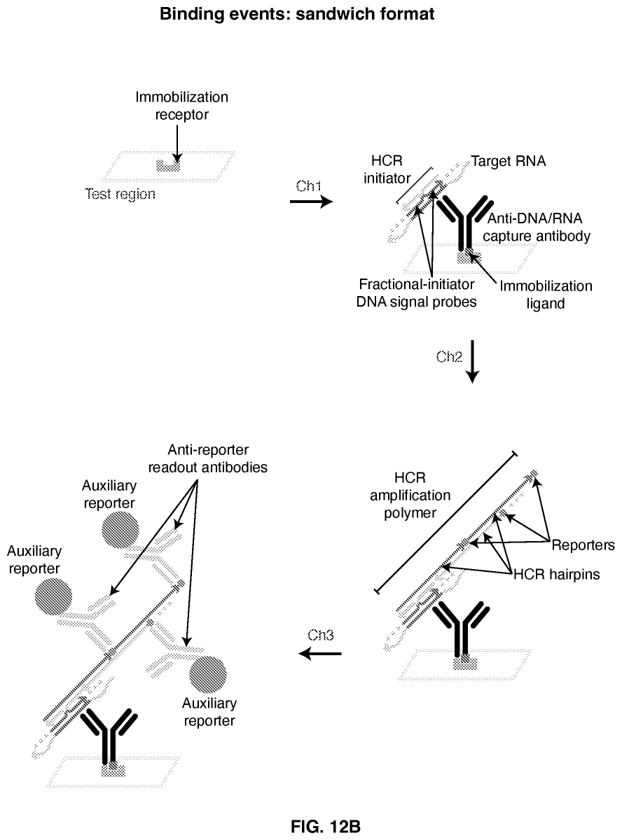

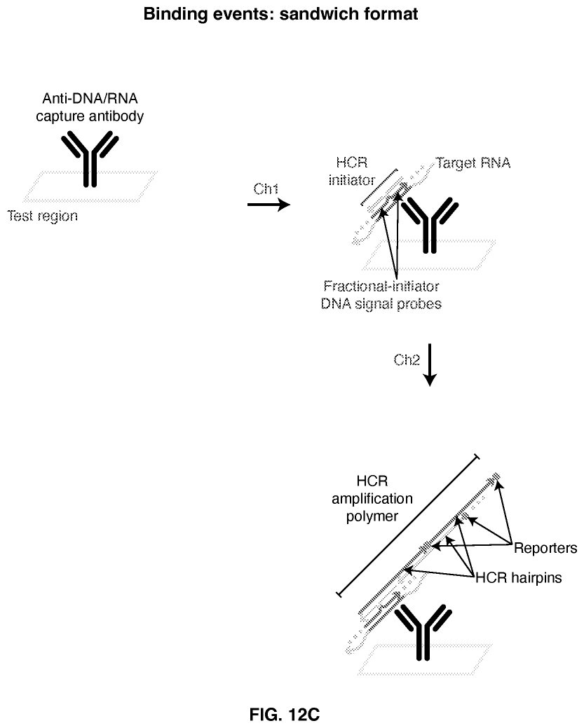

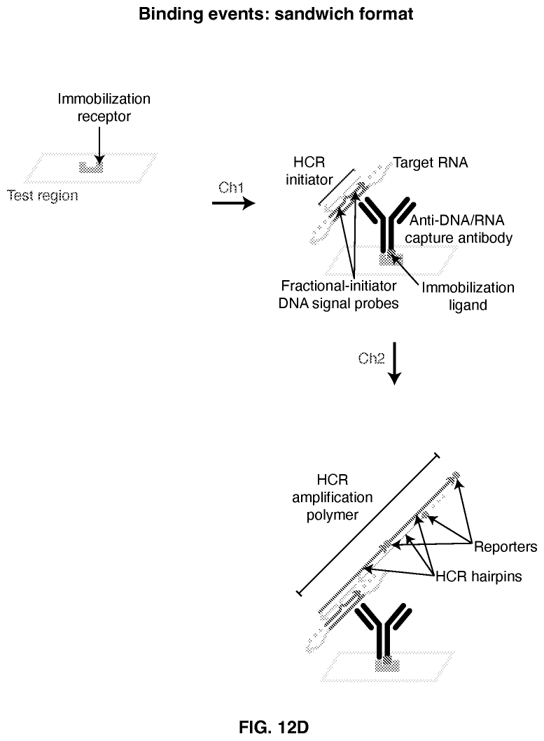

In some embodiments, the device comprises a capture probe set (for example, see FIGS. 7A-7J and 11A-11E). In some embodiments, the capture probe set comprises one or more nucleic acid capture probes configured to bind to different subsequences along the target (for example, see FIGS. 7A-7J and 11D-11E). In some embodiments, the target nucleic acid comprises a target RNA and the signal probe set comprises one or more DNA probes, wherein binding of the one or more DNA probes to cognate binding sites along the target RNA leads to formation of DNA/RNA duplexes. In some embodiments, the capture probe set comprises an anti-DNA/RNA capture antibody that binds DNA/RNA duplexes (for example, see FIGS. 11A-11B). In some embodiments, the capture probe set is pre-immobilized in the test region (for example, see FIGS. 8A, 8C, 12A, and 12C). In some embodiments, the probes comprising the capture probe set are labeled with an immobilization ligand that binds an immobilization receptor that is pre-immobilized in the test region (for example, see FIGS. 8B, 8D, 12B, and 12D). In some embodiments, the immobilization ligand is biotin and the immobilization receptor is a biotin-binding molecule (for example, polystreptavidin R (PR), streptavidin, avidin, NeutrAvidin, CaptAvidin, or another biotin-binding molecule), or the immobilization ligand is a hapten and the immobilization receptor is an anti-hapten antibody, or the immobilization ligand is an anti-hapten antibody and the immobilization receptor is a hapten, or the immobilization ligand is a nucleic acid and the immobilization receptor is a nucleic acid, or the immobilization ligand is an antigen and the immobilization receptor is an anti-antigen antibody, or the immobilization ligand is a molecule that is recognized by an immobilization receptor that is a zinc finger, or the immobilization ligand is another molecule or complex that can be recognized by an immobilization receptor to facilitate generation of a signal. In some embodiments, a capture probe comprises a primary capture probe comprising a target-binding domain and a secondary capture probe comprising an immobilization domain configured to bind directly or indirectly to the test region wherein the secondary capture probe is configured to bind the primary capture probe directly or indirectly (for example, a primary capture probe that is a primary antibody and a secondary capture probe that is a secondary antibody configured to bind the primary antibody).

In some embodiments, the one or more probe sets comprise a readout probe set comprising one or more readout probes, wherein a readout probe comprises a reporter-binding domain configured to bind directly or indirectly to a reporter on an HCR amplification polymer (for example, see FIGS. 7A, 7C-7J and 11A-11E). In some embodiments, a readout probe comprises one or more auxiliary reporters wherein the auxiliary reporters contribute directly or indirectly to the generation of an amplified signal in the test region (for example, see FIGS. 7A, 7C-7J, and 11A-11E). In some embodiments, the amplified signal generated by the auxiliary reporters on the readout probes is visible to the human eye. In some embodiments, a readout probe is an anti-reporter readout antibody labeled with one or more auxiliary reporters (for example, see FIGS. 7D-7F, 7I-7J, and 11A-11E). In some embodiments, a readout probe comprises a primary readout probe comprising a reporter-binding domain and a secondary readout probe comprising an auxiliary reporter (for example, see FIG. 7H) wherein the secondary readout probe is configured to bind the primary readout probe directly or indirectly (for example, a primary readout probe that is a primary antibody and a secondary readout probe that is a secondary antibody configured to bind the primary antibody).

In some embodiments, if the target nucleic acid is present in the sample, the target nucleic acid binds in a sandwich between a capture probe set and a signal probe set (for example, see FIGS. 7A-7J and 11A-11E). In some embodiments, the capture probe set immobilizes the probe/target sandwich in the test region (for example, see FIGS. 8A-8D and 12A-12D). In some embodiments, if the target is not present in the sample, the probe/target sandwich does not form, the signal probes are not immobilized in the test region, the growth of a tethered HCR amplification polymer is not triggered in the test region, and an amplified signal is not generated in the test region. In some embodiments, after the probe/target sandwich is immobilized in the test region, the amplification domains on the signal probes trigger the self-assembly of reporter-labeled HCR hairpins into tethered HCR amplification polymers decorated with reporters (for example, see FIGS. 8A-8D and 12A-12D). In some embodiments, the reporters directly or indirectly lead to generation of an amplified signal in the test region. In some embodiments, auxiliary-reporter-labeled readout probes that comprise one or more auxiliary reporters bind the reporters decorating the tethered HCR amplification polymers that are immobilized in the test region, directly or indirectly generating an amplified signal in the test region (for example, see FIGS. 8A-8B and 12A-12B). In some embodiments, the amplified signal is visible to the human eye. In some embodiments, the signal is read using a fluorescence microscope, a fluorescence scanner, a camera, a mobile phone camera, a mass spectrometer, a mass spectrometry microscope, a radioactive scanner, or another instrument suitable for detecting reporters and/or auxiliary reporters. In some embodiments, a reporter and/or an auxiliary reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a reporter and/or auxiliary reporter is a hapten, a ligand, an oligonucleotide, digoxigenin (DIG), fluorescein isothiocyanate (FITC), a fluorophore, biotin, dinitrophenol, aniline, or another molecule or complex that can be recognized by a binding partner to facilitate generation of a signal. In some embodiments, the reporters and/or auxiliary reporters decorating an HCR amplification polymer directly or indirectly mediate catalytic reporter deposition (CARD) to generate an amplified signal in the vicinity of the HCR amplification polymer within the test region. In some embodiments, CARD is mediated by an enzyme (for example, see FIGS. 25A-25B, 26A-26C, 27A-27B).

In some embodiments an HCR lateral flow device comprises a test region, a capture probe set comprising a target-binding domain and an immobilization domain, a signal probe set comprising a target-binding domain and an amplification domain, an HCR amplifier comprising two or more HCR hairpins wherein one or more HCR hairpins comprise one or more reporters, and optionally a readout probe set comprising a reporter-binding domain and one or more auxiliary reporters (for example, see FIGS. 7A-7J and 11A-11E). In some embodiments, the device is configured so that the reagents automatically arrive in the test region in one or more successive stages, generating an amplified signal in the test region if the target is present in the sample (for example, see FIGS. 8A-8D, 12A-12D, 30A-30B, and 32A-32B).

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 7A-7J and 11A-11E). In some embodiments, the device is configured so that the reagents automatically arrive in the test region in one or more successive stages, generating an amplified signal in the test region if the target is present in the sample (for example, see FIGS. 8A-8D, 12A-12D, 30A-30B, and 32A-32B).

In some embodiments an HCR lateral flow device comprises a test region, a capture probe set, a signal probe set, an HCR amplifier comprising one or more reporters, and optionally a readout probe set comprising one or more auxiliary reporters (for example, see FIGS. 7A-7J and 11A-11E). In some embodiments, the device is configured so that the reagents automatically arrive in the test region in a prescribed order, generating an amplified signal in the test region if the target is present in the sample (for example, see FIGS. 8A-8D, 12A-12D, 30A-30B, and 32A-32B).

In some embodiments, reagents are automatically delivered to the test region in multiple successive stages using a multi-channel membrane (for example, see FIGS. 6, 30A-30B, 32A-32B, and 33A-33C, and 33F). In some embodiments, reagents are automatically delivered to the test region in successive stages using a multi-channel membrane in which channels of different lengths lead into a unified channel before reaching the test region (for example, see FIGS. 6, 30A-30B, 32A-32B, and 33C). In some embodiments, channels of differing lengths deliver reagents to the test region in order according to their lengths, with the reagents from the shortest channel arriving in the test region first, the reagents from the longest channel arriving in the test region last, and channels of intermediate lengths arriving in the test region at intermediate times (for example, see FIGS. 6, 30A-30B, 32A-32B, and 33C). In some embodiments, channels are pre-treated with speed additives that create different flow speeds in different channels, allowing for delivery of reagents to the test region in successive stages. In some embodiments, the speed additives comprise sucrose, fructose, trehalose, a sugar, dextran sulfate, a dissolvable barrier, a wax barrier, water-insoluble ink, sponge, valves, pressurized paper, agarose, gravitational orientation, or another agent. In some embodiments, multiple channels are stacked on top of each other to form a multi-layer membrane (for example, see FIGS. 33B-33C). In some embodiments, the sample arrives in different channels at different times, leading to successive delivery of reagents to the test region. In some embodiments, dissolvable barriers dissolve at different rates to introduce the sample into different channels at different times, leading to successive delivery of reagents to the test region. In some embodiments, different reagents migrate through the same or different channels at different rates leading to successive staged arrival times in the test region. For example, auxiliary-reporter-labeled readout probes labeled with bulky auxiliary reporters migrate through the membrane more slowly than reporter-labeled HCR hairpins labeled with small reporters, allowing for the readout probes to arrive in the test region after the HCR hairpins. In some embodiments, the size of different reagents affects their migration speeds through the same channel, allowing for successive delivery of reagents to the test region. In some embodiments, a multi-channel membrane is configured so that reagents in each channel arrive in the test region (or another location) in a prescribed order (for example, see FIGS. 6, 30A-30B, 32A-32B, and 33A-33C, and 33F).

In some embodiments, reagents for each channel are dried onto separate conjugate pads (for example, see FIGS. 5, 30A-30B, 32A-32B, 33A-33C, and 33E-33F). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user input. In some embodiments, draining of a first conjugate pad allows draining of a second conjugate pad, which in turn allows draining of a third conjugate pad, and so on. (For example, see FIGS. 5, 30A-30B, and 32A-32B). In some embodiments, draining of the first conjugate pad frees a unified channel to allow draining of a second conjugate pad, which in turn frees a unified channel to allow draining of a third conjugate pad, and so on (for example, see FIGS. 5, 30A-30B, and 32A-32B).

In some embodiments, a membrane, a sample pad, a conjugate pad, and/or a wicking pad comprise nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.

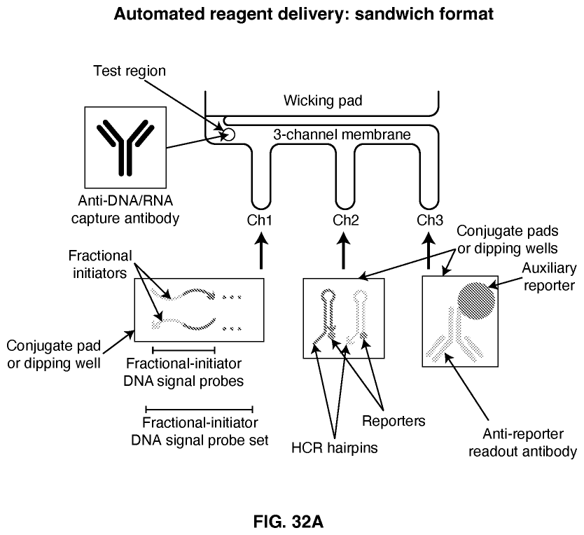

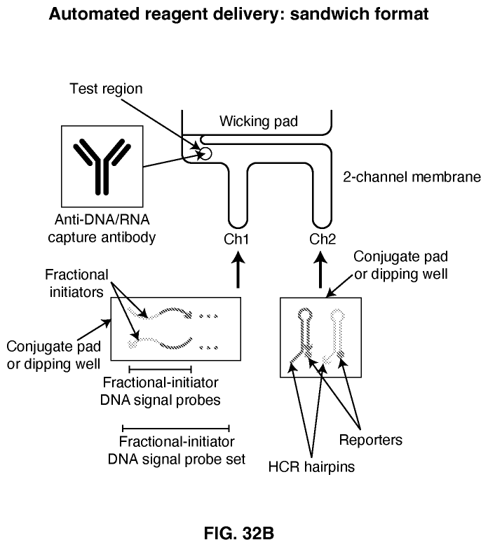

In some embodiments, reagents are automatically delivered to the test region in two successive stages using a two-channel membrane in which channels of different lengths lead into a unified channel before reaching the test region (for example, see FIGS. 30B and 32B). In some embodiments, the signal probe set binds the nucleic acid target and travels via the shorter channel (Channel 1) to reach the test region first where the target additionally binds the capture probe set pre-immobilized in the test region to form an immobilized probe/target sandwich (for example, see FIGS. 8C and 12C). In some embodiments, the signal probe set and capture probe set bind the target nucleic acid and travel via the shorter channel (Channel 1) to reach the test region first, where the probe/target sandwich is immobilized via binding of immobilization-ligand-labeled capture probes to immobilization receptors pre-immobilized in the test region (for example, see FIGS. 8D, 12D, 30B, and 32B). In some embodiments, reporter-labeled HCR hairpins travel via a longer channel (Channel 2) and reach the test region next, where amplification domains on the signal probe set trigger growth of tethered HCR amplification polymers decorated with reporters to generate an amplified signal in the test region (for example, see FIGS. 8C-8D, 12C-12D, 30B, and 32B). In some embodiments, reagents for each channel are dried onto two separate conjugate pads (for example, see FIGS. 30B and 32B). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user interaction. In some embodiments, draining of the first conjugate pad allows draining of the second conjugate pad. In some embodiments, draining of the first conjugate pad frees a unified channel to allow draining of the second conjugate pad. In some embodiments, reagents in the two channels arrive in the test region (or another location) in successive stages in a prescribed order due to channel length, channel geometry, speed additives, dissolvable barriers, or other conditions (for example, see FIGS. 30B and 32B).

In some embodiments, reagents are automatically delivered to the test region in three successive stages using a three-channel membrane in which channels of different lengths lead into a unified channel before reaching the test region (for example, see FIGS. 30A and 32A). In some embodiments, the signal probe set binds the target and travels via the shortest channel (Channel 1) to reach the test region first where the target additionally binds the capture probe set that is pre-immobilized in the test region to form an immobilized probe/target sandwich (for example, see FIGS. 8A and 12A). In some embodiments, the signal probe set and capture probe set bind the target and travel via the shortest channel (Channel 1) to reach the test region first, where the probe/target sandwich is immobilized via binding of immobilization-ligand-labeled capture probes to immobilization receptors pre-immobilized in the test region (for example, see FIGS. 8B, 12B, 30A, and 32A). In some embodiments, reporter-labeled HCR hairpins travel via a channel of intermediate length (Channel 2) and reach the test region next, where amplification domains on the anti-target signal antibody triggers growth of tethered HCR amplification polymers decorated with reporters (for example, see FIGS. 8A-8B, 12A-12B, 30A, and 32A). In some embodiments, an auxiliary-reporter-labeled readout probe set travels via the longest channel (Channel 3) and arrives in the test region last, where it binds the reporters decorating the HCR amplification polymers to generate an amplified signal in the test region (for example, see FIGS. 8A-8B, 12A-12B, 30A, and 32A). In some embodiments, reagents for each channel are dried onto separate conjugate pads (for example, see FIGS. 30A and 32A). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of the first conjugate pad allows draining of the second conjugate pad, which in turn allows draining of the third conjugate pad, and so on. In some embodiments, draining of the first conjugate pad frees the unified channel to allow draining of the second conjugate pad, which in turn frees the unified channel to allow draining of the third conjugate pad. In some embodiments, reagents in the three channels arrive in the test region (or another location) in successive stages in a prescribed order due to channel length, channel geometry, speed additives, dissolvable barriers, or other conditions (for example, see FIGS. 30A and 32A).

In some embodiments, the device comprises a control region (for example, see FIGS. 34A-34B, 35A-35B, and 36A-36B). In some embodiments, the control region is downstream of the test region so that the sample and reagents flow through the test region before reaching the control region. In some embodiments an HCR lateral flow device comprises a test region, a control region, a capture probe set, a signal probe set, a control capture probe set, a control signal probe set, an HCR amplifier comprising one or more reporters, and optionally a readout probe set comprising one or more auxiliary reporters (for example, see FIGS. 34A-34B, 35A-35B, and 36A-36B). In some embodiments, the device is configured so that the reagents automatically arrive in the test region and the control region in one or more successive stages (for example, see FIGS. 36A-36B), generating an amplified signal in the test region if the target is present in the sample, and generating an amplified control signal in the control region if the sample and reagents flow through the device properly. In some embodiments, if the sample and reagents successfully flow through the control region, the control capture probe set binds to and immobilizes the control signal probe set in the control region, the control amplification domains on the control signal probe set trigger the self-assembly of tethered HCR amplification polymers decorated with reporters to directly or indirectly generate an amplified control signal in the control region, and optionally the auxiliary-reporter-labeled readout probe set binds the control reporters to directly or indirectly generate an amplified control signal in the control region. In some embodiments, the test produces a positive result if both the test region and the control region produce visible signals. In some embodiments, the test produces a negative result if signal is observed in the control region but not in the test region. In some embodiments, absence of signal in the control region is an indication that the test did not function properly.

In some embodiments, the device comprises a folding card geometry (for example, see FIG. 5). In some embodiments, one page of the card contains a sample pad and one or more conjugate pads. In some embodiments, the other page of the card contains a membrane comprising one or more channels, a test region, an optional control region, and a wicking pad. In some embodiments, the wicking pad absorbs liquid to induce continued capillary flow through the channels after the sample reaches the wicking pad. In some embodiments, one page is functionalized with prongs that disconnect the sample pad from the one or more conjugate pads upon folding the card. In some embodiments, disconnecting the sample pad from the one or more conjugate pads limits the volume that flows from the one or more conjugate pads and prevents flow between conjugate pads. In some embodiments, the device comprises a membrane comprising one or more channels. In some embodiments, the user runs the test by dipping the ends of one or more channels into one or more solutions containing reagents. In some embodiments, the device takes the form of a comb, wagon wheel, stacked device, half-strip, dipstick, reverse-flow device, any combination thereof, or another geometry.

In some embodiments, the user preps the sample by adding the sample to a prep buffer (for example, see FIG. 38A-38B). In some embodiments, the user preps the sample by adding prep buffer to the sample (for example, see FIG. 38C). In some embodiments, the user preps the sample by heating the sample in solution. In some embodiments, the prep buffer is an extraction buffer that exposes the target (for example, by disrupting a viral envelope or cell membrane). In some embodiments, the prep buffer is a denaturant. In some embodiments, the user adds the prepped sample to a sample pad to start a test. In some embodiments, the user adds the prep buffer to the sample pad and adds the sample to the prepped sample pad (for example, see FIG. 38D). In some embodiments, the user adds sample to the sample pad (for example, by putting a swab in contact with the sample pad) and then adds the prep buffer to the sample and sample pad (for example, see FIG. 38E). In some embodiments, the user adds the sample to the device (for example, see FIG. 38F).

In some embodiments, the user closes a folding card device to start the test (for example, see FIG. 38D). In some embodiments, closing the card creates contacts between the membrane and one or more conjugate pads, initiating the flow of liquid in one or more channels. In some embodiments, the reagents are delivered to the test region in successive stages via one or more channels without user interaction. In some embodiments, the user reads either a positive result (visible signal in the test region) or a negative result (no visible signal in the test region) with the naked eye. In some embodiments, the user or a health care professional reads a result using an instrument to scan the test region (for example, a fluorescence scanner, camera, radiation scanner, mass spectrometer, or other device that allows acquisition of a signal from the test region).

In some embodiments, the target nucleic acid binds a capture probe set comprising one or more capture probes, wherein a capture probe comprises a target-binding domain configured to bind directly or indirectly to the target nucleic acid and an immobilization domain configured to directly or indirectly immobilize the target in a test region (for example, see FIGS. 7A-7J and 11A-11E). In some embodiments, the capture probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above. In some embodiments, the immobilization domain binds directly to the test region. In some embodiments, the capture probe set is pre-immobilized in the test region (for example, see FIGS. 8A, 8C, 12A, and 12C). In some embodiments, the immobilization domain comprises an immobilization ligand. In some embodiments, the immobilization ligand binds an immobilization receptor that is pre-immobilized in the test region (for example, see FIGS. 8B, 8D, 12B, and 12D).

In some embodiments, the target nucleic acid binds a signal probe set comprising one or more signal probes, wherein a signal probe comprises a target-binding domain configured to bind directly or indirectly to the target nucleic acid and an amplification domain that is configured to directly or indirectly initiate a chain reaction in which HCR hairpins self-assemble to form an HCR amplification polymer (for example, see FIGS. 7A-7J and 11A-11E). In some embodiments, the signal probe set comprises: a) one or more initiator-labeled probes wherein an initiator-labeled probe comprises a target-binding domain and an amplification domain comprising one or more HCR initiators (for example, see FIGS. 3B, 7A-7E, 7H, 11B, 18A-18N, and 19A-19F), or b) one or more probe units, each comprising two or more fractional-initiator probes, wherein a fractional-initiator probe comprises a target-binding domain and an amplification domain comprising a fractional initiator (for example, see FIGS. 4A, 7F-7G, 7I-7J, 20A-20E, 21A-21B, 22A, 22B, 22E, and 24A-24R). In some embodiments, a signal probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above.

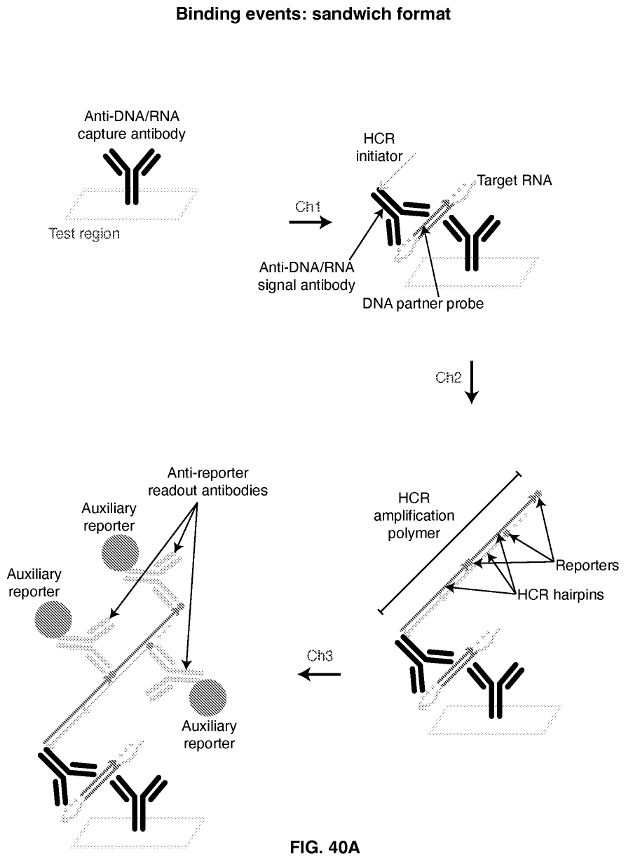

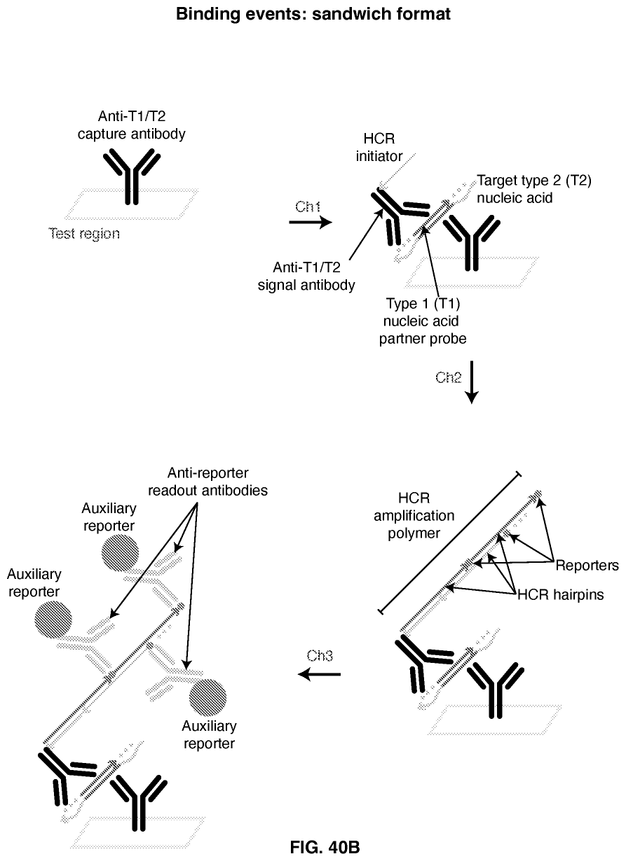

In some embodiments, the target nucleic acid is a target RNA. In some embodiments, a DNA partner probe set comprises one or more DNA partner probes configured to bind to different subsequences along the target RNA (for example, see FIG. 40A). In some embodiments, binding of the DNA partner probes to their cognate binding sites along the target RNA forms DNA/RNA duplexes. In some embodiments, the capture probe set and/or the signal probe set comprise anti-DNA/RNA antibodies that bind DNA/RNA duplexes. In some embodiments, the anti-DNA/RNA capture antibody further comprises an immobilization domain that directly or indirectly immobilizes the probe/target sandwich in the test region. In some embodiments, the anti-DNA/RNA signal antibodies further comprise an amplification domain that directly or indirectly triggers growth of an HCR amplification polymer.

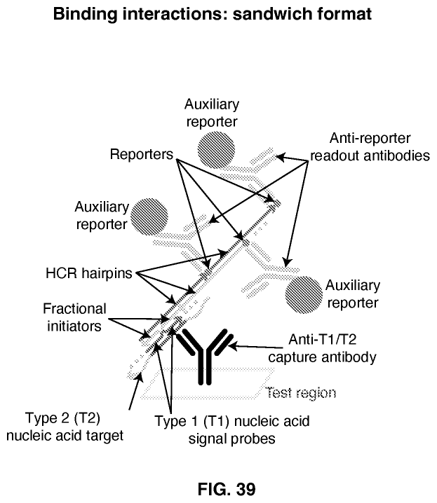

In some embodiments, the signal probe set comprises one or more type 1 (T1) nucleic acid probes (for example, DNA, RNA, 2′OMe-RNA, chemically modified nucleic acid, or any material capable of base-pairing), the target is a type 2 (T2) nucleic acid (for example, RNA, DNA, chemically modified nucleic acid, or any material capable of base-pairing), and the capture probe set comprises one or more anti-T1/T2 antibodies, anti-T1/T2 nanobodies, or any molecule or complex that recognizes T1/T2 duplexes (for example, see FIG. 39). In some embodiments, T1 and T2 are the same type of nucleic acids. In some embodiments, T1 and T2 are different types of nucleic acids.

In some embodiments, the target is bound by a partner probe set comprising one or more type 1 (T1) nucleic acid probes (for example, DNA, RNA, 2′OMe-RNA, chemically modified nucleic acid, or any material capable of base-pairing), the target is a type 2 (T2) nucleic acid (for example, RNA, DNA, chemically modified nucleic acid, or any material capable of base-pairing), and: a) the capture probe set comprises one or more anti-T1/T2 antibodies, anti-T1/T2 nanobodies, or molecules or complexes that recognize T1/T2 duplexes, and/or b) the signal probe set comprises one or more anti-T1/T2 antibodies, anti-T1/T2 nanobodies, or other molecules or complexes that recognize T1/T2 duplexes (for example, see FIG. 40B). In some embodiments, T1 and T2 are the same type of nucleic acids. In some embodiments, T1 and T2 are different types of nucleic acids.

In some embodiments, following sequencing of a new nucleic acid target of interest, new nucleic acid probe sets can be synthesized and tested within days. In some embodiments, days after the emergence of a new pathogen, an amplified HCR lateral flow assay can be developed and tested for a new nucleic acid target, contrasting with the months it can take to produce high-quality antibodies for a new protein target.

### HCR Lateral Flow Assays for Multiplexed Amplified Detection of N Targets in a Sample

In some embodiments an amplified HCR lateral flow device is used to detect N targets in a sample (where N is an integer greater than or equal to 2).

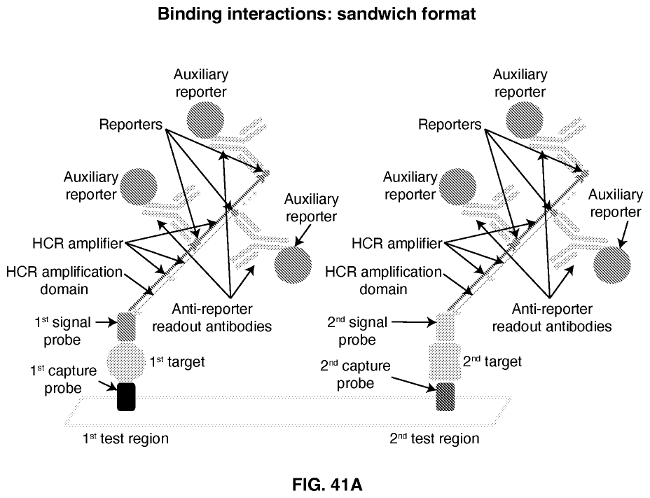

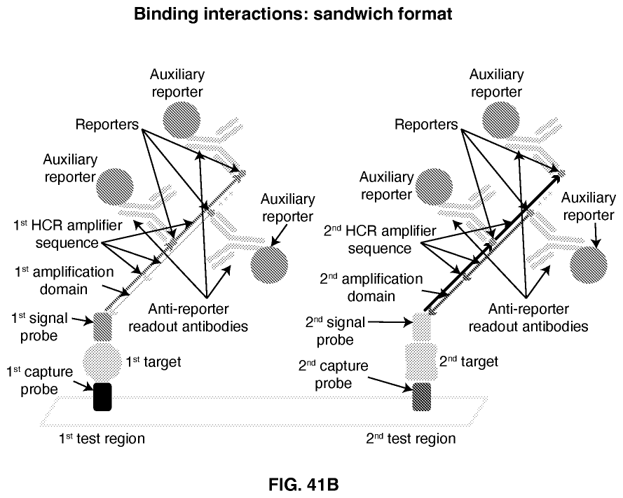

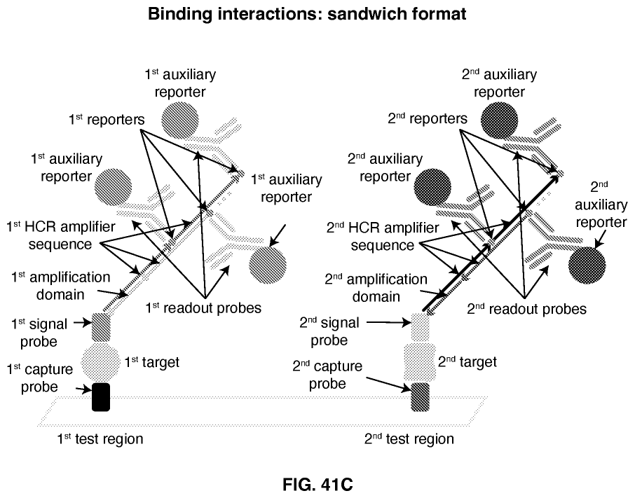

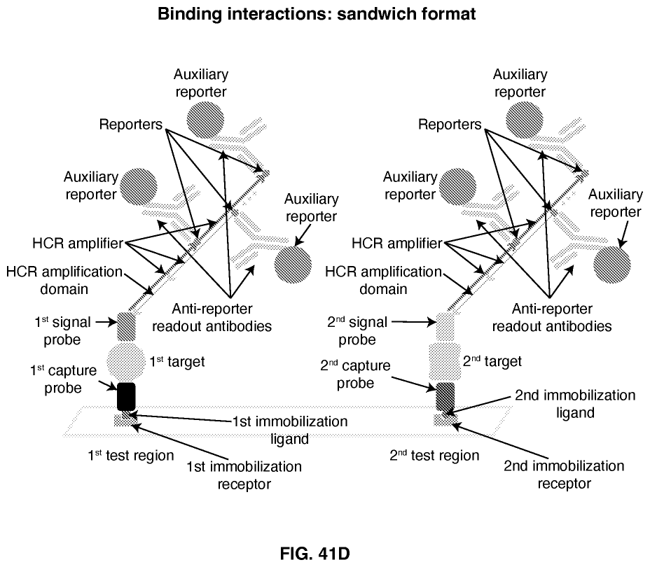

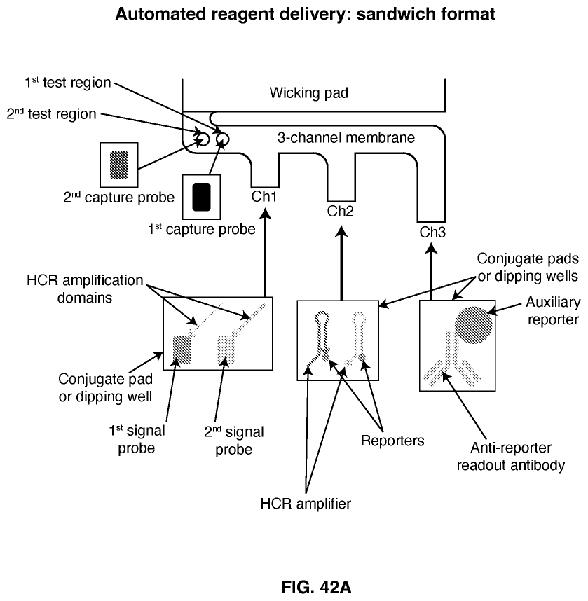

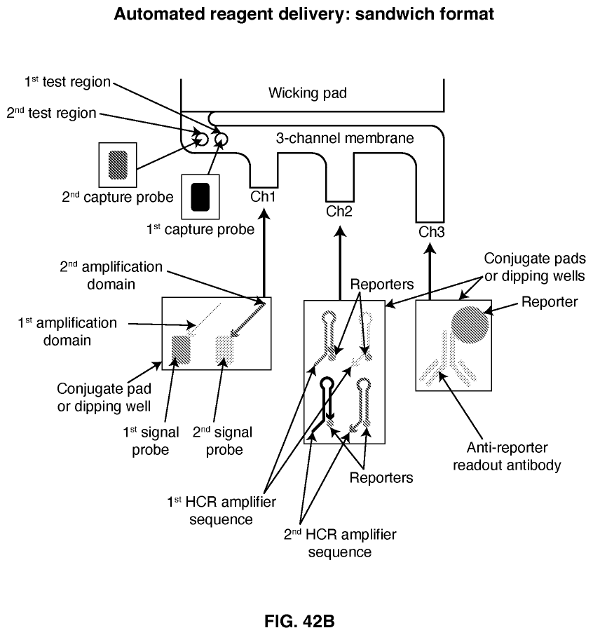

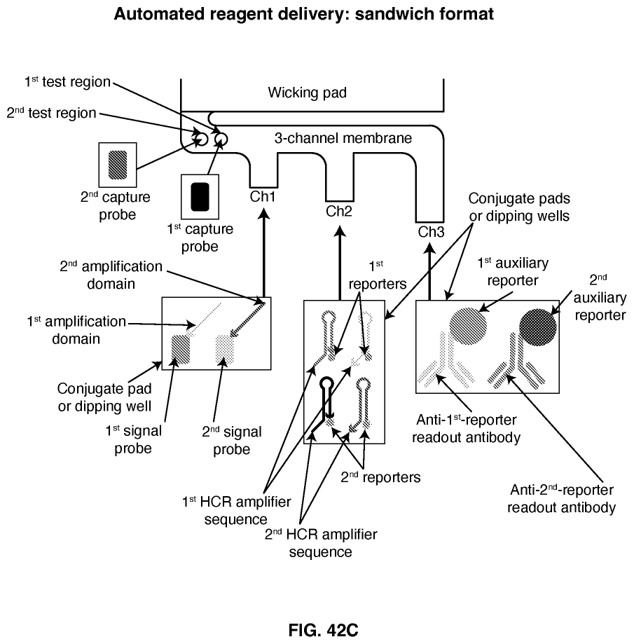

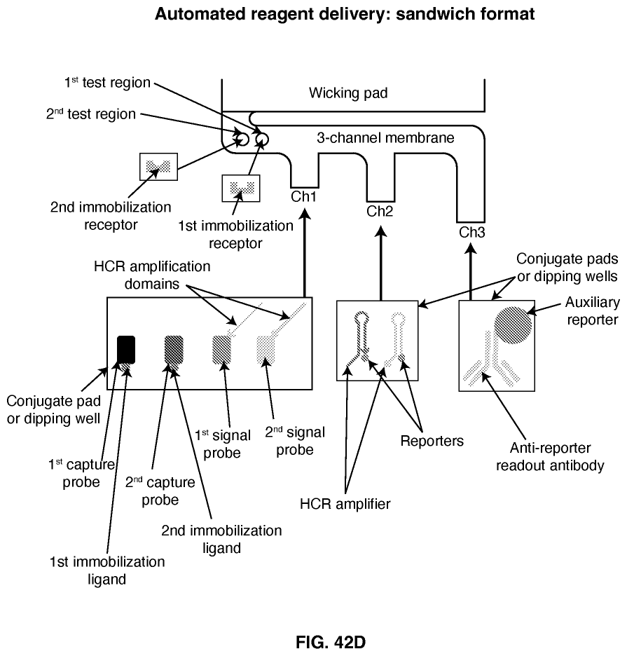

In some embodiments, the device comprises N test regions, wherein the j^{th} test region (j=1, 2, . . . , N) is used to detect the presence or absence of the j^{th} target in the sample (for example, see FIGS. 41A-41D and 42A-42D). In some embodiments, the device comprises N capture probe sets wherein the j^{th} capture probe set (j=1, 2, . . . , N) comprises one or more capture probes, wherein a capture probe comprises a target-binding domain configured to bind directly or indirectly to the j^{th} target and an immobilization domain configured to directly or indirectly bind the j^{th} test region (for example, see FIGS. 41A-41D and 42A-42D). In some embodiments, the j^{th} capture probe set (j=1, 2, . . . , N) is pre-immobilized in the j^{th} test region (for example, see FIGS. 41A-41C and 42A-42C). In some embodiments, there are N immobilization ligands and N immobilization receptors and the capture probes in the je capture probe set j=1, 2, . . . , N) are labeled with the j^{th} immobilization ligand configured to bind the j^{th} immobilization receptor that is pre-immobilized in the j^{th} target region (for example, FIGS. 41D and 42D).

In some embodiments, the device comprises N HCR amplifiers wherein the j^{th} HCR amplifier j=1, 2, . . . , N) comprises two or more HCR hairpins (for example, see FIGS. 41B-41C and 42B-42C). In some embodiments, there are N reporters (for example, see FIGS. 41C and 42C). In some embodiments, one or more of the hairpins in the j^{th} HCR amplifier (j=1, 2, . . . , N) are labeled with the j^{th} reporter. In some embodiments, the HCR hairpins in the j^{th} HCR amplifier j=1, 2, . . . , N) are configured not to polymerize in the absence of the j^{th} HCR initiator. In some embodiments, HCR hairpins in the j^{th} HCR amplifier (j=1, 2, . . . , N) are configured such that detection of the j^{th} HCR initiator (or the colocalized j^{th} full initiator formed from colocalization of the fractional initiators in a probe unit) triggers the HCR hairpins to polymerize to form a tethered j^{th} HCR amplification polymer decorated with j^{th} reporters. In some embodiments, the N HCR amplifier sequences are orthogonal such that the cognate initiator for one amplifier will not trigger HCR signal amplification by any of the other amplifiers (for example, see FIGS. 41B-41C and 42B-42C). In some embodiments, the N reporters are distinct. In some embodiments, some or all of the N HCR amplifiers have identical amplifier sequences for different targets (each HCR amplifier sequence comprising the sequences of two or more HCR hairpins) (for example, see FIGS. 41A, 41D, 42A, and 42D). In some embodiments, some or all of the N reporters labeling the N HCR amplifiers are identical for different targets (for example, see FIGS. 41A-41B, 41D, 42A-42B, and 42D). In some embodiments, the same HCR amplifier is used for all N targets (for example, see FIGS. 41A, 41D, 42A and 42D).

In some embodiments, the device comprises N signal probe sets wherein the j^{th} signal probe set (j=1, 2, . . . , N) comprises one or more signal probes, wherein a signal probe comprises a target-binding domain configured to bind directly or indirectly to the j^{th} target and an amplification domain that is configured to directly or indirectly initiate a chain reaction in which the HCR hairpins in the j^{th} HCR amplifier self-assemble to form a tethered j^{th} HCR amplification polymer (for example, see FIGS. 41A-41D and 42A-42D). In some embodiments, the j^{th} signal probe set (j=1, 2, . . . , N) comprises: a) one or more initiator-labeled probes wherein an initiator-labeled probe comprises a target-binding domain configured to bind directly or indirectly to the j^{th} target and an amplification domain comprising one or more j^{th} HCR initiators (for example, see FIGS. 41A-41D and 42A-42D), or b) one or more probe units, each comprising two or more fractional-initiator probes, wherein a fractional-initiator probe comprises a target-binding domain configured to bind the j^{th} target and an amplification domain comprising a j^{th} fractional initiator. In some embodiments, an individual fractional-initiator probe from the j^{th} signal probe set that binds non-specifically will not trigger the j^{th} HCR amplifier, but specific binding of the probes comprising a probe unit from the j^{th} signal probe set to adjacent cognate binding sites on the j^{th} target colocalizes a j^{th} full HCR initiator capable of triggering the j^{th} HCR amplifier. In some embodiments, some or all of the N signal probe sets have identical amplification domains for different targets (for example, see FIGS. 41A, 41D, 42A, and 42D).

In some embodiments, the device comprises N readout probe sets wherein the j^{th} readout probe set (j=1, 2, . . . , N) comprises one or more readout probes, wherein a readout probe comprises a j^{th}-reporter-binding domain configured to bind directly or indirectly to the j^{th} reporter on a j^{th} HCR amplification polymer (for example, see FIGS. 41C and 42C). In some embodiments, the device comprises N auxiliary reporters wherein the readout probes in the j^{th} readout probe set j=1, 2, . . . , N) comprise the j^{th} auxiliary reporter wherein the j^{th} auxiliary reporter contributes directly or indirectly to the generation of a j^{th} signal (for example, see FIGS. 41C and 42C). In some embodiments, the N auxiliary reporters are distinct. In some embodiments, the j^{th} signal (j=1, 2, . . . , N) generated by the j^{th} auxiliary reporter on the readout probes in the j^{th} readout probe set is visible to the human eye. In some embodiments, some or all of the N readout probe sets are configured to bind to identical reporters for different targets. In some embodiments, some or all of the N auxiliary reporters are identical for different targets. In some embodiments, the same readout probe set is used for all N targets (for example, see FIGS. 41A-41B, 41D, 42A-42B, and 42D).

In some embodiments, the j^{th} target j=1, 2, . . . , N) binds in a j^{th} sandwich between a j^{th} capture probe set and a j^{th} signal probe set (for example, see FIGS. 41A-41C). In some embodiments, the j^{th} capture probe set j=1, 2, . . . , N) immobilizes the j^{th} probe/target sandwich in the j^{th} test region (for example, see FIGS. 41A-41D). In some embodiments, if the j^{th} target is not present in the sample, the j^{th} probe/target sandwich does not form, the j^{th} signal probe set is not immobilized in the j^{th} test region, the growth of a tethered j^{th} HCR amplification polymer is not triggered in the j^{th} test region, and a j^{th} amplified signal is not generated in the j^{th} test region.

In some embodiments, after the j^{th} probe/target sandwich j=1, 2, . . . , N) is immobilized in the j^{th} test region, the amplification domains on the signal probes in the j^{th} probe set trigger the self-assembly of j^{th}-reporter-labeled HCR hairpins from the j^{th} HCR amplifier into tethered j^{th} HCR amplification polymers decorated with j^{th} reporters (for example, see FIGS. 41A-41D). In some embodiments, the j^{th} reporters (j=1, 2, . . . , N) directly or indirectly lead to generation of a j^{th} signal. In some embodiments, j-auxiliary-reporter-labeled readout probes (j=1, 2, . . . , N) from the j^{th} readout probe set bind the j^{th} reporters decorating the j^{th} HCR amplification polymer immobilized in the j^{th} test region to directly or indirectly lead to generation of a j^{th} amplified signal (for example, see FIGS. 41A-41D). In some embodiments, some or all of the N signals are visible to the human eye. In some embodiments, some or all of the N signals are read using a fluorescence microscope, a fluorescence scanner, a camera, a mobile phone camera, a mass spectrometer, a mass spectrometry microscope, a radioactive scanner, or another instrument suitable for detecting reporters. In some embodiments, some or all of the N reporters and/or some or all of the N auxiliary reporters comprise a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, an enzyme, any combination thereof, or any other molecule that facilitates measurement of a signal. In some embodiments, some or all of the N reporters and/or some or all of the auxiliary reporters is a hapten, a ligand, digoxigenin (DIG), fluorescein isothiocyanate (FITC), a fluorophore, biotin, dinitrophenol, aniline, or another molecule or complex that can be recognized by a binding partner to facilitate generation of a signal. In some embodiments, the reporters decorating a j^{th} HCR amplification polymer (j=1, 2, . . . , N) directly or indirectly mediate catalytic reporter deposition (CARD) to generate a j^{th} amplified signal in the vicinity of the j^{th} HCR amplification polymer. In some embodiments, the j^{th} auxiliary reporters (j=1, 2, . . . , N) decorating the j^{th} HCR amplification polymer directly or indirectly mediate catalytic reporter deposition (CARD) to generate a j^{th} amplified signal in the vicinity of the j^{th} HCR amplification polymer in the j^{th} test region. In some embodiments, CARD is mediated by an enzyme.

In some embodiments, reagents are automatically delivered to each of N test regions in multiple successive stages using a multi-channel membrane (for example, see FIGS. 42A-42D). In some embodiments, a multi-channel membrane is configured so that reagents arrive at each of N test regions (or another location) in a prescribed order (for example, see FIGS. 42A-42D).

In some embodiments, reagents for each channel are dried onto separate conjugate pads (for example, see FIGS. 42A-42D). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user input. In some embodiments, draining of a first conjugate pad allows draining of a second conjugate pad, which in turn allows draining of a third conjugate pad, and so on. In some embodiments, draining of the first conjugate pad frees a unified channel to allow draining of a second conjugate pad, which in turn frees a unified channel to allow draining of a third conjugate pad, and so on.

In some embodiments, reagents are automatically delivered to the test region in two successive stages using a two-channel membrane in which channels of different lengths lead into a unified channel before reaching the test region. In some embodiments, the j^{th} signal probe set (j=1, 2, . . . , N) and j^{th} capture probe set bind the j^{th} target and travel via the shortest channel (Channel 1) to reach the j^{th} test region in Stage 1, where the j^{th} probe/target sandwich is immobilized via binding of j^{th}-immobilization-ligand-labeled capture probes to j^{th} immobilization receptors pre-immobilized in the test region. In some embodiments, the j^{th} signal probe set binds the j^{th} target and travels via the shorter channel (Channel 1) to reach the j^{th} test region in Stage 1 where the j^{th} target additionally binds the j^{th} capture probe set that is pre-immobilized in the j^{th} test region to form an immobilized j^{th} probe/target sandwich. In some embodiments, j^{th}-reporter-labeled HCR hairpins (j=1, 2, . . . , N) for the j^{th} HCR amplifier travel via a longer channel (Channel 2) and reach the test region in Stage 2, where amplification domains on the j^{th} signal probe set trigger growth of tethered j^{th} HCR amplification polymers decorated with j^{th} reporters to generate a j^{th} amplified signal in the j^{th} test region. In some embodiments, reagents for each channel are dried onto separate conjugate pads. In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of the first conjugate pad allows draining of the second conjugate pad. In some embodiments, draining of the first conjugate pad frees a unified channel to allow draining of the second conjugate pad. In some embodiments, reagents in the two channels arrive in the test region (or another location) in successive stages in a prescribed order due to channel length, channel geometry, speed additives, dissolvable barriers, or other conditions.

In some embodiments, reagents are automatically delivered to the test region in three successive stages using a three-channel membrane in which channels of different lengths lead into a unified channel before reaching the test region (for example, see FIGS. 42A-42D). In some embodiments, the j^{th} signal probe set (j=1, 2, . . . , N) and j^{th} capture probe set bind the target and travel via the shortest channel (Channel 1) to reach the j^{th} test region in Stage 1, where the j^{th} probe/target sandwich is immobilized via binding of j^{th}-immobilization-ligand-labeled j^{th} capture probe set to j^{th} immobilization receptors pre-immobilized in the j^{th} test region. In some embodiments, the j^{th} signal probe set (j=1, 2, . . . , N) binds the j^{th} target and travels via the shortest channel (Channel 1) to reach the j^{t} test region in Stage 1 where the j^{th} target additionally binds the pre-immobilized j^{th} capture probe set to form an immobilized j^{th} probe/target sandwich. In some embodiments, j^{th}-reporter-labeled HCR hairpins (j=1, 2, . . . , N) from the j^{th} HCR amplifier travel via a channel of intermediate length (Channel 2) and reach the test region in Stage 2, where amplification domains on the j^{th} signal probe set trigger growth of tethered j^{th} HCR amplification polymers decorated with j^{th} reporters. In some embodiments, j^{th}-auxiliary-reporter-labeled j^{th} readout probes (j=1, 2, . . . , N) travel via the longest channel (Channel 3) and arrive in the test region in Stage 3, where they bind the j^{th} reporters decorating the j^{th} HCR amplification polymers to generate a j^{th} amplified signal in the j^{th} test region. In some embodiments, reagents for each channel are dried onto separate conjugate pads. In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, upon rehydration, successive delivery of the reagents to the test region occurs automatically without user input. In some embodiments, draining of the first conjugate pad allows draining of the second conjugate pad, which in turn allows draining of the third conjugate pad, and so on. In some embodiments, draining of the first conjugate pad frees the unified channel to allow draining of the second conjugate pad, which in turn frees the unified channel to allow draining of a third conjugate pad, and so on. In some embodiments, reagents in the three channels arrive in the test region (or another location) in successive stages in a prescribed order due to channel length, channel geometry, speed additives, dissolvable barriers, or other conditions.

In some embodiments, an amplified HCR lateral flow device is used to detect N targets in a sample.

In some embodiments, the device comprises N test regions (one per target), N distinct capture probe sets (one per target), N distinct signal probe sets with distinct amplification domains (one per target), and N distinct HCR amplifier sequences labeled with distinct reporters (one per target). In some embodiments, the device further comprises N distinct readout probe sets labeled with distinct auxiliary reporters (one per target) (for example, see FIG. 42C).

In some embodiments, the device comprises N test regions (one per target), N distinct capture probe sets (one per target), N distinct signal probe sets with distinct amplification domains (one per target), and N distinct HCR amplifier sequences labeled with distinct reporters (one per target). In some embodiments, the device further comprises N distinct readout probe sets (one per target) all labeled with the same auxiliary reporter.

In some embodiments, the device comprises N test regions (one per target), N distinct capture probe sets (one per target), N distinct signal probe sets with distinct amplification domains (one per target), and N distinct HCR amplifier sequences (one per target) all labeled with the same reporter. In some embodiments, the device further comprises a readout probe set labeled with an auxiliary reporter (for example, see FIG. 42B).

In some embodiments, the device comprises N test regions (one per target), N distinct capture probe sets (one per target), N distinct signal probe sets (one per target) all with the same amplification domain, and an HCR amplifier labeled with a reporter. In some embodiments, the device further comprises a readout probe set labeled with an auxiliary reporter (for example, see FIGS. 42A and 42D).

In some embodiments, the device comprises fewer than N test regions. In some embodiments, the color of the amplified signal is different for different targets, enabling two or more targets to be detected with different colors in the same test region.

In some embodiments, an amplified HCR lateral flow device is used to detect two targets in a sample.

In some embodiments, the device comprises two test regions (one per target), two distinct capture probe sets (one per target), two distinct signal probe sets with distinct amplification domains (one per target), and two distinct HCR amplifier sequences labeled with distinct reporters (one per target). In some embodiments, the device further comprises two distinct readout probe sets labeled with distinct auxiliary reporters (one per target) (for example, see FIG. 42C).

In some embodiments, the device comprises two test regions (one per target), two distinct capture probe sets (one per target), two distinct signal probe sets with distinct amplification domains (one per target), and two distinct HCR amplifier sequences labeled with distinct reporters (one per target). In some embodiments, the device further comprises two distinct readout probe sets (one per target) labeled with the same auxiliary reporter.

In some embodiments, the device comprises two test regions (one per target), two distinct capture probe sets (one per target), two distinct signal probe sets with distinct amplification domains (one per target), two distinct HCR amplifier sequences (one per target) labeled with the same reporter. In some embodiments, the device further comprises a readout probe set labeled with an auxiliary reporter (for example, see FIG. 42B).

In some embodiments, the device comprises two test regions (one per target), two distinct capture probe sets (one per target), two distinct signal probe sets (one per target) with the same amplification domain, and an HCR amplifier labeled with a reporter. In some embodiments, the device further comprises a readout probe set labeled with an auxiliary reporter (for example, see FIGS. 42A and 42D).

In some embodiments an HCR lateral flow device for detecting N targets in a sample comprises N test regions and N control regions. In some embodiments, an HCR lateral flow device used to detect N target in a sample comprises N tests regions and one control region; in some embodiments, the one control region is located downstream of the N test regions so that the sample and reagents flow through the N test regions before reaching the control region. In some embodiments, an HCR lateral flow device used to detect N targets in a sample comprises zero, one, or more control regions. In some embodiments, zero, one, or more control regions are located upstream of the N test regions, zero, one, or more control regions are interspersed between the N test regions, and zero, one, or more control regions are located downstream of the N test regions.

In some embodiments, an amplified HCR lateral flow device is used to detect N targets in a sample.

In some embodiments, the device comprises N test regions (one per target) and 1 control region, N distinct capture probe sets (one per target) and 1 distinct control capture probe set, N distinct signal probe sets with distinct amplification domains (one per target) and 1 distinct control signal probe set with a distinct amplification domain, and N distinct HCR amplifier sequences labeled with distinct reporters (one per target) and 1 distinct control HCR amplifier sequence labeled with distinct control reporters. In some embodiments, the device further comprises N distinct readout probe sets labeled with distinct auxiliary reporters (one per target) and 1 distinct control readout probe set labeled with distinct control auxiliary reporters.

In some embodiments, the device comprises N test regions (one per target) and 1 control region, N distinct capture probe sets (one per target) and 1 distinct control capture probe set, N distinct signal probe sets with distinct amplification domains (one per target) and 1 distinct control signal probe set with a distinct amplification domain, and N distinct HCR amplifier sequences labeled with distinct reporters (one per target) and 1 distinct control HCR amplifier sequence labeled with distinct control reporters. In some embodiments, the device further comprises N distinct readout probe sets (one per target) and 1 distinct control readout probe set all labeled with the same auxiliary reporter.

In some embodiments, the device comprises N test regions (one per target) and one control region, N distinct capture probe sets (one per target) and 1 distinct control capture probe set, N distinct signal probe sets with distinct amplification domains (one per target) and 1 distinct control signal probe set with a distinct amplification domain, and N distinct HCR amplifier sequences (one per target) and 1 distinct control HCR amplifier sequence all labeled with the same reporter. In some embodiments, the device further comprises a readout probe set labeled with an auxiliary reporter.

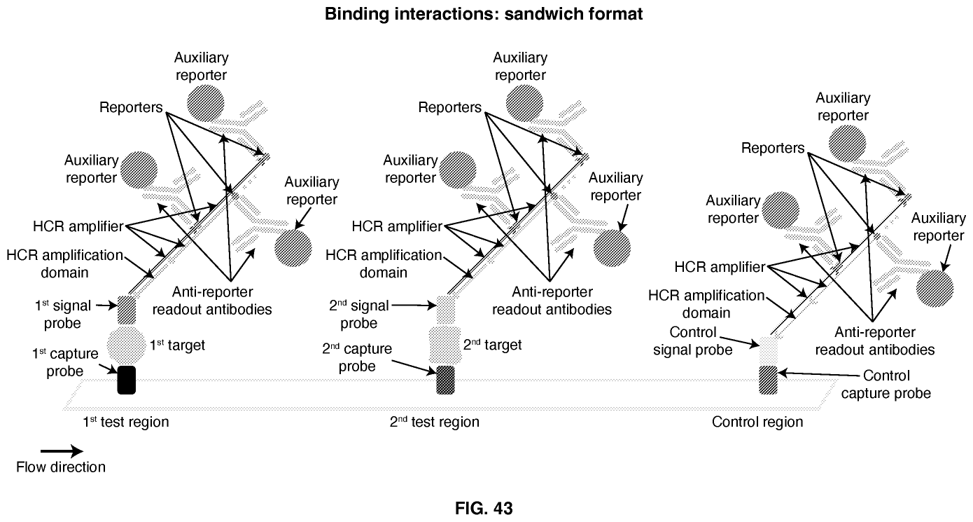

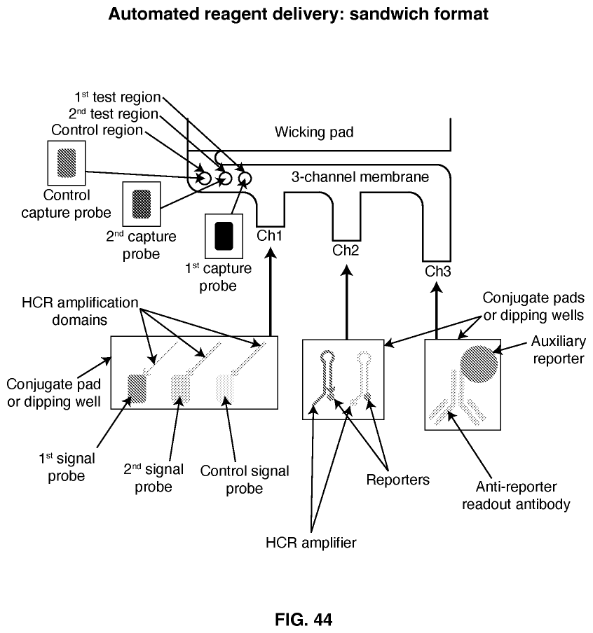

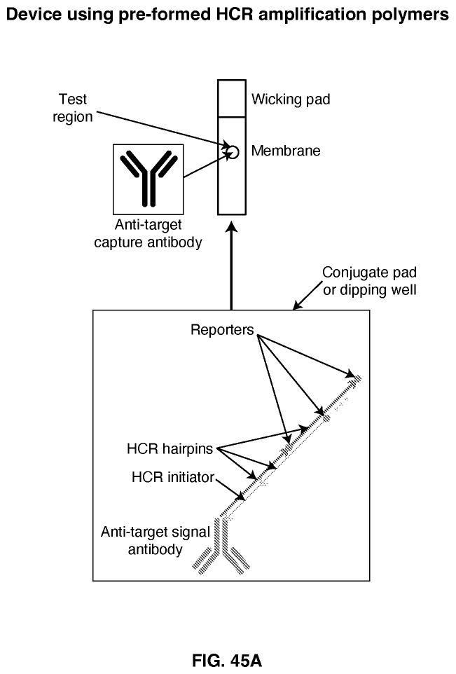

In some embodiments, the device comprises N test regions (one per target) and 1 control region, N distinct capture probe sets (one per target) and 1 distinct control capture probe set, N distinct signal probe sets (one per target) and 1 distinct control signal probe set all with the same amplification domain, and an HCR amplifier labeled with a reporter. In some embodiments, the device further comprises a readout probe set labeled with an auxiliary reporter (for example, see FIGS. 43 and 44).

In some embodiments, an amplified HCR lateral flow device is used to detect two targets in a sample.

In some embodiments, the device comprises two test regions (one per target) and a control region, two distinct capture probe sets (one per target) and one distinct control capture probe set, two distinct signal probe sets with distinct amplification domains (one per target) and one distinct control signal probe set with a distinct amplification domain, and two distinct HCR amplifier sequences labeled with distinct reporters (one per target) and one distinct control HCR amplifier sequence labeled with distinct control reporters. In some embodiments, the device further comprises two distinct readout probe sets labeled with distinct auxiliary reporters (one per target) and one distinct control readout probe set labeled with distinct control auxiliary reporters.

In some embodiments, the device comprises two test regions (one per target) and one control region, two distinct capture probe sets (one per target) and one distinct control capture probe set, two distinct signal probe sets with distinct amplification domains (one per target) and one distinct control signal probe set with a distinct amplification domain, and two distinct HCR amplifier sequences labeled with distinct reporters (one per target) and one distinct control HCR amplifier sequence labeled with distinct control reporters. In some embodiments, the device further comprises two distinct readout probe sets (one per target) and one distinct control readout probe set all labeled with the same auxiliary reporter.

In some embodiments, the device comprises two test regions (one per target) and one control region, two distinct capture probe sets (one per target) and one distinct control capture probe set, two distinct signal probe sets with distinct amplification domains (one per target) and one distinct control signal probe set with a distinct amplification domain, two distinct HCR amplifier sequences (one per target) and one distinct control HCR amplifier sequence all labeled with the same reporter. In some embodiments, the device further comprises a readout probe set labeled with an auxiliary reporter.

In some embodiments, the device comprises two test regions (one per target) and one control region, two distinct capture probe sets (one per target) and one distinct control capture probe set, two distinct signal probe sets (one per target) and one distinct control signal probe set all with the same amplification domain, and an HCR amplifier labeled with a reporter. In some embodiments, the device further comprises a readout probe set labeled with an auxiliary reporter (for example, see FIGS. 43 and 44).

In some embodiments, all capture probe sets, control capture probe sets, signal probe sets, and control signal probe sets are dried down in the same conjugate pad and/or flow through the same membrane channel. In some embodiments, all capture probe sets are pre-immobilized in their respective test regions and all control capture probe sets are pre-immobilized in their respective control regions (for example, see FIG. 44). In some embodiments, all signal probe sets and control signal probe sets are dried down in the same conjugate pad and/or flow through the same membrane channel (for example, see FIG. 44). In some embodiments, all HCR amplifiers and control HCR amplifiers are dried down in a conjugate pad and/or flow through the same membrane channel. In some embodiments, all readout probe sets and control readout probe sets are dried down in the same conjugate pad and/or flow through the same membrane channel. In some embodiments, one readout probe set is used to directly or indirectly generate an amplified signal in all test regions and an amplified control signal in all control regions (for example, see FIG. 44). In some embodiments, one HCR amplifier is used to directly or indirectly generate an amplified signal in all test regions and an amplified control signal in all control regions (for example, see FIG. 44).

Amplified HCR Lateral Flow Assays for Detecting a Target in a Sample in One or More Stages with or without Pre-Formed HCR Amplification Polymers

In some embodiments an HCR lateral flow device comprises a test region, a capture probe set, a signal probe set, a reporter-labeled HCR amplifier, and optionally an auxiliary-reporter-labeled readout probe set. In some embodiments, reagents automatically arrive in the test region in one or more successive stages to generate an amplified signal in the test region without user interaction.

In some embodiments, there are two or three stages that occur in succession after sample is added:

Stage 1: the capture probe set and signal probe set bind the target and immobilize the probe/target sandwich in the test region (for example, see FIGS. 30A-30B, 31A-31B, and 32A-32B).

Stage 2: HCR hairpins arrive in the test region where the amplification domains on the immobilized signal probe set trigger the growth of tethered HCR amplification polymers decorated with reporters that directly or indirectly generate an amplified signal in the test region (for example, see FIGS. 30A-30B, 31A-31B, and 32A-32B).

Stage 3 (optional): the auxiliary-reporter-labeled readout probe set arrives in the test region and binds to the reporters decorating the tethered HCR amplification polymer to directly or indirectly generate an amplified signal in the test region (for example, see FIGS. 30A, 31A, and 32A).

In some embodiments, the device comprises one or more signal-probe-set/HCR-polymer complexes that are pre-formed by using the amplification domains on the signal probe set to trigger self-assembly of tethered reporter-decorated HCR amplification polymers; there are one or two stages:

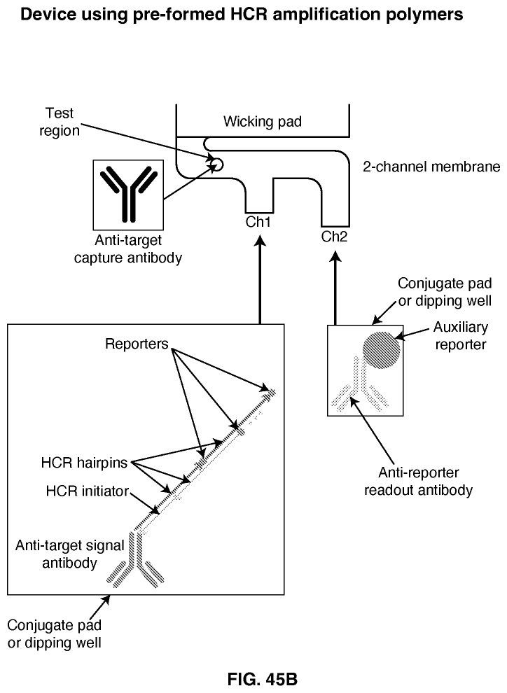

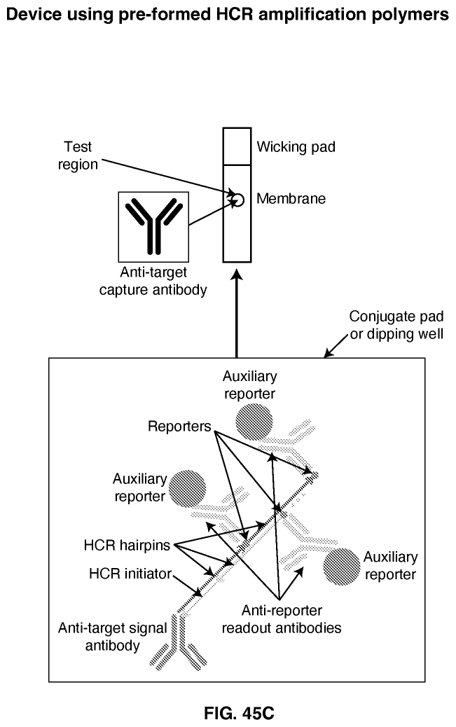

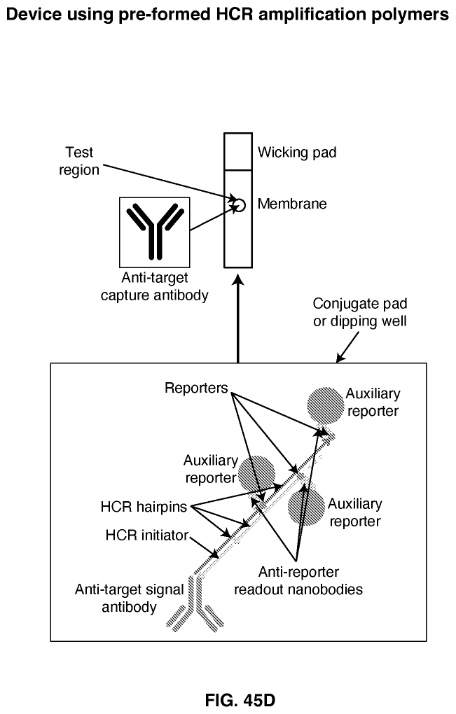

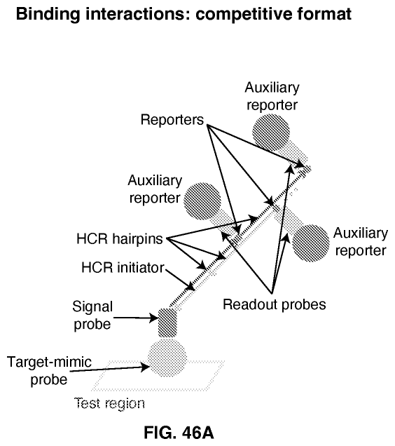

Stage 1: the capture probe set and signal-probe-set/HCR-polymer complex bind the target and immobilize the probe/target sandwich carrying tethered reporter-decorated HCR amplification polymers that directly or indirectly generate an amplified signal in the test region (for example, see FIG. 45A).

Stage 2 (optional): the auxiliary-reporter-labeled readout probe set arrives in the test region and binds to the reporters decorating the tethered HCR amplification polymer to directly or indirectly generate an amplified signal in the test region (for example, see FIG. 45B).

In some embodiments, the device comprises one or more signal-probe-set/HCR-polymer/readout-probe-set complexes that are pre-formed by using the amplification domains on the signal probe set to trigger self-assembly of tethered reporter-decorated HCR amplification polymers that are in turn bound by the auxiliary-reporter-labeled readout probe set; there is one stage:

Stage 1: the capture probe set and signal-probe-set/HCR-polymer/reporter-probe-set complex bind the target and immobilize the probe/target sandwich carrying tethered reporter-decorated HCR amplification polymers bound by the auxiliary-reporter-labeled readout probe set in the test region (for example, see FIGS. 45C-45D).

In some embodiments: 1) the signal probe set comprises a target-binding domain and an amplification domain comprising a binding domain (for example, a hapten, antibody, nanobody, ligand, peptide, receptor, nucleotide, or other binding domain), 2) HCR polymers are pre-formed by using HCR initiators to trigger self-assembly of reporter-decorated HCR amplification polymers wherein the HCR initiators comprise a binding domain configured to bind the binding domain on the signal probe set (for example, the binding domain on the HCR initiator could be a hapten, antibody, nanobody, ligand, peptide, receptor, nucleotide, or other binding domain configured to bind the binding domain on the signal probe set). For example, the binding domain on the signal probe set could comprise polystreptavidin and the HCR initiator could comprise biotin, or the binding domain on the capture probe set could comprise DIG and the HCR initiator could comprise an anti-DIG antibody. There are two or three stages:

Stage 1: the capture probe set and signal probe set bind the target and immobilize the probe/target sandwich in the test region.

Stage 2: the pre-formed reporter-decorated HCR amplification polymers bind the probe-target sandwich to directly or indirectly generate an amplified signal in the test region.

Stage 3 (optional): the auxiliary-reporter-labeled readout probe set arrives in the test region and binds to the reporters decorating the tethered HCR amplification polymer to directly or indirectly generate an amplified signal in the test region.

In some embodiments, any or all of the above stages occur concurrently or in different orders.

In some embodiments, one or more HCR hairpins (of which one or more are reporter-labeled) flow to the test region where they self-assemble into tethered reporter-decorated HCR amplification polymers to directly or indirectly generate an amplified signal in the test region. In some embodiments, pre-formed reporter-labeled HCR amplification polymers flow to the test region where they bind the probe/target complex to directly or indirectly generate an amplified signal in the test region. In some embodiments, pre-formed reporter-labeled HCR amplification polymers bind the signal probe set before flowing toward the test region. In some embodiments, pre-formed signal-probe-set/HCR-polymer complexes flow toward the test region (for example, see FIGS. 45A-45D). In some embodiments, HCR amplification polymers self-assemble during the assay before reaching the test region. In some embodiments, HCR amplification polymers are pre-formed and dried down on a conjugate pad as part of the device (for example, see FIGS. 45A-45D). In some embodiments, HCR amplification polymers are pre-formed, covalently crosslinked into a covalent polymer, and dried down on a conjugate pad as part of the device.

### HCR Lateral Flow Assays for Amplified Detection of a Target in a Sample Using a Competitive Assay Format

In some embodiments an amplified HCR lateral flow device is used to detect a target in a sample using a competitive assay format. In some embodiments, the device comprises one or more membranes, one or more conjugate pads, one or more sample pads, one or more wicking pads, one or more test regions, and/or one or more control regions. In some embodiments, a lateral flow device comprises an optional sample pad, an optional conjugate pad, a membrane, a test region, an optional control region, and a wicking pad.

In some embodiments, the device comprises one or more probe sets, wherein each probe set comprises one or more probes. In some embodiments, a probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, or any combination thereof.

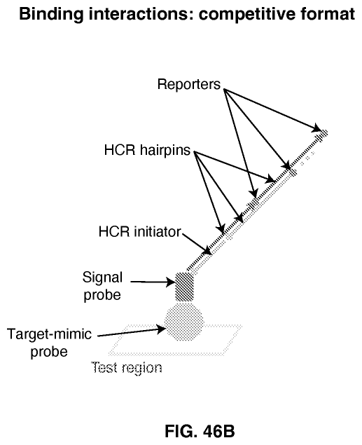

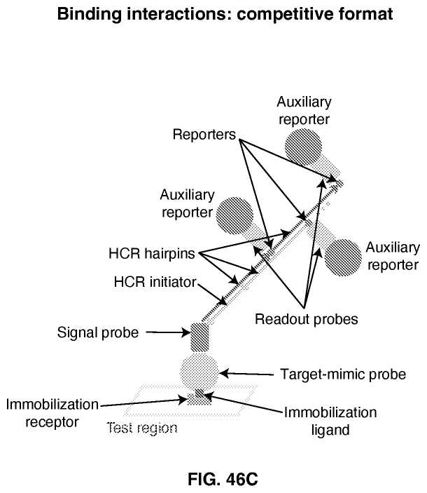

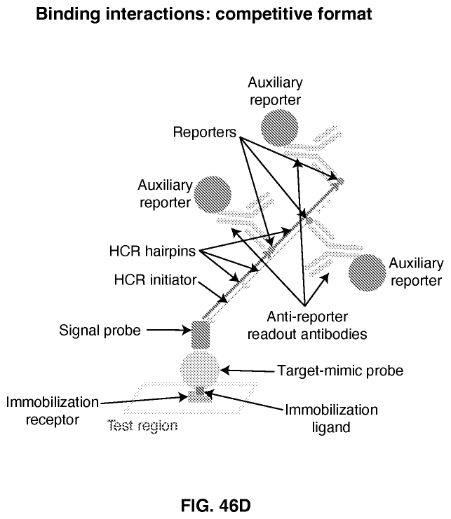

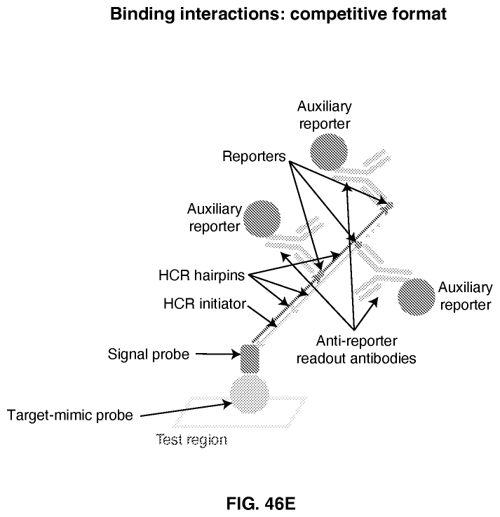

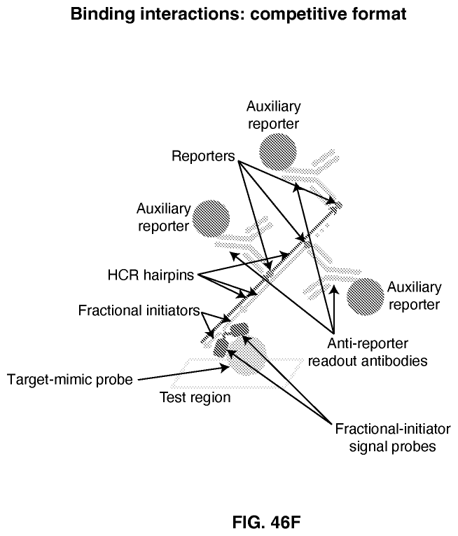

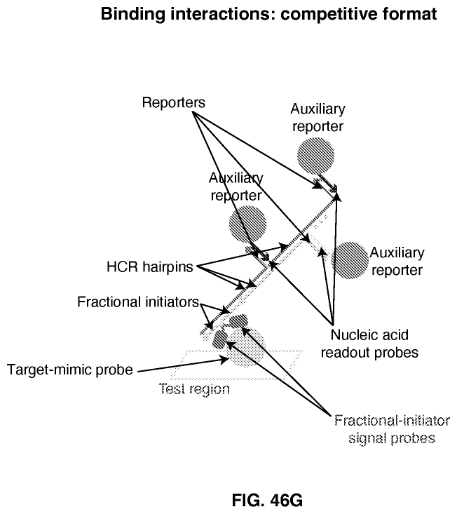

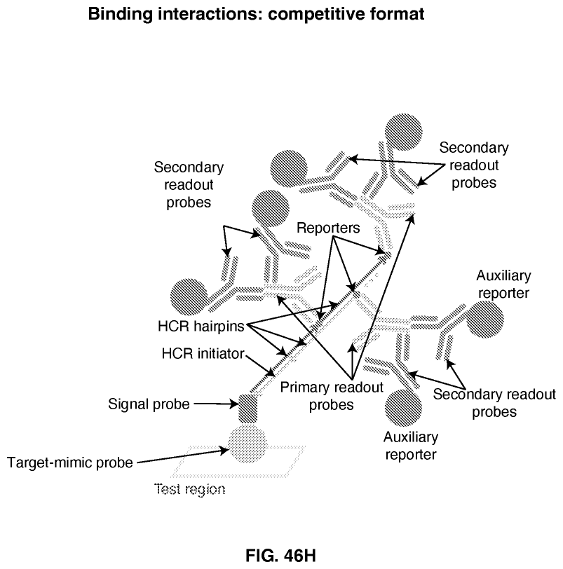

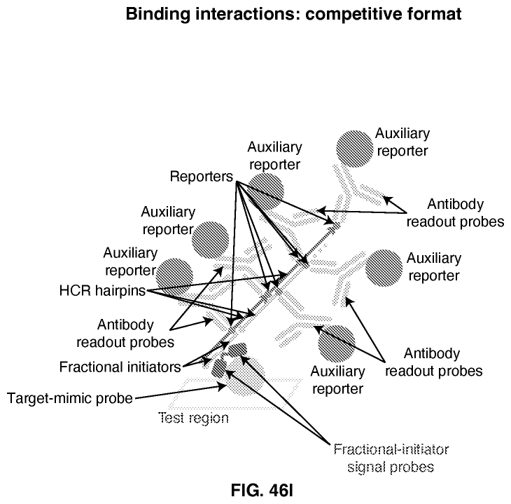

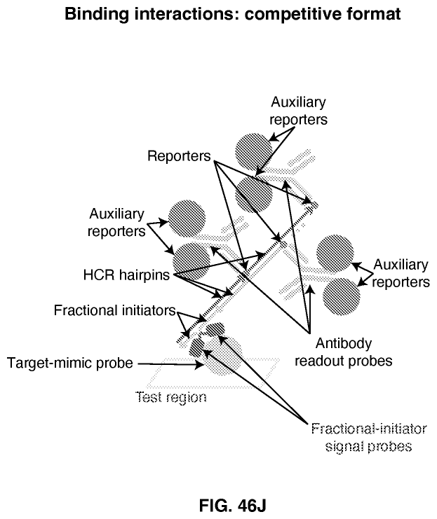

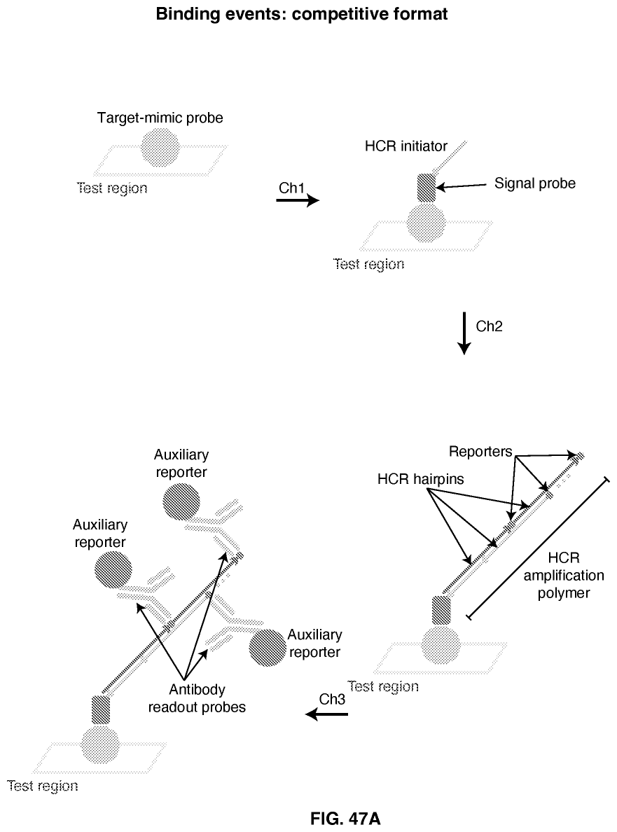

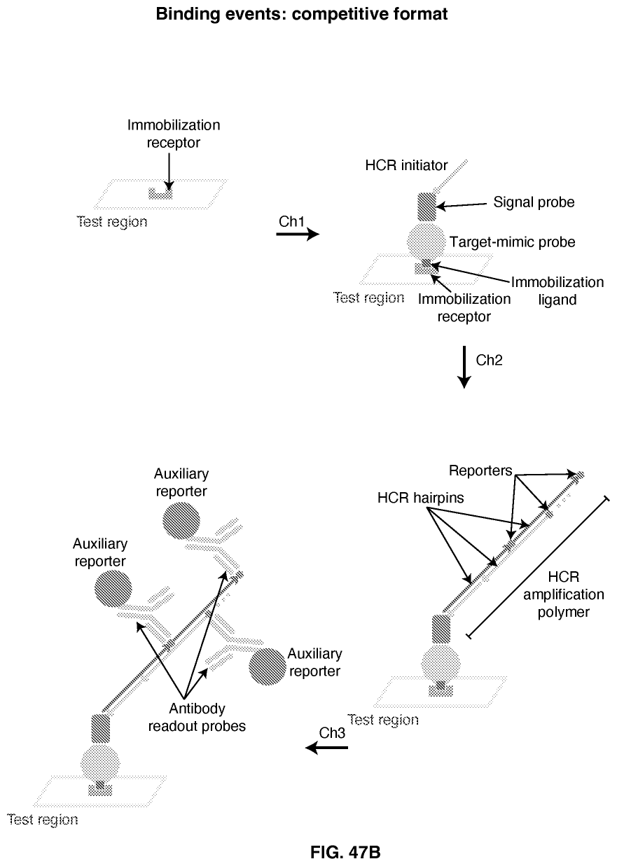

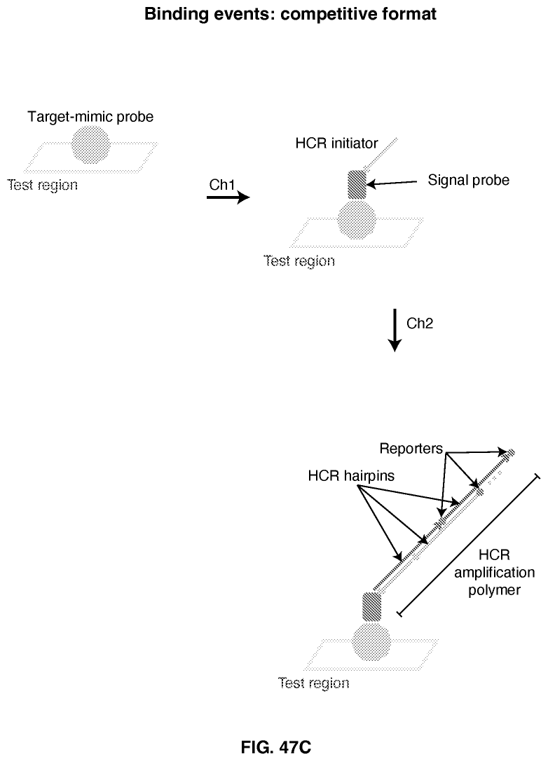

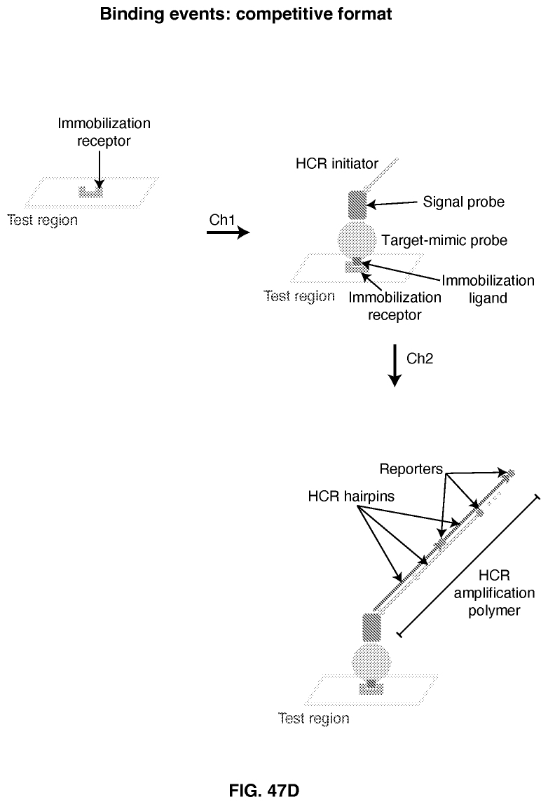

In some embodiments, the device comprises a test region (for example, see FIGS. 46A-46J). In some embodiments, the device comprises a target-mimic probe set comprising one or more target-mimic probes each comprising a competitive domain configured to compete with the target for binding partners, and an immobilization domain configured to bind directly or indirectly to the test region. In some embodiments, the competitive domain comprises the target. In some embodiments, the competitive domain is configured to compete with the target to bind signal probes. In some embodiments, the competitive domain comprises the portion of the target that is configured to bind the target-binding domain of a signal probe. In some embodiments, the competitive domain comprises a molecule configured to bind the target-binding domain of a signal probe. In some embodiments, the one or more target-mimic probes are pre-immobilized in the test region (for example, see FIGS. 47A and 47C). In some embodiments, the one or more target-mimic probes are labeled with an immobilization ligand that binds an immobilization receptor that is pre-immobilized in the test region (for example, see FIGS. 47B and 47D).

In some embodiments, target-mimic probes are pre-immobilized in the test region in a test feature shape that is a straight line, a curve, a zigzag, a compound line, a compound curve, a cross, a filled circle, an open circle, a filled shape, an open shape, or another feature shape (for example, see FIG. 13). In some embodiments, if the target is not present in the sample, the amplified signal generated in the test region takes the form of a straight line, a curve, or another shape depending on the test feature shape. In some embodiments, one test feature shape can be easier for the human eye to detect than another test feature shape.

In some embodiments, the device comprises an HCR amplifier comprising two or more HCR hairpins (for example, see FIGS. 3A, 14, 15, and 16A-16B). In some embodiments, one or more of the HCR hairpins are labeled with one or more reporters (for example, see FIG. 17A-17F). In some embodiments, the HCR hairpins comprising an HCR amplifier are configured not to polymerize in the absence of an HCR initiator. In some embodiments, HCR hairpins are configured such that detection of an HCR initiator triggers the HCR hairpins to polymerize to form a tethered HCR amplification polymer decorated with reporters (for example, see FIGS. 3A, 14, 15, and 16A-16B).

In some embodiments, the one or more probe sets comprise a signal probe set comprising one or more signal probes, wherein a signal probe comprises a target-binding domain configured to bind directly or indirectly the target and an amplification domain that is configured to directly or indirectly trigger HCR signal amplification by initiating a chain reaction in which HCR hairpins self-assemble to form a tethered HCR amplification polymer decorated with reporters (for example, see FIGS. 46A-46J). In some embodiments, if the target is not present in the sample, the signal probes bind the target-mimic probes pre-immobilized in the test region and generate an amplified HCR signal in the test region. In some embodiments, if the target is present in the sample, then the targets compete with the target-mimic probes to bind the signal probes, reducing the availability of signal probes to bind the target-mimic probes as the target concentration increases, and hence decreasing the signal strength in the test region as the target concentration increases. In some embodiments, the signal probe set comprises: a) one or more initiator-labeled probes wherein an initiator-labeled probe comprises a target-binding domain and an amplification domain comprising one or more HCR initiators (for example, see FIGS. 3B, 46A-46E, 46H, 18A-18N, and 19A-19F), or b) one or more probe units, each comprising two or more fractional-initiator probes, wherein a fractional-initiator probe comprises a target-binding domain and an amplification domain comprising a fractional initiator. In some embodiments, an individual fractional-initiator probe that binds non-specifically will not trigger HCR, but specific binding of the probes within a probe unit to adjacent cognate binding sites on the target colocalizes a full HCR initiator capable of triggering HCR (for example, see FIGS. 4A, 46F-46G, 46I-46J, 20A-20E, 21, 22, 24A-24R). In some embodiments, a signal probe comprises a primary signal probe comprising a target-binding domain and a secondary signal probe comprising an amplification domain (for example, see FIGS. 18C-18D, 18K-18L, 21D, 22D, 24D-24I, 24M-24R) wherein the secondary signal probe is configured to bind the primary signal probe directly or indirectly (for example, a primary signal probe that is a primary antibody and a secondary signal probe that is a secondary antibody configured to bind the primary antibody).

In some embodiments, the one or more probe sets comprise a readout probe set comprising one or more readout probes, wherein a readout probe comprises a reporter-binding domain configured to bind directly or indirectly to a reporter on an HCR amplification polymer (for example, see FIGS. 46A, 46C-46J). In some embodiments, readout probes comprise one or more auxiliary reporters wherein the auxiliary reporters contribute directly or indirectly to the generation of an amplified signal in the test region (for example, see FIGS. 46A, 46C-46J). In some embodiments, the amplified signal generated by the auxiliary reporters on the readout probes is visible to the human eye. In some embodiments, a readout probe comprises a primary readout probe comprising a reporter-binding domain and a secondary readout probe comprising an auxiliary reporter (for example, see FIG. 46H) wherein the secondary readout probe is configured to bind the primary readout probe directly or indirectly (for example, a primary readout probe that is a primary antibody and a secondary readout probe that is a secondary antibody configured to bind the primary antibody).

In some embodiments, the target-mimic probe set binds the signal probe set to form a mimic complex (for example, see FIGS. 46A-46J). In some embodiments, if the target is not present in the sample, the target-mimic probe set immobilizes the mimic complex in the test region (for example, see FIGS. 47A-47D). In some embodiments, if the target is present in the sample, the mimic complex does not form, the signal probes are not immobilized in the test region, the growth of a tethered HCR amplification polymer is not triggered in the test region, and an amplified signal is not generated in the test region. In some embodiments, after the mimic complex is immobilized in the test region, the amplification domains on the signal probes trigger the self-assembly of reporter-labeled HCR hairpins into tethered HCR amplification polymers decorated with reporters (for example, see FIGS. 47A-47D). In some embodiments, the reporters directly or indirectly lead to generation of an amplified signal in the test region. In some embodiments, auxiliary-reporter-labeled readout probes that comprise one or more auxiliary reporters bind the reporters decorating the tethered HCR amplification polymers that are immobilized in the test region, directly or indirectly generating an amplified signal in the test region (for example, see FIGS. 47A-47B). In some embodiments, the amplified signal is visible to the human eye. In some embodiments, the signal is read using a fluorescence microscope, a fluorescence scanner, a camera, a mobile phone camera, a mass spectrometer, a mass spectrometry microscope, a radioactive scanner, or another instrument suitable for detecting reporters and/or auxiliary reporters. In some embodiments, a reporter and/or an auxiliary reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a reporter and/or auxiliary reporter is a hapten, a ligand, an oligonucleotide, digoxigenin (DIG), fluorescein isothiocyanate (FITC), a fluorophore, biotin, dinitrophenol, aniline, an enzyme, or another molecule or complex that can be recognized by a binding partner to facilitate generation of a signal. In some embodiments, the reporters and/or auxiliary reporters decorating an HCR amplification polymer directly or indirectly mediate catalytic reporter deposition (CARD) to generate an amplified signal in the vicinity of the HCR amplification polymer within the test region. In some embodiments, CARD is mediated by an enzyme (for example, see FIGS. 25A-25E, 26A-26C, 27A-27B).

In some embodiments, the target comprises (for example, see FIGS. 46A-46J) a protein, a peptide, an amino acid, a nucleic acid, a DNA, an RNA, a chemically modified nucleic acid, a synthetic nucleic acid analog, a material capable of base-pairing, a chemical linker, a molecule, a biomolecule, a chemical, a toxin, a pathogen, a disease marker, a pollutant, a complex of any of the above, a diseased cell, a cancer cell, a human cell, a eukaryotic cell, a prokaryote, a virus, or any combination of the above. In some embodiments, the target is bacterial or viral in origin. Additional targets are discussed below.

In some embodiments, the sample comprises a bodily fluid, milk, mucus, water, urine, blood, serum, saliva, cells, tissue, tissue homogenate, a medical sample, an environmental sample, washwater from an agricultural crop, gray water, brown water, sea water, pollutants, polluted water, a waste sample, sewage, plants, bacteria, sludge, homogenized plants, organic matter, animal feed, an industrial sample, liquid fuel, solid fuel, propellants, a chemical solution, and/or chemicals. In some embodiments, the sample is obtained by swabbing, nasal swabbing, nasopharyngeal swabbing, anterior nasal swabbing, throat swabbing, or swabbing of a material, mixture, solution, or substance to be analyzed.

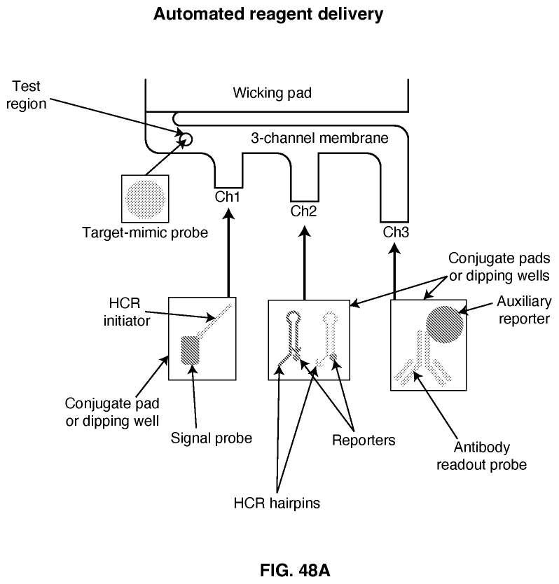

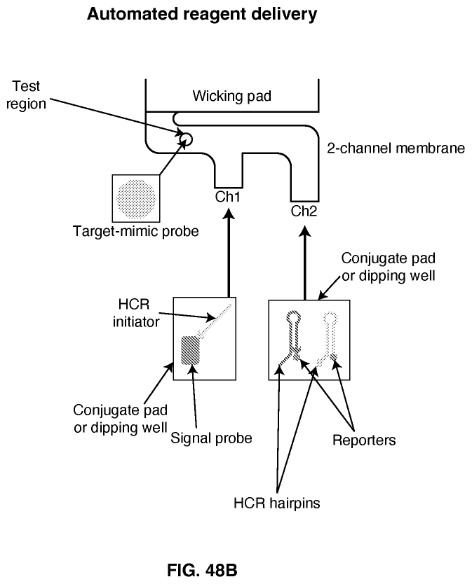

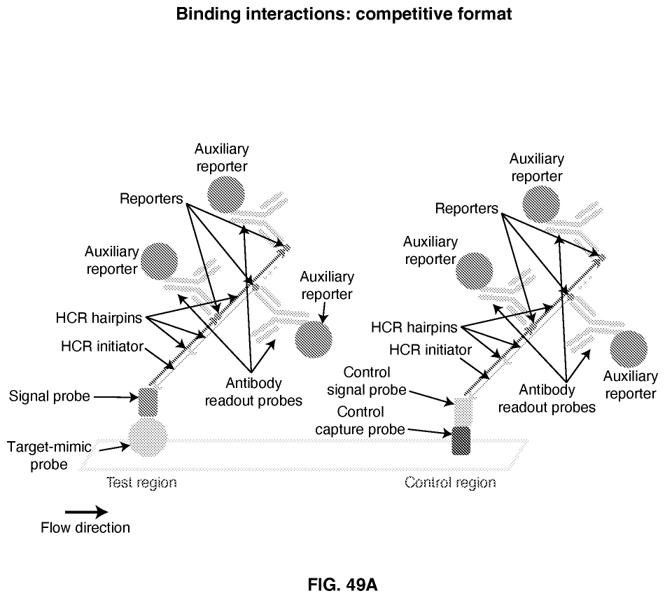

In some embodiments an HCR lateral flow device comprises a test region, a target-mimic probe set, a signal probe set comprising one or more signal probes wherein a signal probe comprises a target-binding domain and an amplification domain, an HCR amplifier comprising two or more HCR hairpins wherein one or more HCR hairpins comprise one or more reporters, and optionally a readout probe set comprising one or more readout probes wherein a readout probe comprises a reporter-binding domain and one or more auxiliary reporters (for example, see FIGS. 46A-46J). In some embodiments, the device is configured so that the reagents automatically arrive in the test region in one or more successive stages, generating an amplified signal in the test region if the target is not present in the sample (for example, see FIGS. 47A-47D and 48A-48B).

In some embodiments an HCR lateral flow device comprises a test region, a target-mimic probe set, a signal probe set, an HCR amplifier comprising one or more reporters, and optionally a readout probe set comprising one or more auxiliary reporters (for example, see FIGS. 46A-46J). In some embodiments, the device is configured so that the reagents automatically arrive in the test region in one or more successive stages, generating an amplified signal in the test region if the target is not present in the sample (for example, see FIGS. 47A-47D, 48A-48B).

In some embodiments an HCR lateral flow device comprises a test region, a target-mimic probe set, a signal probe set, an HCR amplifier comprising one or more reporters, and optionally a readout probe set comprising one or more auxiliary reporters (for example, see FIGS. 46A-46J). In some embodiments, the device is configured so that the reagents automatically arrive in the test region in a prescribed order, generating an amplified signal in the test region if the target is not present in the sample (for example, see FIGS. 47A-47D, 48A-48B).

In some embodiments, reagents are automatically delivered to the test region in multiple successive stages using a multi-channel membrane (for example, see FIGS. 48A-48B, and 73A-73C, and 73F). In some embodiments, reagents are automatically delivered to the test region in successive stages using a multi-channel membrane in which channels of different lengths lead into a unified channel before reaching the test region (for example, see FIGS. 48A-48B, and 73C). In some embodiments, channels of differing lengths deliver reagents to the test region in order according to their lengths, with the reagents from the shortest channel arriving in the test region first, the reagents from the longest channel arriving in the test region last, and channels of intermediate lengths arriving in the test region at intermediate times (for example, see FIGS. 48A-48B, and 73C). In some embodiments, channels are pre-treated with speed additives that create different flow speeds in different channels, allowing for delivery of reagents to the test region in successive stages. In some embodiments, the speed additives comprise sucrose, fructose, trehalose, a sugar, dextran sulfate, a dissolvable barrier, a wax barrier, water-insoluble ink, sponge, valves, pressurized paper, agarose, gravitational orientation, or another agent. In some embodiments, multiple channels are stacked on top of each other to form a multi-layer membrane (for example, see FIGS. 73B-73C). In some embodiments, the sample arrives in different channels at different times, leading to successive delivery of reagents to the test region. In some embodiments, dissolvable barriers dissolve at different rates to introduce the sample into different channels at different times, leading to successive delivery of reagents to the test region. In some embodiments, different reagents migrate through the same or different channels at different rates leading to successive staged arrival times in the test region. For example, auxiliary-reporter-labeled readout probes labeled with bulky auxiliary reporters migrate through the membrane more slowly than reporter-labeled HCR hairpins labeled with small reporters, allowing for the readout probes to arrive in the test region after the HCR hairpins. In some embodiments, the size of different reagents affects their migration speeds through the same channel, allowing for successive delivery of reagents to the test region. In some embodiments, a multi-channel membrane is configured so that reagents in each channel arrive in the test region (or another location) in a prescribed order (for example, see FIGS. 48A-48B, and 73A-73C, and 73F).

In some embodiments, reagents for each channel are dried onto separate conjugate pads (for example, see FIGS. 48A-48B, 73A-73C, and 73E-73F). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user interaction. In some embodiments, draining of a first conjugate pad allows draining of a second conjugate pad, which in turn allows draining of a third conjugate pad, and so on (for example, see FIGS. 48A-48B). In some embodiments, draining of the first conjugate pad frees a unified channel to allow draining of a second conjugate pad, which in turn frees a unified channel to allow draining of a third conjugate pad, and so on (for example, see FIGS. 48A-48B).

In some embodiments, a membrane, a sample pad, a conjugate pad, and/or a wicking pad comprise nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.

In some embodiments, reagents are automatically delivered to the test region in two successive stages using a two-channel membrane in which channels of different lengths lead into a unified channel before reaching the test region (for example, see FIG. 48B). In some embodiments, the signal probes travel via the shorter channel (Channel 1) to reach the test region first where they bind target-mimic probes that are pre-immobilized in the test region to form an immobilized mimic complex (for example, see FIGS. 47C and 48B). In some embodiments, reporter-labeled HCR hairpins travel via a longer channel (Channel 2) and reach the test region next, where amplification domains on the signal probes trigger growth of tethered HCR amplification polymers decorated with reporters to generate an amplified signal in the test region (for example, see FIGS. 47C-47D and 48B). In some embodiments, reagents for each channel are dried onto two separate conjugate pads (for example, see FIG. 48B). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user interaction. In some embodiments, draining of the first conjugate pad allows draining of the second conjugate pad. In some embodiments, draining of the first conjugate pad frees a unified channel to allow draining of the second conjugate pad. In some embodiments, reagents in the two channels arrive in the test region (or another location) in successive stages in a prescribed order due to channel length, channel geometry, speed additives, dissolvable barriers, or other conditions (for example, see FIG. 48B).

In some embodiments, reagents are automatically delivered to the test region in three successive stages using a three-channel membrane in which channels of different lengths lead into a unified channel before reaching the test region (for example, see FIG. 48A). In some embodiments, signal probes travel via the shortest channel (Channel 1) to reach the test region first where they bind target-mimic probes that are pre-immobilized in the test region to form an immobilized mimic complex (for example, see FIGS. 47A and 48A). In some embodiments, reporter-labeled HCR hairpins travel via a channel of intermediate length (Channel 2) and reach the test region next, where amplification domains on the signal probes trigger growth of tethered HCR amplification polymers decorated with reporters (for example, see FIGS. 47A-47B and 48A). In some embodiments, auxiliary-reporter-labeled readout probes travel via the longest channel (Channel 3) and arrive in the test region last, where they bind the reporters decorating the HCR amplification polymers to generate an amplified signal in the test region (for example, see FIGS. 47A-47B and 48A). In some embodiments, reagents for each channel are dried onto separate conjugate pads (for example, see FIG. 48A). In some embodiments, the reagents on each conjugate pad are rehydrated simultaneously when the user adds the sample to a sample pad. In some embodiments, the reagents on each conjugate pad are rehydrated successively when the user adds the sample to a sample pad. In some embodiments, the reagents on each conjugate pad are directly rehydrated when the user adds the sample to the conjugate pads. In some embodiments, upon rehydration, successive delivery of the reagents to the test region occurs automatically without user input. In some embodiments, draining of the first conjugate pad allows draining of the second conjugate pad, which in turn allows draining of the third conjugate pad, and so on. In some embodiments, draining of the first conjugate pad frees the unified channel to allow draining of the second conjugate pad, which in turn frees the unified channel to allow draining of the third conjugate pad. In some embodiments, reagents in the three channels arrive in the test region (or another location) in successive stages in a prescribed order due to channel length, channel geometry, speed additives, dissolvable barriers, or other conditions (for example, see FIG. 48A).

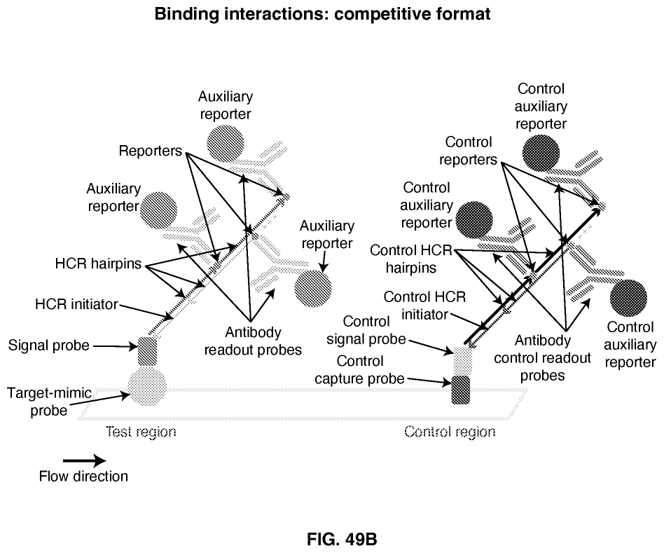

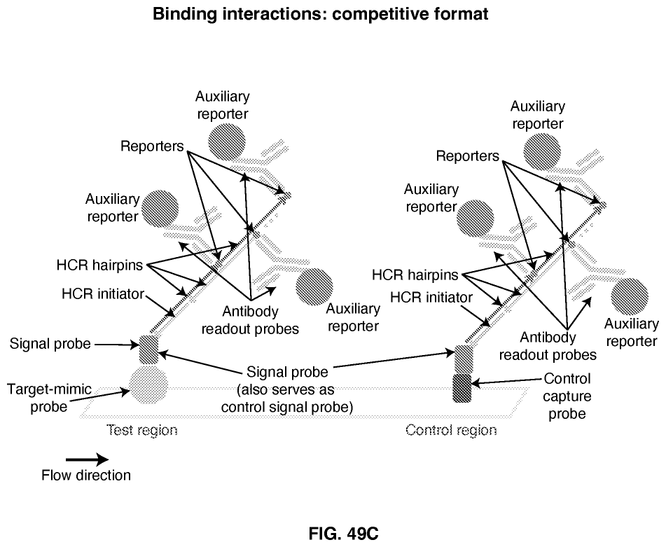

In some embodiments, the device comprises a control region (for example, see FIGS. 49A-49C, 71A-71C, and 72A-72C). In some embodiments, the control region is downstream of the test region so that the sample and reagents flow through the test region before reaching the control region (for example, see FIGS. 49A-49C, 71A-71C, and 72A-72C). In some embodiments an HCR lateral flow device comprises a test region, a target-mimic probe set, a signal probe set, an HCR amplifier comprising one or more reporters, and optionally a readout probe set comprising one or more auxiliary reporters (for example, see FIGS. 49A-49C, 71A-71C, and 72A-72C). In some embodiments the device further comprises a control region, a control capture probe set, a control signal probe set, a control HCR amplifier comprising one or more control reporters, and optionally a control readout probe set comprising one or more control auxiliary reporters (for example, see FIGS. 49A-49C, 71A-71C, and 72A-72C). In some embodiments, the signal probe set and the control signal probe set are the same (for example, see FIGS. 49C, 71C, and 72C). In some embodiments, the amplification domains on the signal probe set and the control signal probe set are the same (for example, see FIGS. 49A, 49C, 71A, 71C, 72A, and 72C). In some embodiments, the HCR amplifier and the control HCR amplifier are the same (for example, see FIGS. 49A, 49C, 71A, 71C, 72A, and 72C). In some embodiments, the optional readout probe set and the optional control readout probe set are the same (for example, see FIGS. 49A, 49C, 71A, 71C, 72A, and 72C).

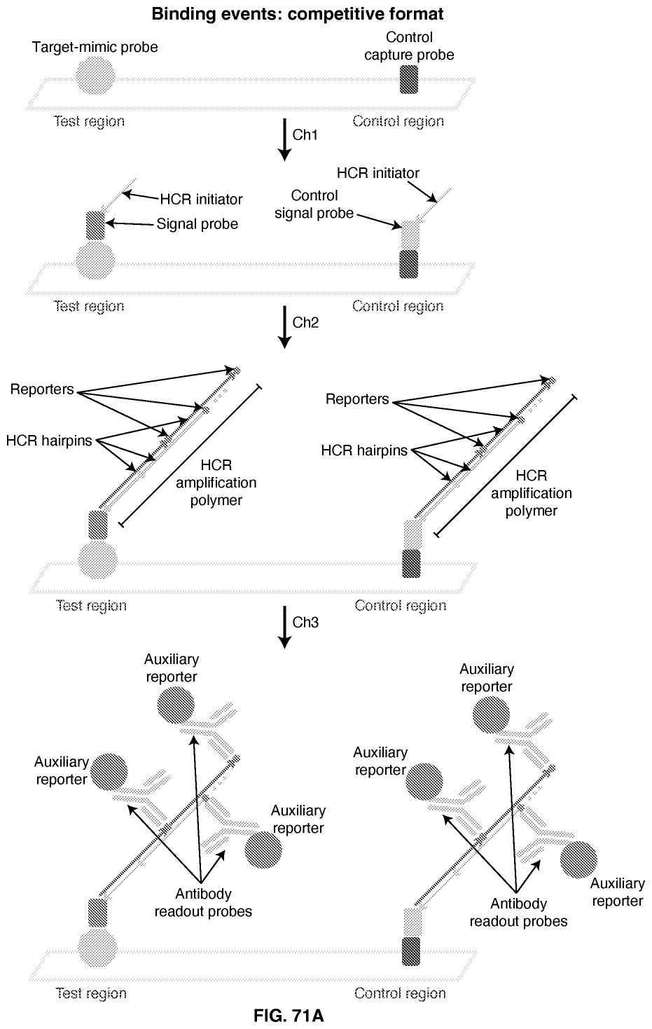

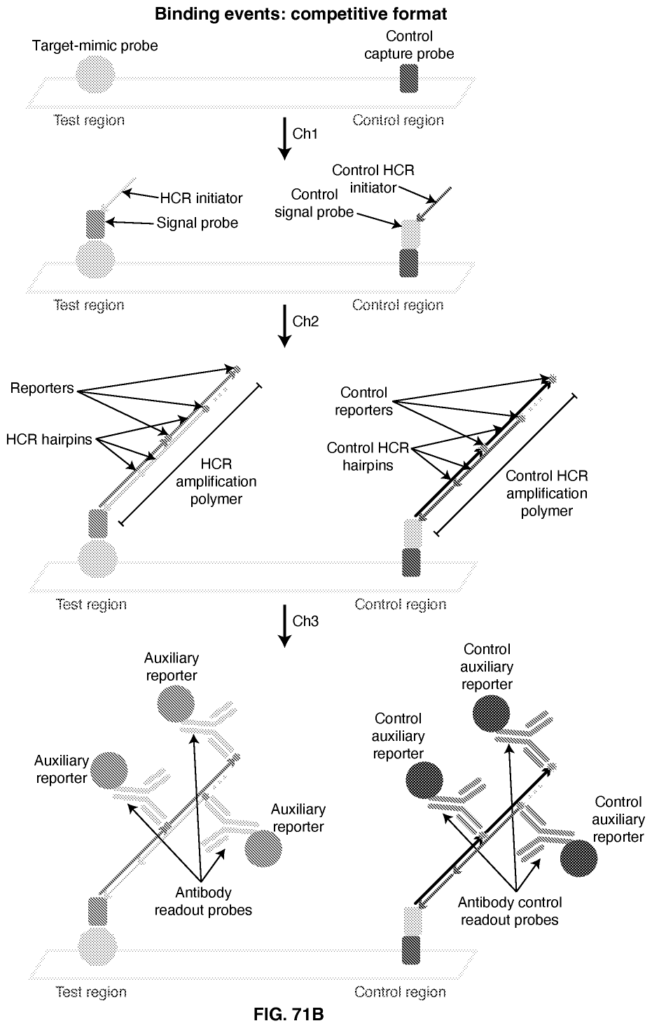

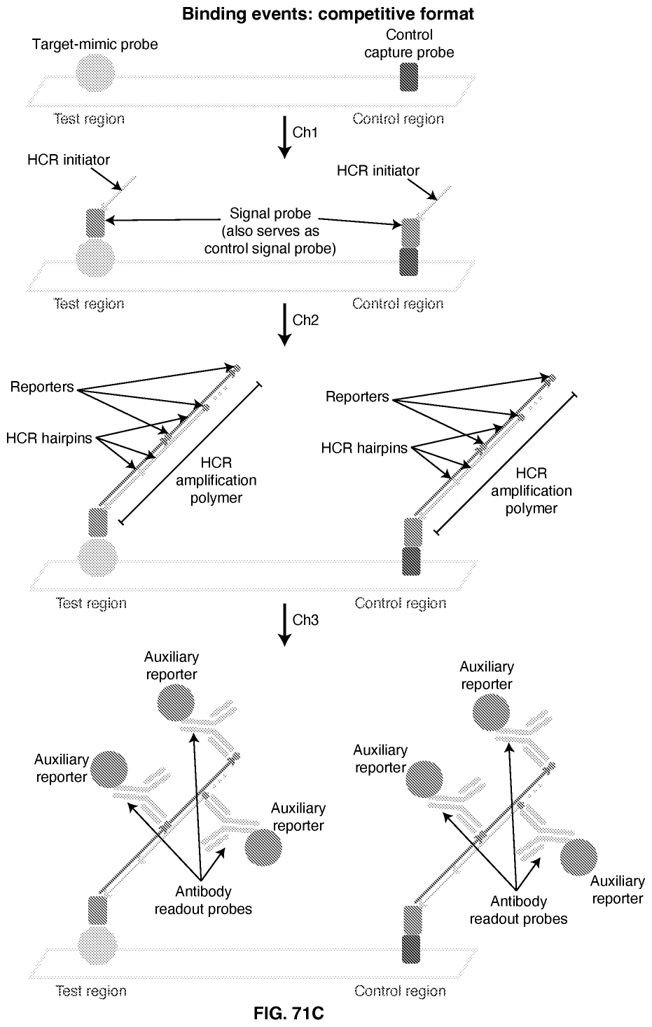

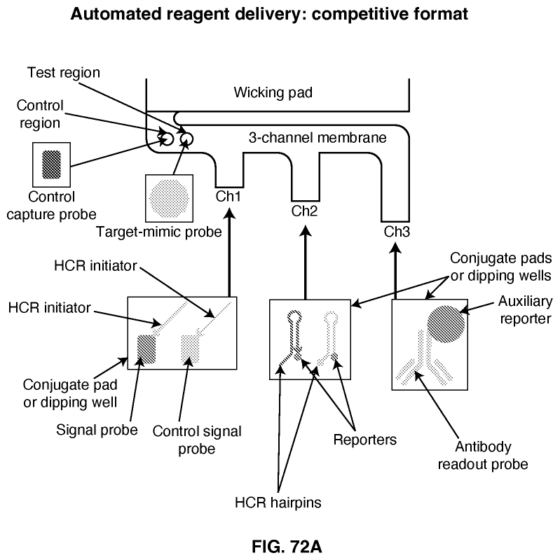

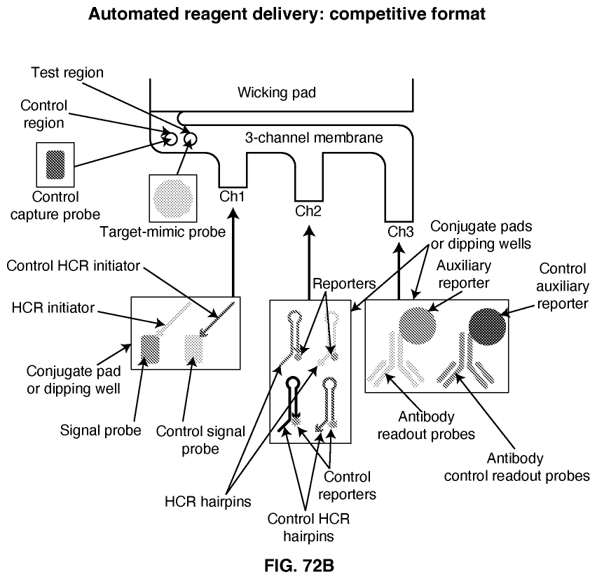

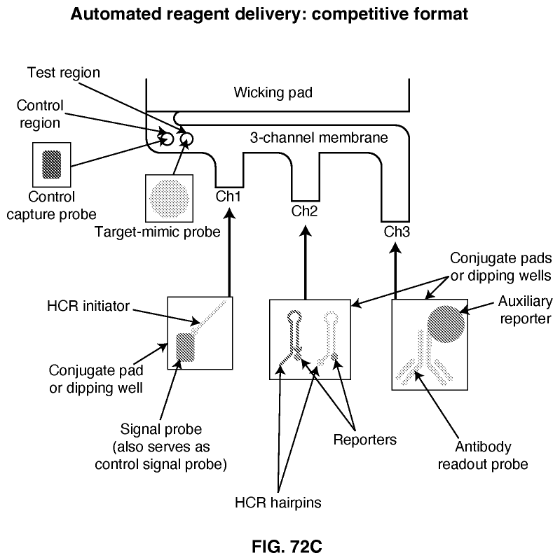

In some embodiments, the device is configured so that the reagents automatically arrive in the test region and the control region in one or more successive stages (for example, see FIG. 72A-72C), generating an amplified signal in the test region if the target is not present in the sample, and generating an amplified control signal in the control region if the sample and reagents flow through the device properly. In some embodiments, if the sample and reagents successfully flow through the control region, the control capture probe set binds to and immobilizes the control signal probe set in the test region, the control amplification domains on the control signal probe set trigger the self-assembly of tethered HCR amplification polymers decorated with reporters to directly or indirectly generate an amplified control signal in the control region, and optionally the auxiliary-reporter-labeled readout probe set binds the reporters to directly or indirectly generate an amplified control signal in the control region (for example, see FIGS. 71A-71C). In some embodiments, the test produces a positive result if a visible signal is produced in the control region but not the test region. In some embodiments, the test produces a negative result if both the test region and the control region produce visible signals. In some embodiments, absence of signal in the control region is an indication that the test did not function properly.

In some embodiments, the device comprises a folding card geometry (for example, see FIG. 5). In some embodiments, one page of the card contains a sample pad and one or more conjugate pads. In some embodiments, the other page of the card contains a membrane comprising one or more channels, a test region, an optional control region, and a wicking pad. In some embodiments, the wicking pad absorbs liquid to induce continued capillary flow through the channels after the sample reaches the wicking pad. In some embodiments, one page is functionalized with prongs that disconnect the sample pad from the one or more conjugate pads upon folding the card. In some embodiments, disconnecting the sample pad from the one or more conjugate pads limits the volume that flows from the one or more conjugate pads and prevents flow between conjugate pads. In some embodiments, the device takes the form of a comb, wagon wheel, stacked device, half-strip, dipstick, reverse-flow device, any combination thereof, or another geometry (for example, see FIGS. 48AB and 73A-73F).

In some embodiments, the user preps the sample by adding the sample to a prep buffer (for example, see FIG. 38A-38B). In some embodiments, the user preps the sample by adding prep buffer to the sample (for example, see FIG. 38C). In some embodiments, the user preps the sample by heating the sample in solution. In some embodiments, the prep buffer is an extraction buffer that exposes the target (for example, by disrupting a viral envelope or cell membrane). In some embodiments, the prep buffer is a denaturant. In some embodiments, the user adds the prepped sample to a sample pad to start a test. In some embodiments, the user adds the prep buffer to the sample pad and adds the sample to the prepped sample pad (for example, see FIG. 38D). In some embodiments, the user adds sample to the sample pad (for example, by putting a swab in contact with the sample pad) and then adds the prep buffer to the sample and sample pad (for example, see FIG. 38E). In some embodiments, the user adds the sample to the device (for example, see FIG. 38F). In some embodiments, the user closes a folding card device to start the test (for example, see FIG. 38D). In some embodiments, closing the card creates contacts between the membrane and one or more conjugate pads, initiating the flow of liquid in one or more channels. In some embodiments, the reagents are delivered to the test region in successive stages via one or more channels without user interaction. In some embodiments, the user reads either a positive result (no visible signal in the test region) or a negative result (visible signal in the test region) with the naked eye. In some embodiments, the user or a health care professional reads a result using an instrument to scan the test region (for example, a fluorescence scanner, camera, radiation scanner, mass spectrometer, or other device that allows acquisition of a signal from the test region).

### Hybridization Chain Reaction (HCR) Signal Amplification

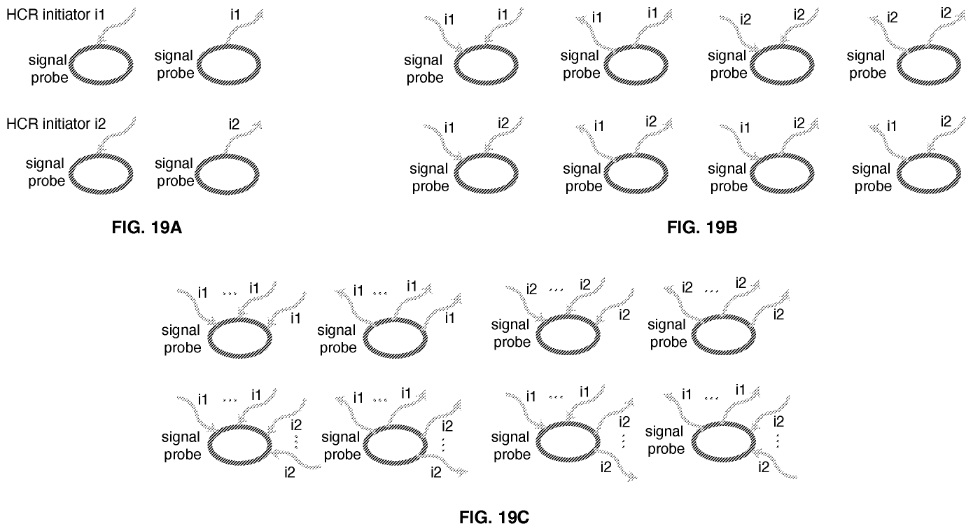

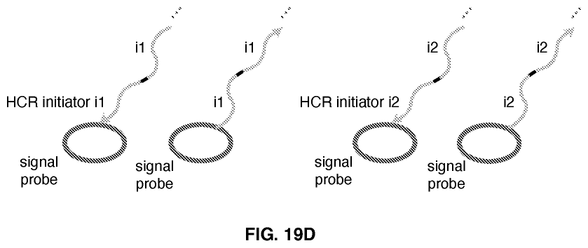

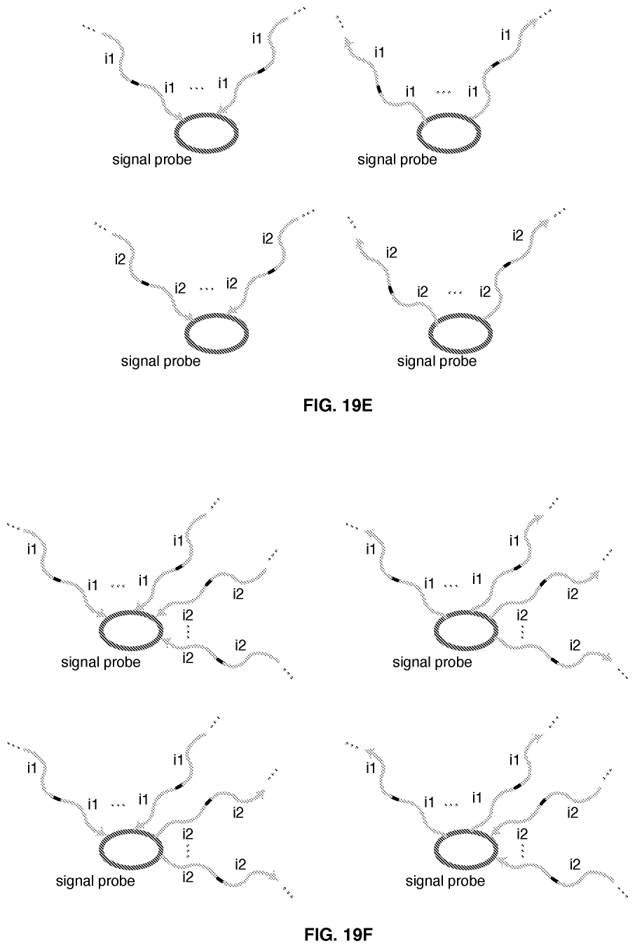

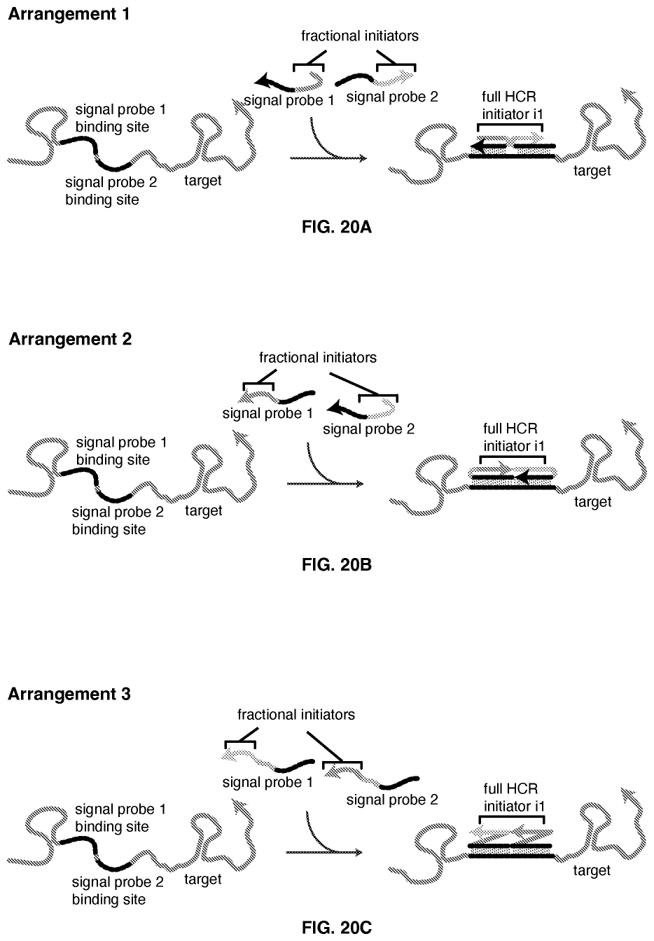

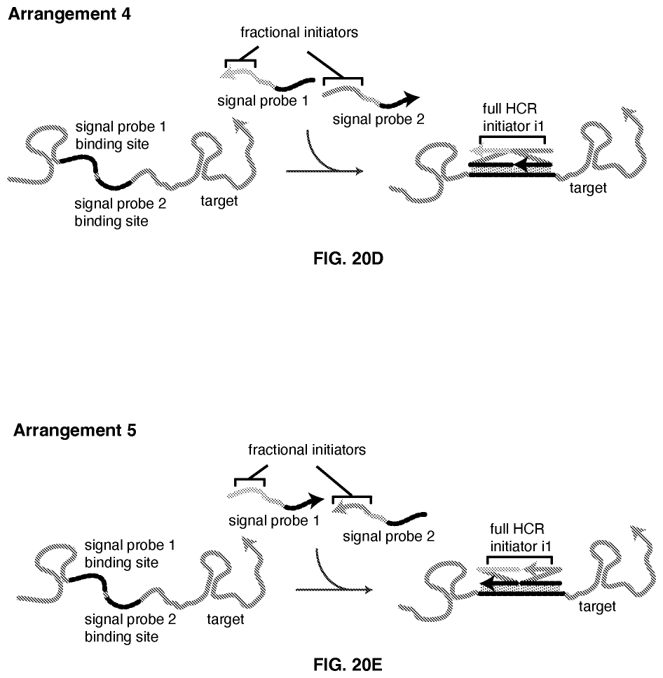

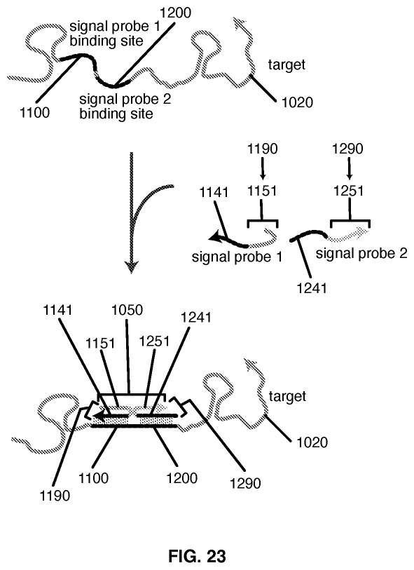

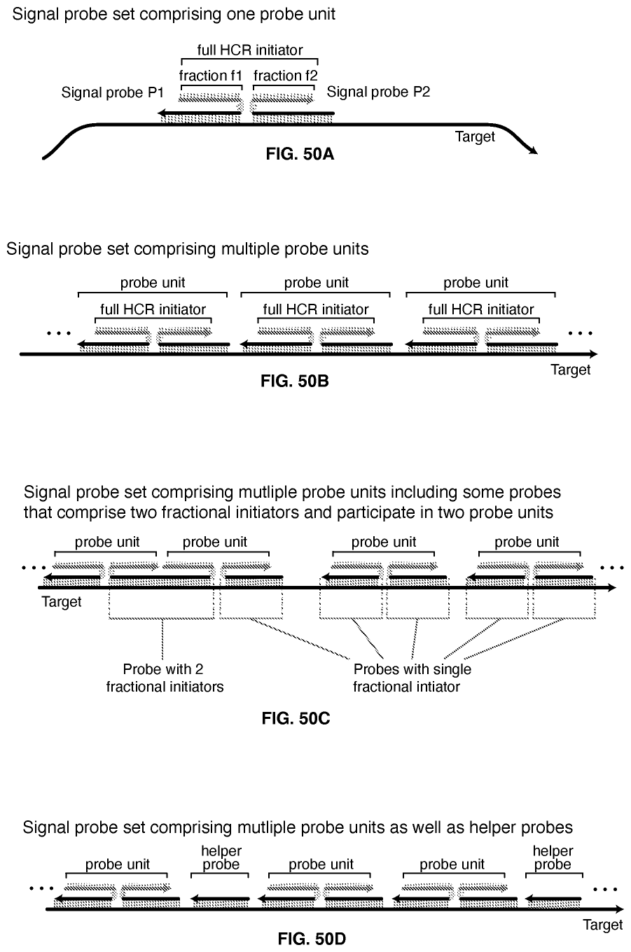

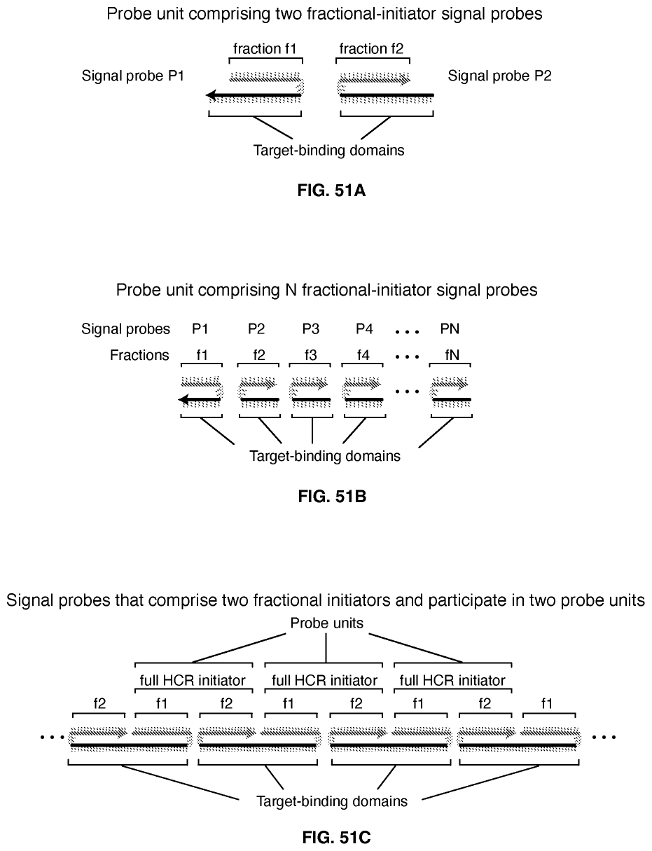

In some embodiments, a target is detected in an HCR lateral flow assay using a signal probe set comprising one or more initiator-labeled probes each comprising a target-binding domain and an amplification domain comprising one or more HCR initiators (for example, FIGS. 18A-18N and 19A-19F). In some embodiments, a target is detected within a sample using a signal probe set comprising one or more probe units (for example see the probe sets of FIGS. 4A-4B and 50A-50D), where a probe unit comprises two or more fractional-initiator probes (for example see the probe units of FIGS. 20A-20E, 21A-21D, 22A-22E, and 51A-51C), where each fractional-initiator probe comprises a target-binding domain and an amplification domain comprising a fractional initiator (for example, see the fractional-initiator probes of FIGS. 23 and 51A-51C). In some embodiments, binding of each probe within a probe unit to adjacent cognate binding sites on the target colocalizes the fractional initiators to form a full HCR initiator (for example, see the full HCR initiators of FIGS. 4A-4B, 20A-20E, 21A-21D, 22A-22E, 50A-50D, and 24) capable of triggering HCR signal amplification.

In some embodiments, HCR signal amplification increase the signal strength by a factor of 2, 5, 10, 15, 20, 25, 30, 40, 50, 75, 100, 500, 1000, 2000, 5000, 10,000, or 20,000-fold, or a value with a range defined by any two of the aforementioned values.

In some embodiments, the target-binding domains within a probe unit are configured to bind to overlapping or non-overlapping regions of the target. In some embodiments, the fractional initiators within a probe unit are designed to hybridize to overlapping or non-overlapping regions of an HCR hairpin. In some embodiments, individual fractional-initiator probes that bind non-specifically do not colocalize a full HCR initiator, suppressing spurious generation of amplified HCR background.

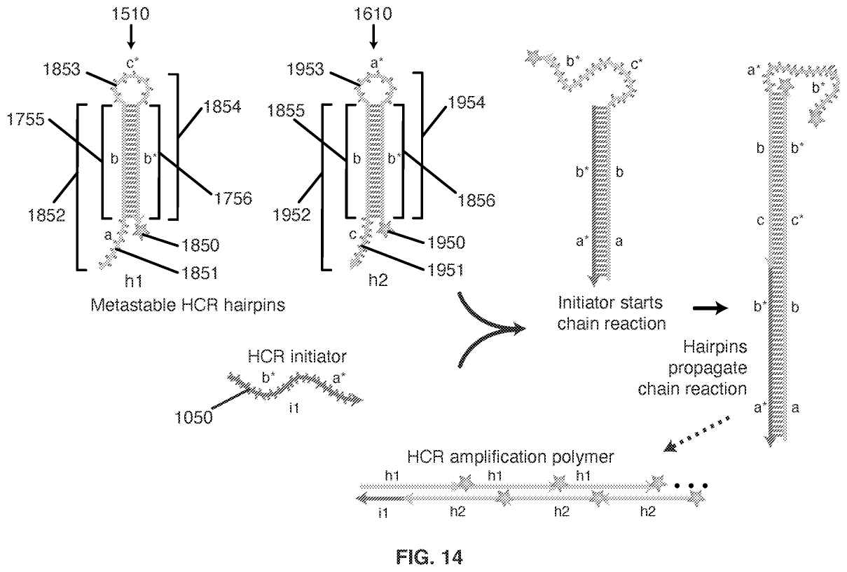

In some embodiments, an HCR amplifier comprises two or more HCR hairpins (for example, see the HCR amplifiers of FIGS. 4A-4B, 17A-17F, and 16A-16B). In some embodiments, each HCR hairpin comprises an input domain with a single-stranded toehold and a stem section, and an output domain with a single-stranded loop and a complement to the stem section (for example, see the HCR hairpins of FIGS. 4A-4B, 14, 15, 17A-17F, and 16A-16B). In some embodiments, the one or more HCR initiators on an initiator-labeled probe each initiate a chain reaction of polymerization steps in which the initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield an HCR amplification polymer tethered to the target (for example, see the amplification polymers of FIGS. 14, 15, 16A-16B, 25A, 25C-25E, and 26A). In some embodiments, in the absence of a full HCR initiator, HCR hairpins are kinetically trapped and do not polymerize, suppressing background. However, if the fractional-initiator probes within a probe unit bind to their adjacent cognate binding sites on the target to colocalize a full HCR initiator, the full HCR initiator initiates a chain reaction of polymerization steps in which the full initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield an HCR amplification polymer tethered to the target (for example, see the amplification polymers of FIGS. 4A, 17C-17F, and 16A-16B).

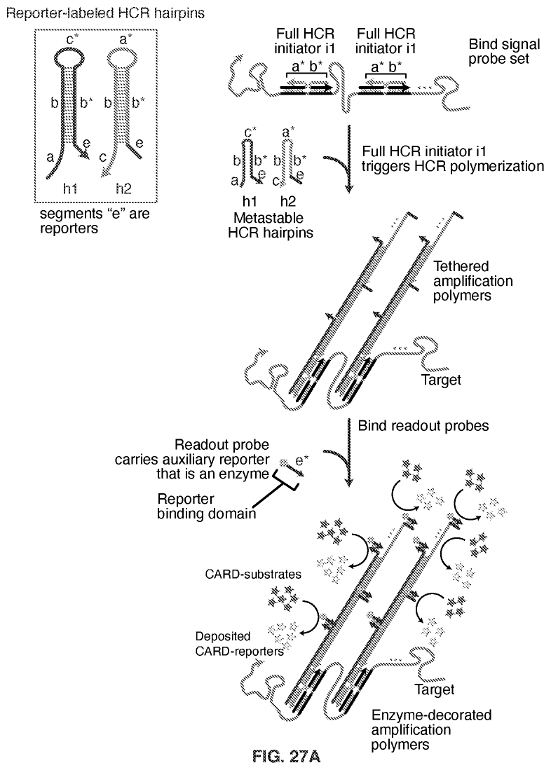

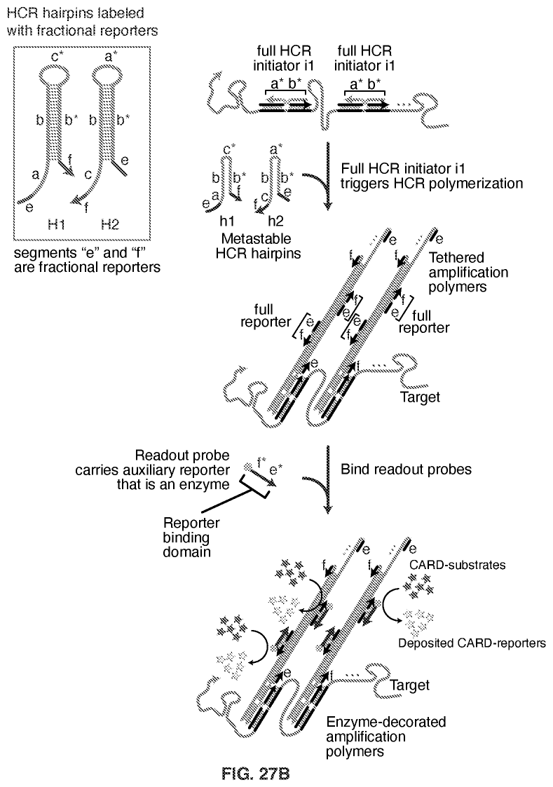

In some embodiments, an HCR hairpin further comprises zero, one, or more reporters that directly or indirectly lead to generation of an amplified signal (for example, see FIG. 17A-17F). In some embodiments, the zero, one, or more reporters on an HCR hairpin serve to mediate an additional layer of signal amplification via catalytic reporter deposition (CARD) (see for example, FIGS. 25A-25E, 26A-26C). In some embodiments, a reporter on an HCR hairpin comprises a fractional reporter such that an auxiliary-reporter-labeled readout probe does not strongly bind the fractional reporter on an individual hairpin, but such that following HCR polymerization, neighboring hairpins in the HCR amplification polymer colocalize a full reporter such that the colocalized full reporter strongly binds an auxiliary-reporter-labeled readout probe (for example, FIGS. 17D and 27B). In some embodiments, a readout probe comprises one or more auxiliary reporters and further comprises a reporter-binding domain configured to bind a reporter on an HCR amplification polymer or configured to bind a full reporter colocalized within an HCR amplification polymer (for example, see the readout probes of FIGS. 27A-27B). In some embodiments, amplified signal is generated by one or more reporters or auxiliary reporters associated with an HCR amplification polymer tethered to the target within the sample. In some embodiments, signal is removed from the test region. In some embodiments, HCR signal is generated, detected, and removed from the test region one or more times.

HCR initiators. In some embodiments, an initiator-labeled probe comprises one or more HCR initiators capable of initiating an HCR polymerization cascade. In some embodiments, an initiator is fully complementary to the input domain of an HCR hairpin such that it hybridizes to the input domain of the hairpin to open the hairpin and initiate the HCR polymerization cascade. In some embodiments, the initiator is partially complementary to the input domain of an HCR hairpin, but sufficiently complementary such that it hybridizes to the input domain of the hairpin to open the hairpin and initiate the HCR polymerization cascade. In some embodiments, the initiator is shorter or longer than the input domain of an HCR hairpin and/or has incomplete complementarity to the input domain of the hairpin, but is able to hybridize to the input domain of the hairpin to open the hairpin and initiate the HCR polymerization cascade. In some embodiments, an HCR initiator might have 60%, 70%, 80%, 90%, or 100% (or any intermediate value between any of these values) complementarity to the input domain of an HCR hairpin, and hybridize to the input domain of the hairpin to open the hairpin and initiate the HCR polymerization cascade. In some situations, initiator-labeled probes comprising one or more initiators may cause increased background due to non-specific binding of initiators to DNA, RNA, proteins, or other molecules within the sample. In some embodiments, the initiators on an initiator-labeled probe are shielded by base-pairing to reduce non-specific binding of the probe within the sample (to reduce background). In some embodiments, the initiator can be shielded by a hairpin structure. In some embodiments, the initiator can be shielded by one or more auxiliary oligos. In some embodiments, the initiator can be shielded by self-complementarity within the oligo comprising an initiator and/or complementarity to one or more auxiliary strands.

Automatic background suppression with HCR fractional-initiator probes. In some embodiments, fractional-initiator probes automatically suppress background because the HCR initiator is split between a pair of probes (for example, see FIG. 8). In some embodiments, if probes bind specifically to the target at proximal cognate binding sites, the target colocalizes the two probes within a probe pair to form a full HCR initiator. In some embodiments, individual fractional-initiator probes that bind non-specifically do not trigger HCR since each probe carries only a fraction of an HCR initiator, and HCR signal amplification is triggered only if the full HCR initiator is colocalized.

Automatic background suppression with HCR hairpins. In some embodiments, HCR hairpins automatically suppress background because HCR hairpins are kinetically trapped so they do not polymerize in the absence of an HCR initiator. In some embodiments, if both probes within a fractional-initiator probe pair bind specifically to their proximal cognate binding sites on the target, the resulting colocalized full HCR initiator triggers growth of a tethered HCR amplification polymer (for example, see FIG. 4A). In some embodiments, individual HCR hairpins that bind non-specifically do not trigger HCR since they are kinetically trapped.

Automatic background suppression with HCR fractional-initiator probes and HCR hairpins. In some embodiments, the combination of HCR fractional-initiator probes for target detection and HCR amplification hairpins for signal amplification provide automatic background suppression throughout the protocol, ensuring that reagents will not generate amplified background even if they bind non-specifically.

Full HCR initiator formed by colocalization of 2 or more fractional-initiator probes. In some embodiments a probe unit can comprise a set of fractional-initiator probes that generate a full HCR initiator (for example, see FIG. 51A-C). In some embodiments, a full HCR initiator is generated by a pair of fractional-initiator probes that each carry a fraction of the full HCR initiator such that together they comprise the full HCR initiator (fraction f1 for probe P1 and fraction f2 for probe P2 such that f1+f2=1); in this case, a probe unit is two fractional-initiator probes (for example, see FIG. 51A). In some embodiments, the fractions f1 and f2 are sufficiently small compared to the full HCR initiator (for example, f1=0.5 with f2=0.5; or f1=0.45 with f2=0.55; or f1=0.4 with f2=0.6; or f1=0.3 with f2=0.7) such that HCR signal amplification is suppressed if the full HCR initiator is not colocalized by the target.

In some embodiments, an HCR initiator (i1 or i2) is split between three fractional-initiator probes (fraction f1 for probe P1, fraction f2 for probe P2, fraction 3 for probe 3 such that f1+f2+B=1); in this case, a probe unit consists of three fractional-initiator probes. In some embodiments, an HCR initiator (i1 or i2) is split between N fractional-initiator probes (fraction f1 for probe P1, fraction f2 for probe P2, . . . , fraction fN for probe PN such that f1+f2+ . . . fN=1; for example, see FIG. 51B) with N=2, 3, 4, or more; in this case, a probe unit consists of N fractional-initiator probes. In some embodiments, for any of these values of N, HCR signal amplification is suppressed if the full HCR initiator is not colocalized by the target.

In some embodiments, a full HCR initiator is generated by colocalization of a pair (or set) of probes that each carry a fraction of an HCR initiator such that the sum of the fraction f1 for probe P1 and the fraction f2 for probe P2 (f1+f2) is sufficiently close to 1 (for example, f1=0.47, f2=0.47, f1+f2=0.94; or f1=0.44, f2=0.42, f1+f2=0.86) such that HCR signal amplification is triggered by the colocalized full initiator that results from binding of the pair of probes to their adjacent cognate binding sites on the target. In some embodiments, the fractional-initiator probes within a probe unit generate a full HCR initiator corresponding to 100% of an HCR initiator. In some embodiments, the fractional-initiator probes within a probe unit generate a sufficient fraction of an HCR initiator to provide efficient HCR signal amplification relative to the rate of signal amplification when no fractional-initiator probes are present or when individual fractional-initiator probes are present but are not colocalized by the target. In some embodiments, the fraction of a full HCR initiator generated by colocalized probes within a probe unit is 99%, 95%, 90%, 80%, or 60%, including any range above any one of the preceding values or defined between any two of the preceding values of a full HCR initiator. In some embodiments, a probe unit comprises 2, 3, 4, 5 or more fractional-initiator probes. In some embodiments, the fractional initiators in the probe unit are sufficient to be functional as an HCR initiator when the probes within the probe unit are colocalized by binding to their adjacent cognate binding sites on the target. In some embodiments, while an HCR initiator may have a sequence of a particular length (e.g., 15 nucleotides), the fractional initiators within a probe unit need not be the exact same length. For example, in some embodiments, their combined length could be 14 or 13 nucleotides, if, when colocalized, they still function as an HCR initiator.

In some embodiments, any two or more fractional initiators can be used, as long as, together, they provide the function of an HCR initiator.

In some embodiments, a full HCR initiator is generated by colocalization of a pair of probes that each carry a fraction of an HCR initiator further comprising one or a few or several sequence modifications such that the sum of the fraction f1 for probe P1 and the fraction f2 for probe P2 (f1+f2) is sufficiently close to 1 (for example, f1=0.45, f2=0.47, f1+f2=0.92) such that HCR signal amplification is triggered by the colocalized full initiator that results from binding of the pair of probes to their adjacent cognate binding sites on the target. In some embodiments, the fractional-initiator probes within a probe unit generate a full HCR initiator that has 100% sequence identity with an HCR initiator. In some embodiments, the fractional-initiator probes within a probe unit generate sufficient sequence identity to an HCR initiator to allow efficient HCR signal amplification relative to the rate of signal amplification when no fractional-initiator probes are present or when individual fractional-initiator probes are present but are not colocalized by the target. In some embodiments, the full HCR initiator generated by colocalized probes within a probe unit has 99%, 95%, 90%, 80%, or 60% sequence identity with an HCR initiator, including any range above any one of the preceding values or defined between any two of the preceding values.

Increasing signal strength using helper probes. In order to maintain selectivity for the target, in some embodiments the probe set size is constrained to be no more than 1 probe or 1 probe unit, or no more than 2 probes or 2 probe units, or no more than N probes or N probe units, where N is less than the number of probes or probe units, N+M, that would be preferentially used to maximize signal strength. In this scenario where the probe set size is constrained by selectivity considerations, the amount of signal generated can be less relative to detection of the same target using N+M probe units both because the probe set has the potential for generating M fewer full initiators for triggering HCR signal amplification and because the absence of the M additional probe units can reduce the binding yield of the N remaining probe units due to cooperative effects.

In some embodiments, signal probes carry one or more initiators or fractional initiators (for example, see FIG. 50A). In some embodiments, helper probes do not carry initiators or fractional initiators. In some embodiments, in order to increase signal without decreasing selectivity, a signal probe set comprising N probe units can be augmented with M helper probes (for example, see FIG. 50D).

In some embodiments, detecting a target such as a target RNA with a unique splice junction, a signal probe set could comprise one probe unit that binds to the target spanning the splice junction, and further comprise 30 (or any number of) helper probes that bind elsewhere to the target RNA to cooperatively improve the binding yield of the probe unit and thus increase signal without decreasing selectivity. In some embodiments, detecting a target RNA or DNA for which selectivity considerations lead to a signal probe set comprising 5 probe units, the probe set can further comprise 45 helper probes. In some embodiments, detecting a target RNA or DNA for which selectivity considerations lead to a signal probe set comprising N probe units (with N less than 40), the signal probe set can further comprise 40-N helper probes. In some embodiments, detecting a target nucleic acid for which selectivity considerations lead to a signal probe set comprising N probe units, and the length of the target nucleic acid restricts the total number of probes to 2N+M, the signal probe set can further comprise M helper probes.

In some embodiments, helper probes will be designed for a target nucleic acid with less stringent selectivity requirements than those used to design signal probes for the same target nucleic acid. In some embodiments, helper probes will be used for a target nucleic acid even though they are equally selective for other off-target nucleic acids within the transcriptomes and/or genomes present in the sample. In some embodiments, the target-binding domain on signal probes will be the same length as the target-binding domain on helper probes. In some embodiments, the target-binding domain on signal probes will be shorter or longer than the target binding domain on helper probes. In some embodiments, the target-binding domain may vary across a range of lengths (number of nucleotides) for different signal and/or helper probes. In some embodiments, the affinity between signal probes and the target nucleic acid will be comparable to the affinity between helper probes and the target nucleic acid. In some embodiments, the affinity between signal probes and the target nucleic acid will be lower or higher than the affinity between helper probes and the target nucleic acid.

In some embodiments, a capture probe set can be augmented by one or more helper probes to improve the binding yield of capture probes with the target.

Increasing signal using signal probes that carry one or two fractional initiators. In some embodiments, a probe unit comprises two fractional-initiator probes that together generate a full HCR initiator (fraction f1 for probe P1 and fraction f2 for probe P2 such that f1+f2=1). In this situation, each probe participates in one probe unit. If both probes within the probe unit bind to a given target RNA molecule, a single full HCR initiator is generated, triggering growth of one HCR amplification polymer tethered to the probe unit. Consider a situation where the length of the target RNA constrains the probe set size to be N probe units corresponding to 2N signal probes. In the case where each signal probe participates in only one probe unit, the maximum number of full HCR initiators would be N, corresponding to the case where the target mRNA was bound by all of the signal probes in the probe set. Hence, the maximum number of HCR polymers tethered to a target RNA would be N. In order to increase the signal per target molecule, now consider a case where a probe can carry two fractional initiators that contribute to two different probe units (for example, see FIG. 50C). In some embodiments, the following three possibilities are contemplated:

In an embodiment, where all 2N signal probes hybridize to adjacent subsequences along the target RNA, the signal probes at the 5′ and 3′ ends of the target each have one neighboring signal probe and all other intervening signal probes have two neighboring signal probes. In this scenario, if each probe except for the 5′-most signal probe and the 3′-most signal probe carries two fractional initiators that contribute to two different probe units, the total number of probe units increases from N to (2N−1). As a result, the maximum number of full HCR initiators increases from N to (2N−1) and the maximum number of HCR polymers tethered to the target RNA increases from N to (2N−1). Hence, the signal generated per target molecule is increased relative to the case where each signal probe carries a single fractional initiator.

In another embodiment, selectivity considerations resulting from the transcriptomes and/or genomes present in the sample may lead to a probe set comprising 2N signal probes and N probe units where no probe unit is proximal to another along the target nucleic acid. In this scenario, each signal probe carries only a single fractional initiator and contributes to only one probe unit so the maximum number of full HCR initiators is N and the maximum number of tethered HCR amplification polymers is N.

In another embodiment, representing an intermediate case, some signal probes will be proximal to more than one neighbor so the 2N signal probes will participate in a number of probe units that is intermediate between N and 2N−1. For example, if there are 2N signal probes and M of them tile one portion of the target (with the 5′ and 3′ of these signal probes carrying one fractional initiator and contributing to one probe unit and the other M−2 signal probes carrying two fractional initiators and contributing to two probe units) and the other L probes (with 2N=M+L with L even) each bind to the target in pairs with each signal probe proximal to only one other signal probe (each of L signal probes carrying one fractional initiator and contributing to one probe unit), then the total number of probe units will be M−1+L/2 which will be intermediate between N and 2N−1. For example, if there are 40 signal probes (i.e., N=20) and 20 of them tile the target proximally (i.e., M=20) and the other 20 bind to the target in proximal pairs (i.e., L=20), the total number of probe units will be M−1+L/2=20−1+20/2=29.

In the related case where a probe unit comprises more than two signal probes, for example three signal probes, or four or more signal probes, the number of probe units can again be increased without increasing the number of signal probes, by using some probes that comprise two fractional initiators and participate in two probe units (one fractional initiator per probe unit). In some embodiments, the above approach can be used analogously for a target DNA. In some embodiments, this approach can be generalized to target proteins and other targets using fractional-initiator probes that carry more than one fractional initiator.

HCR amplifiers with 2 hairpins. In some embodiments, an HCR amplifier comprises two hairpins (h1 and h2; for example, see FIGS. 4A-4B and 17A-17F). In some embodiments, each hairpin comprises an input domain with a single-stranded toehold and a stem section, and an output domain with a single-stranded loop and a complement to the stem section. In the absence of an HCR initiator (i1 or i2), hairpins h1 and h2 coexist metastably, that is, they are kinetically trapped and do not polymerize.

Initiation with initiator i1. In some embodiments, an initiator i1 comprises a domain complementary to the toehold of hairpin h1 and a domain complementary to the stem section of h1 (for example, see FIG. 14). In some embodiments, if an h1 hairpin encounters initiator i1, the initiator i1 hybridizes to the input domain of hairpin h1 via toehold-mediated strand displacement, opening hairpin h1 to expose the output domain of hairpin h1 and form complex i1-h1. In some embodiments, the output domain of hairpin h1 comprises a domain complementary to the toehold of hairpin h2 and a domain complementary to the stem section of h2. In some embodiments, if an h2 hairpin encounters an i1-h1 complex, the exposed output domain of h1 hybridizes to the input domain of hairpin h2 via toehold-mediated strand displacement, opening hairpin h2 to expose the output domain of hairpin h2 and form complex i1-h1-h2. In some embodiments, the output domain of hairpin h2 comprises a domain complementary to the toehold of hairpin h1 and a domain complementary to the stem section of h1. In some embodiments, if an h1 hairpin encounters an i1-h1-h2 complex, the exposed output domain of h2 hybridizes to the input domain of hairpin h1 via toehold-mediated strand displacement, opening hairpin h1 to expose the output domain of hairpin h1 and form complex i1-h1-h2-h1. In some embodiments, this polymerization process can repeat with alternating h1 and h2 polymerization steps to generate polymers of the form i1-h1-h2-h1-h2-h1-h2- . . . , which we may denote i1-(h1-h2)_{N} for a polymer that incorporates N alternating copies of hairpins h1 and h2. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i1-(h1-h2)_{N}-h1 and i1-(h1-h2)_{N}-h1-h2 are both possible, the latter being equivalent to i1-(h1-h2)_{N}+1.

Initiation with initiator i2. In some embodiments, an initiator i2 comprises a domain complementary to the toehold of hairpin h2 and a domain complementary to the stem section of h2 (for example see FIG. 3B). In some embodiments, if an h2 hairpin encounters initiator i2, the initiator i2 hybridizes to the input domain of hairpin h2 via toehold-mediated strand displacement, opening hairpin h2 to expose the output domain of hairpin h2 and form complex i2-h2. In some embodiments, if an h1 hairpin encounters an i2-h2 complex, the exposed output domain of h2 hybridizes to the input domain of hairpin h1 via toehold-mediated strand displacement, opening hairpin h1 to expose the output domain of hairpin h1 and form complex i2-h2-h1. In some embodiments, if an h2 hairpin encounters an i2-h2-h1 complex, the exposed output domain of h1 hybridizes to the input domain of hairpin h2 via toehold-mediated strand displacement, opening hairpin h2 to expose the output domain of hairpin h2 and form complex i2-h2-h1-h2. In some embodiments, this polymerization process can repeat with alternating h2 and h1 polymerization steps to generate polymers of the form i2-h2-h1-h2-h1-h2-h1 . . . , which can be denoted i2-(h2-h1)_{N} for a polymer that incorporates N alternating copies of h2 and h1. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i2-(h2-h1)_{N}-h2 and i2-(h2-h1)_{N}-h2-h1 are both possible, the latter being equivalent to i2-(h2-h1)_{N+1}.

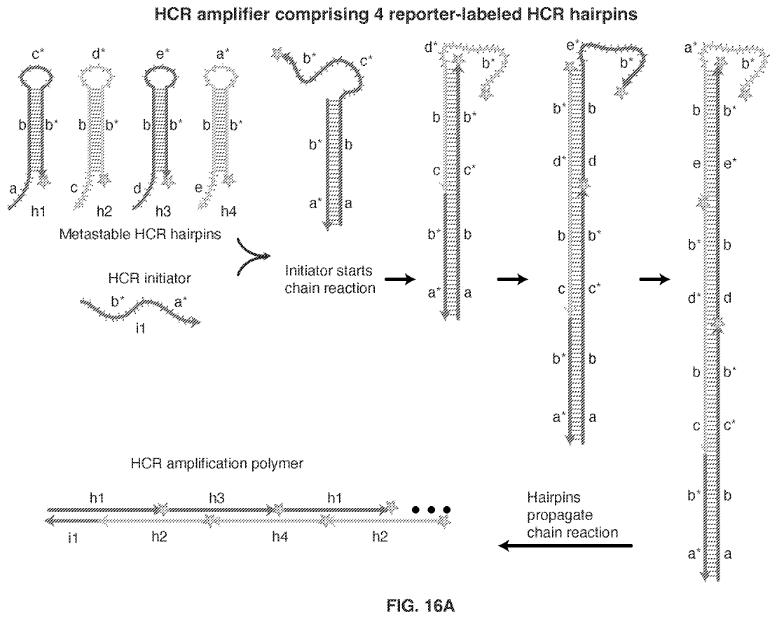

HCR amplifiers with 4 hairpins. In some embodiments, an HCR amplifier can comprise more than 2 hairpins. For example, an HCR amplifier might comprise 4 hairpins h1, h2, h3, h4 (for example, see FIGS. 16A-16B). In some embodiments, just as for 2-hairpin HCR, each hairpin comprises an input domain comprising a single-stranded toehold and a stem section, and an output domain comprising a single-stranded loop and a complement to the stem section. In some embodiments, in the absence of an HCR initiator (i1, i2, i3, or i4), hairpins h1, h2, h3, h4 coexist metastably, that is, they are kinetically trapped and do not polymerize. In some embodiments, the output domain of hairpin h1 comprises a domain complementary to the toehold of hairpin h2 and a domain complementary to the stem section of h2; the output domain of hairpin h2 comprises a domain complementary to the toehold of hairpin h3 and a domain complementary to the stem section of h3; the output domain of hairpin h3 comprises a domain complementary to the toehold of hairpin h4 and a domain complementary to the stem section of h4; the output domain of hairpin h4 comprises a domain complementary to the toehold of hairpin h1 and a domain complementary to the stem section of h1. In some embodiments, initiator i1 comprises a domain complementary to the toehold of hairpin h1 and a domain complementary to the stem section of h1; initiator i2 comprises a domain complementary to the toehold of hairpin h2 and a domain complementary to the stem section of h2; initiator i3 comprises a domain complementary to the toehold of hairpin h3 and a domain complementary to the stem section of h3; initiator i4 comprises a domain complementary to the toehold of hairpin h4 and a domain complementary to the stem section of h4. In some embodiments, analogous to the case of 2-hairpin HCR, if a hairpin h1 encounters a initiator i1, the initiator i1 opens hairpin h1 to form complex i1-h1 with an exposed h1 output domain, which in turn opens hairpin h2 to form complex i1-h1-h2 with an exposed h2 output domain, which in turn opens hairpin h3 with an exposed output domain to form complex i1-h1-h2-h3 with an exposed h3 output domain, which in turn opens hairpin h4 to form complex i1-h1-h2-h3-h4 with an exposed h4 output domain, which in turn opens hairpin h1 to form complex i1-h1-h2-h3-h4-h1 with an exposed h1 output domain, and so on and so forth, leading to polymerization via alternating h1, h2, h3, and h4 polymerization steps to generate polymers of the form i1-h1-h2-h3-h4-h1-h2-h3-h4-h1-h2-h3-h4 . . . , which can be denoted i1-(h1-h2-h3-h4)_{N} for a polymer that incorporates N alternating copies of h1, h2, h3, and h4. In some embodiments, it is possible for a polymer to end with h1, h2, h3, or h4, so i1-(h1-h2-h3-h4)_{N}-h1, i1-(h1-h2-h3-h4)_{N}-h1-h2, i1-(h1-h2-h3-h4)_{N}-h1-h2-h3, and i1-(h1-h2-h3-h4)_{N}-h1-h2-h3-h4 are all possible, the latter being equivalent to i1-(h1-h2-h3-h4)_{N}+1. In some embodiments, it is possible for HCR polymerization to be triggered by any of the cognate initiators (i1, i2, i3, or i4). For example, initiation by initiator i3 could generate polymers of the form i3-(h3-h4-h1-h2)_{N}. In some embodiments, HCR amplifiers with 4 hairpins are convenient for generating a signal that is absent in the unpolymerized state and present in the polymer state (for example, FIG. 16B illustrates FRET pairs that are colocalized to generate a FRET signal only when hairpins are colocalized within an amplification polymer, providing a basis for wash-free methods since unused hairpins that are not washed from the sample will not participate in FRET, and hence will avoid generating background).

HCR amplifiers with 2 or more hairpins. More generally, in some embodiments, an HCR amplifier may comprise M HCR hairpins (h1, h2, . . . , hM) with M an integer of 2 or more. In the absence of an HCR initiator (i1, i2, . . . , iM), hairpins h1, h2, . . . , hM coexist metastably, that is, they are kinetically trapped and do not polymerize. In the presence of a cognate HCR initiator, polymerization occurs via alternating polymerization steps analogous to 2-hairpin or 4-hairpin HCR. For example, initiator i1 would lead to growth of polymers of the form i1-(h1-h2- . . . -hM)_{N} for a polymer that incorporates N alternating copies of h1, h2, . . . , hM. It is possible for a polymer to end with any of h1, h2, . . . , hM, so i1-(h1-h2- . . . -hM)_{N}-h1, i1-(h1-h2- . . . -hM)_{N}-h1-h2, . . . , and i1-(h1-h2- . . . -hM)_{N}-h1-h2- . . . -hM are all possible, the latter being equivalent to i1-(h1-h2- . . . -hM)_{N}+1. It is possible for HCR polymerization to be triggered by any of the cognate initiators (i1, i2, . . . , iM). For example, initiator by full initiator i3 could generate polymers of the form i3-(h3- . . . -hM-h1-h2)_{N}.

Reporter-labeled HCR hairpins. For a given HCR amplifier, each HCR hairpin comprises zero, one, or more reporters. Reporters on different hairpins within an amplifier may be the same or different. For example, an amplifier comprising hairpins h1 and h2 might have: 1) the same reporter on h1 and h2, 2) different reporters on h1 and h2, 3) a reporter on h1 but no reporter on h2, 4) a reporter on h2 but no reporter on h1, 5) no reporter on h1 or h2, 6) zero, one, or more reporters on h1 of which zero, one, or more of them are the same or different as zero, one, or more reporters on h2. Similarly, for an HCR amplifier comprising hairpins h1, h2, h3, h4, each hairpin may comprise zero, one, or more reporters (for example 3, 5, or 10 reporters) of which zero, one, or more of them may be the same as zero, one, or more reporters on each of the other hairpins. In some embodiments, one or more of the reporters for a given hairpin can be unique within a mixture of hairpins and/or hairpin reporters. In some embodiments, there are 1, 10, 100, 1000, 10,000, 100,000 or more unique reporters within a mixture (including any range defined between any two of the previous numbers).

In some embodiments, the one or more reporters on a reporter-labeled HCR hairpin directly or indirectly contributes to the generation, alteration, or elimination of a signal. For example, a reporter could be a fluorophore, a chromophore, a luminophore, a phosphor, a FRET pair, a member of a FRET pair, a quencher, a fluorophore/quencher pair, a rare-earth element or compound, a radioactive molecule, a magnetic molecule, an enzyme, or any other molecule that facilitates measurement of a signal.

In some embodiments, a reporter decorating a tethered HCR amplification polymer immobilized in the test region of an HCR lateral flow device may bind to the reporter-binding domain of a readout probe to directly or indirectly mediate localization of auxiliary reporters in the vicinity of the reporter, which in turn directly or indirectly mediates generation of an amplified signal in the test region. For example:

In some embodiments, the reporter can comprise digoxigenin (DIG) that recruits anti-DIG antibody as the readout probe, where the anti-DIG is directly labeled with one or more auxiliary reporters, or with one or more reporters that serve to directly or indirectly mediate localization of auxiliary reporters in the vicinity of the reporter.

In some embodiments, the reporter can comprise a nucleic acid domain that serves as a substrate with full or partial sequence complementarity to a reporter-binding domain within a readout probe that carries one or more auxiliary reporters (for example, see FIG. 17C),

In some embodiments, the reporter can comprise a nucleic acid domain that serves as a substrate with full or partial sequence complementarity to a reporter-binding domain within a readout probe that carries one or more substrates that serve to mediate localization of auxiliary reporters in the vicinity of the reporter.

In some embodiments, the reporter can comprise a nucleic acid domain that serves as a substrate for a readout probe that directly or indirectly mediates localization of auxiliary reporters in the vicinity of the reporter.

In some embodiments, the reporter can comprise a substrate that serves to recruit a readout probe that indirectly mediates localization of auxiliary reporters in the vicinity of the reporter.

In some embodiments, the reporter can comprise a substrate that serves to recruit a readout probe that comprises an enzyme that mediates catalytic reporter deposition (CARD) in the vicinity of the reporter (for example, see FIG. 27A).

In some embodiments, the reporter can comprise biotin that recruits streptavidin (or another biotin-binding molecule) as the readout probe, where the streptavidin is directly labeled with one or more auxiliary reporters, or with one or more substrates that serve to directly or indirectly mediate localization of auxiliary reporters in the vicinity of the reporter.

In some embodiments, the reporter can comprise a hapten that recruits an anti-hapten antibody readout probe or an anti-hapten nanobody readout probe that directly or indirectly mediates localization of reporters in the vicinity of the reporter via CARD signal amplification. For example, the anti-hapten antibody or nanobody readout probe may comprise an enzyme that mediates CARD (for example, see FIGS. 25A-25E).

In some embodiments, the reporter can comprise a hapten that recruits an anti-hapten that directly or indirectly mediates localization of auxiliary reporters in the vicinity of the hairpin reporter. For example, the anti-hapten readout probe may comprise an enzyme that mediates CARD (for example, see FIGS. 26A-26C).

In some embodiments, the reporter can comprise an enzyme that mediates CARD signal amplification to deposit CARD-reporter molecules in the vicinity of the hairpin.

In some embodiments, the hairpin reporter can comprise zero, one, or more haptens (for example, see FIGS. 17A-17B) that mediate, directly or indirectly, localization of auxiliary reporters in the vicinity of the haptens.

In some embodiments, a reporter can comprise a hapten that recruits an anti-hapten (for example, an antibody, a nanobody, streptavidin, or another molecule) that is labeled with auxiliary reporters.

In some embodiments provided herein, HCR signal amplification is used to mediate catalytic reporter deposition (CARD), leading to even higher signal gain. In some embodiments, the even higher single gain is about 5, 10, 15, 20, 25, 30, 40, 50, 75, 100, 500, 1000, 2000, 5000, 10,000, 20,000, 50,000, or 100,000-fold, or a value with a range defined by any two of the aforementioned values.

Enzymes for HCR-mediated Catalytic Reporter Deposition (CARD). In some embodiments, reporter-decorated HCR amplification polymers mediate signal amplification via catalytic reporter deposition (CARD) by an enzyme that catalyzes a CARD-substrate leading to deposition of CARD-reporters in the vicinity of the HCR amplification polymer (for example, see FIGS. 25A-25E, 26A-26C, 27A-27B). For example:

In some embodiments, the enzyme could be horseradish peroxidase (HRP) (or polymer HRP comprising multiple HRP enzymes) that acts on a CARD-substrate to catalyze deposition a chromogenic CARD-reporter such as AEC, DAB, TMB, or StayYellow, or that catalyzes a CARD-substrate to catalyze deposition of a fluorescent CARD-reporter such as fluorophore-labeled tyramide, or that catalyzes deposition of a hapten-labeled CARD-substrate such as biotin-labeled tyramide, where the hapten serves to mediate localization of CARD-reporters in the vicinity of the reporter-decorated HCR amplification polymer.

In some embodiments, the enzyme could be alkaline phosphatase (AP) (or polymer AP comprising multiple AP enzymes) that acts on a CARD-substrate to catalyze deposition of CARD-reporters, for example a chromogenic CARD-reporter such as but not limited to BCIP/NBT, BCIP/TNBT, Napthol AS-MX phosphate+FastBlue BB, Napthol AS-MX phosphate+FastRed TR, StayGreen.

In some embodiments, the enzyme could be glucose oxidase that acts on a CARD-substrate to catalyze deposition of CARD-reporters, for example NBT.

In some embodiments, the enzyme could be any molecule or complex that directly or indirectly mediates localization of CARD-reporters in the vicinity of a reporter-decorated HCR amplification polymer.

In some embodiments, CARD-reporters deposited in the vicinity of tethered HCR amplification polymers immobilized in the test region are visible to the human eye. In some embodiments, CARD-reporters deposited in the vicinity of tethered HCR amplification polymers immobilized in the test region are scanned with an instrument to read a signal.

In some embodiments, the enzyme that mediates CARD is deactivated (aka inactivated) after CARD-reporter deposition (for example, using chemical or heat denaturation). For example, the enzyme that mediates CARD can be deactivated using any combination of:
1. Heat (for example, 65° C. or above)
2. Fixative (for example, 4% PFA)
3. Acid (for example, 0.1 M glycine-HCl with 1% Tween 20 at pH 2.2, 0.2N HCl, 10% acetic acid, 10 mM HCl)
4. Other chemicals (for example, hydrogen peroxide (H₂O₂), hydrogen peroxide+phenol, sodium azide, DEPC, MAB with 10 mM EDTA)

In some embodiments, HRP is inactivated using H₂O₂. In some embodiments, AP is inactivated using a combination of heat and acid. In some embodiments, AP is inactivated with fixative. In some embodiments, deactivation of the enzyme that mediates CARD allows repeated CARD using the same enzyme in combination with different substrates for different targets to allow multiplexed target analysis using HCR-mediated CARD. In some embodiments, deactivation of the enzyme that mediates CARD allows repeated CARD using different enzymes in combination with different CARD-substrates for different targets to allow multiplexed target analysis using HCR-mediated CARD.

In some embodiments, CARD allows storage of stained samples for 10 or more years to allow reanalysis in compliance with regulatory requirements. In some embodiments, the CARD-stained sample is adequately stable for 1, 2, 3, 4, 5, 6, 7, 8, 9, 10, 11, 12, 13, 14, 15 or more years, and still in compliance with regulatory requirements. In some embodiments, CARD staining provides for long-term storage of archival samples.

Target types. In some embodiments, an initiator-labeled probe comprises a target-binding domain and further comprises one or more HCR initiators.

In some embodiments, an initiator-labeled probe can detect a target comprising any molecule including but not limited to an RNA molecule (for example, mRNA, rRNA, lncRNA, siRNA, shRNA, microRNA, non-coding RNA, synthetic RNA, or modified RNA), a DNA molecule, a non-natural nucleic acid molecule, a protein molecule, a small molecule, a biological molecule, a chemically modified biological molecule, a non-biological molecule.

In some embodiments, an initiator-labeled probe can detect a target comprising any complex of molecules comprising any combination of RNA, DNA, protein, small molecules, biological molecules, and/or non-biological molecules (for example, an RNA/RNA complex, an RNA/protein complex, a DNA/protein complex, an RNA/DNA/protein complex, a protein/protein complex).

In some embodiments, an initiator-labeled probe can detect a target comprising a prokaryotic cell, a eukaryotic cell, a virus.

In some embodiments, an initiator-labeled probe can detect a target comprising any combination of the above.

In some embodiments, a fractional-initiator probe comprises a target-binding domain and further comprises one or more fraction-initiators. In some embodiments, a probe unit comprises two or more fractional-initiator probes such that the fractional-initiator probes in the probe unit combine to create a full HCR initiator. In some embodiments, a probe unit can detect a target comprising any molecule including but not limited to an RNA molecule (for example, mRNA, rRNA, lncRNA, siRNA, shRNA, microRNA, non-coding RNA, synthetic RNA, or modified RNA), a DNA molecule, a non-natural nucleic acid molecule, a protein molecule, a small molecule, a biological molecule, a chemically modified biological molecule, a non-biological molecule,

In some embodiments, a probe unit can detect a target comprising any complex of molecules comprising any combination of RNA, DNA, protein, small molecules, biological molecules, and/or non-biological molecules (for example, an RNA/RNA complex, an RNA/protein complex, a DNA/protein complex, an RNA/DNA/protein complex, a protein/protein complex).

In some embodiments, a probe unit can detect a target comprising any collection of proximal molecules or complexes such that the fractional initiators in the probe unit can colocalize to form a full HCR initiator when the fractional-initiator probes comprising the probe unit are bound to their respective targets within the collection of proximal molecules or complexes.

In some embodiments, a probe unit can detect a target comprising a prokaryotic cell, a eukaryotic cell, a virus.

In some embodiments, a probe unit can detect a target comprising any combination of the above.

In any of the embodiments provided herein, the fractional initiators within a probe unit may be designed to be (or are) complementary to non-overlapping regions of an HCR hairpin (for example, regions separated by 0, 1, 2, or more nucleotides), or are designed to be (or are) complementary to overlapping regions of an HCR hairpin (for example, regions that overlap by 1, 2 or more nucleotides), or are designed to be (or are) substantially complementary to an HCR hairpin (for example, complementary except for 0, 1, 2, a few, or several mismatches), or are configured to bind an HCR hairpin.

In any of the embodiments provided herein, the target-binding regions within a probe unit are configured to bind to non-overlapping regions of the target (for example, regions separated by 0, 1, 2, or more nucleotides or regions separated by 0, 1, 2, or more nanometers), or are configured to bind to overlapping regions of the target (for example, regions that overlap by 1, 2 or more nucleotides, or regions that overlap by 1, 2, or more nanometers).

Signal probe sets for multiplexing. In some embodiments, signal probe sets are designed for multiplexed analysis in which 2, 3, 4, 5, 10, 20, or 100 or more signal probe sets are used to bind to different targets in the same sample, where 1, 2, 3, 4, 5, 10, 20, or 100 or more of the signal probe sets comprise one or more initiator-labeled probes, or one or more probe units each comprising two or more fractional-initiator probes. In some embodiments, signal probe sets are designed for multiplexed experiments in which 2, 3, 4, 5, 10, 20, or 100 or more signal probe sets are used in the same sample, where more than 1%, more than 2%, more than 5%, more than 10%, more than 30%, more than 50%, or 100% of the signal probe sets comprise one or more initiator-labeled probes, or one or more probe units each comprising two or more fractional-initiator probes.

Materials and compositions of initiator-labeled probes. In some embodiments, an initiator-labeled probe comprises one or more target-binding domains and one or more HCR initiators (for example, see FIGS. 18A-18N and 19A-19F). In some embodiments, each domain may comprise one or more materials including DNA, RNA, 2′OMe-RNA, PNA, XNA, chemically modified nucleic acids, synthetic nucleic acid analogs, amino acids, chemical linkers, synthetic amino acid analogs, and/or any other molecule suited for the purpose of the domain. For example:

In some embodiments, an initiator-labeled probe may comprise one or more initiators made of DNA and a target-binding domain made of DNA.

In some embodiments, an initiator-labeled probe may comprise one or more initiators made of DNA, a chemical linker, and a target-binding domain made of amino acids (for example, an antibody or a nanobody or an antibody fragment).

In some embodiments, an initiator-labeled probe may comprise an initiator made of a synthetic nucleic acid analog and a target-binding domain made of a combination of DNA and 2′OMe-RNA.

In some embodiments, an initiator-labeled probe may comprise an initiator made of 2′OMe-RNA and a target-binding domain made of a combination of RNA and protein.

In some embodiments, an initiator-labeled probe may comprise an initiator made of DNA and a target-binding domain made of PNA.

In some embodiments, an initiator-labeled probe may comprise one or more initiators made of any nucleic acid or nucleic acid analog and one or more target-binding domains made of any combination of materials suitable for binding the target molecule.

In some embodiments, an initiator-labeled probe may comprise a single covalently linked molecule or may comprise two or more molecules (each covalently linked) that interact non-covalently to form a complex. For example:

In some embodiments, an initiator-labeled probe may comprise an initiator made of DNA that is covalently linked to a target-binding domain made of DNA.

In some embodiments, an initiator-labeled probe may comprise one or more initiators made of DNA that are covalently linked to dCas9 (or another Cas) which is non-covalently bound to a guide RNA (gRNA) such that the target-binding domain comprises the gRNA:dCas9 complex (or gRNA:Cas complex using another Cas).

In some embodiments, an initiator-labeled probe may comprise one or more initiators made of DNA that are covalently linked to a gRNA that is non-covalently bound to dCas9 (or another Cas) such that the target-binding domain comprises the gRNA:dCas9 complex (or gRNA:Cas complex using another Cas).

In some embodiments, an initiator-labeled probe may comprise an initiator made of a nucleic acid or nucleic acid analog that is covalently linked or non-covalently bound to a target-binding domain comprising one or more molecules.

Materials and composition of fractional-initiator probes. In some embodiments, a fractional-initiator probe comprises one or more target-binding domains and one or more fractional initiators (for example, see FIGS. 20A-20E, 21A-21D, 22A-22E, and 24). In some embodiments, each domain may comprise one or more materials including DNA, RNA, 2′OMe-RNA, PNA, XNA, chemically modified nucleic acids, synthetic nucleic acid analogs, chemical linkers, amino acids, synthetic amino acid analogs, and/or any other molecule suited for the purpose of the domain. For example:

In some embodiments, a fractional-initiator probe may comprise one or more fractional initiators made of DNA and a target-binding domain made of DNA.

In some embodiments, a fractional-initiator probe may comprise one or more fractional initiators made of DNA, a chemical linker, and a target-binding domain made of amino acids (for example, an antibody or a nanobody or an antibody fragment).

In some embodiments, a fractional-initiator probe may comprise a fractional initiator made of a synthetic nucleic acid analog and a target-binding domain made of a combination of DNA and 2′OMe-RNA.

In some embodiments, a fractional-initiator probe may comprise a fractional initiator made of 2′OMe-RNA and a target-binding domain made of a combination of RNA and protein.

In some embodiments, a fractional-initiator probe may comprise a fractional initiator made of DNA and a target-binding domain made of PNA.

In some embodiments, a fractional-initiator probe may comprise one or more fractional initiators made of any nucleic acid or nucleic acid analog and one or more target-binding domains made of any combination of materials suitable for binding the target molecule.

In some embodiments, a fractional-initiator probe may comprise a single covalently linked molecule or may comprise two or more molecules (each covalently linked) that interact non-covalently to form a complex. For example:

In some embodiments, a fractional-initiator probe may comprise a fractional initiator made of DNA that is covalently linked to a target-binding domain made of DNA.

In some embodiments, a fractional-initiator probe may comprise one or more fractional initiators made of DNA that are covalently linked to dCas9 (or another Cas) which is non-covalently bound to a guide RNA (gRNA) such that the target-binding domain comprises the gRNA:dCas9 complex (or gRNA:Cas complex using another Cas).

In some embodiments, a fractional-initiator probe may comprise one or more fractional initiators made of DNA that are covalently linked to a gRNA that is non-covalently bound to dCas9 (or another Cas) such that the target-binding domain comprises the gRNA:dCas9 complex (or gRNA:Cas complex using another Cas).

In some embodiments, a fractional-initiator probe may comprise a fractional initiator made of a nucleic acid or nucleic acid analog that is covalently linked or non-covalently bound to a target-binding domain comprising one or more molecules. Each fractional-initiator probe within a probe unit may have the same or different material compositions from the other fractional-initiator probes in the probe unit. Each fractional-initiator probe within a probe unit may have target-binding regions that bind to different detection sites on the same target molecule, or to different detection sites within a target molecular complex, or to different detection sites within a target collection of proximal molecules or complexes.

Removal of signal. In some embodiments, HCR signal is removed from the test region after detecting the signal. Signal can be removed from the test region by any method that reduces the number of signal-generating reporters and/or auxiliary reporters from the test region. For example:

In some embodiments, signal can be removed from the test region by photobleaching fluorescent reporter molecules using light and/or chemical reagents.

In some embodiments, signal can be removed from the test region by chemically cleaving reporters from HCR hairpins and flowing them from the test region (e.g., TCEP).

In some embodiments, signal can be removed from the test region by chemically cleaving reporters from readout probes and flowing them from the test region.

In some embodiments, signal can be removed from the test region by chemically cleaving hairpins to fragment HCR amplification polymers and flowing the fragments from the test region.

In some embodiments, signal can be removed from the test region by chemically cleaving probes to untether HCR amplification polymers from the target and flowing the untethered amplification polymers from the test region.

In some embodiments, signal can be removed from the test region by using an auxiliary strand to dehybridize hairpins from HCR amplification polymers and flowing the hairpins from the test region.

In some embodiments, signal can be removed from the test region by using an auxiliary strand to dehybridize readout probes from HCR amplification polymers and flowing the readout probes from the test region.

In some embodiments, signal can be removed from the test region by using chemical denaturants and/or elevated temperature to destabilize HCR amplification polymers and then flowing the hairpins from the test region.

In some embodiments, signal can be removed from the test region by using chemical denaturants and/or elevated temperature to destabilize the interaction between probes and their targets and then flowing the untethered amplification polymers from the test region.

In some embodiments, signal can be removed from the test region by using chemical denaturants and/or elevated temperature to destabilize the interaction between readout probes and their substrates and then flowing the readout probes from the test region.

In some embodiments, signal can be removed from the test region by using enzymes to degrade amplification polymers and/or probes and flowing the degraded molecules from the test region.

In some embodiments, signal can be removed from the test region by using DNases to degrade DNA amplification polymers and/or DNA probes and/or DNA targets and flowing the resulting molecules from the test region.

In some embodiments, signal can be removed from the test region by using RNases to degrade RNA targets and then flowing the untethered amplification polymers from the test region.

In some embodiments, signal can be removed from the test region by using proteases to degrade protein targets and then flowing the untethered amplification polymers from the test region.

In some embodiments, signal can be removed from the test region by using a combination of RNases to degrade RNA targets and DNases to degrade DNA amplification polymers and/or DNA probes and flowing the resulting molecules from the test region.

In some embodiments, signal can be removed from the test region by using a combination of proteases to degrade protein targets and DNases to degrade DNA amplification polymers and/or DNA probes and/or DNA targets and flowing the resulting molecules from the test region.

In some embodiments, signal can be removed from the test region by using two or more of the above methods, or any other method for removing signal from the test region, at the same time or at different times.

### ADDITIONAL EMBODIMENTS

Any of the embodiments, compositions, and/or methods provided herein can be employed with, or in the alternative form of, any of the following. Thus, for example, the above noted compositions and/or methods can employ any of the compositions or methods noted below. Similarly, the above noted compositions and/or methods should be understood to also provide methods employing the methods below or as being part of the methods noted below.

Similarly, the embodiments and/or methods provided herein should also be understood to provide embodiments involved in the method, e.g., compositions, components of the method, kits, etc. In some embodiments, any of the ingredients in one or more of the methods and/or steps provided herein can be provided as a kit including one or more of the noted ingredients (and optionally the target or target sequence or sample).

### Compositions

Some embodiments of compositions of HCR monomers are outlined in FIGS. 14 and 23, as well as other figures provided herein. In some embodiments, a composition is provided that includes a first HCR hairpin (1510), comprising: a) a first input domain (1852), comprising a first toehold (1851) and a first stem section (1755), b) a first output domain (1854), comprising a first loop (1853) and a complement to the first stem section (1756), and optionally c) one or more reporters (1850).

In some embodiments, a composition is provided that comprises a signal probe set comprising: a first fractional-initiator probe (1190) that comprises a first fractional initiator (1151), and a second fractional-initiator probe (1290) that comprises a second fractional initiator (1251). In some embodiments, the first and second fractional initiators (1151, 1251) together form a full HCR initiator (1050) capable of binding to and opening the first HCR hairpin (1510). In some embodiments, the target (1020) comprises a first target domain (1100) and a second target domain (1200). In some embodiments, the target comprises additional domains. In some embodiments, the first fractional-initiator probe (1190) further comprises a first target-binding domain (1141) and the second fractional-initiator probe (1290) further comprises a second target-binding domain (1241), wherein the first target-binding domain (1141) is configured to bind to a first target domain (1100) and the second target binding domain (1241) is configured to bind to a second target domain (1200). These target-binding domains are located effectively adjacent on the target molecule, such that when both fractional-initiator probes are bound to both target domains, the first and second fractional initiators (within the fractional-initiator probes) are close enough to form a full HCR initiator (1050) capable of binding to and opening the first HCR hairpin.

In some embodiments, the composition can further include a second HCR hairpin (1610), comprising: a) a second input domain (1952), comprising a second toehold (1951) and a second stem section (1855), b) a second output domain (1954), comprising a second loop (1953) and a complement to the second stem section (1856), and optionally c) one or more reporters (1950).

In some embodiments, a composition is provided that comprises a signal probe set comprising: a first fractional-initiator probe (1190) that comprises a first fractional initiator (1151), and a second fractional-initiator probe (1290) that comprises a second fractional initiator (1251). In some embodiments, the first and second fractional initiators (1151, 1251) together form a full HCR initiator (1050), from which HCR can progress, via first and second HCR hairpins (1510, 1610). In some embodiments, the first fractional-initiator probe (1190) further comprises a first target-binding domain (1141) and the second fractional-initiator probe (1290) further comprises a second target binding domain (1241), wherein the first target-binding domain (1141) is configured to bind to a first target domain (1100) and the second target binding domain (1241) is configured to bind to a second target domain (1200). These target binding domains are located effectively adjacent on the target molecule, such that when both fractional-initiator probes are bound to both target domains, the first and second fractional initiators (within the fractional-initiator probes) are close enough to form a full HCR initiator (1050), from which HCR polymerization can occur.

In some embodiments, a composition is provided comprising a first HCR hairpin (1510), a second HCR hairpin (1610) and a signal probe set comprising a first fractional-initiator probe (1190) comprising a first fractional initiator (1151), and a second fractional-initiator probe (1290) comprising a second fractional initiator (1251). In some embodiments, the first and second fractional initiators (1151, 1251) together form a full HCR initiator (1050), from which HCR polymerization can progress, via the first and second HCR hairpins (1510, 1610).

In some embodiments, a composition is provided comprising a first HCR hairpin (1510), a second HCR hairpin (1610) and a signal probe set comprising a one or more signal probes comprising one or more HCR initiators (for example, see FIGS. 18 and 19), from which HCR polymerization can progress, via the first and second HCR hairpins (1510, 1610).

In some embodiments, a composition is provided that includes a first HCR hairpin (1510), comprising: a) a first input domain (1852), comprising a first toehold (1851) and a first stem section (1755), b) a first output domain (1854), comprising a first loop (1853) and a complement to the first stem section (1756), and optionally c) one or more reporters (1850). In some embodiments, the composition further includes a second HCR hairpin (1610), comprising: a) a second input domain (1952), comprising a second toehold (1951) and a second stem section (1855), b) a second output domain (1954), comprising a second loop (1953) and a complement to the second stem section (1856), and optionally c) one or more reporters (1950). In some embodiments, the composition can further include a signal probe set comprising one or more probe units comprising a) a first fractional-initiator probe (1190) comprising a first fractional initiator (1151), and b) a second fractional-initiator probe (1290) comprising a second fractional initiator (1251).

In some embodiments, the first stem section has the same sequence as the second stem section. In some embodiments, the complement to the first stem section has the same sequence as the complement to the second stem section. In some embodiments, the complement to the first stem section has the same sequence as the complement to the second stem section, and the first stem section has the same sequence as the second stem section. In some embodiments, the toehold sequence of the two hairpin monomers is the same and the loop sequence of the two hairpin monomers is the same (although the polarity of the two hairpin monomers is reversed, so the two hairpin monomers are not identical).

In some embodiments, the first toehold (1851) is complementary to the second loop (1953). In some embodiments, the second toehold (1951) is complementary to the first loop (1853). In some embodiments, this circularity allows HCR polymerization to occur. In some embodiments, the first toehold is not 100% complementary to the second loop but is sufficient to allow hybridization.

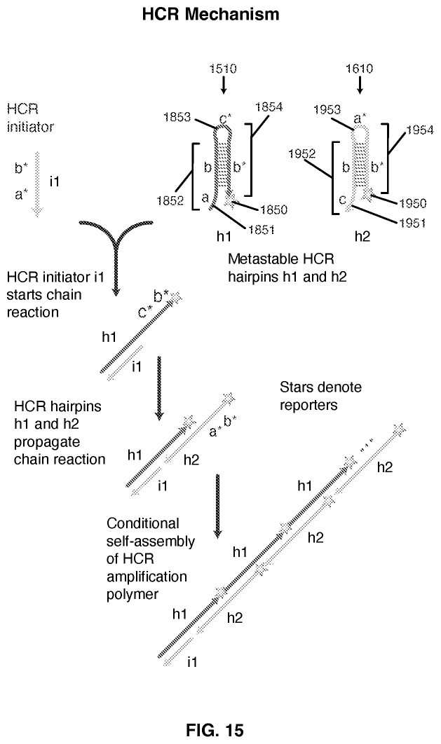

In some embodiments, an HCR amplifier comprises (for example, see FIGS. 3A and 15): 1) a first HCR hairpin (h1) comprising a first input domain (sequence domains “a-b”) comprising a first toehold (sequence domain “a”) and a first stem section (sequence domain “b”), and a first output domain (sequence domains “c*-b*”) comprising a first loop (sequence domain “c*”) and a partner to the first stem section (sequence domain “b*”) that is configured to bind the first stem section, and 2) a second HCR hairpin (h2) comprising a second input domain (sequence domains “b-c”) comprising a second toehold (sequence domain “c”) and a second stem section (sequence domain “b”), and a second output domain (sequence domains “b*-a*”) comprising a second loop (sequence domain “a*”) and a partner to the second stem section (sequence domain “b*”) that is configured to bind the second stem section. In some embodiments, a first initiator (i1; sequence domains “b*-a*”) is configured to bind the first input domain. In some embodiments, binding of the first initiator to the first input domain opens the first HCR hairpin to expose the first output domain. In some embodiments, the exposed first output domain is configured to bind the second input domain. In some embodiments, binding of the exposed first output domain to the second input domain opens the second HCR hairpin to expose the second output domain. In some embodiments, the exposed second output domain is configured to bind the first input domain. In some embodiments, binding of the exposed second output domain to the first input domain leads to HCR polymerization in which first and second HCR hairpins are successively opened and added to the growing polymer in alternating fashion.

In some embodiments, a second initiator (i2; domains “c*-b*”) is configured to bind the second input domain. In some embodiments, binding of the second initiator to the second input domain opens the second HCR hairpin to expose the second output domain. In some embodiments, the exposed second output domain is configured to bind the first input domain. In some embodiments, binding of the exposed second output domain to the first input domain opens the first HCR hairpin to expose the first output domain. In some embodiments, the exposed first output domain is configured to bind the second input domain. In some embodiments, binding of the exposed first output domain to the second input domain leads to HCR polymerization in which second and first HCR hairpins are opened and add to the growing polymer in alternating fashion.

In some embodiments, the first and second HCR hairpins are kinetically trapped so that they do not polymerize except in the presence of the first or second HCR initiators.

In some embodiments, an HCR lateral flow device comprises a test region, a signal probe set comprising one or more HCR initiators, and a first HCR hairpin (h1) comprising a first input domain comprising a first toehold and a first stem section, and a first output domain comprising a first loop and a partner to the first stem section that is configured to bind the first stem section. In some embodiments, the HCR initiator is configured to bind the first input domain. In some embodiments, binding of the HCR initiator to the first input domain opens the first HCR hairpin to expose the first output domain. In some embodiments, the first HCR hairpin is kinetically trapped so that it does not open except in the presence of the HCR initiator. In some embodiments, the exposed first output domain directly or indirectly mediates generation of a signal in the test region.

In some embodiments, the device further comprises a second HCR hairpin (h2) comprising a second input domain comprising a second toehold and a second stem section, and a second output domain comprising a second loop and a partner to the second stem section that is configured to bind the second stem section. In some embodiments, the exposed first output domain is configured to bind the second input domain. In some embodiments, binding of the exposed first output domain to the second input domain opens the second HCR hairpin to expose the second output domain. In some embodiments, the exposed second output domain is configured to bind the first input domain. In some embodiments, binding of the exposed second output domain to the first input domain leads to HCR polymerization in which first and second HCR hairpins are successively opened and added to the growing polymer in alternating fashion. In some embodiments, the first and second HCR hairpins are kinetically trapped so that they do not polymerize except in the presence of the HCR initiator. In some embodiments, the HCR amplification polymer directly or indirectly mediates generation of an amplified signal in the test region.

Embodiments provided herein are further described in the following enumerated aspects.
1. A hybridization chain reaction (HCR) lateral flow device for amplified detection of a target in a sample, wherein the device comprises one or more membranes, a test region on the one or more membranes, one or more probe sets each comprising one or more probes, at least one probe comprising an amplification domain that is configured to trigger a chain reaction in which HCR hairpins self-assemble to form a tethered HCR amplification polymer, and an HCR amplifier comprising two or more HCR hairpins wherein one or more of the HCR hairpins is labeled with one or more reporters, wherein the device is configured such that the sample and one or more reagents flow through the one or more membranes across the test region, and wherein the one or more probe sets and the HCR amplifier are configured to produce tethered reporter-decorated HCR amplification polymers to directly or indirectly generate an amplified signal in the test region.
2. The device of aspect 1, wherein the signal is generated if the target is present in the sample.
3. The device of any one of aspects 1 or 2, wherein the one or more probe sets comprise a capture probe set comprising one or more capture probes, wherein a capture probe comprises a target-binding domain configured to bind directly or indirectly to the target and an immobilization domain configured to bind directly or indirectly to the test region.
4. The device of aspect 1, wherein the signal is generated if the target is not present in the sample.
5. The device of any one of aspects 1 or 4 wherein the one or more probe sets comprise a target-mimic probe set comprising one or more target-mimic probes each comprising a competitive domain configured to compete with the target for binding partners, and an immobilization domain configured to bind directly or indirectly to the test region.
6. The device of any one of the preceding aspects, wherein one or more of the probes comprising the probe set are immobilized on at least one of the one or more membranes.
7. The device of any one of aspects 1 or 2, wherein the one or more probe sets comprise a signal probe set comprising one or more signal probes, wherein a signal probe comprises a target-binding domain configured to bind directly or indirectly to the target and an amplification domain that is configured to directly or indirectly trigger a chain reaction in which HCR hairpins self-assemble to form a tethered HCR amplification polymer decorated with reporters.
8. The device of any one of aspects 1 or 4, wherein the one or more probe sets comprise a signal probe set comprising one or more signal probes, wherein a signal probe comprises a target-binding domain configured to bind directly or indirectly to the target or to the competitive domain of a target-mimic probe, and further comprises an amplification domain that is configured to directly or indirectly trigger a chain reaction in which HCR hairpins self-assemble to form a tethered HCR amplification polymer decorated with reporters.
9. The device any one of aspects 7 or 8, wherein the signal probe set comprises one or more initiator-labeled probes, wherein an initiator-labeled probe comprises a target-binding domain and an amplification domain comprising one or more HCR initiators.
10. The device of any one of aspects 7 or 8, wherein the signal probe set comprises two or more fractional-initiator probes.
11. The device of any one of the preceding aspects, wherein the one or more probe sets comprise one or more readout probes, wherein a readout probe comprises a reporter-binding domain configured to bind directly or indirectly to a reporter on an HCR amplification polymer, and further comprises one or more auxiliary reporters.
12. The device of aspect 11, wherein the readout probe comprising one or more auxiliary reporters binds the reporters decorating the tethered HCR amplification polymers that are immobilized in the test region, directly or indirectly generating an amplified signal in the test region.
13. The device of any one of aspect 1 or 11, wherein the reporters or auxiliary reporters decorating an HCR amplification polymer directly or indirectly mediate catalytic reporter deposition (CARD) to generate an amplified signal in the vicinity of the HCR amplification polymer within the test region.
14. The device of any one of the preceding aspects, wherein the amplified signal in the test region is visible to the human eye.
15. The device of any one of the preceding aspects, comprising a control region on the one or more membranes, wherein the control region is downstream of the test region so that the sample and reagents flow through the test region before reaching the control region.
16. The device of any one of the preceding aspects, further comprising a control capture probe set, a control signal probe set, and a control HCR amplifier comprising one or more control reporters.
17. The device of any one of the preceding aspects, further comprising a control readout probe set comprising one or more control auxiliary reporters.
18. The device of aspect 17, further comprising a readout probe set, wherein the control readout probe set is the same as the readout probe set, wherein the control readout probe set and readout probe set both further comprise one or more auxiliary reporters wherein the auxiliary reporters contribute directly or indirectly to the generation of an amplified signal.
19. The device of aspect 16, wherein the control HCR amplifier is the same as the HCR amplifier, wherein the control HCR amplifier and HCR amplifier both are configured to produce tethered reporter-decorated HCR amplification polymers to directly or indirectly generate an amplified signal.
20. The device of any one of the preceding aspects, further comprising a signal probe set, wherein the control signal probe set is the same as the signal probe set, wherein control signal probe set and the signal probe set both further comprise an amplification domain that is configured to directly or indirectly trigger a chain reaction in which HCR hairpins self-assemble to form a tethered HCR amplification polymer decorated with reporters.
21. The device of aspect 15, wherein absence of signal in the control region is an indication that the test did not function properly.
22. The device of any one of the preceding aspects, wherein a probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above.
23. The device of any one of the preceding aspects, wherein the one or more reporters comprise a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, digoxigenin (DIG), fluorescein isothiocyanate (FITC), biotin, dinitrophenol, aniline, an enzyme, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, any combination thereof, or any other molecule that directly or indirectly facilitates measurement of a signal.
24. The device of any one of the preceding aspects, wherein the target comprises a protein, a peptide, an amino acid, a nucleic acid, a DNA, an RNA, a chemically modified nucleic acid, a synthetic nucleic acid analog, a material capable of base-pairing, a chemical linker, a molecule, a biomolecule, a chemical, a toxin, a pathogen, a disease marker, a pollutant, a complex of any of the above, a diseased cell, a cancer cell, a human cell, a eukaryotic cell, a prokaryote, a bacterium, a virus, or any combination of the above.
25. The device of any one of the preceding aspects, wherein the target is viral or bacterial in origin.
26. The device of any one of the preceding aspects, further comprising at least one of a sample pad, conjugate pad, or wicking pad.
27. The device of any one of the preceding aspects, wherein at least one of the membranes, sample pads, conjugate pads, or wicking pads comprises nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.
28. The device of any one of the preceding aspects, wherein reagents automatically arrive in the test region in two or more successive stages.
29. The device of any one of the preceding aspects, wherein the device comprises a membrane with two or more channels configured to automatically provide reagents to the test region.
30. The device of aspect 29, wherein the two or more channels have different lengths that lead into a unified channel before reaching the test region.
31. The device of aspect 29, wherein the device comprises a separate conjugate pad for each of the two or more channels, each conjugate pad comprising dried down reagents, wherein each conjugate pad is fluidly connected to a single channel.
32. The device of aspect 5, wherein reagents automatically arrive in the control region in two or more successive stages.
33. The device of aspect 5, wherein the device comprises a membrane with two or more channels configured to automatically provide reagents to the control region.
34. The device of aspect 33, wherein the two or more channels have different lengths that lead into a unified channel before reaching the control region.
35. The device of aspect 33, wherein the device comprises a separate conjugate pad for each of the two or more channels, each conjugate pad comprising dried down reagents.
36. The device of any one of the preceding aspects, wherein the device takes the form of a folding card comprising a sample pad, one or more conjugate pads, a membrane comprising two or more channels, and a wicking pad.
37. The device of any one of the preceding aspects, wherein one of the one or more probe sets comprises a signal probe set comprising one or more signal probes, and wherein the signal probe set and the HCR amplifier are configured to produce a tethered reporter-decorated HCR amplification polymer to directly or indirectly generate an amplified signal in the test region if the signal probe set is immobilized in the test region.
38. The device of any one of the preceding aspects, for use in amplified detection of a N targets in a sample, where N is an integer greater than or equal to two, wherein the device comprises one or more membranes, N test regions, N capture probe sets, N signal probe sets, and N HCR amplifiers each labeled with one or more reporters, wherein the capture probe sets, signal probe sets, and HCR amplifiers are configured to produce a tethered reporter-decorated HCR amplification polymer to directly or indirectly generate an amplified signal in a given test region.
39. The device of aspect 38, wherein the signal is generated if the target is present in the sample.
40. The device of aspect 38 further comprising N readout probe sets each labeled with one or more auxiliary reporters, wherein the auxiliary-reporter-labeled readout probe sets are configured to bind the reporters to directly or indirectly generate an amplified signal in a given test region if the corresponding target is present in the sample.
41. The device of any one of the preceding aspects, for use in amplified detection of N targets in a sample, where N is an integer greater than or equal to two, wherein the device comprises one or more membranes, N test regions, N target-mimic probe sets, N signal probe sets, and N HCR amplifiers each labeled with one or more reporters, wherein the target-mimic probe sets, signal probe sets, and HCR amplifiers are configured to produce a tethered reporter-decorated HCR amplification polymer to directly or indirectly generate an amplified signal in a given test region.
42. The device of aspect 41, wherein the signal is generated if the target is not present in the sample.
43. The device of aspect 41, further comprising N readout probe sets each labeled with one or more auxiliary reporters, wherein the auxiliary-reporter-labeled readout probe sets are configured to bind the reporters to directly or indirectly generate an amplified signal in a given test region if the corresponding target is not present in the sample.
44. The device of any one of aspects 40 or 43, wherein the N readout probe sets are the same for all targets.
45. The device of any one of aspects 38 or 41, wherein the N HCR amplifiers are the same for all targets.
46. A method for amplified detection of a target in a sample, wherein a user: 1) adds the sample to a hybridization chain reaction (HCR) lateral flow device comprising one or more membranes, a test region on the one or more membranes, one or more probe sets each comprising one or more probes, at least one probe comprising an amplification domain that is configured to trigger a chain reaction in which HCR hairpins self-assemble to form a tethered HCR amplification polymer, an HCR amplifier comprising two or more HCR hairpins wherein one or more of the HCR hairpins is labeled with one or more reporters, 2) waits for a prescribed time while the sample and reagents flow through the one or more membranes across the test region to produce a tethered reporter-decorated HCR amplification polymer that produces an amplified signal in the test region, 3) and reads out the rest result by examining the test region for a signal.
47. The method of any one of the preceding method aspects, further comprising prepping the sample by mixing the sample with a prep buffer prior to adding the sample to the HCR lateral flow device.
48. The method of any one of the preceding method aspects, wherein the signal is generated if the target is present in the sample.
49. The method of any one of the preceding method aspects, wherein the device comprises a capture probe set comprising one or more capture probes, and a signal probe set comprising one or more signal probes, wherein if a target is present in the sample, the target binds one or more capture probes and one or more signal probes to form a probe/target complex, wherein the one or more capture probes immobilize the probe/target complex in the test region, and wherein amplification domains on the one or more signal probes trigger the self-assembly of reporter-decorated HCR hairpins into tethered reporter-decorated HCR amplification polymers to thereby directly or indirectly generate an amplified signal in the test region.
50. The method of aspect 46, wherein the signal is generated if the target is not present in the sample.
51. The method of aspect 46, wherein the device comprises a target-mimic probe set comprising one or more target-mimic probes, and a signal probe set comprising one or more signal probes, wherein if a target is not present in the sample, the one or more target-mimic probes bind one or more signal probes to form a competitive complex, wherein the one or more target-mimic probes immobilize the competitive complex in the test region, and wherein the amplification domains on the one or more signal probes trigger the self-assembly of reporter-labeled HCR hairpins into tethered HCR amplification polymers decorated with reporters to directly or indirectly generate an amplified signal in the test region.
52. The method of any one of the preceding method aspects, wherein the device additionally comprises a control region on the one or more membranes, a control capture probe set, a control signal probe set, a control HCR amplifier comprising one or more control reporters, and optionally a control readout probe set comprising one or more control auxiliary reporters.
53. The method of any one of the preceding method aspects, wherein if the sample and reagents successfully flow through a control region, a control capture probe set binds to and immobilizes a control signal probe set in the test region, the control signal probe set triggers the self-assembly of tethered control HCR amplification polymers decorated with control reporters to directly or indirectly generate an amplified control signal in the control region, and optionally a control-auxiliary-reporter-labeled control readout probe set binds the control reporters to directly or indirectly generate an amplified control signal in the control region.
54. The method of any one of the preceding method aspects, wherein the sample comprises a bodily fluid, milk, water, urine, blood, serum, saliva, cells, tissue, tissue homogenate, a medical sample, an environmental sample, wash water from an agricultural crop, gray water, brown water, sea water, pollutants, polluted water, a waste sample, sewage, plants, bacteria, sludge, homogenized plants, organic matter, animal feed, an industrial sample, liquid fuel, solid fuel, propellants, a chemical solution, and/or chemicals.
55. The method of any one of the preceding method aspects, wherein the sample is obtained by swabbing, nasal swabbing, nasopharyngeal swabbing, anterior nasal swabbing, throat swabbing, or swabbing of a material, mixture, solution, or substance to be analyzed.
56. The method of any one of the preceding method aspects, wherein the sample is mixed with prep buffer before or after adding the sample to the device.
57. The method of any one of the preceding method aspects, wherein the device comprises one or more conjugate pads comprising dried down reagents.
58. The method of aspect 57, wherein the reagents dried down in each conjugate pad are resuspended when the user adds the sample, prep buffer, or mixture of sample and prep buffer to the device.
59. The method of any one of the preceding method aspects, wherein the device takes the form of a folding card, wherein closing the folding card starts the test.
60. The method of any one of the preceding method aspects, wherein one or more of the probes comprising the one or more probe sets are immobilized on at least one of the one or more membranes.
61. The method of aspect 46, wherein the device comprises a capture probe set comprising one or more capture probes, wherein if a target is present in the sample, a target-binding domain of the capture probe binds directly or indirectly to the target and an immobilization domain of the capture probe binds directly or indirectly to the test region.
62. The method of aspect 46, wherein the device comprises a target-mimic probe set comprising one or more target-mimic probes, wherein if a target is present in the sample, a competitive domain of the target-mimic probe competes with the target for binding partners, and an immobilization domain of the target-mimic probe binds directly or indirectly to the test region.
63. The method of aspect 46, wherein the device comprises a signal probe set comprising one or more signal probes, wherein if a target is present in the sample, a target-binding domain of one or more signal probes binds directly or indirectly to the target and one or more amplification domains of the one or more signal probes directly or indirectly triggers a chain reaction in which HCR hairpins self-assemble to form a tethered HCR amplification polymer decorated with reporters.
64. The method of aspect 46, wherein the device comprises a signal probe set comprising one or more signal probes, wherein if a target is not present in the sample, a target-binding domain of one or more signal probes binds directly or indirectly to the competitive domain of a target-mimic probe, and one or more amplification domains of the one or more signal probes directly or indirectly triggers a chain reaction in which HCR hairpins self-assemble to form a tethered HCR amplification polymer decorated with reporters.
65. The method of any one of aspects 63 or 54, wherein the signal probe set of the device comprises: a) one or more initiator-labeled probes wherein an initiator-labeled probe comprises a target-binding domain and an amplification domain comprising one or more HCR initiators, or b) one or more probe units, each comprising two or more fractional-initiator probes, wherein a fractional-initiator probe comprises a target-binding domain and an amplification domain comprising a fractional initiator.
66. The method of aspect 48, wherein the device comprises a readout probe set comprising one or more readout probes, wherein if a target is present in the sample, a reporter-binding domain of the readout probe binds directly or indirectly to a reporter on an HCR amplification polymer, and further comprises one or more auxiliary reporters.
67. The method of aspect 50, wherein the device comprises a readout probe set comprising one or more readout probes, wherein if a target is not present in the sample, a reporter-binding domain of the readout probe binds directly or indirectly to a reporter on an HCR amplification polymer, and further comprises one or more auxiliary reporters.
68. The method of any one of aspects 66 or 67, wherein the one or more readout probes of the device comprise one or more auxiliary reporters and bind the reporters decorating the tethered HCR amplification polymers that are immobilized in the test region, directly or indirectly generating an amplified signal in the test region.
69. The method of aspect 68, wherein the reporters or auxiliary reporters decorating an HCR amplification polymer directly or indirectly mediate catalytic reporter deposition (CARD) to generate an amplified signal in the vicinity of the HCR amplification polymer within the test region.
70. The method of any one of the preceding method aspects, wherein the device further comprises a control readout probe set comprising one or more control auxiliary reporters.
71. The method of aspect 70, wherein the device further comprises a readout probe set, wherein the control readout probe set is the same as the readout probe set, wherein the control readout probe set and readout probe set both further comprise one or more auxiliary reporters wherein the auxiliary reporters contribute directly or indirectly to the generation of an amplified signal.
72. The method of aspect 52, wherein the control HCR amplifier is the same as the HCR amplifier, wherein the control HCR amplifier and HCR amplifier both are configured to produce tethered reporter-decorated HCR amplification polymers to directly or indirectly generate an amplified signal.
73. The method of aspect 52, wherein the device further comprises a signal probe set, wherein the control signal probe set is the same as the signal probe set, wherein the control signal probe set and the signal probe set both further comprise an amplification domain that is configured to directly or indirectly trigger a chain reaction in which HCR hairpins self-assemble to form a tethered HCR amplification polymer decorated with reporters.
74. The method of aspect 52, wherein absence of signal in the control region is an indication that the test did not function properly.
75. The method of any one of the preceding method aspects, wherein a probe comprises an antibody, a nanobody, a protein, a peptide, a nucleic acid, a synthetic nucleic acid analog, a chemically modified nucleic acid, a chemically modified protein, RNA, DNA, 2′OMe-RNA, PNA, XNA, any other material capable of base-pairing, a carbon atom, a chemical linker not capable of base-pairing, any combination thereof, or a complex of any of the above.
76. The method of any one of the preceding method aspects, wherein the one or more reporters comprise a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, digoxigenin (DIG), fluorescein isothiocyanate (FITC), biotin, dinitrophenol, aniline, an enzyme, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, any combination thereof, or any other molecule that directly or indirectly facilitates measurement of a signal.
77. The method of any one of the preceding method aspects, wherein the target comprises a protein, a peptide, an amino acid, a nucleic acid, a DNA, an RNA, a chemically modified nucleic acid, a synthetic nucleic acid analog, a material capable of base-pairing, a chemical linker, a molecule, a biomolecule, a chemical, a toxin, a pathogen, a disease marker, a pollutant, a complex of any of the above, a diseased cell, a cancer cell, a human cell, a eukaryotic cell, a prokaryote, a bacterium, a virus, or any combination of the above.
78. The method of any one of the preceding method aspects, wherein the target is viral or bacterial in origin.
79. The method of any one of the preceding method aspects, further comprising at least one of a sample pad, conjugate pad, or wicking pad.
80. The method of aspect 79, wherein at least one of the membranes, sample pads, conjugate pads, or wicking pads comprises nitrocellulose, cellulose fiber, cellulose acetate, polyvinylidene fluoride (PVDF), nylon, charge-modified nylon, polyethersulfone (PES), glass fiber, paper, cotton, polyester, polypropylene, sponge, microstructured polymer, sintered polymer, or another permeable substrate.
81. The method of any one of the preceding method aspects, wherein reagents automatically arrive in the test region in two or more successive stages.
82. The method of any one of the preceding method aspects, wherein the device comprises a membrane with two or more channels configured to automatically provide reagents to the test region.
83. The method of any one of the preceding method aspects, wherein the two or more channels have different lengths that lead into a unified channel before reaching the test region.
84. The method of aspect 52, wherein reagents automatically arrive in the control region in two or more successive stages.
85. The method of aspect 52, wherein the device comprises a membrane with two or more channels configured to automatically provide reagents to the control region.
86. The method of aspect 52, wherein the two or more channels have different lengths that lead into a unified channel before reaching the control region.
87. The method of any one of the preceding method aspects, wherein one of the one or more probe sets comprises a signal probe set comprising one or more signal probes, and wherein the signal probe set and the HCR amplifier are configured to produce a tethered reporter-decorated HCR amplification polymer to directly or indirectly generate an amplified signal in the test region if the signal probe set is immobilized in the test region.
88. The method of any one of the preceding method aspects, for use in amplified detection of a N targets in a sample, where N is an integer greater than or equal to two, wherein the device comprises one or more membranes, N test regions, N capture probe sets, N signal probe sets, and N HCR amplifiers each labeled with one or more reporters, wherein the capture probe sets, signal probe sets, and HCR amplifiers are configured to produce a tethered reporter-decorated HCR amplification polymer to directly or indirectly generate an amplified signal in a given test region.
89. The method of any one of the preceding method aspects, wherein the signal is generated if the target is present in the sample.
90. The method of aspect 88, further comprising N readout probe sets each labeled with one or more auxiliary reporters, wherein the auxiliary-reporter-labeled readout probe sets are configured to bind the reporters to directly or indirectly generate an amplified signal in a given test region if the corresponding target is present in the sample.
91. The method of any one of the preceding method aspects, for use in amplified detection of N targets in a sample, where N is an integer greater than or equal to two, wherein the device comprises one or more membranes, N test regions, N target-mimic probe sets, N signal probe sets, and N HCR amplifiers each labeled with one or more reporters, wherein the target-mimic probe sets, signal probe sets, and HCR amplifiers are configured to produce a tethered reporter-decorated HCR amplification polymer to directly or indirectly generate an amplified signal in a given test region.
92. The method of aspect 91, wherein the signal is generated if the target is not present in the sample.
93. The method of aspect 91 further comprising N readout probe sets each labeled with one or more auxiliary reporters, wherein the auxiliary-reporter-labeled readout probe sets are configured to bind the reporters to directly or indirectly generate an amplified signal in a given test region if the corresponding target is not present in the sample.
94. The method of any one of aspects 90 or 93, wherein the N readout probe sets are the same for all targets.
95. The method of any one of aspects 88 or 91, wherein the N HCR amplifiers are the same for all targets.

### EXAMPLES AND DESCRIPTION

### Example 1—Amplified HCR Lateral Flow Assay for Detection of SARS-CoV-2 Nucleocapsid Protein (N)

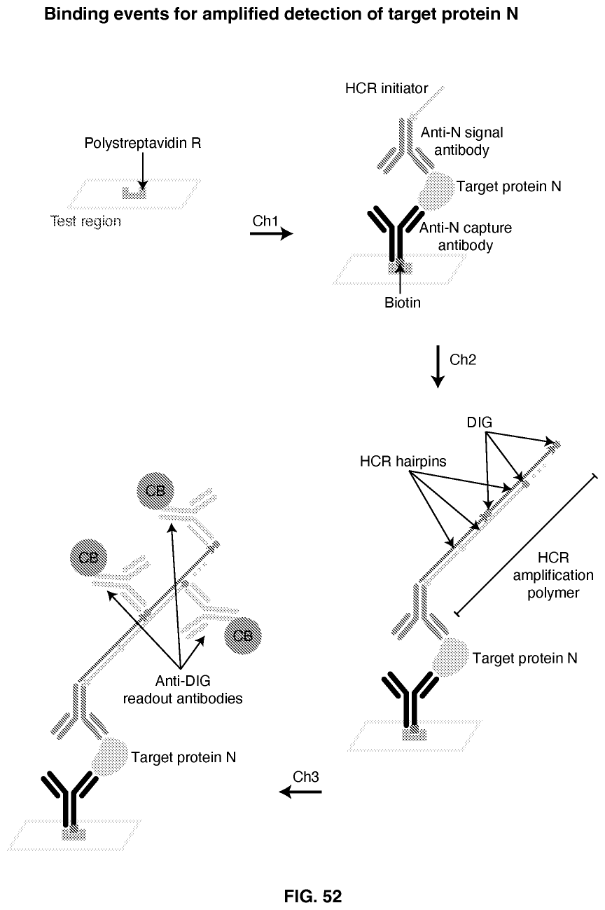

FIG. 52 displays an amplified HCR lateral flow assay for detection of SARS-CoV-2 nucleocapsid protein (N). The target protein N bound in a sandwich between an HCR initiator-labeled anti-N signal antibody that bound a first target epitope and an anti-N capture antibody that bound a second target epitope. The anti-N capture antibody was biotinylated and was itself captured in the test region by immobilized polystreptavidin R (PR). After the antibody/target sandwich was immobilized in the test region, the HCR initiators labeling the anti-N signal antibody triggered the self-assembly of DIG-labeled HCR hairpins into tethered HCR amplification polymers (digoxigenin; DIG). The DIG-decorated HCR amplification polymers immobilized in the test region were then bound by CB-labeled anti-DIG readout antibodies (carbon black; CB).

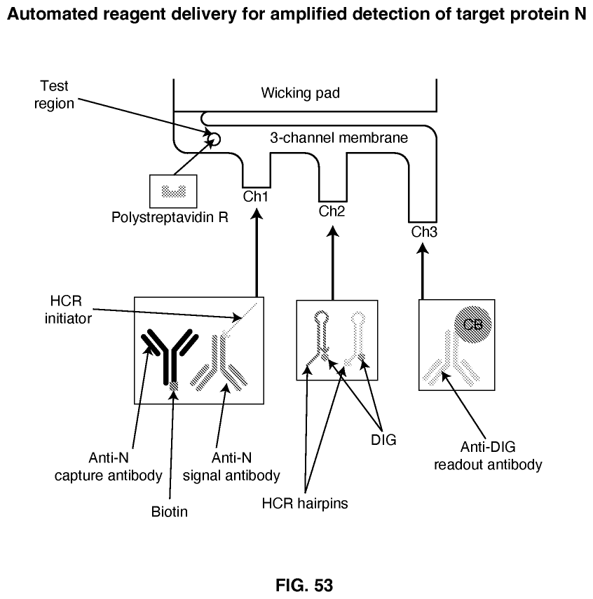

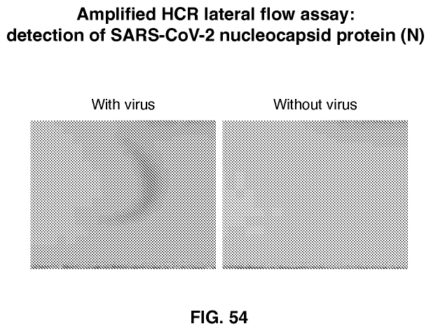

Reagents were automatically delivered to the test region in three successive stages using a three-channel nitrocellulose membrane in which channels of different lengths lead into a unified channel before reaching the test region (see FIG. 53). The anti-N signal and capture antibodies bound the target protein N and traveled via the shortest nitrocellulose channel (Channel 1) to reach the test region first, where the antibody/target sandwich was immobilized via binding of biotinylated anti-N capture antibodies to immobilized polystreptavidin R (PR). DIG-labeled HCR hairpins traveled via a channel of intermediate length (Channel 2) and reached the test region next, where initiators on the anti-N signal antibodies triggered growth of tethered DIG-labeled HCR amplification polymers. CB-labeled anti-DIG readout antibodies traveled via the longest channel (Channel 3) and arrived in the test region last, where they decorated the HCR amplification polymers to generate an amplified colored signal in the test region.

Reagents for each channel were dried onto separate conjugate pads, which were rehydrated simultaneously when the user added the sample to the sample pad. Upon rehydration, successive delivery of the reagents to the test region occurred automatically without user interaction, as draining of the first conjugate pad freed the unified channel for draining of the second conjugate pad, which in turn freed the unified channel for draining of the third conjugate pad.

A lateral flow device can take the form of a folding card (see FIG. 5). The right page of the card contained the sample pad and three conjugate pads. The left page of the card contained the 3-channel nitrocellulose membrane and the wicking pad (which absorbed liquid to induce continued capillary flow through the channels). The left page was functionalized with three prongs which disconnected the sample pad from the conjugate pads upon folding the card, limiting the volume that flowed from each conjugate pad and preventing flow between conjugate pads.

To perform a test, the user added saliva to a tube containing prep buffer (comprising extraction buffer that disrupted the viral envelope of SARS-CoV-2 to expose the target proteins N), added the prepped sample to the sample pad, and closed the card to create contact between the nitrocellulose membrane and the three conjugate pads, initiating the consecutive flow of liquid from Channels 1, 2, and 3. After 60 minutes, the user read either a positive result (black signal) or a negative result (no signal) in the test region with the naked eye (see FIG. 54). Due to automated multi-channel reagent delivery, this amplified HCR lateral flow assay retained the simplicity of conventional commercial unamplified lateral flow assays, requiring only sample addition and card closure before reading the result, with signal amplification occurring unbeknownst to the user.

FIG. 52 displays an amplified HCR lateral flow assay for detection of SARS-CoV-2 nucleocapsid protein (N). Each target protein N was immobilized by PR in the test region in a sandwich between a biotinylated anti-N capture antibody and an anti-N signal antibody labeled with one or more HCR initiators, triggering self-assembly of DIG-labeled HCR hairpins to form a tethered HCR amplification polymer that was subsequently bound by multiple CB-labeled anti-DIG readout antibodies, generating an amplified signal in the test region. (PR: polystreptavidin; R. DIG: digoxigenin). FIG. 53 displays automated delivery of reagents to the test region from Channels 1, 2, and 3 in succession using a 3-channel nitrocellulose membrane. FIG. 5 displays a prototype folding card device. The left page of the device contained the 3-channel nitrocellulose membrane and wicking pad. The right page of the device contained 3 conjugate pads (containing dried reagents for Channels 1, 2, and 3) and a sample pad. To perform the test, the user added the sample to the sample pad, closed the device, and read the result after 60 minutes. FIG. 54 displays results for SARS-CoV-2 tests with or without gamma-irradiated virus spiked into a mixture of human saliva and extraction buffer at 1000 copies/μL.

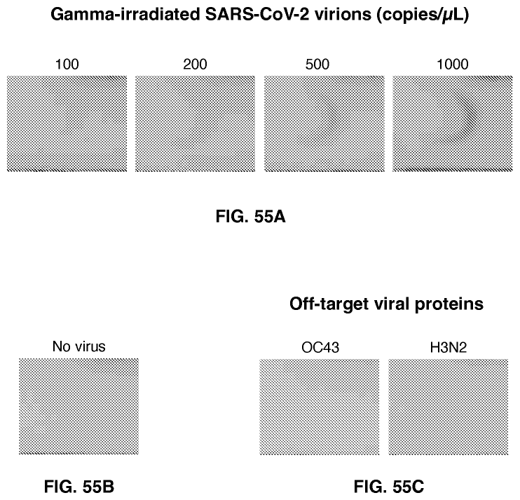

Using the human eye for readout, amplified HCR lateral flow assays run on gamma-irradiated SARS-CoV-2 virus spiked into a mixture of human saliva and extraction buffer at a range of concentrations revealed a limit of detection of 200 virus copies/μL (see FIG. 55A). No background staining was observed in the test region for experiments run without spiked-in virus (see FIG. 55B). For cross-reactivity experiments run with spiked-in recombinant N protein from a different betacoronavirus (OC43) or with spiked-in nucleoprotein from Influenza Type A (H3N2), no staining was observed in the test region even with off-target proteins at high concentration (equivalent to virions at ˜10⁶ copies/μL^{20,21}) (see FIG. 55C).

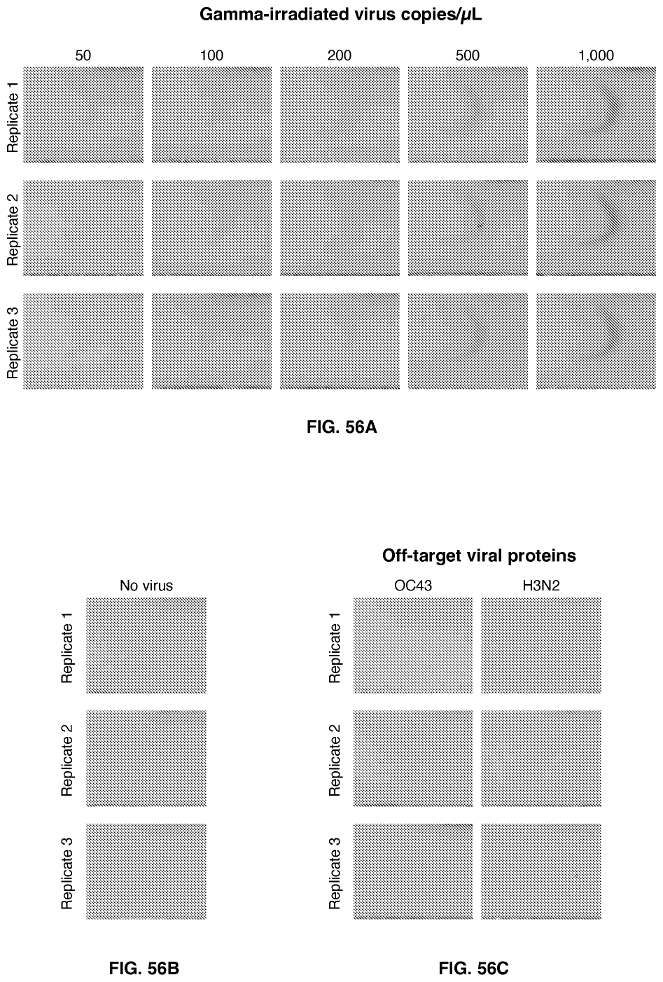

FIG. 56A displays replicate sensitivity studies for detection of SARS-CoV-2 nucleocapsid protein (N). Gamma-irradiated SARS-CoV-2 was spiked into a mixture of saliva and extraction buffer at the target concentration and loaded onto the sample pad before closing the folding card device to start the test. The test region was photographed after 60 minutes. N=3 replicate assays at each target concentration. Signal was visible in the test region in all three replicates down to a limit of detection of 200 copies/μL.

FIGS. 56B-56C display replicate background and cross-reactivity studies for detection of SARS-CoV-2 nucleocapsid protein (N). No virus (to measure background) or off-target recombinant viral OC43 N protein (83.74 ng/mL) or Influenza A (H3N2) (50.43 ng/mL) nucleoprotein (to test cross-reactivity) were spiked into a mixture of human saliva and extraction buffer and loaded onto the sample pad before closing the folding card device to start the test. For the cross-reactivity tests, the off-target viral proteins were spiked in at high concentrations equivalent to ˜10⁶ virions/μL.^{26,27} The test region was photographed after 60 minutes. N=3 replicate assays at each target concentration. For each target type, no staining was visible in the test region for all three replicates, indicating that there was no visible background when SARS-CoV-2 was absent, and no visible cross-reactivity with off-target OC43 or H3N2 proteins.

The disposable folding card device shown in FIG. 5 was printed with a 3D printer. The nitrocellulose membrane and wicking pad were overlapped on an adherent backing material and cut into a 3-channel geometry with a laser cutter. The sample pad was cut with a laser cutter, blocked, dried, and adhered to the right page of the card device. The conjugate pads were cut with a laser cutter, blocked, loaded with reagents (Channel 1: anti-N signal and capture antibodies; Channel 2: DIG-labeled HCR hairpins h1 and h2; Channel 3: CB-labeled anti-DIG readout antibody), dried, and adhered to the right page of the device. The nitrocellulose membrane was spotted with polystreptavidin R (PR) in the test region, dried, and adhered to the left page of the device. To run the assay, gamma-irradiated SARS-CoV-2 virus (or off-target viral protein) was spiked into a mixture of human saliva and extraction buffer to create the test sample, and then spotted on the sample pad before closing the folding card device to start the test. After 60 minutes, the test region was photographed.

### Example 2—a Folding Card Device for Performing Amplified HCR Lateral Flow Assays

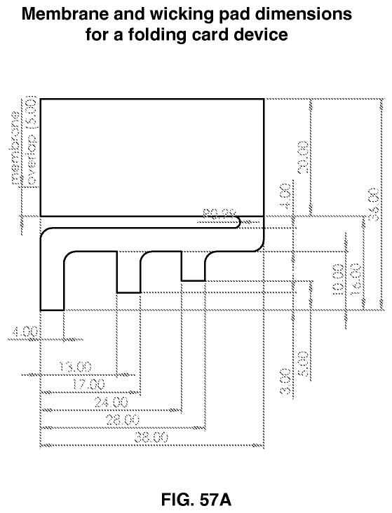

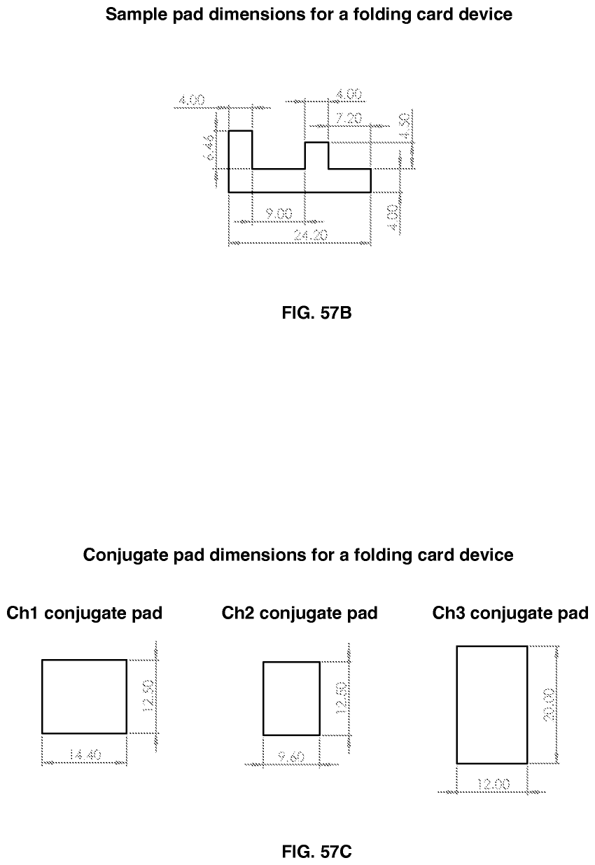

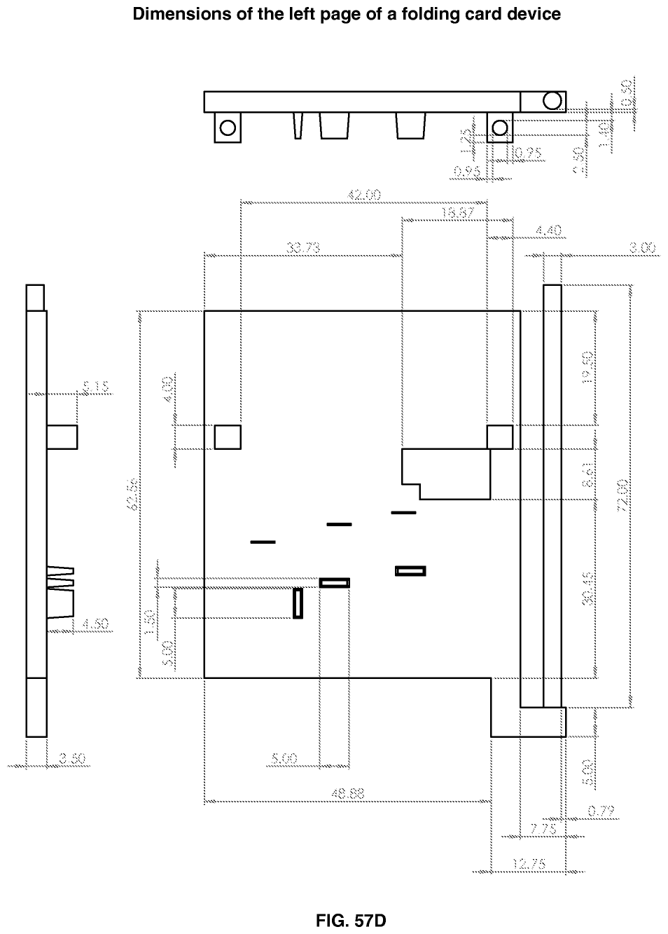

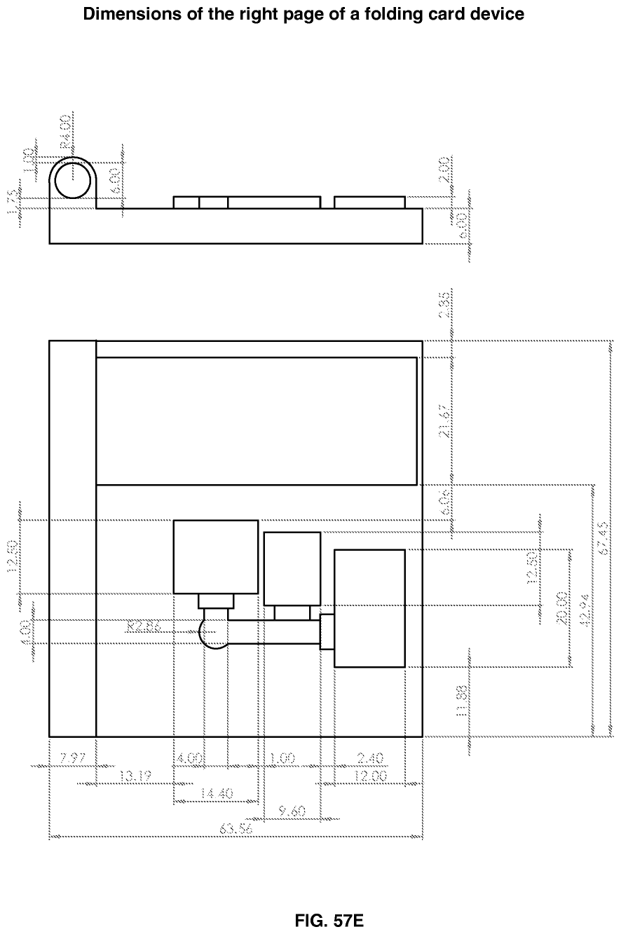

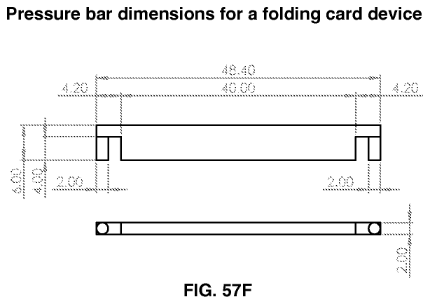

FIG. 57A displays dimensions for a nitrocellulose membrane and wicking pad. FIG. 57B displays sample pad dimensions and FIG. 57C displays conjugate pad dimensions. FIG. 57D displays dimensions for the left page of a prototype folding card device. FIG. 57E displays dimensions for the right page of a folding card device. FIG. 57F displays dimensions for a pressure bar that creates pressure between the wicking pad and the nitrocellulose membrane. All dimensions are shown in millimeters (R: radius). All curved lines have a radius of 2.00 mm unless otherwise indicated.

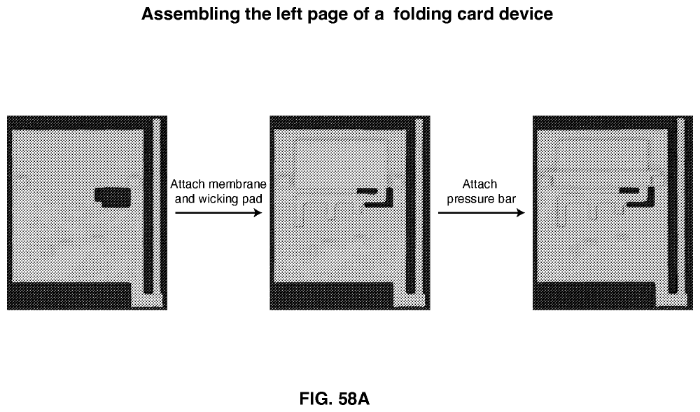

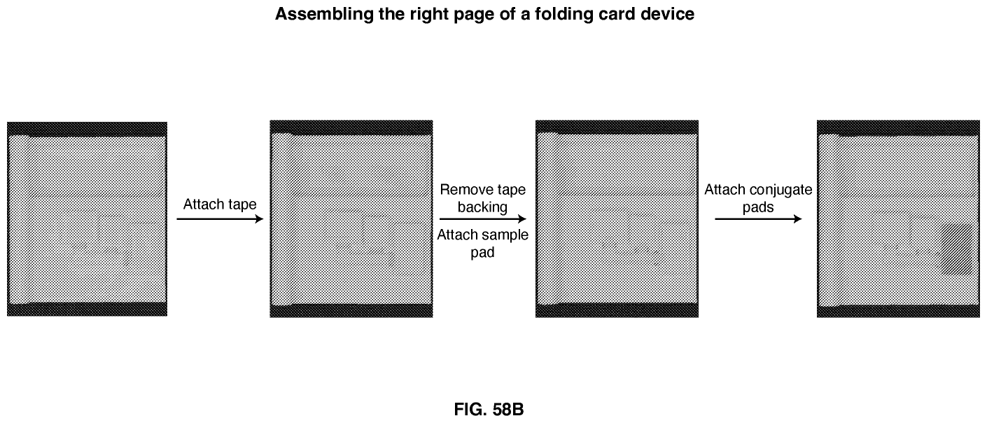

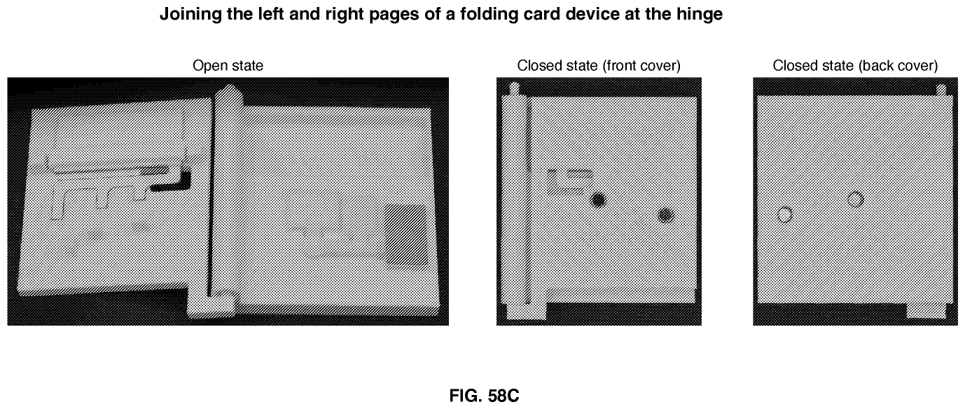

FIGS. 58A-58C demonstrate the steps for assembling the folding card device. FIG. 58A displays assembly of the left page of the folding card device. FIG. 58B displays assembly of the right page of the device. FIG. 58C displays joining of the left and right pages of the folding card device at the hinge. Images depict the device in the open state (left) and in the closed state viewing either the front cover (middle) or back cover (right). Each replicate SARS-CoV-2 test for FIGS. 54, 55A-55C, and 56A-56C was performed using a different disposable copy of this prototype folding card device.

### Example 3—Sensitivity of Five Commercial Unamplified Lateral Flow Assays for Detection of SARS-CoV-2 Nucleocapsid Protein (N)

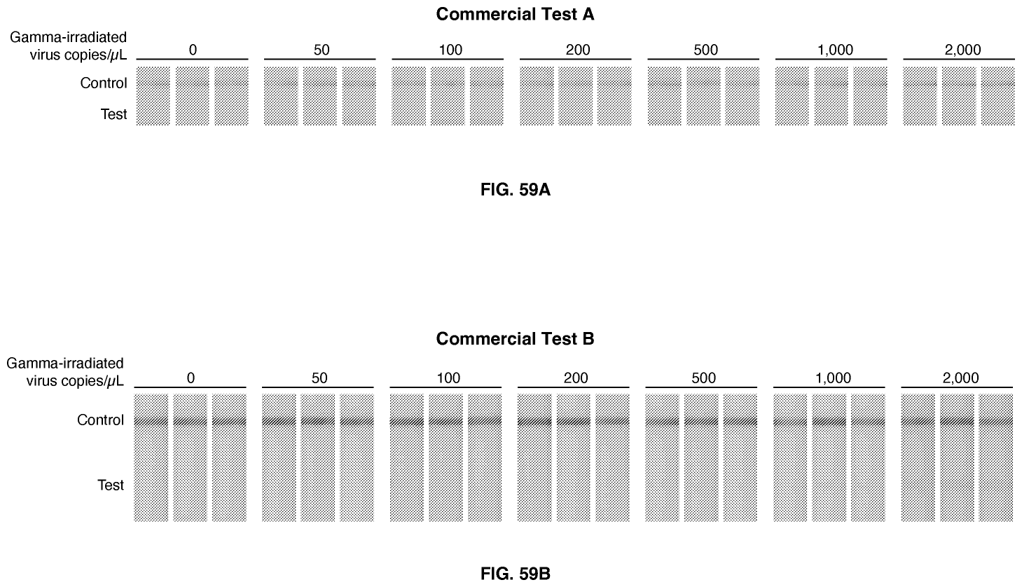

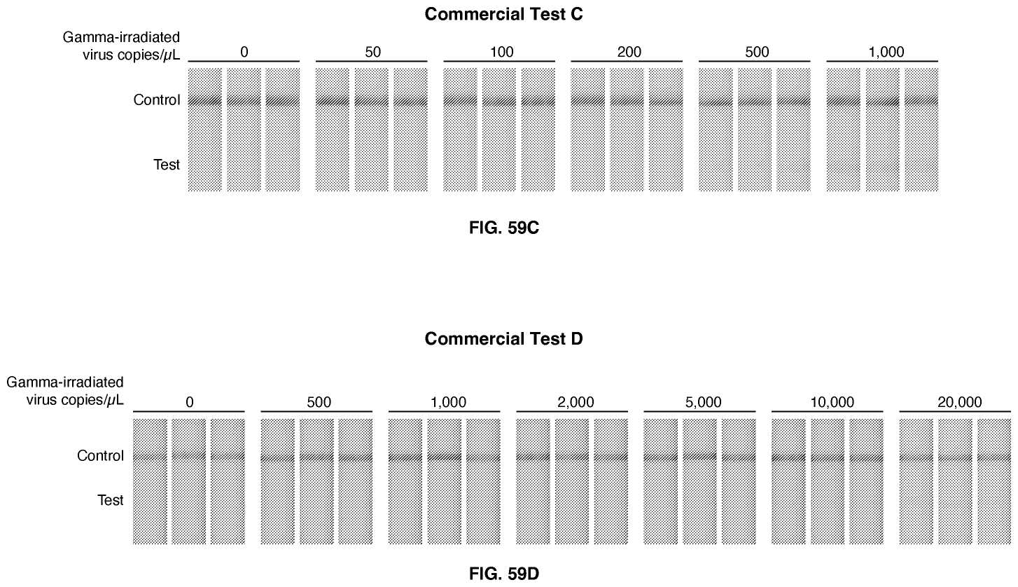

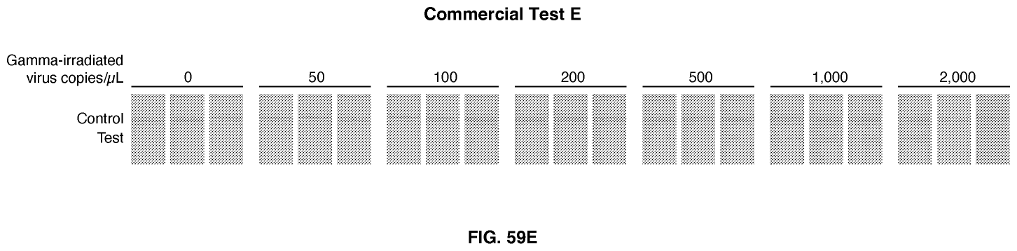

The sensitivity of five commercial SARS-CoV-2 lateral flow assays was benchmarked by spiking gamma-irradiated SARS-CoV-2 into the extraction buffer included with each test kit and loading the extracted sample according to the manufacturer's instructions. N=3 replicate tests were performed at each target concentration for each commercial kit. The test region was photographed at the minimum time recommended by the manufacturer. The limit of detection for a given kit was defined as the lowest tested concentration for which all three replicates had a visible signal. While the amplified HCR lateral flow assay of FIGS. 5, 52, and 53 detected gamma-irradiated SARS-CoV-2 at 200 copies/μL (see FIGS. 55A and 56A), none of the commercial unamplified tests detected SARS-CoV-2 at this concentration. The limits of detection for the five commercial kits were 2000 copies/μL (Commercial Test A photographed after 15 minutes; FIG. 59A), 2000 copies/μL (Commercial Test B photographed after 10 minutes; FIG. 59B), 500 copies/μL (Commercial Test C photographed after 15 minutes; FIG. 59C), 20,000 copies/μL (Commercial Test D photographed after 15 minutes; FIG. 59D), 1000 copies/μL (Commercial Test E photographed after 10 minutes; FIG. 59E).

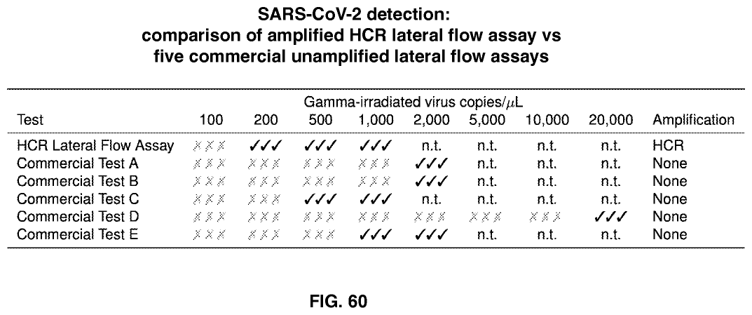

FIG. 60 compares the sensitivity of the amplified HCR lateral flow assay (FIGS. 55A and 56A) to the sensitivity of five commercial unamplified lateral flow assays (FIGS. 59A-59E). Gamma-irradiated virus was spiked into a mixture of human saliva and extraction buffer (amplified HCR lateral flow assay) or manufacturer-provided extraction buffer (commercial tests). N=3 replicates were performed for each concentration. The photograph of each replicate was judged by eye for a positive result (check mark) or negative result (x mark). Not tested (n.t.).

### Example 4—Amplified HCR Lateral Flow Assay for Detection of the SARS-CoV-2 RNA Genome

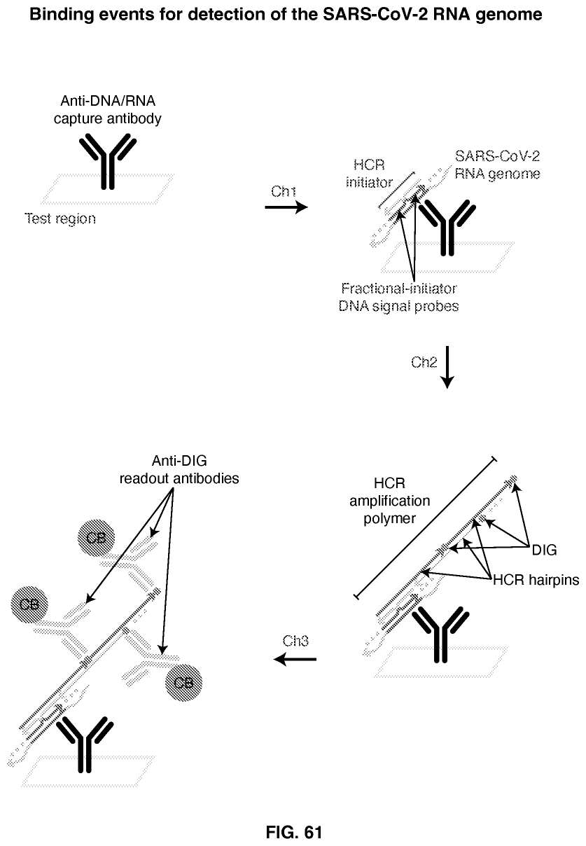

FIG. 61 displays an amplified HCR lateral flow assay for detection of the SARS-CoV-2 RNA genome. The target RNA was detected by a DNA signal probe set comprising 198 probe units, each comprising two fractional-initiator probes that bind to adjacent subsequences along the ˜30,000 nt target, avoiding subsequences shared by other coronaviruses (with the exception of SARS-CoV, which has high sequence similarity to SARS-CoV-2, causes severe disease, and is not circulating²⁸). Using fractional-initiator probes, any individual probe that bound non-specifically in the test region did not trigger HCR, but specific hybridization of the pair of probes within a probe unit to adjacent cognate binding sites along the target RNA colocalized a full HCR initiator capable of triggering HCR signal amplification.

By hybridizing to the RNA target, the DNA signal probes created a DNA:RNA duplex at each target binding site. The probe-decorated target was captured in the test region by an immobilized anti-DNA:RNA capture antibody that bound to DNA:RNA duplexes. While the capture antibody bound DNA:RNA duplexes independent of sequence, any captured RNA that did not include specifically bound fractional-initiator DNA probe pairs did not trigger HCR, and hence did not contribute to background. After immobilization of the probe-decorated target in the test region, colocalized full HCR initiators triggered the self-assembly of DIG-labeled HCR hairpins into tethered HCR amplification polymers decorated with DIG, which were in turn detected by CB-labeled anti-DIG readout antibodies (DIG: digoxigenin, CB: carbon black).

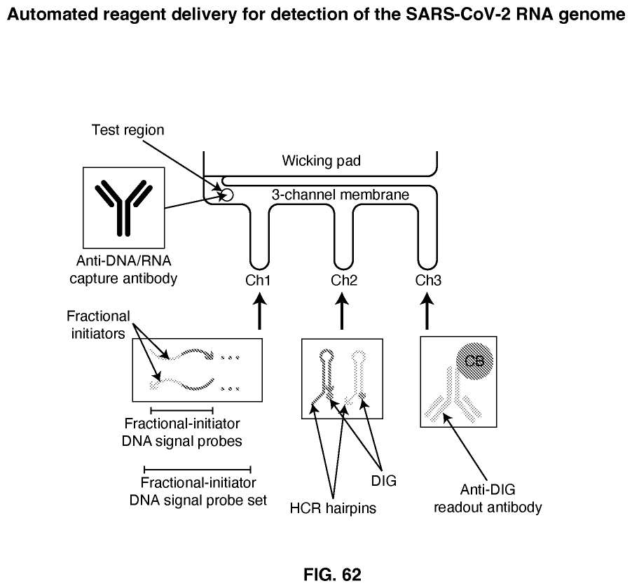

Reagents were automatically delivered to the test region in three successive stages using a 3-channel nitrocellulose membrane (FIG. 62). The DNA signal probe set bound the RNA target and traveled via the shortest nitrocellulose channel (Channel 1) to reach the test region first, where the probe-decorated target was captured by pre-immobilized anti-DNA:RNA capture antibodies. DIG-labeled HCR hairpins traveled via a channel of intermediate length (Channel 2) and reached the test region next, where colocalized full initiators formed by specifically bound fractional-initiator probe pairs triggered growth of tethered HCR amplification polymers decorated with DIG. CB-labeled anti-DIG reporter antibodies traveled via the longest channel (Channel 3) and arrived in the test region last, where they decorated the HCR amplification polymers with CB to generate an amplified colored signal in the test region (DIG: digoxigenin, CB: carbon black).

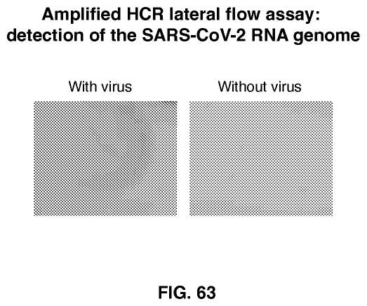

Heat denaturation removed the viral envelope and the nucleocapsid (N) proteins decorating the RNA genome, as well as disrupted native secondary structure in the single-stranded RNA genome prior to detection by the DNA probe set. This RNA test used a half-strip assay format with reagents in solution. To perform a test, the sample was added to extraction buffer containing the DNA signal probe set (Channel 1 reagents), heated to 65° C. for 15 minutes, and then loaded into a well (Channel 1 well) on a 96-well plate proximal to wells containing the DIG-labeled HCR hairpins (Channel 2 well) and CB-labeled anti-DIG reporter antibodies (Channel 3 well; see FIG. 6). To start the lateral flow assay, the ends of the three nitrocellulose channels were simultaneously submerged into the three wells, leading to automated successive delivery of the Channel 1, 2, and 3 reagents to the test region without user interaction. After 90 minutes, the result was read as either a positive result (black signal) or a negative result (no signal) in the test region with the naked eye (FIG. 63).

FIG. 61 displays an amplified HCR lateral flow assay for detection of the SARS-CoV-2 RNA genome. A signal probe set comprising multiple probe units each comprising a pair of fractional-initiator probes hybridized to cognate binding sites on the viral RNA genome to colocalize full HCR initiators; the resulting DNA/RNA duplexes were captured in the test region by anti-DNA/RNA capture antibodies; colocalized full HCR initiators triggered self-assembly of DIG-labeled HCR hairpins to form tethered HCR amplification polymers decorated with DIG, which were subsequently bound by CB-labeled anti-DIG reporter antibodies, generating an amplified signal in the test region. (DIG: digoxigenin; CB: carbon black). FIG. 62 displays automated delivery of reagents to the test region from Channels 1, 2, and 3 in succession using a 3-channel nitrocellulose membrane. FIG. 63 shows SARS-CoV-2 test results with or without gamma-irradiated virus spiked into extraction buffer at 1000 copies/μL.

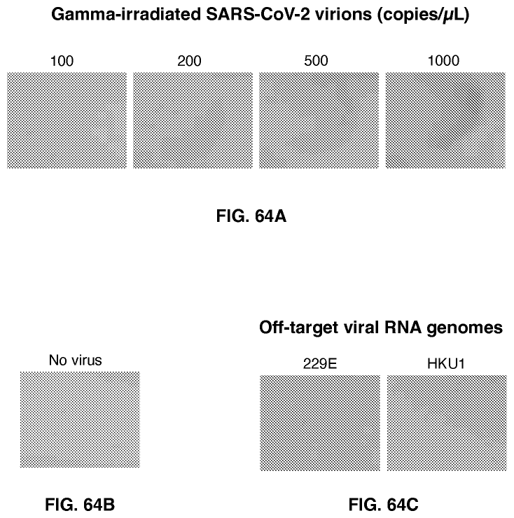

To characterize sensitivity, gamma-irradiated SARS-CoV-2 virus was spiked into extraction buffer with the DNA signal probe set, heated to 65° C. for 15 minutes, and run on the amplified HCR lateral flow assay, revealing a limit of detection of 200 virions/μL (see FIG. 64A). No background staining was observed in the test region for experiments run without spiked-in virus (see FIG. 64B). To characterize cross-reactivity, experiments were run with synthetic RNA genomes from other coronaviruses spiked-in at high concentration (7,200 copies/μL for 229E and 10,000 copies/μL for HKU1); no staining was observed in the test region (see FIG. 64C). This amplified HCR lateral flow assay for detecting the viral RNA genome has a lower limit of detection than all five commercial unamplified lateral flow assays evaluated in FIGS. 59A-59E.

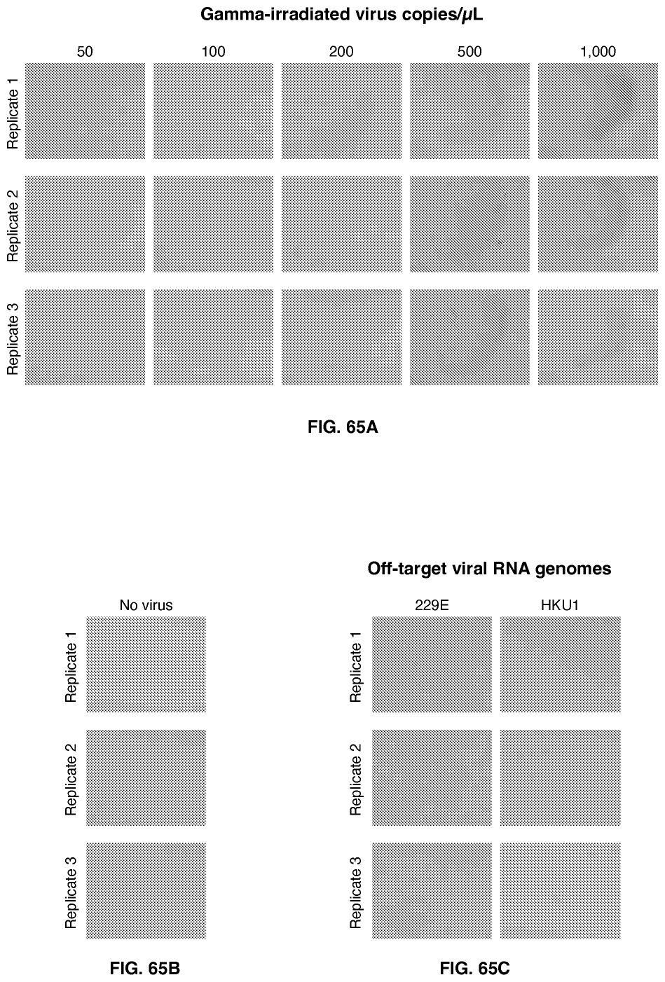

FIG. 65A displays replicate sensitivity studies for detection of the SARS-CoV-2 RNA genome. Gamma-irradiated SARS-CoV-2 was spiked into extraction buffer with the DNA signal probe set and heated at 65° C. for 15 minutes; this solution was added to the Channel 1 well in a 96-well plate and the ends of the three nitrocellulose channels (Channel 1, Channel 2, Channel 3) were simultaneously submerged in their respective wells in the 96-well plate to start the test. The test region was photographed after 90 minutes. N=3 replicate assays at each target concentration. The test region is visible in all three replicates down to a limit of detection of 200 copies/μL.

FIGS. 65B-65C display replicate background and cross-reactivity studies for detection of the SARS-CoV-2 RNA genome. No virus (to measure background) or off-target synthetic RNA genomes from coronaviruses 229E or HKU1 (to test cross-reactivity) were spiked into extraction buffer with the DNA signal probe set and heated at 65° C. for 15 minutes; this solution was added to the Channel 1 well in a 96-well plate and the ends of the three nitrocellulose channels (Channel 1, Channel 2, Channel 3) were simultaneously submerged in their respective wells in the 96-well plate to start the test. For the cross-reactivity tests, the off-target viral RNA genomes were spiked in at high concentration (7,200 copies/μL for 229E and 10,000 copies/μL for HKU1). The test region was photographed after 90 minutes. N=3 replicate assays at each target concentration. For each target type, no staining was visible in the test region for all three replicates, indicating that there is no visible background when SARS-CoV-2 is absent, and no visible cross-reactivity with off-target 229E or HKU1 coronavirus RNA genomes.

The nitrocellulose membrane and wicking pad were overlapped on an adherent backing material and cut into a 3-channel geometry with a laser cutter. The nitrocellulose membrane was spotted with anti-DNA/RNA capture antibody in the test region and dried. Gamma-irradiated SARS-CoV-2 virus (or off-target synthetic viral RNA) was mixed with extraction buffer and the DNA signal probe set, heated on a heat block at 65° C. for 15 min, and loaded into a well (Channel 1) proximal to wells containing reagents for Channel 2 (DIG-labeled HCR hairpins h1 and h2) and Channel 3 (CB-labeled anti-DIG reporter antibody). The ends of the three nitrocellulose channels were simultaneously immersed into the three wells to start the test. After 90 minutes, the test region was photographed.

### Example 5—A Device for Performing Amplified HCR Lateral Flow Assays

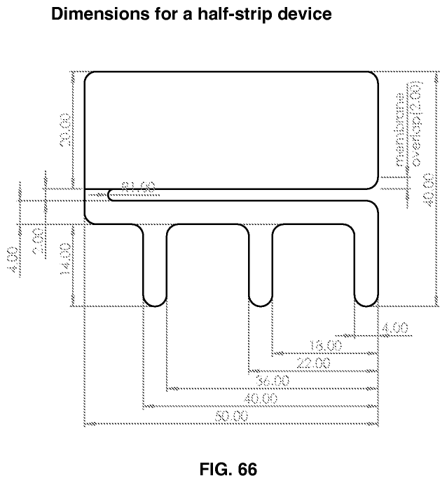

FIG. 66 displays dimensions for a nitrocellulose membrane and wicking pad. All dimensions are shown in millimeters (R: radius). All curved lines have a radius of 2.00 mm unless otherwise indicated. FIG. 6 displays a photograph of a lateral flow device with three channels resting within their respective wells on a 96-well plate. Each replicate SARS-CoV-2 test for FIGS. 64A-64C and 65A-65C was performed using a different disposable copy of this device.

### Example 6—HCR Polymerization at Short Time Scales

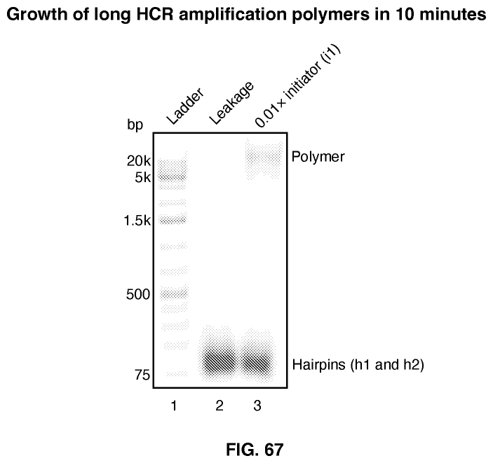

The gel study of FIG. 67 demonstrates that an HCR initiator can trigger self-assembly of HCR polymers in excess of ˜20,000 bp (>500 HCR hairpins per polymer) in 10 minutes with HCR hairpins at 0.5 μM. HCR initiator (i1) triggers the self-assembly of HCR hairpins (h1 and h2) into amplification polymers. Each HCR hairpin (h1 and h2) at 0.5 μM with initiator i1 at 0.01×. Initiator i1 was omitted from the leakage lane to demonstrate that HCR hairpins are kinetically trapped and do not polymerize in the absence of initiator. Lane 1: GeneRuler 1 kb Plus DNA Ladder pre-stained with SYBR Gold. Lanes 2 and 3: fluorescence from Alexa647-labeled HCR hairpins h1 and h2.

### Example 7—Measurement of HCR Polymer Length Using Fluorophore-Labeled HCR Hairpins in the Context of an Amplified HCR Lateral Flow Assay

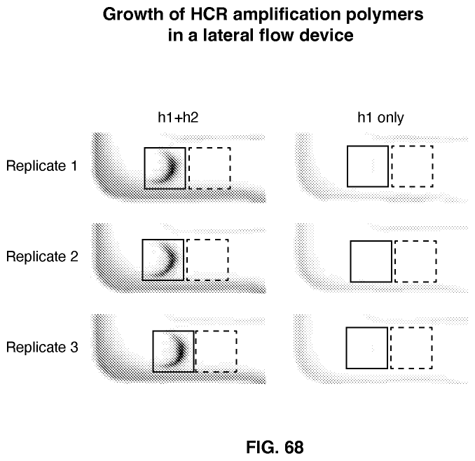

To quantify HCR polymer length in the context of an HCR lateral flow assay, FIG. 68 compares two types of experiments for detection of SARS-CoV-2 nucleocapsid protein N: amplified assays (using both HCR hairpins h1 and h2, so that HCR polymerization can proceed) vs unamplified assays (using only HCR hairpin h1, permitting only a single h1 binding event with no polymerization due to the absence of h2; this situation emulates the unamplified signal of a conventional lateral flow assay where each detected target generates one detectable signal). HCR hairpins h1 and h2 were labeled with Alexa647. To avoid interference with the fluorescent hairpin signal, the CB-labeled anti-DIG readout antibody was omitted from the Channel 3 conjugate pad solution. Gamma-irradiated SARS-CoV-2 virus was spiked into a mixture of human saliva and extraction buffer at 20,000 copies/μL for all tests. After 1 hour, the test strip was removed from the folding card device, and fluorescence from the Alexa647-labeled hairpins and amplification polymers was imaged with a fluorescent scanner. N=3 replicate assays for each condition. The depicted signal boxes (solid boundary) and background boxes (dashed boundary) were used to calculate background-subtracted signal for each type of experiment. The average HCR polymer length, calculated as ratio of the background-subtracted amplified signal to background-subtracted unamplified signal, is 41±8 (mean±estimated standard error of the mean via uncertainty propagation for N=3 replicate assays for each experiment type).

### Example 8—Quantification of HCR Amplification Gain within a Lateral Flow Assay for Detection of SARS-CoV-2 Nucleocapsid Protein (N)

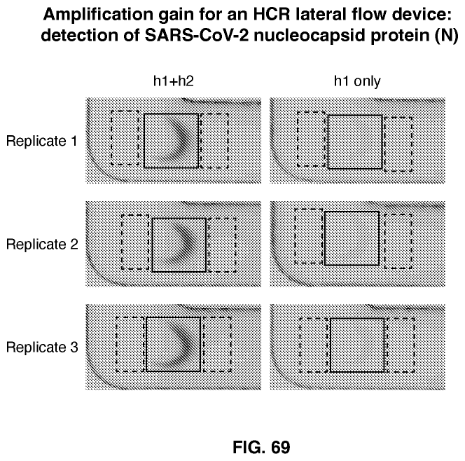

To quantify HCR amplification gain in the context of an HCR lateral flow assay, FIG. 69 compares two types of experiments for detection of SARS-CoV-2 nucleocapsid protein N: amplified assays (using both HCR hairpins h1 and h2, so that HCR polymerization can proceed) vs unamplified assays (using HCR hairpin h1 only, permitting only a single h1 binding event with no polymerization due to the absence of h2; this situation emulates the unamplified signal of a conventional lateral flow assay where each detected target generates one detectable signal). Gamma-irradiated SARS-CoV-2 virus was spiked into a mixture of human saliva and extraction buffer at 5000 copies/μL for all tests. N=3 replicate assays for each condition. The depicted signal boxes (solid boundary) and background boxes (dashed boundary) were used to calculate back-grounded subtracted signal for each type of experiment. The amplification gain, calculated as the ratio of the background-subtracted amplified signal to background-subtracted unamplified signal, is 13.7±0.8 (mean±estimated standard error of the mean via uncertainty propagation for N=3 replicate assays for each experiment type).

### Example 9—Quantification of HCR Amplification Gain within a Lateral Flow Assay for Detection of the SARS-CoV-2 RNA Genome

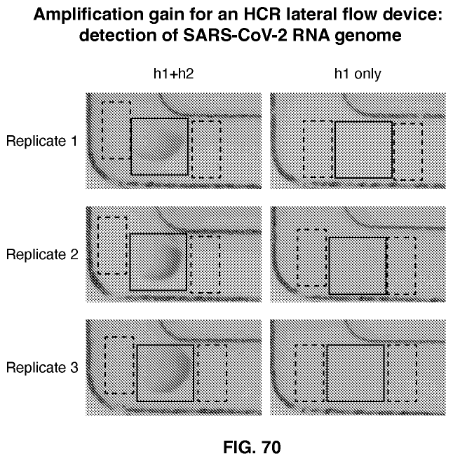

To quantify HCR amplification gain in the context of an HCR lateral flow assay, FIG. 70 compares two types of experiment for detection of the SARS-CoV-2 RNA genome: amplified assays (using both HCR hairpins h1 and h2, so that HCR polymerization can proceed) vs unamplified assays (using HCR hairpin h1 only, permitting only a single h1 binding event with no polymerization due to the absence of h2; this situation emulates the unamplified signal of a conventional lateral flow assay where each detected target generates one detectable signal). Gamma-irradiated SARS-CoV-2 virus was spiked into a mixture of human saliva and extraction buffer at 5000 copies/μL for all tests. N=3 replicate assays for each condition. The depicted signal boxes (solid boundary) and background boxes (dashed boundary) were used to calculate back-grounded subtracted signal for each type of experiment. The amplification gain, calculated as the ratio of the background-subtracted amplified signal to background-subtracted unamplified signal, is 10±3 (mean±estimated standard error of the mean via uncertainty propagation for N=3 replicate assays for each experiment type).

### Example 10—Amplified HCR Lateral Flow Assays for Detection of the Zika RNA Genome or the MERS RNA Genome

FIGS. 11A and 12A display an amplified HCR lateral flow assay for detection of a target RNA. To detect the Zika RNA genome, the target RNA was detected by a DNA signal probe set comprising 86 probe units, each comprising two fractional-initiator probes that bind to adjacent subsequences along the ˜11,000 nt target, avoiding subsequences shared by other targets. To detect the MERS RNA genome, the target RNA was detected by a DNA signal probe set comprising 206 probe units, each comprising two fractional-initiator probes that bind to adjacent subsequences along the ˜30,000 nt target, avoiding subsequences shared by other targets. Using fractional-initiator probes, any individual probe that bound non-specifically in the test region did not trigger HCR, but specific hybridization of the pair of probes within a probe unit to adjacent cognate binding sites along the target RNA colocalized a full HCR initiator capable of triggering HCR signal amplification.

By hybridizing to the RNA target, the DNA signal probes created a DNA:RNA duplex at each target binding site. The probe-decorated target was captured in the test region by an immobilized anti-DNA:RNA capture antibody that bound to DNA:RNA duplexes. While the capture antibody bound DNA:RNA duplexes independent of sequence, any captured RNA that did not include specifically bind fractional-initiator DNA probe pairs did not trigger HCR, and hence did not contribute to background. After immobilization of the probe-decorated target in the test region, colocalized full HCR initiators triggered the self-assembly of DIG-labeled HCR hairpins into tethered HCR amplification polymers decorated with DIG, which were in turn detected by CB-labeled anti-DIG readout antibodies (DIG: digoxigenin, CB: carbon black).

Reagents were automatically delivered to the test region in three successive stages using a 3-channel nitrocellulose membrane (FIG. 32A). The DNA signal probe set bound the RNA target and traveled via the shortest nitrocellulose channel (Channel 1) to reach the test region first, where the probe-decorated target was captured by pre-immobilized anti-DNA:RNA capture antibodies. DIG-labeled HCR hairpins traveled via a channel of intermediate length (Channel 2) and reached the test region next, where colocalized full initiators formed by specifically bound fractional-initiator probe pairs triggered growth of tethered HCR amplification polymers decorated with DIG. CB-labeled anti-DIG reporter antibodies traveled via the longest channel (Channel 3) and arrived in the test region last, where they decorated the HCR amplification polymers with CB to generate an amplified colored signal in the test region (DIG: digoxigenin, CB: carbon black).

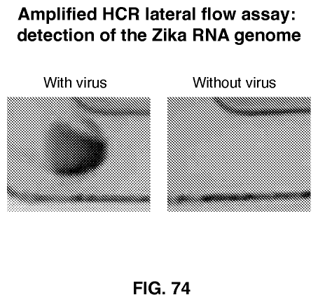

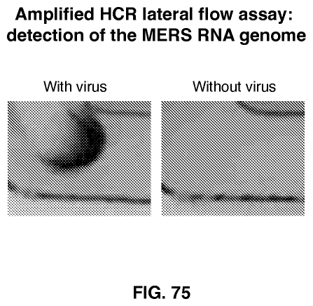

Heat denaturation removed the viral envelope and any nucleocapsid (N) proteins decorating the RNA genome, as well as disrupted native secondary structure in the single-stranded RNA genome prior to detection by the DNA probe set. This RNA test used a half-strip assay format with reagents in solution. To perform a test, the sample was added to extraction buffer containing the DNA signal probe set (Channel 1 reagents), heated to 65° C. for 15 minutes, and then loaded into a well (Channel 1 well) on a 96-well plate proximal to wells containing the DIG-labeled HCR hairpins (Channel 2 well) and CB-labeled anti-DIG reporter antibodies (Channel 3 well; see FIG. 6). To start the lateral flow assay, the ends of the three nitrocellulose channels were simultaneously submerged into the three wells, leading to automated successive delivery of the Channel 1, 2, and 3 reagents to the test region without user interaction. After 90 minutes, the result was read as either a positive result (black signal) or a negative result (no signal) in the test region with the naked eye (see FIG. 74 for detection of the Zika RNA genome and see FIG. 75 for detection of the MERS RNA genome).

FIGS. 11A and 12A display an amplified HCR lateral flow assay for detection of a target RNA. A signal probe set comprising multiple probe units each comprising a pair of fractional-initiator probes hybridized to cognate binding sites on the viral RNA genome and colocalized full HCR initiators; the resulting DNA/RNA duplexes were captured in the test region by anti-DNA/RNA capture antibodies; colocalized full HCR initiators triggered self-assembly of DIG-labeled HCR hairpins to form tethered HCR amplification polymers decorated with DIG, which were subsequently bound by CB-labeled anti-DIG reporter antibodies, generating an amplified signal in the test region. (DIG: digoxigenin; CB: carbon black). FIG. 62 displays automated delivery of reagents to the test region from Channels 1, 2, and 3 in succession using a 3-channel nitrocellulose membrane. FIG. 74 shows test results with or without gamma-irradiated Zika virus spiked into extraction buffer at 110,000 copies/μL. FIG. 75 shows test results with or without MERS gamma-irradiated virus spiked into extraction buffer at 27,000 copies/μL.

The nitrocellulose membrane and wicking pad were overlapped on an adherent backing material and cut into a 3-channel geometry with a laser cutter. The nitrocellulose membrane was spotted with anti-DNA/RNA capture antibody in the test region and dried. Gamma-irradiated virus was mixed with extraction buffer and the DNA signal probe set, heated on a heat block at 65° C. for 15 min, and loaded into a well (Channel 1) proximal to wells containing reagents for Channel 2 (DIG-labeled HCR hairpins h1 and h2) and Channel 3 (CB-labeled anti-DIG reporter antibody). The ends of the three nitrocellulose channels were simultaneously immersed into the three wells to start the test. After 90 minutes, the test region was photographed.

### Example 11—Amplified HCR Lateral Flow Assays for Detection of H3N2 (Influenza A) Nucleocapsid Protein (NP) or H5N1 (Bird Flu) Hemagglutinin Protein

FIG. 10B displays an amplified HCR lateral flow assay for detection of a target protein. To detect the H3N2 (Influenza A) nucleocapsid protein (NP), the target protein bound in a sandwich between an HCR initiator-labeled anti-target signal antibody that bound a first target epitope and an anti-target capture antibody that bound a second target epitope. To detect the H5N1 (bird flu) hemagglutinin protein, the target protein bound in a sandwich between an HCR initiator-labeled anti-target signal antibody that bound a first target epitope and an anti-target capture antibody that bound a second target epitope.

The anti-target capture antibody was biotinylated and was itself captured in the test region by immobilized polystreptavidin R (PR). After the antibody/target sandwich was immobilized in the test region, the HCR initiators labeling the anti-target signal antibody triggered the self-assembly of DIG-labeled HCR hairpins into tethered HCR amplification polymers (digoxigenin; DIG). The DIG-decorated HCR amplification polymers immobilized in the test region were then bound by CB-labeled anti-DIG readout antibodies (carbon black; CB).

Reagents were automatically delivered to the test region in three successive stages using a three-channel nitrocellulose membrane in which channels of different lengths lead into a unified channel before reaching the test region (see FIG. 31A). The anti-target signal and capture antibodies bound the target protein and traveled via the shortest nitrocellulose channel (Channel 1) to reach the test region first, where the antibody/target sandwich was immobilized via binding of biotinylated anti-target capture antibodies to immobilized polystreptavidin R (PR). DIG-labeled HCR hairpins traveled via a channel of intermediate length (Channel 2) and reached the test region next, where initiators on the anti-target signal antibodies triggered growth of tethered DIG-labeled HCR amplification polymers. CB-labeled anti-DIG readout antibodies traveled via the longest channel (Channel 3) and arrived in the test region last, where they decorated the HCR amplification polymers to generate an amplified colored signal in the test region.

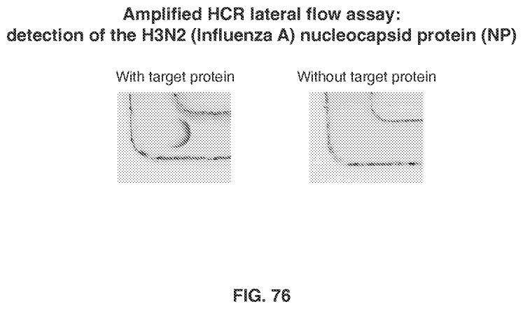

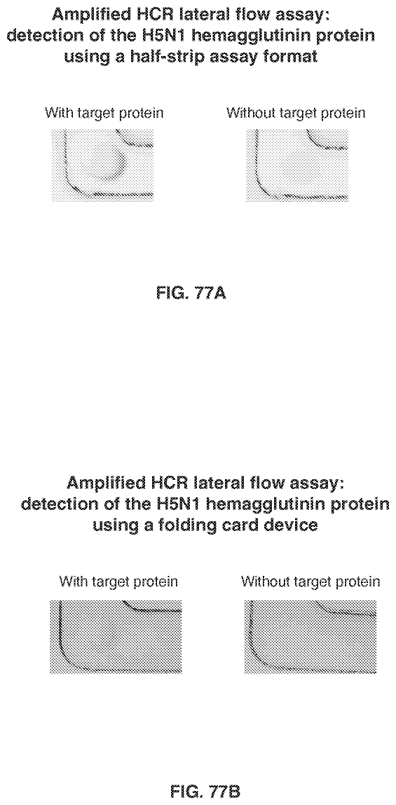

In some embodiments, the assay used a half-strip assay format with reagents in solution. The nitrocellulose membrane was spotted with polystreptavidin R (PR) in the test region and dried. To perform a test, the sample was added to into a well containing extraction buffer, anti-target signal antibody, and anti-target capture antibody (Channel 1 well) on a 96-well plate proximal to wells containing the DIG-labeled HCR hairpins (Channel 2 well) and CB-labeled anti-DIG reporter antibodies (Channel 3 well; see FIG. 6). To start the lateral flow assay, the ends of the three nitrocellulose channels were simultaneously submerged into the three wells, leading to automated successive delivery of the Channel 1, 2, and 3 reagents to the test region without user interaction. After 90 minutes, the test region was photographed. FIG. 76 demonstrates detection of the H3N2 nucleocapsid protein (NP) using tests with or without NP spiked into extraction buffer at 50 ng/mL. FIG. 77A demonstrates detection of the H5N1 hemagglutinin protein using tests with or without hemagglutinin protein spiked into extraction buffer at 50 ng/mL.

In some embodiments, the protein test employed a folding card device (see FIG. 5), with the reagents for each channel dried onto separate conjugate pads which were rehydrated simultaneously when the sample was added to the sample pad. Upon rehydration, successive delivery of the reagents to the test region occurred automatically without user interaction, as draining of the first conjugate pad freed the unified channel for draining of the second conjugate pad, which in turn freed the unified channel for draining of the third conjugate pad. The left page of the device contained the 3-channel nitrocellulose membrane and wicking pad. The right page of the device contained 3 conjugate pads (containing dried reagents for Channels 1, 2, and 3) and a sample pad. To perform the test, the sample was added to the sample pad, the device was closed, and the test region was photographed after 60 minutes. FIG. 77B demonstrates detection of the H5N1 hemagglutinin protein using tests with or without the target protein spiked into extraction buffer at 50 ng/mL.

### Example 12—Amplified HCR Lateral Flow Assay for Multiplex Detection of SARS-CoV-2 Nucleocapsid Protein (N) and H3N2 (Influenza A) Nucleocapsid Protein (NP)

FIG. 41D displays an amplified HCR lateral flow assay for 2-plex detection of two target proteins. Target 1 was SARS-CoV-2 nucleocapsid protein (N). The target protein N bound in a sandwich between an HCR initiator-labeled anti-N 1^{st} signal antibody that bound a first target epitope and an anti-N 1^{st} capture antibody that bound a second target epitope. The anti-N 1^{st} capture antibody was conjugated to a 1^{st} immobilization ligand (FITC) and was itself captured in a 1st test region by an immobilized 1^{st} immobilization receptor (anti-FITC antibody). Target 2 was H3N2 (Influenza A) nucleocapsid protein (NP). The target protein NP bound in a sandwich between an HCR initiator-labeled anti-NP 2^{nd} signal antibody that bound a first target epitope and an anti-NP 2^{nd} capture antibody that bound a second target epitope. The anti-NP 2^{nd} capture antibody was conjugated to a 2^{nd} immobilization ligand (biotin) and was itself captured in a 2^{nd} test region by an immobilized 2^{nd} immobilization receptor (polystreptavidin; PR). After the 1^{st} antibody/target sandwich was immobilized in the 1^{st} test region and the 2^{nd} antibody/target sandwich was immobilized in the 2^{nd} test region, the HCR initiators labeling the anti-N 1^{st} signal antibody and the HCR initiators labeling the anti-NP 2^{nd} signal antibody triggered the self-assembly of DIG-labeled HCR hairpins into tethered HCR amplification polymers (digoxigenin; DIG). The DIG-decorated HCR amplification polymers immobilized in the 1^{st} test region and/or 2^{nd} test region were then bound by CB-labeled anti-DIG readout antibodies (carbon black; CB).

Reagents were automatically delivered to the 1^{st} test region and 2^{nd} test region in three successive stages using a three-channel nitrocellulose membrane in which channels of different lengths lead into a unified channel before reaching the test region (see FIG. 42D). The anti-N 1^{st} signal antibody and anti-N 1^{st} capture antibody bound the target protein N and the anti-NP 2^{nd} signal antibody and anti-NP 2^{nd} capture antibody bound the target protein NP and traveled via the shortest nitrocellulose channel (Channel 1) to reach the test regions. The 1^{st} antibody/target sandwich was immobilized via binding of FITC-conjugated anti-target 1^{st} capture antibodies to immobilized anti-FITC antibody in the 1^{st} test region. The 2^{nd} antibody/target sandwich was immobilized via binding of biotinylated anti-target 2^{nd} capture antibodies to immobilized polystreptavidin R (PR) in the 2^{nd} test region. DIG-labeled HCR hairpins traveled via a channel of intermediate length (Channel 2) and reach the test regions next, where initiators on the anti-target signal antibodies triggered growth of tethered DIG-labeled HCR amplification polymers. CB-labeled anti-DIG readout antibodies traveled via the longest channel (Channel 3) and arrived in the test region last, where they decorated the HCR amplification polymers to generate an amplified colored signal in the test region.

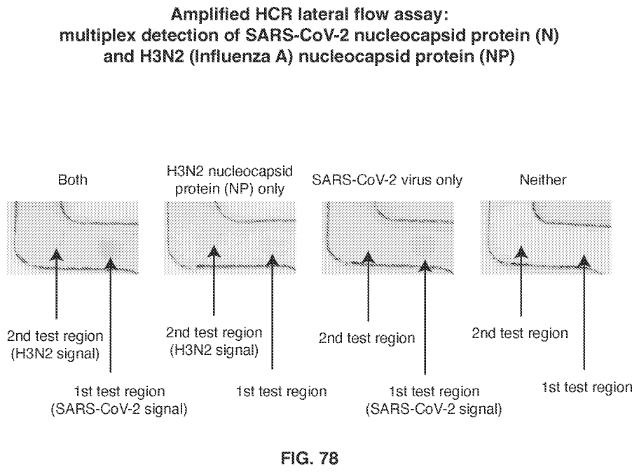

This multiplexed protein test used a half-strip assay format with reagents in solution. The nitrocellulose membrane was spotted with anti-FITC antibody in the 1^{st} test region and polystreptavidin R (PR) in the 2^{nd} test region and dried. To perform a test, gamma-irradiated SARS-CoV-2 virus and H3N2 nucleocapsid protein (NP) were added to a well containing extraction buffer, anti-N 1^{st} signal antibody, anti-N 1^{st} capture antibody, anti-NP 2^{nd} signal antibody, and anti-NP 2^{nd} capture antibody (Channel 1 well) on a 96-well plate proximal to wells containing the DIG-labeled HCR hairpins (Channel 2 well) and CB-labeled anti-DIG reporter antibodies (Channel 3 well; see FIG. 6). To start the lateral flow assay, the ends of the three nitrocellulose channels are simultaneously submerged into the three wells, leading to automated successive delivery of the Channel 1, 2, and 3 reagents to the test region without user interaction. After 90 minutes, the test region was photographed. FIG. 78 demonstrates detection of the SARS-CoV-2 nucleocapsid protein (N) and H3N2 nucleocapsid protein (NP) using 2-plex tests with samples containing either gamma-irradiated SARS-CoV-2 virus, H3N2 nucleocapsid protein (NP), both, or neither (spiked into extraction buffer at 1000 copies/μL for gamma-irradiated SARS-CoV-2 virus and at 50 ng/mL for H3N2 nucleocapsid protein (NP)).

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). It is to be understood that both the general description and the detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

In some embodiments, any one or more of the compositions or steps provided in any of the figures provided herein can be combined with any of the other compositions or steps provided herein. As used herein, a generic reference to a set of figures (for example, FIG. 17) denotes all of the different figures contained within that number (for example, FIGS. 17A-17F), each combined together, one or more of them, or each in the alternative, unless otherwise denoted.

In some embodiments, any one or more of the optional elements of any one or more of the figures herein can be combined with any one or more of the other optional elements of any one or more of the figures herein.

### REFERENCES

(1) Tiplady, S. Lateral Flow and Consumer Diagnostics. In The Immunoassay Handbook; Elsevier, 2013; pp 533-536.
(2) Mahmoudinobar, F.; Britton, D.; Montclaire, J. K. Protein-Based Lateral Flow Assays for COVID-19 Detection. Protein Eng. Des. Sel. 2021, 34, 1-10.
(3) Larsen, J.; Buchanan, P.; Johnson, S.; Godbert, S.; Zinaman, M. Human Chorionic Gonadotropin as a Measure of Pregnancy Duration. Int. J. Gynecol. Obstet. 2013, 123 (3), 189-195.
(4) Gnoth, C.; Johnson, S. Strips of hope: Accuracy of home pregnancy tests and new developments. Geburtshilfe Frauenheilkd. 2014, 74 (07), 661-669.
(5) Khelifa, L.; Hu, Y.; Jiang, N.; Yetisen, A. K. Lateral Flow Assays for Hormone Detection. Lab. Chip 2022, 22 (13), 2451-2475.
(6) To, K. K.-W.; Tsang, O. T.-Y.; Yip, C. C.-Y.; Chan, K.-H.; Wu, T.-C.; Chan, J. M.-C.; Leung, W.-S.; Chik, T. S.-H.; Choi, C. Y.-C.; Kandamby, D. H.; Lung, D. C.; Tam, A. R.; Poon, R. W.-S.; Fung, A. Y.-F.; Hung, I. F.-N.; Cheng, V. C.-C.; Chan, J. F.-W.; Yuen, K.-Y. Consistent Detection of 2019 Novel Coronavirus in Saliva. Clin. Infect. Dis., 2020, ciaal49.
(7) To, K. K.-W.; Tsang, O. T.-Y.; Leung, W.-S.; Tam, A. R.; Wu, T.-C.; Lung, D. C.; Yip, C. C.-Y.; Cai, J.-P.; Chan, J. M.-C.; Chik, T. S.-H.; Lau, D. P.-L.; Choi, C. Y.-C.; Chen, L.-L.; Chan, W.-M.; Chan, K.-H.; Ip, J. D.; Ng, A. C.-K.; Poon, R. W.-S.; Luo, C.-T.; Cheng, V. C.-C.; Chan, J. F.-W.; Hung, I. F.-N.; Chen, Z.; Chen, H.; Yuen, K.-Y. Temporal Profiles of Viral Load in Posterior Oropharyngeal Saliva Samples and Serum Antibody Responses during Infection by SARS-CoV-2: An Observational Cohort Study. Lancet Infect. Dis. 2020, 20, 565-574.
(8) Corman, V. M.; Landt, O.; Kaiser, M.; Molenkamp, R.; Meijer, A.; Chu, D. K. W.; Bleicker, T.; Brunink, S.; Schneider, J.; Schmidt, M. L.; Mulders, D. G. J. C.; Haagmans, B. L.; van der Veer, B.; van den Brink, S.; Wijsman, L.; Goderski, G.; Romette, J.-L.; Ellis, J.; Zambon, M.; Peiris, M.; Goossens, H.; Reusken, C.; Koopmans, M. P. G.; Drosten, C. Detection of 2019 Novel Coronavirus (2019-NCoV) by Real-Time RT-PCR. Eurosurveillance 2020, 25 (3).
(9) Chan, J. F.-W.; Yip, C. C.-Y.; To, K. K.-W.; Tang, T. H.-C.; Wong, S. C.-Y.; Leung, K.-H.; Fung, A. Y.-F.; Ng, A. C.-K.; Zou, Z.; Tsoi, H.-W.; Choi, G. K.-Y.; Tam, A. R.; Cheng, V. C.-C.; Chan, K.-H.; Tsang, O. T.-Y.; Yuen, K.-Y. Improved Molecular Diagnosis of COVID-19 by the Novel, Highly Sensitive and Specific COVID-19-RdRp/Hel Real-Time Reverse Transcription-Polymerase Chain Reaction Assay Validated \emphin Vitro and with Clinical Specimens. J Clin Microbiol, 2020, JCM.00310-20, jcm; JCM.00310-20v1.
(10) Caruana, G.; Croxatto, A.; Kampouri, E.; Kritikos, A.; Opota, O.; Foerster, M.; Brouillet, R.; Senn, L.; Lienhard, R.; Egli, A.; Pantaleo, G.; Carron, P.-N.; Greub, G. Implementing SARS-CoV-2 Rapid Antigen Testing in the Emergency Ward of a Swiss University Hospital: The INCREASE Study. Microorganisms 2021, 9 (4), 798.
(11) Wölfl-Duchek, M.; Bergmann, F.; Jorda, A.; Weber, M.; Müller, M.; Seitz, T.; Zoufaly, A.; Strassl, R.; Zeitlinger, M.; Herkner, H.; Schnidar, H.; Anderle, K.; Derhaschnig, U. Sensitivity and Specificity of SARS-CoV-2 Rapid Antigen Detection Tests Using Oral, Anterior Nasal, and Nasopharyngeal Swabs: A Diagnostic Accuracy Study. Microbiol. Spectr. 2022, 10 (1), e02029-21.
(12) Dinnes, J.; Sharma, P.; Berhane, S.; van Wyk, S. S.; Nyaaba, N.; Domen, J.; Taylor, M.; Cunningham, J.; Davenport, C.; Dittrich, S.; Emperador, D.; Hooft, L.; Leeflang, M. M. G.; McInnes, M. D. F.; Spijker, R.; Verbakel, J. Y.; Takwoingi, Y.; Taylor-Phillips, S.; Van den Bruel, A.; Deeks, J. J.; Cochraine COVID-19 Diagnostics Test Accuracy Group. Rapid, Point-of-care Antigen Tests for Diagnosis of SARS-CoV-2 Infection. Cochrane Database Syst. Rev. 2022, 7.
(13) Tufekci, Z. An Even Deadlier Pandemic Could Soon Be Here. New York Times. Feb. 3, 2023.
(14) Fu, E.; Liang, T.; Spicar-Mihalic, P.; Houghtaling, J.; Ramachandran, S.; Yager, P. Two-Dimensional Paper Network Format That Enables Simple Multistep Assays for Use in Low-Resource Settings in the Context of Malaria Antigen Detection. Anal. Chem. 2012, 84 (10), 4574-4579.
(15) Ramachandran, S.; Fu, E.; Lutz, B.; Yager, P. Long-Term Dry Storage of an Enzyme-Based Reagent System for ELISA in Point-of-Care Devices. The Analyst 2014, 139 (6), 1456-1462.
(16) Zhang, C.; Zheng, T.; Wang, H.; Chen, W.; Huang, X.; Liang, J.; Qiu, L.; Han, D.; Tan, W. Rapid One-Pot Detection of SARS-CoV-2 Based on a Lateral Flow Assay in Clinical Samples. Anal. Chem. 2021, 93 (7), 3325-3330.
(17) Chen, X.; Zhou, Q.; Li, S.; Yan, H.; Chang, B.; Wang, Y.; Dong, S. Rapid and Visual Detection of SARS-CoV-2 Using Multiplex Reverse Transcription Loop-Mediated Isothermal Amplification Linked with Gold Nanoparticle-Based Lateral Flow Biosensor. Front. Cell. Infect. Microbiol. 2021, 11, 581239.
(18) Xiong, D.; Dai, W.; Gong, J.; Li, G.; Liu, N.; Wu, W.; Pan, J.; Chen, C.; Jiao, Y.; Deng, H.; Ye, J.; Zhang, X.; Huang, H.; Li, Q.; Xue, L.; Zhang, X.; Tang, G. Rapid Detection of SARS-CoV-2 with CRISPR-Cas12a. PLoS Biol. 2020, 18 (12), e3000978.
(19) Broughton, J. P.; Deng, X.; Yu, G.; Fasching, C. L.; Servellita, V.; Singh, J.; Miao, X.; Streithorst, J. A.; Granados, A.; Sotomayor-Gonzalez, A.; Zorn, K.; Gopez, A.; Hsu, E.; Gu, W.; Miller, S.; Pan, C.-Y.; Guevara, H.; Wadford, D. A.; Chen, J. S.; Chiu, C. Y. CRISPR-Cas12-Based Detection of SARS-CoV-2. Nat. Biotechnol. 2020, 38 (7), 870-874.
(20) Dirks, R. M.; Pierce, N. A. Triggered Amplification by Hybridization Chain Reaction. Proc. Natl. Acad. Sci. U.S.A 2004, 101 (43), 15275-15278.
(21) Choi, H. M. T.; Chang, J. Y.; Trinh, L. A.; Padilla, J. E.; Fraser, S. E.; Pierce, N. A. Programmable in Situ Amplification for Multiplexed Imaging of MRNA Expression. Nat. Biotechnol. 2010, 28 (11), 1208-1212.
(22) Choi, H. M. T.; Beck, V. A.; Pierce, N. A. Next-Generation in Situ Hybridization Chain Reaction: Higher Gain, Lower Cost, Greater Durability. ACS Nano 2014, 8 (5), 4284-4294.
(23) Choi, H. M. T.; Schwarzkopf, M.; Fornace, M. E.; Acharya, A.; Artavanis, G.; Stegmaier, J.; Cunha, A.; Pierce, N. A. Third-Generation in Situ Hybridization Chain Reaction: Multiplexed, Quantitative, Sensitive, Versatile, Robust. Development 2018, 145, dev165753.
(24) Schwarzkopf, M.; Liu, M. C.; Schulte, S. J.; Ives, R.; Husain, N.; Choi, H. M. T.; Pierce, N. A. Hybridization Chain Reaction Enables a Unified Approach to Multiplexed, Quantitative, High-Resolution Immunohistochemistry and in Situ Hybridization. Development 2021, 148 (22), dev199847.
(25) Choi, H. M. T.; Calvert, C. R.; Husain, N.; Huss, D.; Barsi, J. C.; Deverman, B. E.; Hunter, R. C.; Kato, M.; Lee, S. M.; Abelin, A. C. T.; Rosenthal, A. Z.; Akbari, O. S.; Li, Y.; Hay, B. A.; Sternberg, P. W.; Patterson, P. H.; Davidson, E. H.; Mazmanian, S. K.; Prober, D. A.; van de Rijn, M.; Leadbetter, J. R.; Newman, D. K.; Readhead, C.; Bronner, M. E.; Wold, B.; Lansford, R.; Sauka-Spengler, T.; Fraser, S. E.; Pierce, N. A. Mapping a Multiplexed Zoo of MRNA Expression. Development 2016, 143, 3632-3637.
(26) Bar-On, Y. M.; Flamholz, A.; Phillips, R.; Milo, R. SARS-CoV-2 (COVID-19) by the Numbers. eLife 2020, 9, e57309.
(27) Hutchinson, E. C.; Charles, P. D.; Hester, S. S.; Thomas, B.; Trudgian, D.; Martinez-Alonso, M.; Fodor, E. Conserved and Host-Specific Features of Influenza Virion Architecture. Nat. Commun. 2014, 5 (1), 4816.
(28) Raoult, D.; Zumla, A.; Locatelli, F.; Ippolito, G.; Kroemer, G. Coronavirus Infections: Epidemiological, Clinical and Immunological Features and Hypotheses. Cell Stress 2020, 4 (4), 66-75.

1. A hybridization chain reaction (HCR) lateral flow device for amplified detection of a target in a sample, wherein the device comprises one or more membranes, a test region on the one or more membranes, one or more probe sets each comprising one or more probes, at least one probe comprising an amplification domain that is configured to trigger a chain reaction in which HCR hairpins self-assemble to form a tethered HCR amplification polymer, and an HCR amplifier comprising two or more HCR hairpins wherein one or more of the HCR hairpins is labeled with one or more reporters, wherein the device is configured such that the sample and one or more reagents flow through the one or more membranes across the test region, and wherein the one or more probe sets and the HCR amplifier are configured to produce tethered reporter-decorated HCR amplification polymers to directly or indirectly generate an amplified signal in the test region.

2. The device of claim 1, wherein the signal is generated if the target is present in the sample.

3. The device of claim 2 wherein the one or more probe sets comprise a capture probe set comprising one or more capture probes, wherein a capture probe comprises a target-binding domain configured to bind directly or indirectly to the target and an immobilization domain configured to bind directly or indirectly to the test region.

4. The device of claim 2, wherein the one or more probe sets comprise a signal probe set comprising one or more signal probes, wherein a signal probe comprises a target-binding domain configured to bind directly or indirectly to the target and an amplification domain that is configured to directly or indirectly trigger a chain reaction in which HCR hairpins self-assemble to form a tethered HCR amplification polymer decorated with reporters.

5. The device of claim 4, wherein the signal probe set comprises one or more initiator-labeled probes, wherein an initiator-labeled probe comprises a target-binding domain and an amplification domain comprising one or more HCR initiators.

6. The device of claim 4, wherein the signal probe set comprises two or more fractional-initiator probes.

7-11. (canceled)

12. The device of claim 1, wherein one or more of the probes comprising the probe set are immobilized on at least one of the one or more membranes.

13. The device of claim 1, wherein the one or more probe sets comprise one or more readout probes, wherein a readout probe comprises a reporter-binding domain configured to bind directly or indirectly to a reporter on an HCR amplification polymer, and further comprises one or more auxiliary reporters.

14. The device of claim 13, wherein the readout probe comprising one or more auxiliary reporters binds the reporters decorating the tethered HCR amplification polymers that are immobilized in the test region, directly or indirectly generating an amplified signal in the test region.

15. The device of claim 14, wherein the reporters or auxiliary reporters decorating an HCR amplification polymer directly or indirectly mediate catalytic reporter deposition (CARD) to generate an amplified signal in the vicinity of the HCR amplification polymer within the test region.

16-26. (canceled)

27. The device of claim 1, wherein reagents automatically arrive in the test region in two or more successive stages.

28. The device of claim 27, wherein the device comprises a membrane with two or more channels configured to automatically provide reagents to the test region.

29. The device of claim 28, wherein the two or more channels have different lengths that lead into a unified channel before reaching the test region.

30. The device of claim 28, wherein the device comprises a separate conjugate pad for each of the two or more channels, each conjugate pad comprising dried down reagents, wherein each conjugate pad is fluidly connected to a single channel.

31. The device of claim 1, wherein the device takes the form of a folding card comprising a sample pad, one or more conjugate pads, a membrane comprising two or more channels, and a wicking pad.

32-42. (canceled)

43. A method for amplified detection of a target in a sample, wherein a user: 1) adds the sample to a hybridization chain reaction (HCR) lateral flow device comprising one or more membranes, a test region on the one or more membranes, one or more probe sets each comprising one or more probes, at least one probe comprising an amplification domain that is configured to trigger a chain reaction in which HCR hairpins self-assemble to form a tethered HCR amplification polymer, an HCR amplifier comprising two or more HCR hairpins wherein one or more of the HCR hairpins is labeled with one or more reporters, 2) waits for a prescribed time while the sample and reagents flow through the one or more membranes across the test region to produce a tethered reporter-decorated HCR amplification polymer that produces an amplified signal in the test region, 3) and reads out the rest result by examining the test region for a signal.

44. The method of claim 43, further comprising prepping the sample by mixing the sample with a prep buffer prior to adding the sample to the HCR lateral flow device.

45. The method of claim 43, wherein the signal is generated if the target is present in the sample.

46. The method of claim 45, wherein the device comprises a capture probe set comprising one or more capture probes, and a signal probe set comprising one or more signal probes, wherein if a target is present in the sample, the target binds one or more capture probes and one or more signal probes to form a probe/target complex, wherein the one or more capture probes immobilize the probe/target complex in the test region, and wherein the amplification domains on the one or more signal probes trigger the self-assembly of reporter-decorated HCR hairpins into tethered reporter-decorated HCR amplification polymers to thereby directly or indirectly generate an amplified signal in the test region.

47-56. (canceled)

57. The method of claim 43, wherein the device takes the form of a folding card, wherein closing the folding card starts the test.

58. The method of claim 43, wherein one or more of the probes comprising the one or more probe sets are immobilized on at least one of the one or more membranes.

59. The method of claim 45, wherein the device comprises a capture probe set comprising one or more capture probes, wherein if a target is present in the sample, a target-binding domain of the capture probe binds directly or indirectly to the target and an immobilization domain of the capture probe binds directly or indirectly to the test region.

60. The method of claim 45, wherein the device comprises a signal probe set comprising one or more signal probes, wherein if a target is present in the sample, a target-binding domain of one or more signal probes binds directly or indirectly to the target and one or more amplification domains of the one or more signal probes directly or indirectly triggers a chain reaction in which HCR hairpins self-assemble to form a tethered HCR amplification polymer decorated with reporters.

61. The method of claim 60, wherein the signal probe set of the device comprises: a) one or more initiator-labeled probes wherein an initiator-labeled probe comprises a target-binding domain and an amplification domain comprising one or more HCR initiators, or b) one or more probe units, each comprising two or more fractional-initiator probes, wherein a fractional-initiator probe comprises a target-binding domain and an amplification domain comprising a fractional initiator.

62-64. (canceled)

65. The method of claim 45, wherein the device comprises a readout probe set comprising one or more readout probes, wherein if a target is present in the sample, a reporter-binding domain of the readout probe binds directly or indirectly to a reporter on an HCR amplification polymer, and further comprises one or more auxiliary reporters.

66. The method of claim 65 wherein the one or more readout probes of the device comprise one or more auxiliary reporters and bind the reporters decorating the tethered HCR amplification polymers that are immobilized in the test region, directly or indirectly generating an amplified signal in the test region.

67. The method of claim 66, wherein the reporters or auxiliary reporters decorating an HCR amplification polymer directly or indirectly mediate catalytic reporter deposition (CARD) to generate an amplified signal in the vicinity of the HCR amplification polymer within the test region.

68-81. (canceled)

82. The method of claim 43, wherein reagents automatically arrive in the test region in two or more successive stages.

83. The method of claim 82, wherein the device comprises a membrane with two or more channels configured to automatically provide reagents to the test region.

84. The method of claim 83, wherein the two or more channels have different lengths that lead into a unified channel before reaching the test region.

85-98. (canceled)
